SEQUENCE LISTING (23 sequences)

SEQ ID NO: 1 - protein, 5262 aa, Artificial Sequence
  GenBank Accession No. AAC51734.1
MDSQNLAGEDKDSQPAADGPAASEDPSATESDLPNPHVGEVSVLSSGSPRLQETPQDCSGGPVRRCALCNCGEPALHGQRELRRFELPFDWPRCPVVSPGGSPGPNEAVLPSEDLSQIGFPEGLTPAHLGEPGGSCWAHHWCAAWSAGVWGQEGPQLCGVDKAIFSGISQRCSHCTRLGASIPCRSPGCPRLYHFPCATASGSFLSMKTLQLLCPEHSEGAAYLEEARCAVCEGPGELCDLFFCTSCGHHYHGACLDTALTARKRAGWQCPECKVCQACRKPGNDSKMLVCETCDKGYHTFCLKPPMEELPAHSWKCKACRVCRACGAGSAELNPNSEWFENYSLCHRCHKAQGGQTIRSVAEQHTPVCSRFSPPEPGDTPTDEPDALYVACQGQPKGGHVTSMQPKEPGPLQCEAKPLGKAGVQLEPQLEAPLNEEMPLLPPPEESPLSPPPEESPTSPPPEASRLSPPPEELPASPLPEALHLSRPLEESPLSPPPEESPLSPPPESSPFSPLEESPLSPPEESPPSPALETPLSPPPEASPLSPPFEESPLSPPPEELPTSPPPEASRLSPPPEESPMSPPPEESPMSPPPEASRLFPPFEESPLSPPPEESPLSPPPEASRLSPPPEDSPMSPPPEESPMSPPPEVSRLSPLPVVSRLSPPPEESPLSPPALSPLGELEYPFGAKGDSDPESPLAAPILETPISPPPEANCTDPEPVPPMILPPSPGSPVGPASPILMEPLPPQCSPLLQHSLVPQNSPPSQCSPPALPLSVPSPLSPIGKVVGVSDEAELHEMETEKVSEPECPALEPSATSPLPSPMGDLSCPAPSPAPALDDFSGLGEDTAPLDGIDAPGSQPEPGQTPGSLASELKGSPVLLDPEELAPVTPMEVYPECKQTAGRGSPCEEQEEPRAPVAPTPPTLIKSDIVNEISNLSQGDASASFPGSEPLLGSPDPEGGGSLSMELGVSTDVSPARDEGSLRLCTDSLPETDDSLLCDAGTAISGGKAEGEKGRRRSSPARSRIKQGRSSSFPGRRRPRGGAHGGRGRGRARLKSTASSIETLVVADIDSSPSKEEEEEDDDTMQNTVVLFSNTDKFVLMQDMCVVCGSFGRGAEGHLLACSQCSQCYHPYCVNSKITKVMLLKGWRCVECIVCEVCGQASDPSRLLLCDDCDISYHTYCLDPPLLTVPKGGWKCKWCVSCMQCGAASPGFHCEWQNSYTHCGPCASLVTCPICHAPYVEEDLLIQCRHCERWMHAGCESLFTEDDVDHAPDEGFDCVSCQPYVVKPVAPVAPPELVPMKVKEPEPQYFRFEGVWLTETGMALLRNLTMSPLHKRRQRRGRLGLPGEAGLEGSEPSDALGPDDKKDGDLDTDELLKGEGGVEHMECEIKLEGPVSPDVEPGKEETEESKKRKRKPYRPGIGGFMVRQRKSHTRTKKGPAAQAEVLSGDGQPDEVIPADLPAEGAVEQSLAEGDEKKKQQRRGRKRSKLEGMFPAYLQEAFFGKELLDLSRKALFAVGVGRPSFGLGTPKAKGDGGSERKELPTSQKGDDGPDIADEESRGLEGKADTPGPEDGGVKASPVPSDPEKPGTPGEGMLSSDLDRISTEELPKMESKDLQQLFKDVLGSEREQHLGCGTPGLEGSRTPLQRPFLQGGLPLGNLPSSSPMDSYPGLCQSPFLDSRERGGFFSPEPGEPDSPWTGSGGTTPSTPTTPTTEGEGDGLSYNQRSLQRWEKDEELGQLSTISPVLYANINFPNLKQDYPDWSSRCKQIMKLWRKVPAADKAPYLQKAKDNRAAHRINKVQKQAESQINKQTKVGDIARKTDRPALHLRIPPQPGALGSPPPAAAPTIFIGSPTTPAGLSTSADGFLKPPAGSVPGPDSPGELFLKLPPQVPAQAPSQDPFGLAPAYPLEPRFPTAPPTYPPYPSPTGAPAQPPMLGASSRPGAGQPGEFHTTPPGTPRHQPSTPDPFLKPRCPSLDNLAVPESPGVGGGKASEPLLSPPPFGESRKALEVKKEELGASSPSYGPPNLGFVDSPSSGTHLGGLELKTPDVFKAPLTPRASQVEPQSPGLGLRPQEPPPAQALAPSPPSHPDIFRPGSYTDPYAQPPLTPRPQPPPPESCCALPPRSLPSDPFSRVPVSPQSQSSSQSPLTPRPLSAEAFCPSPVTPRFQSPDPYSRPPSRPQSRDPFAPLHKPPRPQPPEVAFKAGSLAHTSLGAGGFPAALPAGPAGELHAKVPSGQPPNFVRSPGTGAFVGTPSPMRFTFPQAVGEPSLKPPVPQPGLPPPHGINSHFGPGPTLGKPQSTNYTVATGNFHPSGSPLGPSSGSTGESYGLSPLRPPSVLPPPAPDGSLPYLSHGASQRSGITSPVEKREDPGTGMGSSLATAELPGTQDPGMSGLSQTELEKQRQRQRLRELLIRQQIQRNTLRQEKETAAAAAGAVGPPGSWGAEPSSPAFEQLSRGQTPFAGTQDKSSLVGLPPSKLSGPILGPGSFPSDDRLSRPPPPATPSSMDVNSRQLVGGSQAFYQRAPYPGSLPLQQQQQQLWQQQQATAATSMRFAMSARFPSTPGPELGRQALGSPLAGISTRLPGPGEPVPGPAGPAQFIELRHNVQKGLGPGGTPFPGQGPPQRPRFYPVSEDPHRLAPEGLRGLAVSGLPPQKPSAPPAPELNNSLHPTPHTKGPTLPTGLELVNRPPSSTELGRPNPLALEAGKLPCEDPELDDDFDAHKALEDDEELAHLGLGVDVAKGDDELGTLENLETNDPHLDDLLNGDEFDLLAYTDPELDTGDKKDIFNEHLRLVESANEEAEREALLRGVEPGPLGPEERPPPAADASEPRLASVLPEVKPKVEEGGRHPSPCQFTIATPKVEPAPAANSLGLGLKPGQSMMGSRDTRMGTGPFSSSGHTAEKASFGATGGPPAHLLTPSPLSGPGGSSLLEKFELESGALTLPGGPAASGDELDKMESSLVASELPLLIEDLLEHEKKELQKKQQLSAQLQPAQQQQQQQQQHSLLPAPGPAQAMSLPHEGSSPSLAGSQQQLSLGLAVARQPGLPQPLMPTQPPAHALQQRLAPSMAMVSNQGHMLSGQHGGQAGLVPQQSSQPVLSQKPMGTMPPSMCMKPQQLAMQQQLANSFFPDTDLDKFAAEDIIGPIAKAKMVALKGIKKVMAQGSIGVAPGMNRQQVSLLAQRLSGGPSSDLQNHVAAGSGQERSAGDPSQPRPNPPTFAQGVINEADQRQYEEWLFHTQQLLQMQLKVLEEQIGVHRKSRKALCAKQRTAKKAGREFPEADAEKLKLVTEQQSKIQKQLDQVRKQQKEHTNLMAEYRNKQQQQQQQQQQQQQQHSAVLALSPSQSPRLLTKLPGQLLPGHGLQPPQGPPGGQAGGLRLTPGGMALPGQPGGPFLNTALAQQQQQQHSGGAGSLAGPSGGFFPGNLALRSLGPDSRLLQERQLQLQQQRMQLAQKLQQQQQQQQQQQHLLGQVAIQQQQQQGPGVQTNQALGPKPQGLMPPSSHQGLLVQQLSPQPPQGPQGMLGPAQVAVLQQQHPGALGPQGPHRQVLMTQSRVLSSPQLAQQGQGLMGHRLVTAQQQQQQQQHQQQGSMAGLSHLQQSLMSHSGQPKLSAQPMGSLQQLQQQQQLQQQQQLQQQQQQQLQQQQQLQQQQLQQQQQQQQLQQQQQQQLQQQQQQLQQQQQQQQQQFQQQQQQQQMGLLNQSRTLLSPQQQQQQQVALGPGMPAKPLQHFSSPGALGPTLLLTGKEQNTVDPAVSSEATEGPSTHQGGPLAIGTTPESMATEPGEVKPSLSGDSQLLLVQPQPQPQPSSLQLQPPLRLPGQQQQQVSLLHTAGGGSHGQLGSGSSSEASSVPHLLAQPSVSLGDQPGSMTQNLLGPQQPMLERPMQNNTGPQPPKPGPVLQSGQGLPGVGIMPTVGQLRAQLQGVLAKNPQLRHLSPQQQQQLQALLMQRQLQQSQAVRQTPPYQEPGTQTSPLQGLLGCQPQLGGFPGPQTGPLQELGAGPRPQGPPRLPAPPGALSTGPVLGPVHPTPPPSSPQEPKRPSQLPSPSSQLPTEAQLPPTHPGTPKPQGPTLEPPPGRVSPAAAQLADTLFSKGLGPWDPPDNLAETQKPEQSSLVPGHLDQVNGQVVPEASQLSIKQEPREEPCALGAQSVKREANGEPIGAPGTSNHLLLAGPRSEAGHLLLQKLLRAKNVQLSTGQGSEGLRAEINGHIDSKLAGLEQKLQGTPSNKEDAAARKPLTPKPKRVQKASDRLVSSRKKLRKEDGVRASEALLKQLKQELSLLPLTEPAITANFSLFAPFGSGCPVNGQSQLRGAFGSGALPTGPDYYSQLLTKNNLSNPPTPPSSLPPTPPPSVQQKMVNGVTPSEELGEHPKDAASARDSERALRDTSEVKSLDLLAALPTPPHNQTEDVRMESDEDSDSPDSIVPASSPESILGEEAPRFPHLGSGRWEQEDRALSPVIPLIPRDSIPVFPDTKPYGALGLEVPGKLPVTTWEKGKGSEVSVMLTVSAAADKNLNGVMVAVAELLSMKIPNSYEVLFPESPARGGTEPKKGEAEGPGGKEKGLEGKSPDTGPDWLKQFDAVLAGYTLKRQLDILSLLKQESPAPEPPTQHRYTYNVSNLDVRQLSAPPPEEPSPPPSPLAPSPASPPTEPLVELPTEPLAEPPVPSPLPLASSPESARPKPRARPPEEGEDTRPPRLKKWKGVRWKRLRLLLTIQKGSGRQEDEREVAEFMEQLGTALRPDKVPRDMRRCCFCHEEGDGATDGPARLLNLDLDLWVHLNCALWSTEVYETQGGALMNVEVALHRGLLTKCSLCQRTGATSSCNRMRCPNVYHFGCAIRAKCMFFKDKTMLCPMHKIKGPCEQELSSFAVFRRVYIERDEVKQIASIIQRGERLHMFRVGGLVFHAIGQLLPHQMADFHSATALYPVGYEATRIYWSLRTNNRRCCYRCSIGENNGRPEFVIKVIEQGLEDLVFTDASPQAVWNRIIEPVAAMRKEADMLRLFPEYLKGEELFGLTVHAVLRIAESLPGVESCQNYLFRYGRHPLMELPLMINPTGCARSEPKILTHYKRPHTLNSTSMSKAYQSTFTGETNTPYSKQFVHSKSSQYRRLRTEWKNNVYLARSRIQGLGLYAAKDLEKHTMVIEYIGTIIRNEVANRREKIYEEQNRGIYMFRINNEHVIDATLTGGPARYINHSCAPNCVAEVVTFDKEDKIIIISSRRIPKGEELTYDYQFDFEDDQHEIPCHCGAWNCRKWMN

SEQ ID NO: 2 - DNA, 15789 bp, Artificial Sequence
  GenBank Accession No. AF010403.1
atggacagccagaacctggctggtgaggataaagattcacaaccggcagctgatggacctgcagcctctgaggacccaagtgccactgagtcagacctgcccaacccacatgtgggagaggtctctgtccttagttctgggagtcccaggcttcaggagactcctcaggactgcagtgggggtccggtgcggcgttgtgctctctgtaactgcggggagcccgctctacacgggcagcgggagctacggcgctttgagttgccatttgattggccccggtgtccagtggtgtcccctggggggagcccagggcccaatgaggcagtgctgcccagtgaggacctatcacagattggtttccctgagggccttacacctgcccacctaggagaacctggagggtcctgctgggctcaccattggtgtgctgcatggtcggcaggcgtatgggggcaggagggcccacaactatgtggtgtggacaaggccatcttctcagggatctcacagcgctgctcccactgcaccaggctcggtgcctccatcccttgccgctcacctggatgtccacggctttaccacttcccctgcgcgactgccagcggttccttcctatccatgaaaacactgcagctgctatgcccagagcacagtgagggggctgcatatctggaggaggctcgctgtgcagtgtgtgaggggccaggggagctgtgtgacctgttcttctgtaccagctgtgggcatcactatcacggggcctgcctggacactgctctgactgcccgcaaacgtgctggctggcagtgccctgaatgcaaagtgtgccaagcctgcaggaaacctgggaatgactctaagatgttggtttgcgagacgtgtgacaaaggataccatactttctgcctaaaaccacccatggaggaactgcctgctcactcttggaagtgcaaggcgtgccgggtgtgccgggcctgtggggcgggctcagcagaactgaatcccaactcggagtggtttgagaactactctctctgtcaccgctgtcacaaagcccagggaggtcagactatccgctccgttgctgagcagcataccccggtgtgtagcagattttcacccccagagcctggcgatacccccactgacgagcccgatgctctgtacgttgcatgccaagggcagccaaagggtgggcacgtgacctctatgcaacccaaggaaccagggcccctgcaatgtgaagccaaaccactagggaaagcaggggtccaacttgagccccagttggaggcccccctaaacgaggagatgccactgctgcccccacctgaggagtcacccctgtccccaccacctgaggaatcacccacgtccccaccacctgaggcatcacgcctgtcgccaccacctgaggaattgcccgcatccccacttcctgaggcattgcacctgtcccggccgctggaggaatcgcccctctctccgccgcctgaggagtctcctctgtctcccccacctgaatcatcacctttttctccactggaggagtcgcccttgtctccaccggaagagtcacccccatctcctgcacttgagacgcctctatccccaccacctgaagcatcgcccctgtccccaccatttgaagaatctcctttgtccccgccacctgaggaattgcccacttccccgccacctgaagcatctcgcctgtctccaccacctgaggagtcacccatgtcccctccacctgaagagtcacccatgtctccaccaccggaggcatctcgtctgttcccaccatttgaagagtctcctctgtcccctccacctgaggagtctcccctttccccaccacctgaggcatcacgcctgtccccaccacctgaggactcgcctatgtccccaccacctgaagaatcacctatgtcccccccacctgaggtatcgcgcctatcccccctgcctgtggtgtcacgcctgtctccaccgcctgaggaatctcccttgtccccaccggccctgtctcctttgggggagttagagtacccctttggtgccaaaggggacagtgaccctgagtcaccgttggctgcccccatcctggagacacccatcagccctccaccagaagctaactgcactgaccctgagcctgtcccccctatgatccttcccccatctccaggctccccagtggggccggcttctcccatcctgatggagccccttcctcctcagtgttcgccactccttcagcattccctggttccccaaaactcccctccttcccagtgctctcctcctgccctaccactgtccgttccctccccgttgagtcccatagggaaggtagtgggggtctcagatgaggctgagctgcacgagatggagactgagaaagtttcagaacctgaatgcccagccttggaacccagtgccaccagtcctctcccttccccaatgggggacctttcctgccccgcccccagccctgccccagccctggatgacttctctggcctaggggaagacacagcccctctggatgggattgatgctccgggttcacagccagagcctggacagacccctggcagtttggctagtgaacttaaaggctcccctgtgctcctggaccccgaggagctggcccctgtgacccctatggaggtctaccccgaatgcaagcagacagcagggcggggctcaccatgtgaagaacaggaagagccacgtgcaccggtggcccccacaccacccactctcatcaaatccgacatcgttaacgagatctctaatctgagccagggtgatgccagtgccagttttcctggctcagagcccctcctgggctctccagacccggaggggggtggctccctgtccatggagttgggggtctctacggatgttagtccagcccgagatgagggctccctacggctctgtactgactcactgccagagactgatgactcactattgtgcgatgctgggacagctatcagcggaggcaaagctgagggggagaaggggcggcggcgcagctccccagcccgttcccgcatcaaacagggtcgcagcagcagtttcccaggaagacgccggcctcgtggaggagcccatggagggcgtggtagaggacgggcccggctaaagtcaactgcttcttccattgagactctggtagttgctgacattgatagctctcccagtaaggaggaggaggaagaagatgatgacaccatgcagaataccgtggttctcttctccaacacagacaaatttgtcctaatgcaggacatgtgtgtggtatgtggcagctttggccggggggcagagggccacctccttgcctgttcgcagtgctctcagtgctatcacccttactgtgtcaacagcaagatcaccaaggtgatgctgctcaagggctggcgttgtgtggagtgtattgtgtgtgaggtgtgtggccaggcctccgacccctcacgcctgctgctctgtgatgactgtgatattagctaccacacatactgcctggaccccccactgctcaccgtccccaagggcggctggaagtgcaagtggtgtgtgtcctgtatgcagtgtggggctgcttcccctggcttccactgtgaatggcagaatagttacacacactgtgggccctgtgccagcctggtgacctgccctatctgtcatgctccttacgtagaagaggacctactaatccagtgccgccactgtgaacggtggatgcatgcaggctgtgagagcctcttcacagaggacgatgtggaccacgcacccgatgaaggctttgactgtgtctcctgccagccctacgtggtaaagcctgtggcgcctgttgcacctccagagctggtgcccatgaaggtgaaagagccagagccccagtactttcgcttcgaaggcgtgtggctgacagaaactggcatggccttgctgcgtaacctgaccatgtcaccactgcacaagcggcgccaacggcgaggacggcttggcctcccaggcgaggcaggattggagggttctgagccctcagatgcccttggccctgatgacaagaaggatggggacctggacaccgatgagctgctcaagggtgaaggtggtgtggagcacatggagtgcgaaattaaactggagggccccgtcagccctgatgtggagcctggcaaagaggagaccgaggaaagcaaaaaacgcaagcgtaaaccatatcggcctggcattggtggtttcatggtgcgacagcggaaatcccacacacgcacgaaaaaggggcctgctgcacaggcggaggtgttgagtggggatgggcagcccgacgaggtgatacctgctgacctgcctgcagagggcgccgtggagcagagcttagctgaaggggatgagaagaagaagcaacagcggcgagggcgcaagaggagcaaactggagggcatgttccctgcttacttgcaggaagccttctttgggaaggagctgctggacctgagccgtaaggccctttttgcagttggggtgggccggccaagctttggactagggaccccaaaagccaagggagatggaggctcagaaaggaaggaactccccacatcgcagaaaggagatgatggtccagatattgcagatgaagaatcccgtggcctcgagggcaaagccgatacaccaggacctgaggatgggggcgtgaaggcatccccagtgcccagtgaccctgagaagccaggcaccccaggtgaagggatgcttagctctgacttagacaggatttccacagaagaactgcccaagatggaatccaaggacctgcagcagctcttcaaggatgttctgggctctgaacgagaacagcatctgggttgtggaacccctggcctagaaggcagccgtacgccactgcagaggccctttcttcaaggtggactccctttgggcaatctgccctccagcagcccaatggactcctacccaggcctctgccagtccccgttcctggattctagggagcgcgggggcttctttagcccggaacccggtgagcccgacagcccctggacgggctcaggtggcaccacgccctccacccccacaacccccaccacggagggtgagggcgacggactctcctataaccagcggagtcttcagcgctgggagaaggatgaggagttgggccagctgtccaccatctcgcctgtgctctatgccaacattaattttcctaatctcaagcaagactacccagactggtcaagccgttgcaaacaaatcatgaagctctggagaaaggttccagcagctgacaaagccccctacctgcaaaaggccaaagataaccgggcagctcaccgcatcaacaaggtgcagaagcaggctgagagccagatcaacaagcagaccaaggtgggcgacatagcccgtaagactgaccgaccggccctacatctccgcattcccccgcagccaggggcactgggcagcccgccccccgctgctgcccccaccattttcattggcagccccactacccccgccggcttgtctacctctgcggacgggttcctgaagccgccggcgggctcggtgcctggccctgactcgcctggtgagctcttcctcaagctcccaccccaggtgcccgcccaagcgccttcgcaggacccctttggactggcccctgcctatcccctggagccccgcttccccacggcaccgcccacctatcccccctatcctagtcctacgggggcccctgcgcagcccccgatgctgggcgcctcatctcgtcctggggctggccagccaggggaattccacactaccccacctggcacccccagacaccagccctccacacctgacccgttcctcaaaccccgctgcccctcgctggataacttggctgtgcctgagagccctggggtagggggaggcaaagcttccgagcccctgctctcgcccccaccttttggggagtcccggaaggccctagaggtgaagaaggaagagcttggggcatcctctcctagctatgggcccccaaacctgggctttgttgactcaccctcctcaggcacccacctgggtggcctggagttaaagacacctgatgtcttcaaagcccccctgacccctcgggcatctcaggtagagccccagagcccgggcttgggcctaaggccccaggagccaccccctgcccaggctttggcaccttctcctccaagtcacccagacatctttcgccctggctcctacactgacccatatgctcagcccccattgactcctcggccccaacctccgccccctgagagctgctgtgctctgccccctcgctcactgccctccgaccctttctcccgagtgcctgtcagtcctcagtcccagtccagctcccagtctccactgacaccccggcctctgtctgctgaagctttttgcccatcacccgttacccctcgcttccagtcccctgacccttattctcgcccaccctcacgccctcagtcccgtgacccatttgccccattgcataagccaccccgaccccagccccctgaagttgcctttaaggctgggtctctagcccacacttcgctgggggctggggggttcccagcagccctgcccgcggggccagcaggtgagctccatgccaaggtcccaagtgggcagccccccaattttgtccggtcccctgggacgggtgcatttgtgggcaccccctctcccatgcgtttcactttccctcaggcagtaggggagccttccctaaagccccctgtccctcagcctggtctcccgccaccccatgggatcaacagccattttgggcccggccccaccttgggcaagcctcaaagcacaaactacacagtagccacagggaacttccacccatcgggcagccccctggggcccagcagcgggtccacaggggagagctatgggctgtccccactacgccctccgtcggttctgccaccacctgcacccgacggatccctcccctacctgtcccatggagcctcacagcgatcaggcatcacctctcctgtcgaaaagcgagaagacccagggactggaatgggtagctctttggcgacagctgaactcccaggtacccaggacccaggcatgtccggccttagccaaacagagctggagaagcaacggcagcgccagcggctacgagagctgctgattcggcagcagatccagcgcaacaccctgcggcaggagaaggaaacagctgcagcagctgcgggagcagtggggcctccaggcagctggggtgctgagcccagcagccctgcctttgagcagctgagtcgaggccagaccccctttgctgggacacaggacaagagcagccttgtggggttgcccccaagcaagctgagtggccccatcctggggccagggtccttccctagcgatgaccgactctcccggccacctccaccagccacgccttcctctatggatgtgaacagccggcaactggtaggaggctcccaagctttctatcagcgagcaccctatcctgggtccctgcccttacagcagcaacagcaacaactgtggcagcaacaacaggcaacagcagcaacctccatgcgatttgccatgtcagctcgctttccatcaactcctggacctgaacttggccgccaagccctaggttccccgttggcgggaatttccacccgtctgccaggccctggtgagccagtgcctggtccagctggtcctgcccagttcattgagctgcggcacaatgtacagaaaggactgggacctgggggcactccgtttcctggtcagggcccacctcagagaccccgtttttaccctgtaagtgaggacccccaccgactggctcctgaagggcttcggggcctggcggtatcaggtcttcccccacagaaaccctcagccccaccggcccctgaattgaacaacagtcttcatccaacaccccacaccaagggtcctaccctgccaactggtttggagctggtcaaccggcccccgtcgagcactgagcttggccgccccaatcctctggccctggaagctgggaagttgccctgtgaggatcccgagctggatgacgattttgatgcccacaaggccctagaggatgatgaagagcttgctcacctgggtctgggtgtggatgtggccaagggtgatgatgaacttggcaccttagaaaacctggagaccaatgacccccacttggatgacctgctcaatggagacgagtttgacctgctggcatatactgatcctgagctggacactggggacaagaaggatatcttcaatgagcacctgaggctggtagaatcggctaatgaggaggctgaacgggaggccctgctgcggggggtggagccaggacccttgggccctgaggagcgccctccccctgctgctgatgcctctgaaccccgcctggcatctgtgctccctgaggtgaagcccaaggtggaggagggtggacgccacccttctccttgccaattcaccattgctacccccaaggtagagcccgcacctgctgccaattcccttggcctggggctaaagccaggacagagcatgatgggcagccgggatacccggatgggcacagggccattttctagcagtgggcacacagctgagaaggcctcctttggggccacgggagggccaccagctcacctgctgacccccagcccactgagtggcccaggaggatcctccctgctggaaaagtttgagctcgagagtggggctttgaccttgcctggtggacctgcagcatctggggatgagctagacaagatggagagctcactggtagccagcgagttacccctgctcattgaggacctgttggagcatgagaagaaggagctgcagaagaagcagcagctttcagcacagttgcagcctgcccagcagcagcagcaacagcagcagcagcattccctactgcctgcaccaggccctgcccaggccatgtctttgccacatgagggctcttctcccagtttggctgggtcccaacagcagctttccctgggtcttgcagttgcccgacagccaggtttgccccagccactgatgcccacccagccaccagctcatgccctccagcaacgcctggctccatccatggctatggtgtccaatcaagggcatatgctaagtgggcagcatggagggcaggcaggcttggtaccccagcagagctcacagccagtgctatcacagaagcccatgggcaccatgccaccttccatgtgcatgaagccgcagcaattggcaatgcagcagcagctggcaaacagcttcttcccagatacagacctggacaaatttgctgcagaagatatcattggtcccattgcaaaggccaagatggtggctttgaaaggcatcaagaaagtgatggctcagggcagcattggggtggcacctggtatgaacagacagcaagtgtctctgctagcccagaggctctcggggggacctagcagtgatctgcagaaccatgtggcagctgggagtggccaggagcggagtgctggtgatccctcccagcctcgtcccaacccgcccacttttgctcagggagtgatcaatgaagctgaccagcggcagtatgaggagtggctgttccatacccagcagctcctacagatgcagctgaaggtgctagaggagcagattggtgtacaccgcaagtcccggaaggctctgtgtgccaagcagcgcactgccaaaaaagctggccgtgagttcccagaagctgatgctgagaagctcaagctggttacagagcagcagagcaagatccagaaacaactggatcaggtccggaaacagcagaaggagcacactaatctcatggcagaatatcggaacaagcagcagcaacaacagcagcagcagcagcaacaacagcaacagcactcagctgtgctggctctcagcccttcccagagtccccggctgctcaccaagctccctggtcagctgctccctggccatgggctgcagccaccacaggggcctccgggtgggcaagccggaggtcttcgcctgacccctgggggtatggcactacctggacagcctggtggccccttccttaatacagctctggcccaacagcagcaacagcaacattctggtggggctggatccctggctggcccttcagggggcttcttccctggcaaccttgctcttcgaagcctcggacctgattcaaggcttttacaggaaaggcagctgcagctgcagcagcaacgtatgcagctggcccagaaactgcagcagcagcagcagcagcaacagcagcagcagcaccttctaggacaggtggcaatccagcagcaacagcagcagggtcctggagtacagacaaaccaagctctgggtcccaagccccagggccttatgcctcccagcagccaccaaggcctcctggtccagcagctgtcccctcaaccaccccaggggccccagggcatgctgggccctgcccaggtggctgtgttgcagcagcagcaccctggagctttgggcccccagggccctcacagacaggtgcttatgacccagtcccgggtgctcagttccccccagctggcacagcagggtcagggccttatgggacacaggctggtcacagcccagcagcagcagcagcaacaacagcaccaacagcaagggtccatggcagggctgtcccatcttcagcaaagtctgatgtcacacagtgggcagcccaaactgagcgctcagcccatgggctctttacagcagcttcagcagcagcagcagctgcaacagcaacagcaacttcagcagcagcagcagcagcagctacaacagcaacagcaacttcagcagcaacagcttcaacagcagcaacagcagcagcagcttcaacaacagcagcagcaacagcttcaacagcagcaacagcagctacaacagcaacagcaacaacaacagcagcagtttcaacagcagcagcaacagcagcagatgggccttttaaaccagagtcgaactttactgtcccctcagcaacaacagcagcagcaagtggcacttggccctggcatgccagcaaagcctcttcaacacttttctagccctggagccctgggtccaaccctcctcctgacgggcaaggaacaaaacaccgtagacccagccgtttcttcagaggccactgaggggccctctacacatcagggagggccgttagcaataggaactacccctgagtcaatggccactgaaccaggagaggtaaagccctcactctctggggactcacaactcctgcttgtccaaccccagccccagcctcagcccagctctctgcagctgcagccacctctgaggcttccaggacaacagcagcagcaagttagcctgctccacacagcaggtggaggaagccatgggcagctaggcagtggatcatcttctgaggcctcatctgtgccccacctgctggctcagccctctgtttccttaggggatcagcctgggtccatgacccagaaccttctgggcccccaacagcccatgctagagcggcccatgcaaaataatacagggccacaacctcccaaaccaggacctgtcctccagtctgggcagggtctgcctggggttggaatcatgcctacggtgggtcagcttcgagcacagctccaaggagtcctggccaaaaacccacagctgcggcacttaagtcctcagcagcagcagcagctacaggcactcctcatgcagcggcagctgcagcagagtcaggcagtacgccagaccccaccctaccaggagcctgggacccagacctctcccctccagggcctcctgggctgccaacctcaacttgggggcttccctggaccacagacaggccccctccaggagctaggggcagggcctcgacctcagggcccaccccggctccctgccccaccaggagccttatctacaggaccagtccttggccctgtccatcccacacctccaccatccagccctcaagagccaaagagaccttcacaattaccttcccccagctcccagcttcccactgaggcccagctccctcccacccatccagggacccccaaacctcaggggccaaccttggagccgcctcctgggagggtctcacctgctgctgcccagcttgcagataccttgtttagcaagggtctgggaccttgggatcccccagacaacctagcagaaacccagaagccagagcagagcagcctggtacctgggcatctggaccaggtgaatggacaggtggtgcctgaggcatcccaactcagcatcaagcaggaacctcgggaagagccatgtgccctgggagcccagtcagtgaagagggaggccaatggggagccaataggggcaccaggaaccagcaaccacctcctgctggcaggccctcgctcagaagctgggcatctgctcttgcagaagctactccgggcaaagaatgtgcaactcagcactgggcaggggtccgaggggctgcgagctgagatcaacgggcacattgacagcaagctggctgggctggagcagaaactacagggtacccccagcaacaaggaggatgcagcagcaaggaagcctttgacaccgaagcccaagcgggtacagaaggcaagcgacaggttggtgagctcccgaaagaagctgcggaaggaggacggcgtcagggccagcgaggccttgctgaaacagctgaaacaggagctgtccctgctgcccctaacggagcctgctatcaccgccaattttagcctctttgccccctttggcagtggctgcccagtcaatgggcagagccagctgaggggggcctttggaagtggggcgctgcccactggccctgactactattcccagctgcttaccaagaataacctgagtaacccgccgacaccaccctcgtcgctgccccccaccccacccccatcggtgcagcagaagatggtgaatggcgtcaccccatctgaagagctgggggagcaccccaaggatgctgcctctgcccgggatagtgaaagggcactgagggatacttcagaggtgaagagtctagacctgctggctgccttgcctacaccccctcacaatcagactgaggatgtcaggatggagagtgatgaggatagcgattctcctgacagcattgtgccagcttcatcccctgagagcatcttgggggaggaggcccctcgtttccctcatctgggctcaggccggtgggagcaagaggaccgggccctctcccctgtcatccccctcattcctcgggacagcatcccagtcttcccagataccaaaccttatggggcccttggcctggaggtccctggaaagctgcctgtcacaacttgggaaaagggcaaaggaagtgaggtgtcagtcatgctcacagtctctgctgctgcagacaagaacctgaatggcgtgatggtggcagtggcggagctgctgagcatgaagatccccaactcctatgaggtgctgttcccagagagccccgcccggggaggcactgagccaaagaagggggaagctgagggtcctggtgggaaggaaaagggtctggaaggcaagagcccagacactggccctgattggctgaagcagtttgatgcagtgttggctggctataccctgaagaggcaactagacatcttgagcctcctgaaacaggagagccccgccccagagccacccactcagcacaggtatacctacaatgtctccaatctggatgtgcgacagctctcggccccacctcctgaagaaccctccccgcccccttcccccttggcaccttctcctgccagtccccctactgagcccttggttgaacttcccaccgaacccttggctgagccacccgtcccctcacctctgccactggcctcatcccctgaatcagcccgacccaagccccgtgcccggccccctgaagaaggtgaagatacccgtcctcctcgcctcaagaaatggaaaggagtgcgctggaagcggcttcggctgctgctgaccatccagaagggcagtggacggcaggaggatgagcgggaagtggcagagtttatggagcagcttggcacagccttgcgacctgacaaggtaccgcgagacatgcgtcgctgctgtttctgtcatgaggagggtgacggggccactgatgggcctgcccgtctgctgaacctggacctggacctgtgggtgcacctcaactgtgccctttggtccacggaggtgtatgagacccagggcggagcactgatgaatgtggaggttgccctgcaccgaggactgctaaccaagtgctccctgtgccagcgaactggtgccaccagcagctgcaatcgcatgcgttgccccaatgtctaccattttggttgtgccatccgcgccaagtgcatgttcttcaaggacaagaccatgctgtgtccaatgcataagatcaaggggccctgtgagcaagagctgagctcttttgctgtcttccggcgggtctacattgagcgggacgaggtgaagcaaatcgctagcatcattcagcggggagaacggctgcacatgttccgtgtgggggggcttgtgttccacgccatcggacagctgctgcctcaccagatggctgactttcatagtgccactgccctctatcccgtgggctacgaggccacgcgcatctattggagcctccgcaccaacaatcgtcgctgctgctatcgctgttctattggtgagaacaacgggcggccggagtttgtaatcaaagtcatcgagcagggcctggaggacctggtcttcactgacgcctctccccaggccgtgtggaatcgcatcattgagcctgtggctgccatgagaaaagaggctgacatgctgcgactcttccctgagtatctgaagggcgaggagctctttgggctgacggtgcatgccgtgcttcgcatagctgaatcactgcccggggtggagagctgtcaaaactatttattccgctatgggcgccacccccttatggagctgccactcatgatcaaccccactggctgtgcccgatcagagcctaaaatcctcacacactacaaacggccccataccctgaacagcaccagcatgtctaaggcatatcagagcaccttcacaggcgagaccaacaccccctacagcaagcagtttgtgcactccaagtcatctcagtaccggcggctgcgcaccgaatggaagaacaacgtgtacctggctcgctcccgtatccagggcctggggctctatgcagccaaggacctagaaaagcacacaatggttatcgagtacattggcaccatcattcggaacgaggtggccaaccggcgggagaaaatctacgaagagcagaatcgaggcatctacatgttccgaataaacaatgaacatgtgattgatgctacgttgaccggcggccctgccaggtacattaaccattcctgtgcccctaactgtgtggccgaagtcgtgacatttgacaaagaggacaaaatcatcatcatctccagccggcgaatccccaaaggagaggagctaacctatgactatcagtttgattttgaggacgatcagcacgagatcccctgccactgtggagcctggaattgtcggaaatggatgaactaa

SEQ ID NO: 3 - DNA, 24 bp, Artificial Sequence
  Component of JH probe
tatggactactggggtcaaggaac

SEQ ID NO: 4 - DNA, 23 bp, Artificial Sequence
  Component of JH probe
ccaactacagccccaactatccc

SEQ ID NO: 5 - DNA, 32 bp, Artificial Sequence
  Component of 3′Smu probe
ccatgggctgcctagcccgggacttcctgccc

SEQ ID NO: 6 - DNA, 37 bp, Artificial Sequence
  Component of 3′Smu probe
atctgtggtgaagccagattccacgagcttcccatcc

SEQ ID NO: 7 - DNA, 22 bp, Artificial Sequence
  Reverse primer
aggctctgagatccctagacag

SEQ ID NO: 8 - DNA, 20 bp, Artificial Sequence
  TNFAIP3 (A20) Forward Primer
gtgctgccatcccccaaata

SEQ ID NO: 9 - DNA, 19 bp, Artificial Sequence
  TNFAIP3 (A20)  Reverse Primer
agctttcccatgagccact

SEQ ID NO: 10 - DNA, 21 bp, Artificial Sequence
  SOCS3 Forward Primer
acctggctagactgaggtcat

SEQ ID NO: 11 - DNA, 20 bp, Artificial Sequence
  SOCS3 Reverse Primer
ttagaggcgctctggttcct

SEQ ID NO: 12 - DNA, 20 bp, Artificial Sequence
  TRAF3 Forward Primer
tccaagggaagatgaggcca

SEQ ID NO: 13 - DNA, 20 bp, Artificial Sequence
  TRAF3 Reverse Primer
cctcgggggccataatacag

SEQ ID NO: 14 - DNA, 20 bp, Artificial Sequence
  SGK1 Forward Primer
gaccgattgggaaagcaggt

SEQ ID NO: 15 - DNA, 20 bp, Artificial Sequence
  SGK1 Reverse Primer
gagttggctctggcttccat

SEQ ID NO: 16 - DNA, 20 bp, Artificial Sequence
  IKBKB Forward Primer
aggtcaacaaggagtcagcc

SEQ ID NO: 17 - DNA, 20 bp, Artificial Sequence
  IKBKB Reverse Primer
aggagggaggggagctttat

SEQ ID NO: 18 - DNA, 19 bp, Artificial Sequence
  TNS4 (negative control loci) Forward Primer
ttatttggctgggtgtggt

SEQ ID NO: 19 - DNA, 22 bp, Artificial Sequence
  TNS4 (negative control loci) Reverse Primer
gtagagacgggatttcaccatg

SEQ ID NO: 20 - DNA, 21 bp, Artificial Sequence
  shRNA to mouse Kmt2d (#1)
gactggtctagccgatgtaaa

SEQ ID NO: 21 - DNA, 21 bp, Artificial Sequence
  shRNA to mouse Kmt2d (#2)
tgaatctttatcttcagcagg

SEQ ID NO: 22 - DNA, 29 bp, Artificial Sequence
  IgL-V lambda 1 primer
gccatttccccaggctgttgtgactcagg

SEQ ID NO: 23 - DNA, 28 bp, Artificial Sequence
  IgL-J lambda 1,3 reverse primer
actcacctaggacagtcagcttggttcc

(19) United States
(12) Patent Application Publication
(10) Pub. No.: US 2018/0223368 A1
(43) Pub. Date: Aug. 9, 2018

(54) METHODS FOR DIAGNOSING AND TREATING FOLLICULAR LYMPHOMA

(71) Applicants: Memorial Sloan Ketteting Cancer Center, New York, NY (US); CORNELL UNIVERSITY, Ithaca, NY (US)

(72) Inventors: Hans-Guido WENDEL, New York, NY (US); Ari MELNICK, New York, NY (US); Ana ORTEGA MOLINA, New York, NY (US); Isaac BOSS, Yonkers, NY (US)

(73) Assignees: Memorial Sloan Ketteting Cancer Center, New York, NY (US); CORNELL UNIVERSITY, Ithaca, NY (US)

(21) Appl. No.: 15/559,046

(22) PCT Filed: Mar. 17, 2016
(86) PCT No.: PCT/US16/22953

§ 371 Date: Sep. 17, 2017

Related U.S. Application Data
(60) Provisional application No. 62/135,040, filed on Mar. 18, 2015.
(60) Provisional application No. 62/201,390, filed on Aug. 5, 2015.

(51) Int. Cl. C12Q 1/6886

(52) U.S. Cl. CPC C12Q 1/6886; C12Q 2600/106; C12Q 2600/156

(57) ABSTRACT

The invention relates generally to methods for diagnosis and treatment of follicular lymphoma or diffuse large B cell lymphoma. Specifically, the invention relates to detecting a lysine (K)-specific methyltransferase 2D (KMT2D) alteration to diagnose or treat follicular lymphoma or diffuse large B cell lymphoma.

### CROSS-REFERENCE TO RELATED APPLICATIONS

This application claims priority under 35 USC 119(e) to U.S. patent application Ser. No. 62/135,040, filed Mar. 18, 2015, and to U.S. patent application Ser. No. 62/201,390, filed Aug. 5, 2015, both of which are incorporated herein by reference in their entireties.

### GOVERNMENT SUPPORT

This invention was made with government support under grants CA183876, CA019038, CA187109, GM110174, CA150265, DP2OD007447 and CA008748 from the National Institutes of Health, and Grant 11557134 from the Department of Defense. The government has certain rights in the invention.

### FIELD OF THE INVENTION

The invention relates generally to methods for diagnosis and treatment of follicular lymphoma. Specifically, the invention relates to detecting the presence or absence of a lysine (K)-specific methyltransferase 2D (KMT2D) alteration to diagnose or treat follicular lymphoma.

### BACKGROUND OF THE INVENTION

Lymphoma is the most common blood cancer. There are two main forms of lymphoma, which are Hodgkin lymphoma and non-Hodgkin lymphoma (NHL). The body has two main types of lymphocytes that can develop into lymphomas. They are: B-lymphocytes (B-cells) and T-lymphocytes (T-cells). Follicular lymphoma (FL), a B-cell lymphoma, is the most common form of B-cell lymphoma. It is a slow-growing lymphoma. It is also called an “indolent” lymphoma for its slow nature, in terms of its behavior and how it looks under the microscope. Follicular lymphoma is subtle, with minor warning signs that often go unnoticed for a long time. Often, people with follicular lymphoma have no obvious symptoms of the disease at diagnosis. Follicular lymphoma remains incurable despite recent advances in lymphoma therapy. Follicular lymphoma arises from germinal center B-cells and the disease is typically triggered by the translocation t(14; 18) that activates the anti-apoptotic BCL2 oncogene. However, the t(14; 18) translocation is also detectable in many healthy adults who never develop the disease. This indicates that additional genetic and epigenetic events contribute to lymphomagenesis. Indeed, recent genome sequencing studies have catalogued many recurrent mutations in human B-cell lymphoma.

Accordingly, there exists a need to understand the genetics and molecular mechanisms of follicular lymphoma, and thereby develop improved methods for diagnosis and treatment.

### SUMMARY OF THE INVENTION

In one embodiment, the invention provides a method for diagnosing a follicular lymphoma, in a subject, the method comprising the steps of: obtaining a biological sample from said subject; and testing said biological sample to detect the presence or absence of a lysine (K)-specific methyltransferase 2D (KMT2D) alteration in said biological sample, wherein the presence of said KMT2D alteration indicates a diagnosis of said follicular lymphoma in said subject. In one embodiment, the invention provides a method for diagnosing responsiveness of a follicular lymphoma in a subject to therapy, the method comprising the steps of: obtaining a biological sample from said subject; and testing said biological sample to detect the presence or absence of a lysine (K)-specific methyltransferase 2D (KMT2D) alteration in said biological sample, wherein the presence of said KMT2D alteration indicates a poor responsiveness or contraindication of said follicular lymphoma in said subject of the therapy. In an exemplary embodiment, said KMT2D alteration is a mutation in KMT2D. In another exemplary embodiment, the response to therapy is said subject's response or responsiveness to an immunotherapy, for example, said subject's tumor response to immunotherapy. In one embodiment the therapy is B cell therapy. In one embodiment, a patient with a KMT2D alteration may not be effectively treated with anti-CD40 therapy. In another embodiment, anti-CD40 therapy is contraindicated in a patient found to have a KMT2D alteration. In another embodiment, methods for treating follicular lymphoma include a determination of KMT2D alteration and guiding therapy away from anti-CD40 in the presence of an altered KMT2D. The use or non-use of anti-CD40 therapy may be in conjunction with the use or non-use of anti-IgM therapy.

In another embodiment, the invention provides a method of determining a treatment outcome for treating a follicular lymphoma, in a subject, the method comprising the steps of: obtaining a biological sample from said subject; and testing said biological sample to detect the presence or absence of a KMT2D alteration in said biological sample, wherein the presence of said KMT2D alteration indicates a response to a therapy, thereby determining said treatment outcome for treating said follicular lymphoma in said subject. In one embodiment, a patient with a KMT2D alteration may not be effectively treated with anti-CD40 therapy. In another embodiment, anti-CD40 therapy is contraindicated in a patient found to have a KMT2D alteration. In another embodiment, methods for treating follicular lymphoma include a determination of KMT2D alteration and guiding therapy away from anti-CD40 in the presence of an altered KMT2D. The use or non-use of anti-CD40 therapy may be in conjunction with the use or non-use of anti-IgM therapy.

In another embodiment, the invention provides a method for treating a follicular lymphoma, in a subject, the method comprising: (a) obtaining a biological sample from said subject; and testing said biological sample to detect the presence or absence of a KMT2D alteration in said biological sample, wherein the presence of said KMT2D alteration indicates a response to a therapy; (b) based on the determination of said response to said therapy, administering an effective amount of a therapeutic agent to treat said follicular lymphoma, thereby treating said follicular lymphoma in said subject. In one embodiment, a patient with a KMT2D alteration may not be effectively treated with anti-CD40 therapy. In another embodiment, anti-CD40 therapy is contraindicated in a patient found to have a KMT2D alteration. In another embodiment, methods for treating follicular lymphoma include a determination of KMT2D alteration and guiding therapy away from anti-CD40 in the presence of an altered KMT2D. The use or non-use of anti-CD40 therapy may be in conjunction with the use or non-use of anti-IgM therapy.

In another embodiment, the invention provides a method for identifying a molecule that increases sensitivity of a follicular lymphoma in a subject to immunotherapy, the method comprising: providing a plurality of molecules; and screening said plurality of molecules to identify a molecule that effectively enhances the level of a KMT2D, thereby identifying said molecule that effectively increases sensitivity of said follicular lymphoma in said subject to immunotherapy.

In another embodiment, the invention provides a method for treating a follicular lymphoma in a subject, the method comprising: administering to said subject a molecule that effectively enhances the level of a KMT2D in said subject, in combination with anti-CD40 antibodies, thereby treating said follicular lymphoma in said subject.

In any of the foregoing embodiments, therapy is B cell therapy, such as but not limited to anti-CD40 antibody, anti-CD20 antibody or anti-IgM therapy, or any combination thereof.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

This application claims priority under 35 USC 119(e) to U.S. patent application Ser. No. 62/135,040, filed Mar. 18, 2015, and to U.S. patent application Ser. No. 62/201,390, filed Aug. 5, 2015, both of which are incorporated herein by reference in their entireties.

### BRIEF DESCRIPTION OF THE DRAWINGS

FIGS. 1A-1G show that Kmt2d deficiency accelerates B cell lymphoma development in mice. (FIG. 1A) Diagram of the adoptive transfer model of FL using the VavP-Bcl2 transgenic mouse and retroviral transduction of HPCs followed by reconstitution into lethally irradiated, syngeneic, female mice. WT, wild type. MLS-shKmt2d, MSCV-GFP encoding shRNA against Kmt2d (FIG. 1B) Kaplan-Meier curve of C57BL/6 mice transplanted with VavP-Bcl2 HPCs transduced with MSCV-GFP retrovirus (black, n=37), MSCV-GFP encoding shRNAs against Kmt2d (red, n=30) or MSCV-GFP encoding c-Myc (gray, n=16). Statistical significance of survival difference was determined by the log-rank test: shKmt2d versus vector, P=0.03; c-Myc versus vector, P<0.001). (FIG. 1C) Mice were killed 5 months after injection and splenic lymphoma cells of mice that had been injected with VavP-Bcl2 HPCs (transduced with retrovirus encoding either GFP only or coexpressing one of two independent Kmt2d-specific shRNAs (shKmt2d#1 and shKmt2d#2) and GFP) were compared by flow cytometry to the same VavP-Bcl2 HPCs before injection into mice. (FIG. 1D) Recipient mice were killed 5 months after HPC injection and Kmt2d mRNA levels from MACS-sorted B220⁺ lymphoma B cells were quantified by qRT-PCR (vector, n=4; shKmt2d #1, n=5). Values correspond to average ±s.d. (FIG. 1E) Spleen weights (normalized to body weight) of the indicated recipient mice that were killed 5 months after HPC injection (vector, n=9; shKmt2d#1, n=11; c-Myc n=5). Representative images of spleens are shown on the right. Scale bars represent 0.5 cm. Values correspond to average ±s.d. Statistical significance in d and e was determined by the two-tailed Student's t-test, *P<0.05, ***P<0.001. (FIG. 1F) Upon sacrifice, tissue was extracted from recipient mice and stained with H&E and antibodies specific for B220, Ki67, PNA or TUNEL. Scale bars, 400 μm. (FIG. 1G) Representative images of flow cytometry analysis for the cellular composition of whole spleens from recipient mice that were killed 5 months after injection with HPCs. Four tumors of each genotype were analyzed.

FIG. 2 shows that Kmt2d deficiency affects physiological B cell behavior. (a) Schematic diagram of SRBC immunization study (SRBC, sheep red blood cell). (b) Representative spleen sections harvested 16 weeks after SRBC immunization from WT C57BL/6 females transplanted with HPCs expressing either MSCV-GFP (n=3) or MSCV-GFP-shKmt2d#1 (n=3) and stained with H&E and the indicated markers. Red asterisks indicate PNA⁺ cells. Images correspond to 10× magnification. (c) Quantification of Ki67 staining from FIG. 2b. Values represent mean±s.d. (n=3 females per genotype; two-tailed Student's t-test; ***P<0.001). (d,e) Representative plots (d) and quantitation (e) of flow cytometry analysis of splenocytes harvested from Kmt2d^{+/+} (WT, n=4, 2 males and 2 females; 1.5-2 months old) or Kmt2d^{-/-} mice (n=4, 3 males and 1 female; 1.5-2 months old) 6 d after NP-CGG immunization. Cells were first gated on live (7ADD⁻) B220⁺ lymphocytes to determine percentage of GC B cells (CD95⁺GL7⁺), transitional B cells (TR, CD21⁻CD23⁻), follicular zone B cells (FO, CD23⁺CD21^{lo}), marginal zone B cells (MZ, CD23^{lo}CD21⁺) and intermediate plasma cells (IPC, B220⁺CD138⁺). Plasma cells (PC, B220⁻ CD138⁺) cells were gated on live cells (7ADD⁻). Values represent mean±s.d. Two-tailed Student's t-test was used to determine statistical significance; *P<0.05, **P<0.01. The antibodies used are described in Online Methods. Values represent mean±s.d. (n=4 mice per genotype; WT: 2 males and 2 females, Kmt2d^{-/-}: 3 males and 1 female; 1.5-2 months old). Two-tailed Student's t-test was used to determine statistical significance (*P<0.05, **P <0.01). Antibodies used are described in Online Methods. (f) NP-specific IgM and IgG1 serum levels from WT or Kmt2d^{-/-} mice before (dashed lines) and 6 d after NP-CGG immunization, as determined by ELISA. The bars show mean±s.d. (n=4 mice per genotype; same as in d,e). Two-tailed Student's t-test was used to determine statistical significance; *P<0.05. Data correspond to one representative assay from a total of two independent assays. (g) Schematic diagram of the B cell differentiation assay (see also Online Methods). (h) Flow cytometry analysis of IgG1 class switch recombination in B cells from WT and Kmt2d^{-/-} mice 96 h post-stimulation in vitro with LPS, IL-4 and CD180-specific antibody. (i) Quantification of B220⁺ IgG1⁺ cells for two independent experiments. Values represent mean±s.d. (n=5 mice per genotype, 2 females and 3 males, 2.5-5 months old). Two-tailed Student's t-test was used to determine statistical significance; ***P<0.001.

FIG. 3 shows the consequences of KMT2D mutations in human FL and DLBCL. (a) Percentage of FL (n=104) specimens carrying KMT2D mutations according to the type of mutation. Exome refers to exome sequencing. Targeted refers to targeted sequencing. See Online Methods for further details. (b) Schematic diagram of the KMT2D mutations in FL specimens. PHD, pleckstrin homology domain; FYRN, phenylalanine- and tyrosine-rich domain N-terminal; FYRC, phenylalanine- and tyrosine-rich domain C-terminal; SET, Su(var)3-9, Enhancer-of-zeste and Trithorax domain. (c,d) Kaplan-Meier curves representing overall (c) and progression-free survival (d) of individuals with DLBCL, classified into three groups according to the KMT2D mutation status. Significance was estimated with the log-rank test. (e) Supervised analysis of the 100 most differentially expressed genes between human FL specimens that are WT (KMT2D^{wt}; n=12) and mutant (KMT2D^{mut}; n=7) for KMT2D. Columns represent individual FL specimens, rows correspond to the different genes indicated, with the expression value z-scores of rpkm (reads per kilobase per million mapped reads; scaled by row) as shown in the color bar. The two columns on the right represent a summary of all WT and mutant FL specimens, respectively. (f) Supervised analysis, as in e, comparing the 100 most differentially expressed mRNAs in MACS-purified mouse B220⁺ B cells from VavP-Bcl2-vector (n=4) and VavP-Bcl2-shKmt2d mouse lymphomas (n=5). (g) GSEA of 333 genes significantly downregulated in KMT2D^{mut} FLs (p-val<0.05) as compared to genes ranked log₂-fold change in VavP-Bcl2-shKmt2d versus VavP-Bcl2-vector B220⁺ lymphoma B cells. (h) GSEA analysis as in g of downregulated genes (n=820) in VavP-Bcl2-shKmt2d versus VavP-Bcl2-vector B220⁺ B cell lymphomas (P_{adj}<0.1) as compared to genes ranked log₂-fold change in KMT2D^{wt} FLs versus KMT2D^{mut} FLs. NES, normalized enrichment score; FDR, false discovery rate. (i) Pathway analysis of downregulated genes (n=347) identified in the GSEA leading-edge analyses from c,d and compared to signatures from the Lymphochip database and MysigDB. The background included around 24,000 genes from Ref-seq gene annotation. Statistical significance was determined by hypergeometric tests and is shown in the color key. The red color indicates (in log₁₀) the over-represented P values and the blue shows under-representation.

FIG. 4 shows the epigenetic effects of KMT2D on target genes in mouse lymphomas. (a) Average H3K4me1-H3K4me2 read density plot at promoters and enhancers in MACS-purified B220⁺ B cells from VavP-Bcl2-vector and VavP-Bcl2-shKmt2d lymphomas identified by ChIP-seq. (b) Proportion of H3K4me1 and H3K4me2 peaks by location near promoters or enhancers based on ChIP-seq from purified mouse B220⁺ cells from VavP-Bcl2-vector and VavP-Bcl2-shKmt2d lymphomas. The proportion of affected promoters and enhancers is shown for the indicated thresholds (***P<0.001 by chi-squared test). (c,d) GSEA of genes with a ≥25. % reduction in H3K4me1 and H3K4me2 marks at enhancers (P<0.05) in mouse B220⁺ cells from VavP-Bcl2-vector and VavP-Bcl2-shKmt2d lymphomas, as compared to ranked gene expression changes (log₂-fold) in B220⁺ mouse lymphomas (c) or in human FL specimens with WT and mutant KMT2D (d); NES, normalized enrichment score. (e). Pathway analysis of downregulated genes (n=322) identified in the GSEA leading-edge analyses in b,c compared to genes in lymphoid signature database from the Staudt Lab (http://lymphochip.nih.gov/signaturedb/) and MysigDB. The background included around 24,000 genes from Ref-seq gene annotation. Statistical significance was determined by hypergeometric test and shown in the color key. The red color indicates (in log₁₀) the over-represented P values and the blue shows under-representation. (f). Normalized UCSC (University of California Santa Cruz) read-density tracks of H3K4me1-H3K4me2 ChIP-seq peaks from B220⁺ mouse lymphomas with sh-Kmt2d (red) or vector (black).

FIG. 5 depicts the identification of KMT2D target genes in human lymphoma cells. (a) Proportion of H3K4me1-H3K4me2 peaks near promoters or enhancers by ChIP-seq in OCI-LY1 (containing KMT2D^{mut}) versus OCI-LY7 (containing KMT2D^{wt}) cells for the indicated thresholds (***P<0.001 by chi-squared test). (b) GSEA of genes with a ≥5.0% reduction in H3K4me1-H3K4me2 read density in OCI-LY1 versus OCI-LY7 cell lines, as compared to genes ranked by logₑ-fold change in FL specimens with WT versus mutant KMT2D. (c) Genomic distribution of KMT2D peaks located at transcription start sites (TSS), inside gene bodies (intragenic) or upstream or downstream of the closest gene in OCI-LY7 cells. (d) GSEA of genes with KMT2D binding in OCI-LY7 cells and >50% reduction in the H3K4me1-H3K4me2 mark in OCI-LY1 (KMT2D^{mut}) versus OCI-LY7 (KMT2D^{Wt}), as compared to genes ranked by log₂-fold change in human FL specimens with WT and mutant KMT2D. (e) Pathway analysis of downregulated genes in FL subjects (P<0.05 by Wald test) with KMT2D binding and >50% reduction in the H3K4me1-H3K4me2 mark in OCI-LY1 versus OCI-LY7 cells (n=1,248), as compared to those in the lymphoid signature database from the Staudt Lab (http://lymphochip.nih.gov/signaturedb/) and MysigDB. The background included around 24,000 genes from Ref-seq gene annotation. Statistical signficance was determined by hypergeometric test and is shown in the color key. The red color indicates (in log₁₀) the over-represented P values and the blue shows under-representation. (f) Normalized UCSC read-density tracks of KMT2D ChIP-seq peaks in OCI-LY7 (black) and OCI-LY1 (red) cells.

FIG. 6 shows that KMT2D inactivation affects growth and survival pathways in lymphoma cells. (a) mRNA levels, as measured by qRT-PCR, in the isogenic OCI-LY7 pairs expressing either a vector control or an shRNA against KMT2D. Values correspond to the average of three replicates ±s.d.; two-tailed Student's t-test was used to determine statistical significance; *P<0.05, **P<0.01, ***P<0.001. (b) qChIP analysis for H3K4me1-H3K4me2 occupancy loss in enhancer regions of the specified KMT2D target genes after KMT2D knockdown in OCI-LY7 cells. A genomic region (TNS4) with no KMT2D binding and H3K4me1-H3K4me2 was used as a negative control. Values correspond to mean percentage of input enrichment ±s.d. of triplicate qPCR reactions of a single replicate. Two-tailed Student's t-test was used to determine statistical significance; ***P<0.001. Data correspond to one representative assay from a total of 2 or 3 independent assays. (c) Immunoblot of the indicated proteins in vector- or shKMT2D-expressing OCI-LY7 cells upon 48 h of IL-21 stimulation. Actin was used as a loading control. (d) TNFAIP3 mRNA levels in OCI-LY7 and SU-DHL4 cells transduced with vector or shKMT2D upon 48 h of stimulation with antibodies to CD40 and IgM. Values correspond to the average of three experimental replicates ±s.d., and statistical significance was determined by the two-tailed Student's t-test; **P<0.01, *P<0.001. (e) Flow cytometric analysis of cell death induced by treatment with antibodies to CD40 and IgM in OCI-LY7 lymphoma cells that were transduced with a lentivirus containing vector alone or shKMT2D. (f) Proliferation of OCI-LY7 (KMT2D^{Wt}) and OCI-LY1 (KMT2D^{mut}) lymphoma cell lines upon stimulation with antibodies to CD40 alone or to both CD40 and IgM. Values correspond to the average of three experimental replicates relative to day 0±s.d. Two-tailed Student's t-test was used to determine statistical significance; *P<0.05, **P<0.01. (g,h) Viability assays in lymphoma cell lines upon stimulation with CD40-specific antibody for 96 h. Representative plots (g) and quantification (h), as analyzed by flow cytometry using annexin-V and DAPI exclusion. Bars correspond to the average of three experimental replicates ±s.d. Two-tailed Student's t-test was used to determine statistical significance. *P<0.05, **P<0.01. (i) TNFAPI3 (A20) (left) and NFKBIZ (right) mRNA levels in WT KMT2D-containing OCI-LY7, HT and SU-DHL4 and mutant KMT2D-containing OCI-LY1 and NU-DUL1 lymphoma cell lines upon stimulation with antibodies to CD40 alone or to both CD40 and IgM (24 h). Bars represent the mean of three biological replicates (two biological replicates for NU-DUL1 treated with antibodies to CD40+IgM; white bar)±s.d. Two-tailed Student's t-test was used to determine statistical significance; *P<0.05, **P<0.01. Red labels represent KMT2D-mutant cell lines and black labels represent cell lines with WT KMT2D.

FIG. 7 shows thatKmt2d deficiency accelerates B cell lymphoma development in mice. (a). Relative Kmt2d mRNA levels by qRT-PCR in FL512 mouse lymphoma cells transduced with vector or different shRNAs against KMT2D (#1 and #2). Bars represent mean of 2 biological replicates, error bars indicate standard deviation; **p<0.01, ***p<0.001 by two-tailed t-test. (b). Quantification of flow cytometry data showed in FIG. 1f. Values represent mean±SD (n=4 mice per genotype). Bars represent mean±SD (n=4 tumors per genotype). Two-tailed Student's t-test was used to determine statistical significance. No statistical significance was found. (c). High power image (100×) of H&E stained VavPBc12-vector and VavPBc12-shKmt2d lymphoma cells. (d). Representative histologic sections stained with H&E and immunohistochemical detection of B220+ lymphoma cells in the liver (left) and lung (right) of diseased mice with control (vector) and Kmt2d shRNA. Scale bars are 100 μm. (e). Tumor clonality analysis on VavPBc12/vector and VavPBcl2/sh-Kmt2d tumors, each lane corresponds to one tumor. PCR analysis of Vλ1-Jλ light chain rearrangements was performed on cDNA of B220+ lymphoma cells. (f). Table summarizing the results of the analysis of SHM in DNA from VavPBcl2/vector and VavPBcl2/sh-Kmt2d lymphomas. (g). Kaplan-Meier analysis of disease free survival of Kmt2d^{+/+} (Kmt2d^{+/+} CD19-Cre-=8, 3 females and 5 males), Kmt2d^{-/-} (Kmt2d^{f/f} CD19-Cre+, n=43, 22 females and 21 males) (p value Kmt2d^{+/+} vs Kmt2d^{-/-}=0.0158); AID-Tg (Kmt2d^{+/+}; IgκAID-Tg n=14, 6 females and 8 males) and Kmt2d^{-/-}; AID-Tg (Kmt2d^{f/f}; CD19-Cre+; AID-Tg n=7, 2 females and 5 males) cohorts. (p value AID-Tg vs Kmt2d^{-/-}; AID-Tg<0.0001). (h). Representative histologic sections stained with H&E and immunohistochemical detection of B220, CD3, Ki67, PNA and TUNEL of Kmt2d^{-/-} tumors. Asterisk represents red pulp infiltration by monotonous atypical B lymphocytes. (i). Representative FCM analysis of Kmt2d^{-/-} and Kmt2d^{-/-}; AID-Tg tumors, using antibodies against B220, IgM, IgD, IgL (Igκ+Igλ), CD19 and CD138 as indicated (see also Table 2). (j). Representative histologic sections stained with H&E and immunohistochemical detection of PNA, B220, Igκ, CD3, and Ki67, in Kmt2d−/−; AID-Tg tumors. (k). Schema of the IgH and Igκ loci showing restriction sites and probes used. (l). Southern blots showing clonal rearrangements in the JH (left) and Jκ (right) loci for the indicated tumors. (m). Southern blot analysis for detection of rearrangements in the Sμ region of DNA from indicated tumors, probes and restriction enzyme used are indicated at the bottom right of each panel. Position of the germ-line bands is shown. DNA from MEFS was used as control. Dotted lines represent the AID-induced DNA damage in switch regions during CSR. (n). Table summarizing the results of the analysis of SHM in DNA from Kmt2d^{-/-} and Kmt2d^{-/-}; AID-Tg tumors. The diagram on the top shows the region of the IgH locus used for PCR amplification and sequencing. Asterisks represent the mutations caused by AID in VDJ region during SHM.

FIG. 8 shows that KMT2D deficiency affects physiological B cell behavior (a). RNAseq analysis of KMT2D gene expression in different mature B cell populations from human tonsils. Each red dot represents a separate human tonsil and the mean expression is represented in TPM (transcripts per million). NB=Naïve B cells, CB=centroblasts, CC=centrocytes, TPC=Tonsil Plasma Cells, BMPC=Bone Marrow Plasma Cells, MEM=Memory cells. (b). Characterization of B cell populations in Kmt2d^{-/-} mice. Representative FCM analysis on wt and Kmt2d^{-/-} spleens to determine different B cell populations using antibodies against B220, IgM, CD5, CD23 and CD21 as indicated. TR: transitional, FO: follicular, MZ: marginal zone. (c). Table summarizing the total number of B cells and percentages for each B cell population relative to total number of live B220+ cells (7ADD−, B220+) in wt and Kmt2d^{-/-} spleens. (d). Characterization of B cell populations in Kmt2d^{-/-} mice. Representative FCM analysis on wt and Kmt2d^{-/-} spleens (same mice as in c,d) to determine different B cell populations using antibodies against B220, IgM, CD138, CD95 and GL7 as indicated, GC: Germinal center cells. (e). Table summarizing the total number of B cells and percentages for each B cell population relative to total number of live B220+ cells (7ADD−, B220+, except plasma cells) in wt and Kmt2d^{-/-} spleens. The percentage of plasma cells was calculated relative to total number of live cells (7ADD−). Values in (c) and (e) represent mean±SD (3 wt (2 females and 1 male) and 4 females Kmt2d^{-/-} were used; 4-5.5 months old). Two-tailed Student's t-test was used to determine statistical significance and was calculated using each population percentage.

FIG. 9 depicts the consequences of KMT2D mutations in human FL and DLBCL. (a). Table summarizing KMT2D mutations found in FL patients and the grade of the disease. Fisher's exact tests were performed in order to determine correlation between mutation type and grade. Overall, no significant correlation was found. (b). Percentage of cases with DLBCL carrying KMT2D mutations by type of mutation and DLBCL subtype (ABC: activated B cell; n=107; GCB: germinal center B cell; n=193). P value for nonsense mutations in GC versus ABC type=0.038 (*) by Fisher Exact test. (c) and (d) Kaplan-Meier curves representing disease specific survival (DSS) (c), and time to progression (TTP) (d) in years from DLBCL cases for three groups according to KMT2D mutation status (wt, n=215; nonsense mutation, n=37; missense mutation, n=43). Significance was estimated with the log-rank test. (e). Percentage of up or down-regulated genes in the top 100/200/350/500 differentially expressed genes in KMT2D^{mut} FL patients vs. KMT2D^{wt} FL patients (ranked by p-val). (f). Percentage in top 100/200/350/500/1073 differentially expressed genes and corresponding minimum p-val in KMT2D^{mut} FL patients vs. KMT2D^{wt} FL patients (ranked by p-val). (g). Percentage of up or down-regulated genes in top 100/200/350/500 differentially expressed genes in VavPBc12-shKmt2d vs. VavPBc12-vector B220+ lymphoma B cells (ranked by p-val). (h). Percentage of up or down-regulated genes in top 100/200/350/500/3210 differentially expressed genes and corresponding minimum p-val in VavPBc12-shKmt2d vs. VavPBc12-vector B220⁺ lymphoma B cells. (i). GSEA of differentially expressed genes ranked by log 2 fold change in KMT2Dmut FL samples versus KMT2Dwt FL samples compared to Plasma cell differentiation signature gene set. (j). GSEA of differentially expressed genes ranked by log 2 fold change in VavPBcl2/sh-Kmt2d vs. VavPBcl2/vector B220+ lymphoma B cells compared to Plasma cell differentiation signature gene set. NES, normalized enrichment score. FDR, false discovery rate.

FIG. 10 shows the epigenetic effects of KMT2D on target genes in mouse lymphomas. (a). Immunoblot of total lysates of B220⁺ lymphoma cells isolated from VavPbcl2-vector and VavPbcl2-shKmt2d tumors. (b) Quantification of global H3K4me1, H3K4me2 and H3K4me3 by ImageJ software. (c). Immunoblot of histone lysates of B220⁺ cells isolated from wild type and Kmt2d−/− mice. (d) Quantification of global H3K4me1, H3K4me2 and H3K4me3 by ImageJ software. (e) and (f). GSEA analysis of genes with a >25% reduction in H3K4me1/2 read density at promoters (p-value<0.05) in Kmt2d knockdown tumors compared to ranked log 2 fold change levels identified by RNA-seq in Kmt2d B220 knockdown tumors or KMT2Dmut FL patients. NES, normalized enrichment score. FDR, false discovery rate. (g). Pathway analysis of down-regulated genes with a >25% reduction in H3K4 me1/me2 read density at promoters (p<0.05) identified by GSEA leading edge analysis (n=321) in sh-Kmt2d B220+ tumors and KMT2Dmut FL patients compared to lymphoid signature database from the Staudt Lab (http://lymphochip.nih.gov/signaturedb/) and MysigDB. The background included around 24,000 genes from Refseq gene annotation. Statistical signficance was determined by hypergeometric tests and shown in the color key. The red color indicates (in log 10) the over-represented p-values and the blue shows under-representation (h). Normalized UCSC read density tracks of H3K4me1/me2 ChIP-seq peaks from MACS-sorted B220 positive lymphoma B cells in VavPBc12-vector (vector) and VavPBc12-shKmt2d (sh-Kmt2d) lymphomas for the indicated genes.

FIG. 11 depicts the identification of KMT2D target genes in human lymphoma cells. (a). Immunoblot of histone lysates from KMT2D wild type (HT, DOHH2, SU-DHL4) and KMT2D mutant (Toledo, Karpas422) DLBCL cell lines. (b). Quantification of global H3K4me1, H3K4me2 and H3K4me3 by ImageJ software. (c). Quantitative Mass spectrometry analysis of mono-, di-, tri-methylated histone H3K4. Represented as the percentage of global H3K4 post-translational modification (% PTM) in KMT2D wt (black) and KMT2D nonsense mutant (red) DLBCL cell lines (average of two biological replicates). (d). Normalized UCSC read density tracks of KMT2D ChIP-seq peaks in OCI-LY7 (black) and OCI-LY1 (red) and H3K4me1/2 ChIP-seq peaks in OCI-LY7 (black) and OCI-LY1 (red) for indicated genes.

FIG. 12 shows that KMT2D inactivation affects growth and survival pathways in lymphoma cells (a) and (b). Proliferation of isogenic OCI-LY7 (a) and SU-DHL4 (b) lymphoma cells transduced with vector control or an shRNA against KMT2D. Values represent mean of 3 replicates, error bars indicate standard deviation; *p<0.05, **p<0.01, ***p<0.001 by two-tailed t-test. (c). Relative mRNA levels by qRT-PCR of KMT2D targets in MACS-sorted B220⁺ lymphoma B cells from VavPBc12-vector (vector) and VavPBc12-shKmt2d (sh-Kmt2d) lymphomas. Bars represent mean of 4-5 biological replicates ±s.d; Two-tailed Student's t-test was used to determine statistical significance: *p<0.05, **p<0.01, or number indicating p-value. (d) and (e). Relative mRNA levels by qRT-PCR of KMT2D targets in isogenic OCI-LY7 (d) and SU-DHL4 (e) lymphoma cells transduced with vector or different shRNAs against KMT2D. Bars represent mean of 3-6 biological replicates, error bars indicate standard deviation; *p<0.05, **p<0.01, ***p<0.001 by two-tailed t-test. (f). Flow cytometry analysis of CD40 receptor expression (CD40r) in the indicated KMT2D wild type and mutant cell lines. Red line represents isotype control, blue line represents anti-CD40r. (g). Growth curves for indicated cell lines treated with anti-CD40 or anti-CD40/anti-IgM for 4 days. Data correspond to one representative assay from a total of 3 independent assays. (h). Gene expression analysis in KMT2D wild type or mutant lymphoma cell lines upon anti-CD40 or anti-CD40/IgM treatment for 24 h. Bars represent mean of 3 biological replicates (2 biological replicates for NU-DUL1 anti-CD40+IgM)±s.d. Two-tailed Student's t-test was used to determine statistical significance *p<0.05, **p<0.01, ***p<0.001 or number indicating p-value. Red labels represent KMT2Dmut cell lines and black labels represent KMT2Dwt cell lines.

FIG. 13 is a schematic diagram indicating KMT2D target genes in relation to the affected signaling pathways. KMT2D targets identified by direct ChIP binding and verified by knockdown are marked by a star. These targets are both positive and negative regulators of IL21, BCR, and CD40 signaling pathways.

### DETAILED DESCRIPTION OF THE INVENTION

The invention relates generally to methods for diagnosis and treatment of follicular lymphoma. Specifically, the invention relates to detecting the presence of, or the normal or an altered presence, activity, or expression of lysine (K)-specific methyltransferase 2D (KMT2D) to diagnose or treat follicular lymphoma.

The gene encoding the lysine-specific histone methyltransferase KMT2D has emerged as one of the most frequently mutated genes in follicular lymphoma and diffuse large B cell lymphoma; however, the biological consequences of KMT2D mutations on lymphoma development are not known. In one embodiment, KMT2D is shown to function as a bona fide tumor suppressor and that its genetic ablation in B cells promotes lymphoma development in mice. In one embodiment, KMT2D deficiency also delays germinal center involution and impedes B cell differentiation and class switch recombination. Integrative genomic analyses indicate that KMT2D affects methylation of lysine 4 on histone H3 (H3K4) and expression of a specific set of genes, including those in the CD40, JAK-STAT, Toll-like receptor and B cell receptor signaling pathways. Other KMT2D target genes include frequently mutated tumor suppressor genes such as TNFAIP3, SOCS3 and TNFRSF14. In one embodiment, KMT2D mutations promote malignant outgrowth by perturbing the expression of tumor suppressor genes that control B cell-activating pathways.

Thus, the inventors of the instant application have surprisingly and unexpectedly found that KMT2D is a bona fide tumor suppressor and KMT2D deficiency promotes follicular lymphoma development in vivo. In addition, the inventors have surprisingly and unexpectedly found that KMT2D mutations contribute to lymphoma development. Furthermore, the inventors found that the presence of a KMT2D alteration adversely affects the normally tumor suppressive effects of anti-CD40, thereby reducing the effectiveness of anti-CD40 therapies when an alteration in KMT2D is present or potentially stimulating disease progression thereby. This finding is useful to help determine the response or responsiveness of a patient's tumor to a particular therapy, or lack thereof, thereby guiding the optimal course of therapy for a patient with follicular lymphoma, in particular whether antiCD40 or related therapy may be effective, or should be avoided because patients may do worse with such treatment. Thus, in one embodiment, a patient with a KMT2D alteration may not be effectively treated with anti-CD40 therapy. In another embodiment, anti-CD40 therapy is contraindicated in a patient found to have a KMT2D alteration. In another embodiment, methods for treating follicular lymphoma include a determination of KMT2D alteration and guiding therapy away from anti-CD40 in the presence of an altered KMT2D. In any of the foregoing embodiments, the guidance for the use or non-use of anti-CD40 therapy may be in conjunction with the respective use or non-use of anti-IgM therapy.

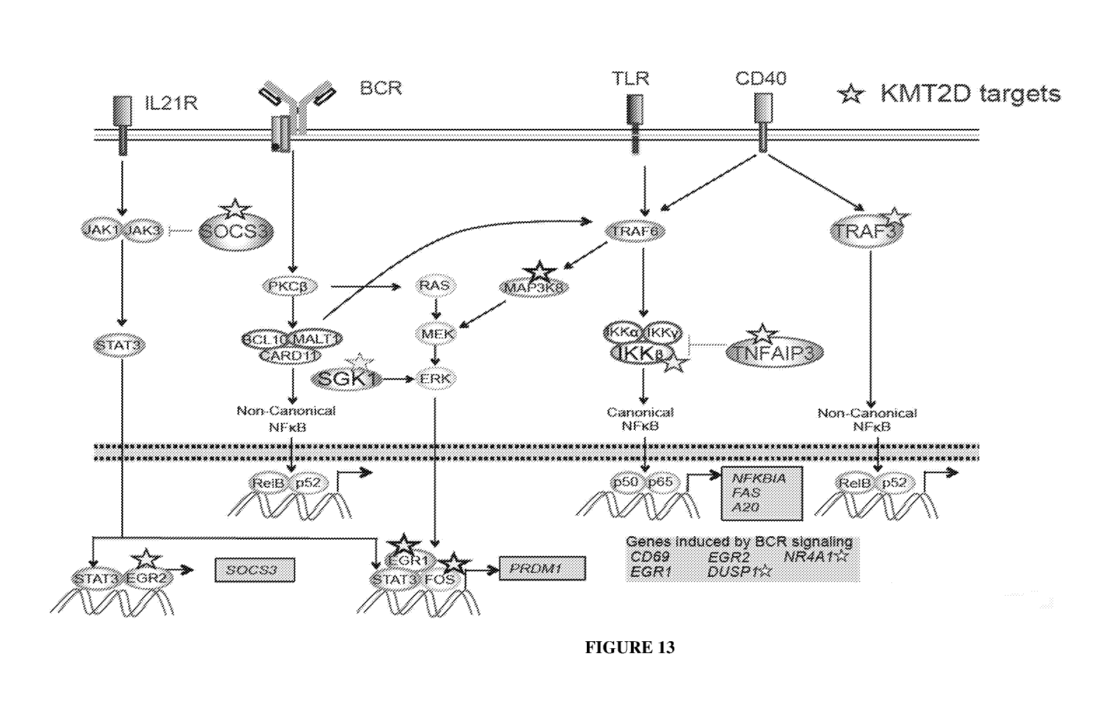

The results described herein establish the tumor suppressor function of KMT2D in germinal center B cells. The H3K4 methyltransferase KMT2D is one of the most frequently mutated genes in DLBCL and FL^{3,4}, and we show that it controls the expression of multiple key regulators of the CD40, TLR and BCR signaling pathways (FIG. 13). Bona fide KMT2D target genes include lymphoid tumor suppressor genes such as TNFAIP3, SOCS3, SGK1, TRAF3, TNFRSF14 and ARID1A^{15,16,21} KMT2D also contributes to the normal B cell response, and KMT2D-deficient mice show an abnormal persistence of germinal centers, a defect in class switch recombination and reduced antibody production reminiscent of the reported immune defect seen in the heritable Kabuki syndrome, which has been most often linked to KMT2D mutations. Collectively these data show that KMT2D somatic mutations may drive GC expansion due to enhanced proliferation and impaired terminal differentiation of B cells and to loss of H3K4 mono- and dimethylation at key B cell enhancer regions and some promoters. Our results are consistent with genomic evidence indicating that KMT2D mutations are early lesions in GC lymphomas^{3,4}. Notably, even in the absence of Bcl2 activation, KMT2D deficiency is sufficient to trigger B cell malignancy in mice. Clinically, KMT2D mutations are not associated with the outcome of R-CHOP chemotherapy in DLBCL. However, it is not yet known how KMT2D status would affect the responses of lymphomas to targeted signal inhibitors that are entering the clinic. In this regard, our results indicate the deregulation of multiple immune signaling pathways in KMT2D-mutant lymphoma cells and the altered responses to CD40 and BCR activation. Recently, histone deacetylase (HDAC) inhibitors were shown to ameliorate the developmental defects in a model of Kabuki syndrome²². Similarly, inhibition of H3K4 demethylase activities, such as those of JARID1 and LSD1, may be able to reverse some of the epigenetic changes seen in KMT2D-deficient lymphomas²³.

Therapy or immunotherapy in one embodiment is B cell therapy. Therapy or immunotherapy in another embodiment is anti-CD40 antibody, anti-CD20 antibody or anti-IgM therapy, or any combination thereof.

The terms “KMT2D alteration,” as used herein, refer to any genetic change in KMT2D structure or its molecular expression. In one aspect, KMT2D alteration refers to a mutation in KMT2D. In another aspect, KMT2D alteration refers to a change in the expression level of KMT2D mRNA or KMT2D protein, or activity of the KMT2D protein, relative to a predetermined level (i.e., control level) of a healthy subject. Activity of the KMT2D protein may be enzymatic activity or histone binding activity, by KMT2D directly or by proteins associated with or complexed therewith. Activity may also include regulation of gene transcription activity.

The terms “mutation,” as used herein, refer to the presence of a mutation in KMT2D. In one aspect, the mutation refers to a change in the KMT2D gene with respect to the standard wild-type sequence. Mutations can be inherited, or they can occur in one or more cells during the lifespan of an individual. In some embodiments, the KMT2D mutation is homozygous. In other embodiments, the KMT2D mutation is heterozygous. The KMT2D mutation can be any type of mutation, for example, but not limited to, a non-sense mutation, a missense mutation, an insertion mutation, a deletion mutation, a replacement mutation, a point mutation, or a combination thereof.

As used herein, a “biological sample” is a sample that contains cells or cellular material. Non-limiting examples of biological samples include urine, blood, plasma, serum, cerebrospinal fluid, pleural fluid, sputum, peritoneal fluid, bladder washings, secretions (e.g., breast secretion), oral washings, tissue samples, tumor samples, touch preps, or fine-needle aspirates. A biological sample can be obtained using any suitable method. For example, a blood sample (e.g., a peripheral blood sample) can be obtained from a subject using conventional phlebotomy procedures. Similarly, plasma and serum can be obtained from a blood sample using standard methods.

KMT2D protein of the invention may comprise the amino acid sequence set forth in SEQ ID NO.: 1 (GenBank Accession No.: AAC51734.1). In one example, KMT2D protein comprises a homolog, a variant, an isomer, or a functional fragment of SEQ ID NO: 1. In another example, the amino acid sequence is approximately 60%, 70%, 80%, 85%, 90%, 95%, 98%, or 99% identical to SEQ ID NO.: 1. Each possibility represents a separate embodiment of the present invention.

KMT2D protein of the invention may be encoded by the nucleic acid sequence set forth in SEQ ID NO.: 2 (GenBank Accession No.: AF010403.1). In one example, KMT2D nucleic acid sequence comprises a homolog, a variant, an isomer, or a functional fragment of SEQ ID NO: 2. In another example, the nucleic acid sequence is approximately 60%, 70%, 80%, 85%, 90%, 95%, 98%, or 99% identical to SEQ ID NO.: 2. Each possibility represents a separate embodiment of the present invention.

In one aspect, the invention provides methods for detecting the KMT2D mutation. The KMT2D mutation in a sample can be detected using any technique that is suitable for detecting a mutation or genetic variation in a biological sample. Suitable techniques for detecting mutations or genetic variations in cells from a biological sample are well known to those of skill in the art. Examples of such techniques include, but are not limited to, PCR, Southern blot analysis, microarrays, and in situ hybridization. In a particular embodiment, a high-throughput system, for example, a microarray, is used to detect the KMT2D mutation.

In one aspect, nucleic acids can be isolated from the biological sample. The isolated nucleic acids can include a KMT2D nucleic acid sequence. In some embodiments, the KMT2D nucleic acid sequence can include a nucleotide sequence variant of SEQ ID NO: 2. As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-KMT2D proteins). The term “isolated” as used herein with respect to nucleic acids also includes any non-naturally-occurring nucleic acid sequence since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

The nucleic acid molecules provided herein can be between about 8 and about 15,789 nucleotides in length. In one example, a nucleic acid can be 8, 9, 10, 11, 12, 13, 14, 15, 16, 17, 18, 19, 20, 21, 22, 23, 24, 25, 26, 27, 28, 29, 30, 32, 34, 36, 38, 40, 45, or 50 nucleotides in length. Alternatively, the nucleic acid molecules provided herein can be greater than 50 nucleotides in length (e.g., 55, 60, 65, 70, 75, 80, 85, 90, 95, 100, 500 or more than 500 nucleotides in length). Nucleic acid molecules can be in a sense or antisense orientation, can be complementary to a KMT2D reference sequence (e.g., the sequence shown in GenBank Accession No. AF010403.1), and can be DNA, RNA, or nucleic acid analogs. Nucleic acid analogs can be modified at the base moiety, sugar moiety, or phosphate backbone to improve, for example, stability, hybridization, or solubility of the nucleic acid.

The isolated nucleic acid molecules provided herein can be produced using standard techniques including, without limitation, chemical synthesis.

Nucleic acids of the invention can also be isolated using a commercially available kit. In one embodiment, DNA from a peripheral blood sample can be isolated using a DNeasy DNA isolation kit, a QIAamp DNA blood kit, or a PAXgene blood DNA kit from Qiagen Inc. (Valencia, Calif.). DNA from other tissue samples also can be obtained using a DNeasy DNA isolation kit. Any other suitable DNA extraction and purification technique also can be used, including liquid-liquid and solid-phase techniques ranging from phenol-chloroform extraction to automated magnetic bead nucleic acid capture systems.

In one aspect, once nucleic acid has been obtained, it can be contacted with at least one oligonucleotide (e.g., a primer) that can result in specific amplification of a mutant KMT2D gene, if the mutant KMT2D gene is present in the biological sample. In another embodiment, the nucleic acid also can be contacted with a second oligonucleotide (e.g., a reverse primer) that hybridizes to either a mutant or a wild-type KMT2D gene. The nucleic acid sample and the oligonucleotides can be subjected to conditions that will result in specific amplification of a portion of the mutant KMT2D gene if the mutant KMT2D gene is present in the biological sample.

Once the amplification reactions are completed, the presence or absence of an amplified product can be detected using any suitable method. Such methods include, without limitation, those known in the art, such as gel electrophoresis with or without a fluorescent dye (depending on whether the product was amplified with a dye-labeled primer), a melting profile with an intercalating dye, and hybridization with an internal probe. Alternatively, the amplification and detection steps can be combined in a real time PCR assay. In some embodiments, the detection of an amplified product indicates that cells containing the KMT2D mutation were present in the biological sample, while the absence of an amplified product indicates that cells containing the KMT2D mutation were not present in the biological sample.

In another aspect, the methods provided herein also can include contacting the nucleic acid sample with a third oligonucleotide that can result in specific amplification of a wild-type KMT2D gene without detectable amplification of a mutant KMT2D. These methods can further include subjecting the nucleic acid and the oligonucleotides to conditions that will result in specific amplification of a wild-type KMT2D sequence if a wild-type KMT2D gene is present in the biological sample. The presence or absence of an amplified product containing a wild-type KMT2D sequence can be detected using any suitable method, including those disclosed above. Methods that include using oligonucleotides for amplification of both mutant and wild-type KMT2D sequences also can include quantifying and comparing the amounts of amplified product for each sequence. The relative levels of mutant and wild-type products can indicate the fraction of cells in the biological sample that contain a mutant KMT2D gene.

In some embodiments, the methods disclosed herein can further include a first, universal amplification step. Such methods can include contacting nucleic acids obtained from a biological sample with, for example, a cocktail of degenerate primers, and using standard PCR procedures for an overall amplification of the DNA. This preliminary amplification can be followed by specific amplification and detection of products, as described herein.

In another embodiment, the KMT2D mutation is detected by Southern blot hybridization. Suitable probes for Southern blot hybridization of a given sequence can be produced from the nucleic acid sequences of the KMT2D. Methods for preparation of labeled probes, and the conditions for hybridization thereof to target nucleotide sequences, are well known in the art and are described in Molecular Cloning: A Laboratory Manual, J. Sambrook et al., eds., 2nd edition, Cold Spring Harbor Laboratory Press, 1989, Chapters 10 and 11.

In another embodiment, the KMT2D mutation can be detected by a technique of in situ hybridization. This technique requires fewer cells than the Southern blotting technique, and involves depositing whole cells onto a microscope cover slip and probing the nucleic acid content of the cell with a solution containing radioactive or otherwise labeled nucleic acid probes. This technique is particularly well-suited for analyzing tissue biopsy samples from subjects. The practice of the in situ hybridization technique is described in more detail in U.S. Pat. No. 5,427,916, the disclosure of which is incorporated herein by reference. In an exemplary embodiment, the in situ hybridization technique is a FISH (fluorescent in situ hybridization) technique.

In another embodiment, detection the KMT2D mutation, for example, a mutation in KMT2D, can be accomplished by micro array techniques. The microarray may be fabricated using techniques known in the art. For example, probe oligonucleotides of an appropriate length are 5′-amine modified and printed using commercially available microarray systems, e.g., the GENEMACHINE, OMNIGRID 100 MICROARRAYER and AMERSHAM CODELINK activated slides. The microarray can be processed by direct detection of the tagged molecules using, e.g., STREPTAVIDIN-ALEXA647 conjugate, and scanned utilizing conventional scanning methods.

Other techniques for detecting the KMT2D mutation are also within the skill in the art, and include various techniques for detecting genetic variations.

In another aspect, KMT2D alteration is detected by measuring a change in the expression level of KMT2D mRNA or KMT2D protein, relative to a predetermined level (i.e., control level) of a healthy subject.

In one example, the invention features agents which are capable of detecting KMT2D polypeptide or mRNA such that the presence of KMT2D is detected. As defined herein, an “agent” refers to a substance which is capable of identifying or detecting KMT2D in a biological sample (e.g., identifies or detects KMT2D mRNA, KMT2D DNA, KMT2D protein, KMT2D activity). In one embodiment, the agent is a labeled or labelable antibody which specifically binds to KMT2D polypeptide. As used herein, the phrase “labeled or labelable” refers to the attaching or including of a label (e.g., a marker or indicator) or ability to attach or include a label (e.g., a marker or indicator). Markers or indicators include, but are not limited to, for example, radioactive molecules, colorimetric molecules, and enzymatic molecules which produce detectable changes in a substrate.

In one embodiment the agent is an antibody which specifically binds to all or a portion of a KMT2D protein. As used herein, the phrase “specifically binds” refers to binding of, for example, an antibody to an epitope or antigen or antigenic determinant in such a manner that binding can be displaced or competed with a second preparation of identical or similar epitope, antigen or antigenic determinant. In an exemplary embodiment, the agent is an antibody which specifically binds to all or a portion of the human KMT2D protein.

In yet another embodiment the agent is a labeled or labelable nucleic acid probe capable of hybridizing to KMT2D mRNA. For example, the agent can be an oligonucleotide primer for the polymerase chain reaction which flank or lie within the nucleotide sequence encoding human KMT2D. In a preferred embodiment, the biological sample being tested is an isolate, for example, RNA. In yet another embodiment, the isolate (e.g., the RNA) is subjected to an amplification process which results in amplification of KMT2D nucleic acid. As defined herein, an “amplification process” is designed to strengthen, increase, or augment a molecule within the isolate. For example, where the isolate is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Detection of RNA transcripts may be achieved by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR). Any suitable known amplification method known to one skilled in the art can be used. In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

In another aspect of the invention pertains to measuring a change in the level of KMT2D protein, for example, using anti-KMT2D antibodies. The term “antibody” as used herein refers to immunoglobulin molecules and immunologically active portions of immunoglobulin molecules, i.e., molecules that contain an antigen binding site which specifically binds (immunoreacts with) an antigen, such as KMT2D. The invention provides polyclonal and monoclonal antibodies that bind KMT2D.

It is generally preferred to use antibodies, or antibody equivalents, to detect KMT2D protein. Methods for the detection of protein are well known to those skilled in the art, and include ELISA (enzyme linked immunosorbent assay), RIA (radioimmunoassay), Western blotting, and immunohistochemistry. Immunoassays such as ELISA or RIA, which can be extremely rapid, are more generally preferred.

Immunohistochemistry may also be used to detect expression of human KMT2D in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

The invention also encompasses kits for detecting the presence of KMT2D in a biological sample. In one aspect, the kit can comprise a labeled or labelable agent capable of detecting KMT2D or its mutation. In another aspect, the kit can comprise a labeled or labelable agent capable of detecting KMT2D protein or mRNA in a biological sample and a means for determining the amount of KMT2D in the sample. The kit may also include instructions for the detections.

The step of detection of the invention can be performed prior to or after a treatment by one or more therapeutic modalities, for example, but not limited to, an immunotherapy, a chemotherapy, a radiation therapy, and a combination thereof. Therapy in one embodiment is B cell therapy, such as but not limited to anti-CD40 antibody, anti-CD20 antibody or anti-IgM therapy, or any combination thereof. In one embodiment, the detection step is performed prior to administering an antibody (e.g., an anti-CD40 antibody, an anti-CD20 antibody—rituximab) to treat a follicular lymphoma. Coadministration with anti-IgM is also embodied herein. In another embodiment, the detection step is performed after administering an antibody to treat a follicular lymphoma. In another embodiment, the detection step is performed prior to administering a chemotherapy agent to treat a follicular lymphoma. In another embodiment, the detection step is performed after administering a chemotherapy agent to treat a follicular lymphoma. In another embodiment, the detection step is performed prior to a radiation therapy to treat a follicular lymphoma. In another embodiment, the detection step is performed after a radiation therapy to treat a follicular lymphoma.

In another aspect, provided herein is a method of determining a treatment outcome for treating a follicular lymphoma, in a subject, the method comprising the steps of: obtaining a biological sample from said subject; and testing said biological sample to detect the presence or absence of a KMT2D alteration in said biological sample, wherein the presence of said KMT2D alteration indicates a response (e.g., a tumor response) to a therapy, thereby determining said treatment outcome for treating said follicular lymphoma in said subject. In another aspect, provided herein is a method for treating a follicular lymphoma, in a subject, the method comprising: (a) obtaining a biological sample from said subject; and testing said biological sample to detect the presence or absence of a KMT2D alteration in said biological sample, wherein the presence of said KMT2D alteration indicates a response to a therapy; (b) based on the determination of said tumor response to said therapy, administering an effective amount of a therapeutic agent to treat said follicular lymphoma, thereby treating said follicular lymphoma in said subject. In all embodiments herein, a response may include a lack of a response.

As noted herein above, the presence of a KMT2D alteration adversely affects the normally tumor suppressive effects of anti-CD40, thereby reducing the effectiveness of anti-CD40 therapies when an alteration in KMT2D is present or potentially stimulating disease progression thereby. Therefore, a response to therapy relates to, in one embodiment, whether antiCD40 or related therapy may be effective, or should be avoided because patients may do worse with such treatment. Thus, in one embodiment, a patient with a KMT2D alteration may not be effectively treated with anti-CD40 therapy. In another embodiment, anti-CD40 therapy is contraindicated in a patient found to have a KMT2D alteration. In another embodiment, methods for treating follicular lymphoma include a determination of KMT2D alteration and guiding therapy away from anti-CD40 in the presence of an altered KMT2D. In any of the foregoing embodiments, the guidance for the use or non-use of anti-CD40 therapy may be in conjunction with the respective use or non-use of anti-IgM therapy. In another embodiment, an effective therapeutic agent to treat follicular lymphoma may be one or more agents excluding anti-CD40, anti-CD20 or anti-IgM therapy (and any combination thereof) but other chemotherapeutic agents such as but not limited to cyclophosphamide, vincristine, prednisone, doxorubicin, bortezomib, everolimus, idelalisib, ibrutinib, lenalidomide, ofatumumab, or panobinostat, or combinations thereof, by way of non-limiting examples.

In yet another aspect, provided herein is a method for treating a follicular lymphoma in a subject, the method comprising: administering to said subject a molecule that effectively enhances the level of a KMT2D in said subject, thereby treating said follicular lymphoma in said subject.

As used herein, “response” can refer to the outcome or responsiveness, or predicted outcome or responsiveness, of a patient's disease or cancer to a particular therapy, i.e., whether the patient will benefit from or the cancer will be treated by the therapy, whether the patient or cancer will have little or no effect from the therapy, or whether the therapy may exacerbate the disease or cause the patient to do worse as a result of use of a particular therapy. In one embodiment, a response can mean no response or a lack of a response.

As used herein, the terms “treat” and “treatment” refer to therapeutic treatment, wherein the object is to prevent or slow down (lessen) an undesired physiological change associated with a disease or disorder. Beneficial or desired clinical results include alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. “Treatment” can also mean prolonging survival as compared to expected survival if not receiving treatment. Those in need of treatment include those already with the disease or disorder as well as those prone to having the disease or disorder.

In one aspect, the treatment includes administering a KMT2D protein. In another aspect, the treatment includes administering a nucleic acid sequence encoding the KMT2D protein. In yet another aspect, the treatment includes administering an agent that enhances the activity of KMT2D.

The treatment compositions of the invention may be administered alone (monotherapy), or in combination with one or more therapeutically effective agents or treatments (combination therapy).

Cancers treated by the invention include, but are not limited to, a Grade 1, 2, or 3 follicular lymphoma and a Stage 1, 2, 3, or 4 follicular lymphoma.

In another aspect, provided herein is a method for identifying a molecule that effectively treats a follicular lymphoma in a subject, the method comprising: providing a plurality of molecules; and screening said plurality of molecules to identify a molecule that effectively enhances the level of a KMT2D, thereby identifying said molecule that effectively treats said follicular lymphoma in said subject.

The terms “subject” and “individual” are defined herein to include animals, such as mammals, including but not limited to, primates, cows, sheep, goats, horses, dogs, cats, rabbits, guinea pigs, rats, mice or other bovine, ovine, equine, canine, feline, rodent, or murine species.

All patents and literature references cited in the present specification are hereby incorporated by reference in their entirety.

The following examples are presented in order to more fully illustrate the preferred embodiments of the invention. They should in no way be construed, however, as limiting the broad scope of the invention.

### EXAMPLES

### Example 1. Methods

Measurement of KMT2D mRNA Expression in Human B Cells.

The human tonsil and bone marrow samples were obtained in Pamplona (Spain) at the Clinica Universidad de Navarra and the obtention of these samples was approved by the ethical committee of Clinica Universidad de Navarra (Spain). Cells from tonsils and bone marrow were immunophenotyped using eight-color antibody combination: CD20-Pacific Blue (PB), CD45-Oranje Chrome 515 (00515), CD38-fluorescein isothiocyanate (FITC), CXCR4-phycoerythrin (PE), CD3-peridinin chlorophyll protein-cyanin 5.5 (PerCP-Cy5.5), CD10-PE-cyanin 7 (PE-Cy7), CD27-allophycocyanin (APC) and CD44-APCH7 aimed at the identification and high-purity (□97%) FACS-sorting (FACSAria II, Becton Dickinson Biosciences, San Jose, Calif.) of the following B cell (CD3−CD20+CD45+) subsets (after careful exclusion of CD3+CD20−CD45+ T cells): naive B cells (CD10−CD44+CD27−CD38−), germinal center (CD10+CD44loCD38+) centrocytes (CXCR4−) and centroblasts (CXCR4+), memory B cells (CD10−CD27+CD44+) and new-born plasmablasts (CD10−CD27hiCD38hiCD44hi). The strand-specific RNA-seq was performed in naive B cells (n=5 samples), centroblasts (n=7), centrocytes (n=7), memory cells (n=8), tonsilar plasma B cells (n=5) and purified plasma B cells from bone marrow of healthy donors (n=3). Each red dot represents a separate human tonsil and the mean expression is represented in TPM (transcripts per million).

Characterization of Human FL Samples.

The Institutional Review Board (IRB) of Weill Cornell Medical College (IRB#0107004999) approved the study protocol. The specimens were derived from excess diagnostic materials that were banked in the lymphoma repository. A waiver of informed consent has been obtained for this retrospective study. The IRB-approved protocol permitted association of these specimens with a particular individual, allowing review of the medical records for the minimum information necessary to complete the study. All of the data that were provided to investigators were stripped of protected health information.

Sample preparation. Frozen single-cell suspensions of individual tumor samples were first thawed in a 37° C. water bath and then resuspended in RPMI+10% FBS and incubated in an incubator (37° C. and 5% CO2) for 1 h. Half of the sample was used to isolate B cells by using EasySep Human B Cell Enrichment Kit (STEMCELL Technologies, Vancouver, Canada), and the other half was used to isolate T cells with Easy Sep Human T Cell Isolation Kit. DNA was extracted from isolated cell populations by using PureLink Genomic DNA kit (LifeTechnologies, Grand Island, N.Y.). Total RNA was extracted using the Qiagen RNeasy Mini Kit (Valencia, Calif.). The quantity of DNA and RNA samples was measured by a Qubit Fluorometer (LifeTechnologies, Grand Island, N.Y.), and the quality of DNA and RNA samples was assessed by a bioanalyzer (Agilent Technologies, Santa Clara, Calif.).

Exome sequencing. For each tumor sample and the respective T cell control sample, 3 □g of high-molecular-weight genomic DNA was used to prepare exome sequencing libraries using the Aglient SureSelectXT Human All Exon 50 Mb Target Enrichment System for Illumina Pair-End Sequencing Library kit (Agilent Technologies, Santa Clara, Calif.). Each library was sequenced on one entire lane of a flow cell on an Illumina HiSeq 2000. Sequence information of 75 bp on each end of the DNA library fragment (PE75) was collected.

Targeted resequencing. A targeted-enrichment panel was designed by RainDance Technologies (Billerica, Mass.) for 36 of the most commonly mutated lymphoma genes including, ARID1A, ATP6AP1, B2M, BCL2, BCL6, BTG1, BTG2, CARD11, CD79B, CREBBP, EB1, EEF1A1, EP300, EZH2, GNAl3, HIST1H1B, HIST1H1C, HVCN1, IRF4, IRF8, KLHL6, KMT2D, MEF2B, MYD88, PCGFS, PDSSA, PIM1, POU2F2, PRDM1, SGK1, STAT6, SZT2, TBL1XR1, TNFAIP3, TP53 and XPOT. The entire coding regions of this set of genes were targeted by overlapping PCR amplicons averaging 200 bp. DNA (200 ng) was first sheared to around 3 kb by using a Covaris S220 Focused ultrasonicator (Woburn, Mass.) and then merged with primer pairs in a picoliter-droplet format on a Raindance ThunderStorm system. Targeted regions were amplified with the addition of specific tailed primers. A second round of PCR was performed to add indexed adaptor sequences for Illumina sequencing. Final indexed products from 48 samples were multiplexed together and sequenced on one entire lane of flow cell on Illumina HiSeq 2500 by using the fast mode setting. Sequence information of 100 bp on each end of the library fragment (PE100) was collected.

Discovery of single-nucleotide variants (SNV). Sequencing reads were aligned to human genome assembly GRCh37/hg19 using the BWA aligner24. After filtering duplicated paired reads, variants were detected as previously described25-27. Novel coding region SNVs were defined as those that were not present in SNP132. These SNVs were then further filtered by sequencing depth (□20×) and variant percentage (□25%). To obtain the list of somatic mutations in each tumor sample, we compared the variant ratio of each novel coding SNV between tumor B cells and their respective control T cells and estimated the statistical significance of the difference using a chi-squared test, corrected with multiple hypothesis testing (Benjamini-Hochberg corrected P<0.1).

Characterization of DLBCL Samples.

We analyzed 347 newly diagnosed DLBCL cases, in which individuals were treated with R-CHOP (given with curative intent) at the BC Cancer Agency (Vancouver). Subject sample use was approved by the University of British Columbia, British Columbia Cancer Agency, Research Ethics Board (REB #H13-01478). The cases were selected on the basis of the following criteria: 16 years of age or older; histologically confirmed de novo DLBCL according to the 2008 WHO classification; available DNA extracted from fresh-frozen biopsy material (tumor content >30%). All cases were centrally classified by A.M. and R.D.G., who were blinded for sample identity to determine the diagnosis. Individuals were excluded if they were younger than 16 years old and had DLBCL that was not de novo DLBCL (primary mediastinal large B cell lymphoma, primary central nervous system lymphoma and a previous diagnosis of an indolent lymphoproliferative disorder) and positive HIV serology.

Targeted Resequencing in DLBCL Samples.

Targeted resequencing of the coding exons of KMT2D in 347 DLBCL cases was performed using a Truseq Custom Amplicon assay (Illumina) and libraries were run on the MiSeq (Illumina). Mutation calling was done with Mutascope pipeline. Cell of origin (COO) classification was available in 331 cases according to gene expression profiling by the Lymph2Cx assay using the NanoString platform²⁸ in 299 subjects, as well as Hans algorithm²⁹ in 32 cases with low tumor content. 194 cases were assigned to GCB subtype, 107 cases to the ABC (non-GCB) subtype and 30 were unclassifiable.

Correlation Between KMT2D Mutation Status with Disease Progression and Survival. Baseline characteristics were compared between the groups with KMT2D mutation type using the chi-squared test.

We measured the endpoints from the time of the initial pathologic diagnosis to the following events: overall survival (OS; the date of death from any cause or to the last follow-up); progression-free survival (PFS; the date of progression, relapse or death from any cause); disease-specific survival (DSS; the date from lymphoma or acute treatment toxicity) and time-to-progression (TTP; the date of progression, relapse or death from lymphoma or acute treatment toxicity). OS, PFS, DSS and TTP were estimated using the Kaplan-Meier method and differences in outcome between groups were assessed using the log-rank test. Two-sided P<0.05 was considered significant. Data were analyzed using SPSS software (SPSS version 14.0; SPSS Inc, IL).

Generation of Mice.

Kmt2d^{fl/fl} mice were previously described⁷ and here we bred them with CD19-Cre mice (Jackson no. 006785) where Cre is expressed from the pre-B cell stage and removes exons 16-19 of Kmt2d causing an open reading frame shift that creates a stop codon in exon 20. Kmt2d^{fl/fl}×CD19-Cre mice were maintained in a mixed C57BL/6; 129 background. Mice were monitored for tumor formation once a week for the first 4 months and every day after then. All mice were housed in the Frederick National Laboratory and treated with procedures approved by the US National Institutes of Health (NIH) Animal Care and Use Committee.

The VavP-Bcl2 mouse model of FL⁹ was adapted to the adoptive transfer approach using retrovirally transduced HPCs. HPC isolation and transduction were performed as in ref. 30. 8- to 10-week-old lethally irradiated (4.5 Gy twice) C57BL/6 females were used as recipients for all transplantation experiments. shRNAs to mouse Kmt2d were designed using Designer of Small Interfering RNA (DSIR, http://biodev.extra.cea.fr/DSIR/) and are based on MSCV³¹: shKmt2d #1 (mouse), GACTGGTCTAGCCGATGTAAA (SEQ ID NO:20) and shKmt2d #2 (mouse), TGAATCTTTATCTTCAGCAGG (SEQ ID NO:21).

Mouse B220⁺ Tumor Sample Preparation.

B220⁺ cells were purified from mouse lymphoma tumors by immunomagnetic enrichment with CD45R (B220) microbeads (Miltenyi Biotech). RNA extraction was performed using TRIzol (Ambion) using the manufacturer's protocol.

Histology.

Mouse tissues were fixed overnight in formalin, embedded in paraffin blocks and sectioned. Tissue sections were stained with hematoxilin and eosin (H&E) or with Ki67, TUNEL, B220 or PNA following standard procedures^{32,33}.

Flow Cytometric Analysis.

Vavp-Bcl2 Tumors.

Tumor cell suspensions of representative tumors for each genotype were stained as described³⁰. The antibodies used were B220 (CD45R; BD PharMingen, #553092) or IgG1 (BD PharMingen #560089), which were conjugated with APC, and to B220 (CD45R; BD PharMingen, #553090), CD19 (BD PharMingen, #557399), IgM (PharMingen, #553409), Thy1 (CD90; Cedarlane, #CL8610PE), CD8 (PharMingen, #553032), Sca-1 (PharMingen, #553108), IgD (BD PharMingen #558597) and GL7 (BD PharMingen #561530), which were conjugated with phycoerythrin. Analysis was performed with a BD LSRFortessa cell analyzer and FlowJo software (Tree Star).

Kmt2d^{-/-} Tumors.

Single-cell suspensions were obtained from spleens according to standard procedures. Red blood cells were lysed with ACK Lysing Buffer (Quality Biological) and surface markers on tumor cells were analyzed on FACSCalibur (BD Biosciences) using the following fluorochrome-cojugated antibodies: IgM-PE (BD Pharmingen, clone R6-60.2 #553409), IgM-FITC (BD Pharmingen, clone R6-60.2 #553408), IgD-FITC (BD Pharmingen, clone 11-26c.2a #553439), FITC-conjugated Ig, λ1, λ2 and λ3 (BD Pharmingen, clone R26-46 #553434), Igκ-FITC (BD Pharmingen, clone 187.1 #550003), CD19-APC (BD Pharmingen, clone 1D3 #550992), B220-PE (BD Pharmingen, clone RA3-6B2 #553090), B220-PE (BD Pharmingen, clone RA3-6B2 #553088), CD138-PE (BD Pharmingen, clone 281.2 #553714), CD24-FITC (BD Pharmingen, clone M1/69 #553261), CD11b-APC (BD Pharmingen, clone M1/70 #553312), CD4-PE (Biolengend, clone GK1.5 #100408), CD8-FITC (BD Pharmingen, clone 53-6.7 #553031), CD3-PE (BD Pharmingen, clone 500A2 #553240), and CD43-biotin (BD Pharmingen, clone S7 #553269) and B220-biotin (BD Pharmingen RA3-6B2 #553085) followed by Streptavidin-APC (BD Pharmingen). Analysis was performed with FlowJo software (Tree Star).

Characterization of Nonmalignant B Cell Populations in Kmt2d^{-/-} Mice.

To identify the different B cell populations, two stains were performed in splenocytes from 4- to 5.5-month-old mice (two female and one male wild-type mice and four female Kmt2d^{-/-} mice). First, to identify transitional, follicular and marginal zone populations, cells were stained with the following antibodies: CD21-FITC (Biolegend, clone 7E9, #123407), CD5-PE (eBioscience, clone 53-7.3 #12-0051-81), CD23-PECY7 (Biolegend, clone B3B4 #101613), IgM-APC (Biolegend, clone RMM-1 #406509) or B220-Alexa700 (Biolegend, clone RA3 #103232). To identify intermediate plasma cells or plasmablasts (IPC), plasma cells (PC) and germinal center populations, cells were stained with the following antibodies: GL7-FITC (Biolegend, clone GL7 #144003), CD138-PE (Biolegend, clone 281-2 #142503), CD95-APC (eBioscience, clone 15A7 #17-0951-80) or B220-Alexa700 (Biolegend, clone RA3 #103232). To determine the percentages of cell populations, values were normalized by percentage of B220⁺ single live cells (single cells, 7-AAD⁻B220⁺; 7-AAD (Life Technologies) was used to identify dead cells). Data acquisition was performed in a BD LSR II Flow Cytometer (BD Biosciences) and analysis was performed with FlowJo software (Tree Star).

DLBCL Cell Lines.

CD40R expression on DLBCL cell lines was measured using FITC-conjugated anti-CD40 (BD clone C53 #B555588). DLBCL cell line viability was measured by APC-conjugated anti-annexin V (BD #B550474) and DAPI exclusion. Data were acquired on MacsQuant flow cytometer (Miltenyi Biotec) and analyzed using FlowJo software package (TreeStar).

IgVH Rearrangement Analysis.

PCR to evaluate IgVH rearrangements was performed on cDNA of VavP-Bcl2 lymphoma cells with a set of a forward primer that anneal to the framework region of the most abundantly used IgVL gene families and a reverse primer located in the Jλ1,3 gene segment (IgL-Vλ1: GCCATTTCCCCAGGCTGTTGTGACTCAGG [SEQ ID NO:22] and IgL-Jλ1,3: ACTCACCTAGGACAGTCAGCTTGGTTCC; SEQ ID NO:23)³⁴.

Class Switch Recombination (CSR) in Kmt2d^{□/□} Tumors.

Genomic DNA isolated from tumors cell suspensions and MEFS as a germinal band control were restricted and for Southern blot hybridization was performed with the following probes: JH probe (PCR amplified with 5′-TATGGACTACTGGGGTCAAGGAAC-3′ [SEQ ID NO:3] and 5′-CCAACTACAGCCCCAACTATCCC-3′ [SEQ ID NO:4], 3′Smu probe (PCR amplified with 5′-CCATGGGCTGCCTAGCCCGGGACTTCCTGCCC [SEQ ID NO:5] and 5′-ATCTGTGGTGAAGCCAGATTCCACGAGCTTCCCATCC-3′; SEQ ID NO:6) and IgκIII a EcoRI/SacI fragment downstream Jκ5 at Igκ locus.

Somatic Hypermutation.

The genomic sequences from VH to the intron downstream of JH4 were PCR-amplified from tumor DNA using degenerate forward primers for the different VH families³⁵ and a reverse primer (5′-AGGCTCTGAGATCCCTAGACAG-3′; SEQ ID NO:7)³⁶ downstream of JH4. Proofreading polymerase (Phusion High Fidelity, NEB) was used for amplification with previously published PCR conditions³⁵. Amplification products were isolated from agarose gels and submitted to Sanger sequencing. Sequences were compared with reference and mutation rate calculated using IMGT/V-QUEST³⁷ and UCSC BLAT. PCR amplification and sequencing was repeated two or three times for each sample. As a negative and a positive control, DNA extracted from mouse embryonic fibroblasts (MEFS) and Igκ-AID B cells, respectively, were used in parallel.

Characterization of Mouse B Cell Differentiation and Antibody Production.

Germinal Center Assessment in Mice.

HPCs from C57BL/6 mice were retrovirally transduced with empty vector or shKmt2d and adoptive transfer approach was performed in 2-month-old C57BL/6 females irradiated with 4.5 Gy (n=3 or 4 per group). After 4 and 7 weeks after injection of HPCs, females were immunized intraperitoneally with 0.5 ml of 2% sheep red blood cell (SRBC) suspension in PBS (Cocalico Biologicals). Nine weeks later spleens were collected for histology and immunohistochemistry analysis. Ki67-positive cells were quantified using Metamorph software.

For analysis of the formation of GCs in Km2d^{-/-} mice, four mice for each genotype (1.5- to 2-month-old, wild-type: 2 males and 2 females; Kmt2d^{-/-} mice: 3 males and 1 female) were immunized intraperitoneally with 100 μg of NP21-CGG (Biosearch Technologies) in Imject alum (Pierce). On day 6 after immunization, splenocytes were harvested and B cell populations were analyzed by flow cytometry as above (see Characterization of B cell populations in Kmt2d^{-/-} mice).

ELISA Analysis of NP-Specific Antibody Production.

Serum from NP-CGG-immunized Kmt2d^{+/+} (wild-type) or Kmt2d^{-/-} mice was analyzed for NP-specific IgM or IgG1 titer using the SBA Clonotyping System-HRP (SouthernBiotech). Plates were coated with 10 ug/ml NP(20)-BSA (Biosearch Technologies) and serum from immunized or nonimmunized mice was added to 96-well assay plates (Costar) at increasing dilutions in PBS with 1% BSA. Bound antibodies were detected with HRP-labeled goat anti-mouse IgG1 or IgM antibodies. The optical density of each well was measured at 405 nm.

In vitro class-switch recombination. For class switch recombination to IgG1, resting splenic B cells were isolated from 2.5- to 5-month-old Kmt2d^{+/+} CD19-Cre⁻ (wild-type, 2 females and 3 males) and Kmt2d^{fl/fl} CD19-Cre⁺ (Kmt2d^{-/-}; 2 females and 3 males) mice by immunomagnetic depletion with anti-CD43 MicroBeads (anti-Ly48, Miltenyi Biotech), and cultured at 0.5×10⁶ cells/ml with LPS (25 μg/ml; Sigma), IL-4 (5 ng/ml; Sigma) and RP105 (anti-mouse CD180; 0.5 μg/ml; BD Pharmingen) for 4 d. B cells were infected at 24 and 48 h in culture with pMX-Cre-IRES-GFP as described³⁸ to enhance Kmt2d^{fl/fl} deletion. Class switching to IgG1 was measured at 96 h in the GFP⁺ population (>90%) by flow cytometry using the following antibodies: IgG1-biotin (BD Pharmingen, clone A85-1 #553441) following streptavidin-Pacific Blue (Molecular Probes) and B220-Alexa700 (Biolegend, clone RA3 #103232). Data acquisition was performed on the BD LSR II Flow Cytometer (BD Biosciences) equipped with CellQuest software (Becton Dickinson). Analysis was performed with FlowJo software (Tree Star).

mRNA-Seq Library Preparation and Sequencing Analysis.

RNA was purified using the RNAeasy Plus Kit (QIAGEN) that included a genomic DNA elimination step. RNA size, concentration and integrity were verified using Agilent 2100 Bioanalyzer (Agilent Technologies). Libraries were generated using Illumina's TruSeq RNA sample Prep Kit v2, following the manufacturer's protocol. Sequencing of 8-10 pM of each library was done on the HiSeq2500 sequencer as 50-bp single-read runs. RNA-seq data from mouse B220 cells were aligned to the mm9 genome using STAR. RNA-seq data from FL subjects were aligned to the hg19 genome using TopHat. −2.0.10 with default parameters except −r 150 (TopHat2: accurate alignment of transcriptomes in the presence of insertions, deletions and gene fusions). Read counts were derived from HTSeq.scripts.count module in HTSeq-0.6.0 with default parameters (HTSeq—a Python framework to work with high-throughput sequencing data). Differentially expressed genes were generated by DESeq2-1.6.3 in R [moderated estimation of fold change and dispersion for RNA-seq data with DESeq2].

ChIP and ChIP-Seq Library Preparation and Sequencing Analysis.

H3K4me1 and H3K4me2 ChIP was performed as previously described³⁹. Briefly, 4×10⁶ mouse B220⁺ cells or DLBCL cells were fixed with 1% formaldehyde, lysed and sonicated (Branson Sonicator; Branson) leading to a DNA average size of 200 bp. 4 ul of H3K4me1 and H3K4me2-specific antibody (Abcam 32356 lot GR106705-5), tested for specificity by histone-peptide array (Active Motif 13001), was added to the precleared sample and incubated overnight at 4° C. The complexes were purified using protein-A beads (Roche) followed by elution from the beads and reverse cross-linking. DNA was purified using PCR purification columns (QIAGEN).

H3K4me1 and H3K4me2 ChIP-seq libraries were prepared using 10 ng of DNA and Illumina's TruSeq ChIP sample prep, according to the manufacturer. Libraries were validated using the Agilent Technologies 2100 Bioanalyzer and Quant-iT dsDNA HS Assay (Life Technologies) and 8-10 pM was sequenced on a HiSeq2500 sequencer as 50-bp single-read runs. ChIP-seq data was aligned to the hg18 and hg19 genomes using STAR. Peak calling and read density in peak regions were performed by ChIPseeqer-2.1 with default parameters (an integrated ChIP-seq analysis platform with customizable workflows).

KMT2D ChIP assays were performed as previously described⁴⁰. Briefly, 3-5×10⁷ cells were cross-linked with 1% paraformaldehyde at room temperature for 15 min and sonicated to generate chromatin fragments of 200-600 bp. Fragmented chromatin was then immunoprecipitated overnight with in-house-generated human KMT2D antibody specific for the N terminus previously described⁵, followed by washes and elution. ChIP-sequencing libraries were prepared with KAPA HTP ChIP-seq sample prep kit (KAPA Bioystems) for further high-throughput sequencing.

H3K4me1 and H3K4me2 ChIP DNA from OCI-LY7 cells transduced with KMT2D shRNA or empty vector control lentivirus were quantified by qPCR. Primers were designed to amplify loci with KMT2D peaks in OCI-LY7 and H3K4me1 and H3K4me2 depletion in OCI-LY1. Enrichment was calculated relative to input. The primers used were:

TNFAIP3 (A20), Forward:
(SEQ ID NO: 8)
GTGCTGCCATCCCCCAAATA,
Reverse:
(SEQ ID NO: 9)
AGCTTTCCCATGAGCCACT;
SOCS3, Forward:
(SEQ ID NO: 10)
ACCTGGCTAGACTGAGGTCAT,
Reverse:
(SEQ ID NO: 11)
TTAGAGGCGCTCTGGTTCCT;
TRAF3, Forward:
(SEQ ID NO: 12)
TCCAAGGGAAGATGAGGCCA,
Reverse:
(SEQ ID NO: 13)
CCTCGGGGGCCATAATACAG;
SGK1, Forward:
(SEQ ID NO: 14)
GACCGATTGGGAAAGCAGGT,
Reverse:
(SEQ ID NO: 15)
GAGTTGGCTCTGGCTTCCAT;
IKBKB, Forward:
(SEQ ID NO: 16)
AGGTCAACAAGGAGTCAGCC,
Reverse:
(SEQ ID NO: 17)
AGGAGGGAGGGGAGCTTTAT;
TNS4 (negative control loci), Forward:
(SEQ ID NO: 18)
TTATTTGGCTGGGTGTGGT,
Reverse:
(SEQ ID NO: 19)
GTAGAGACGGGATTTCACCATG.

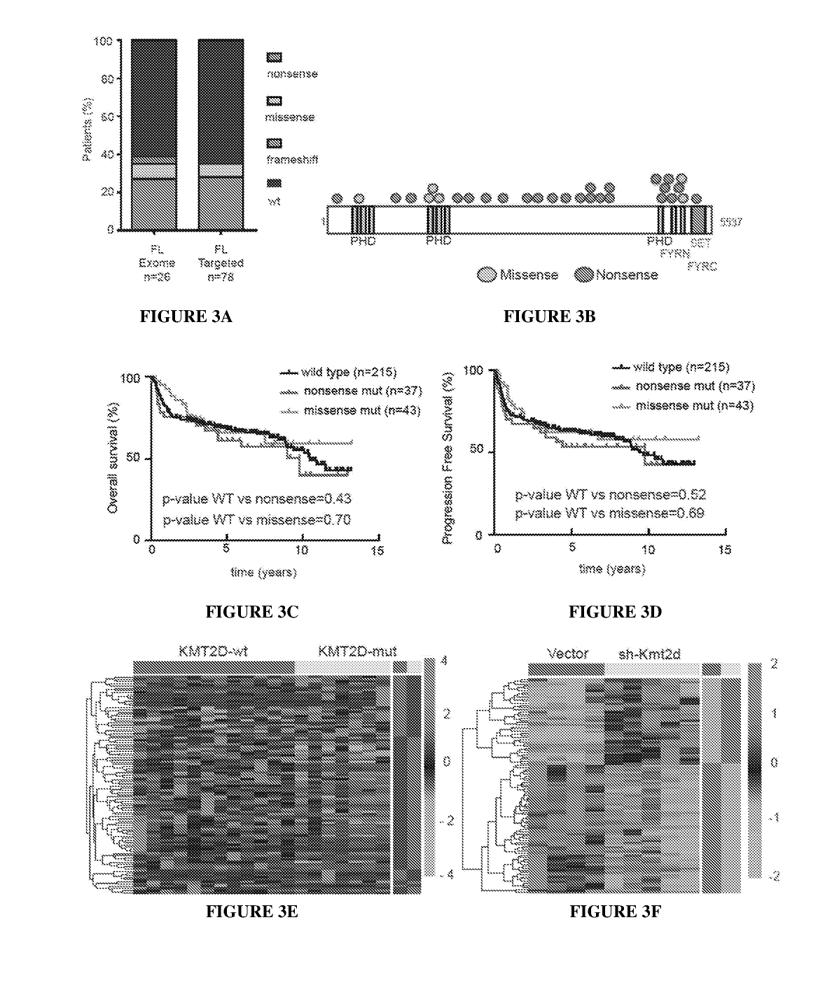

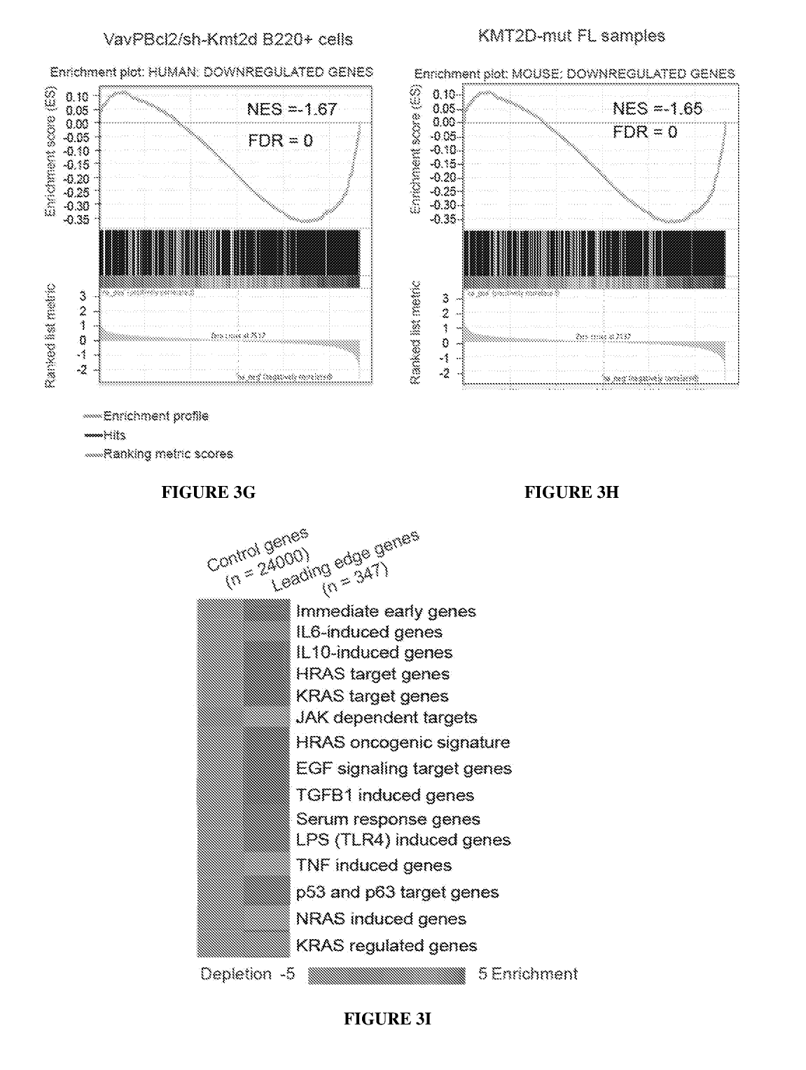

Human_Downregulated_Genes are downregulated genes (log fold change (log FC)<0, P val<0.05, n=519) in FL subjects with nonsense KMT2D mutations versus those with wild-type KMT2D, FIG. 3e) based on RNA-seq data. Mouse_Downregulated_Genes were downregulated genes (log FC<0, P adjust<0.1, Benjamini-Hochberg method, n=1,016) in mouse B220⁺ cells, shKmt2d versus empty vector, FIG. 3f) based on RNA-seq data. We also determined an RNA-seq leading-edge gene set (n=347, FIG. 3i). This gene set is the union of two gene subsets: (i) top 200 downregulated genes in Human_Downregulated_Genes (ranked by log FC derived from B220 RNA-seq) and (ii) top 200 downregulated genes in Mouse_Downregulated_Genes (ranked by log FC derived from FL RNA-seq).

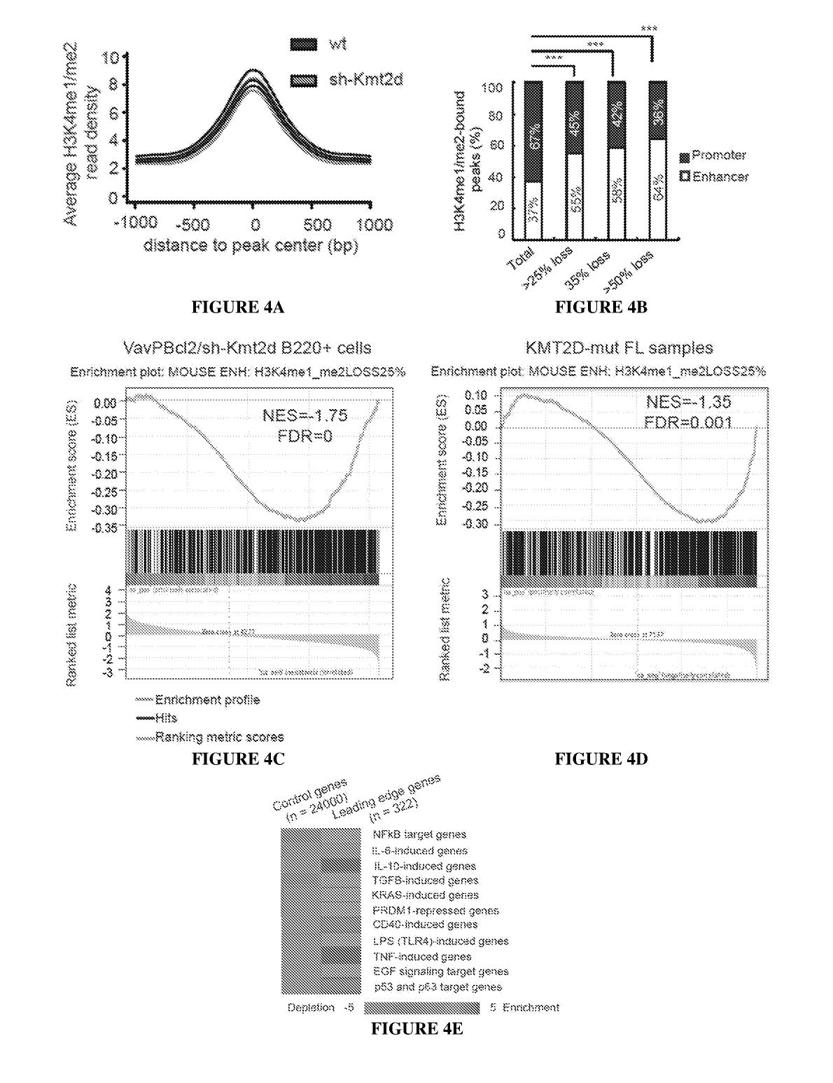

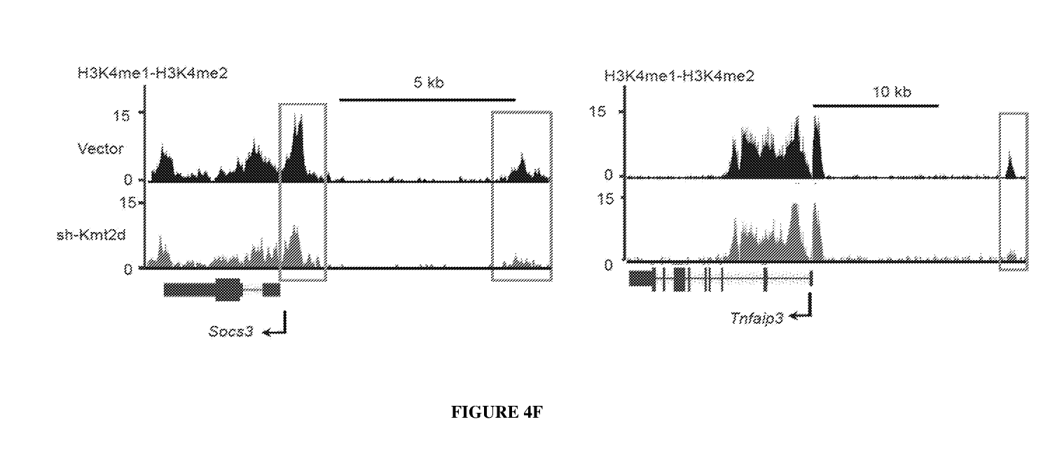

For H3K4me1 and H3K4me2 ChIP data from mouse B220⁺ cells, candidate peaks were the union of the peaks called from each control replicate (n=3) with ChIPseeqer. We defined peaks that overlapped with promoters (defined as ±2 kb windows centered on RefSeq transcription start sites (TSS)). Peaks that didn't overlap with promoters, gene bodies and exons were treated as enhancer peaks. Enhancer peaks inside gene bodies were identified as intragenic enhancer peaks. Intergenic enhancer peaks were defined as being within a 50-kb window from the corresponding genes. TSS Mouse_Enh_H3K4^{me1/me2}_Loss were genes identified with H3K4me1 and H3K4me2 depletion (>25% read density loss and P val<0.05, t-test, n=680) at enhancer peaks in shKmt2d (n=3) (FIG. 4c,d). We also determined a mouse H3K4me1-H3K4me2 ChIP-seq enhancer leading-edge gene set (n=322, FIG. 4e), which is the union of two gene subsets: (i) top 200 downregulated genes in Mouse_Enh_H3K4^{me1/me2}_Loss gene set (ranked by log FC derived from B220 RNA-seq) and (ii) top 200 downregulated genes in Mouse_Enh_H3K4^{me1/me2} Loss gene set (ranked by log FC derived from FL RNA-seq).

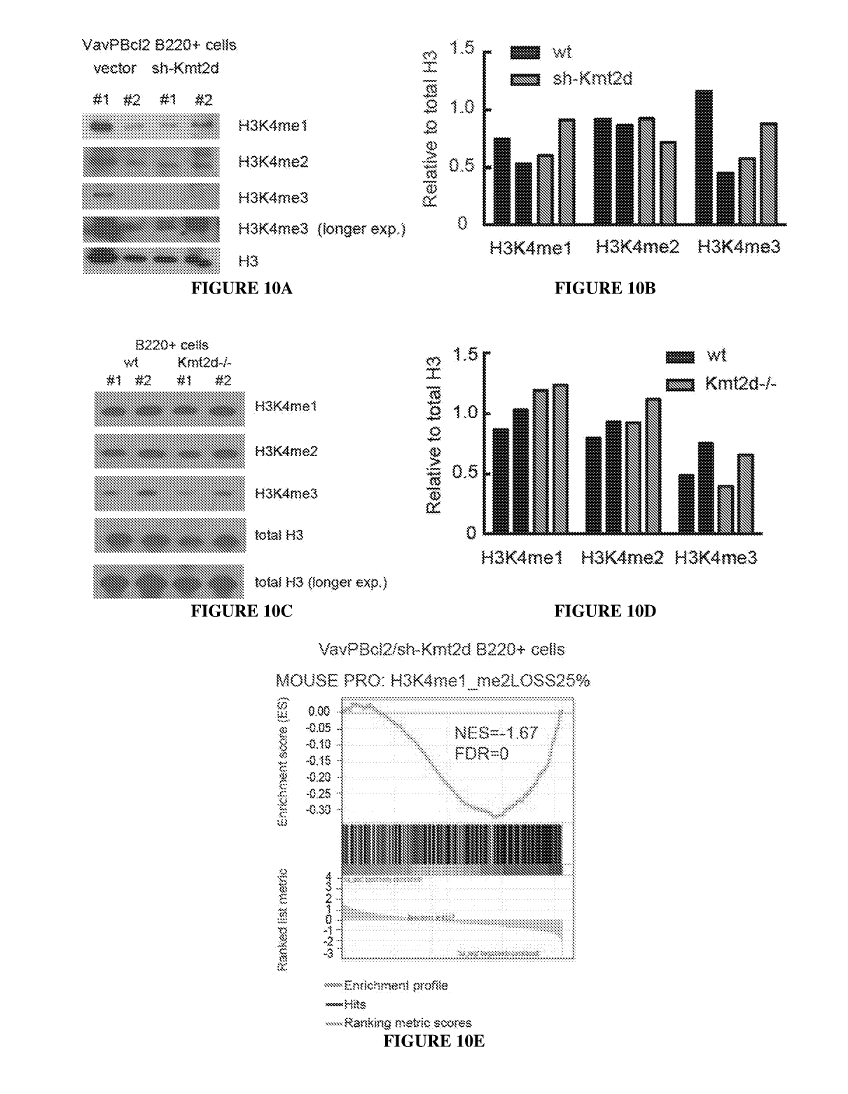

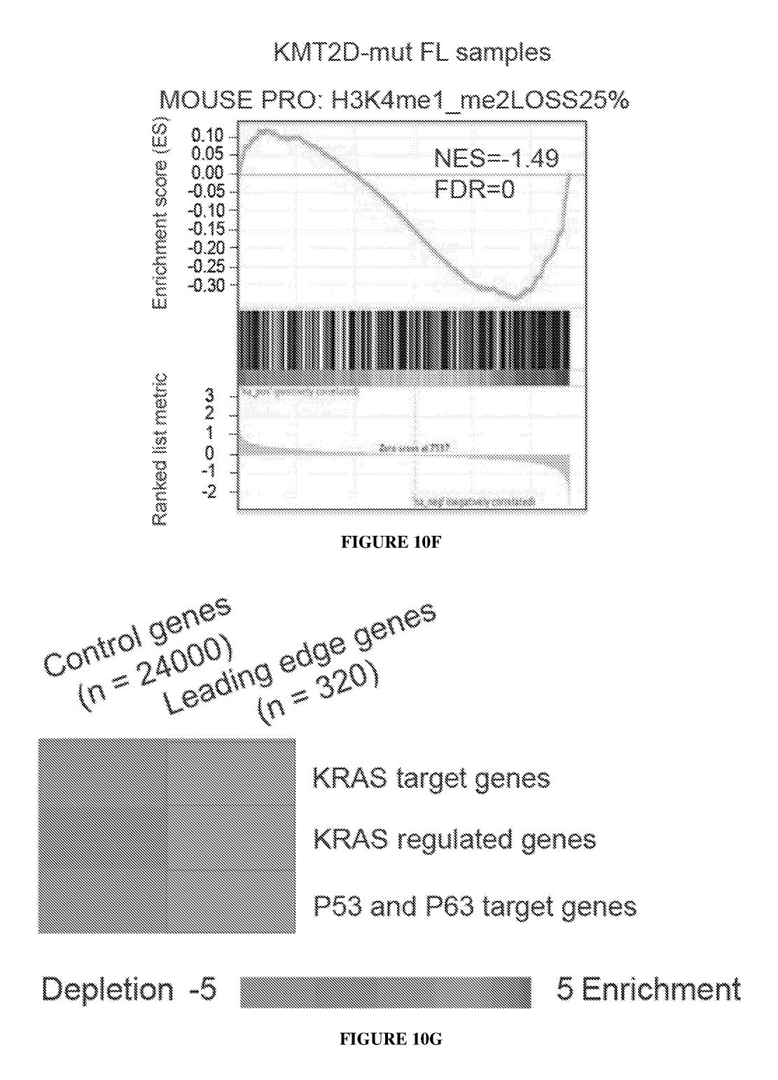

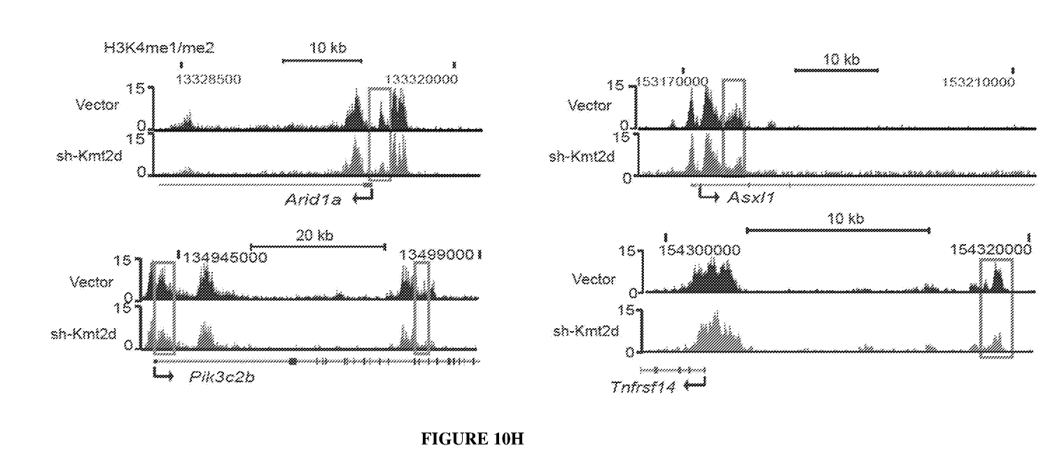

We derived Mouse_Pro_H3K4^{me1/me2} Loss gene sets (n=602, FIG. 10e,f) and mouse H3K4me1 and H3K4me2 ChIP-seq promoter leading-edge genes (n=321, FIG. 10g) in the same way as that for enhancers, described above.

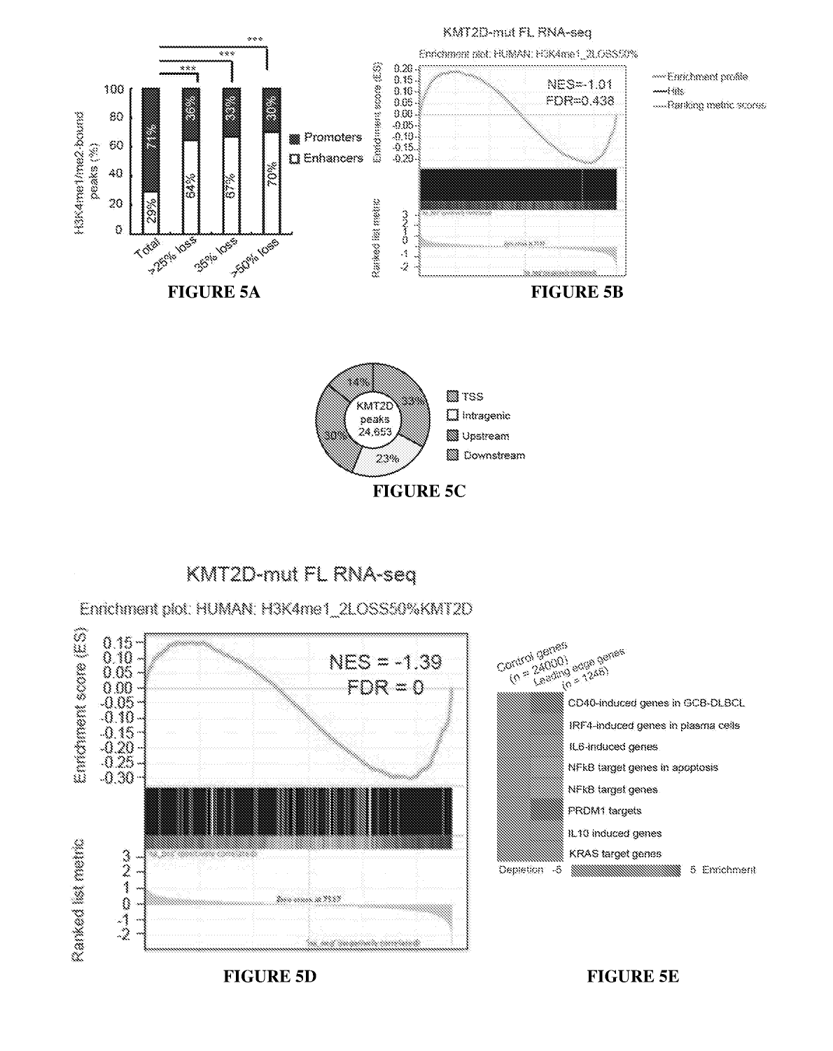

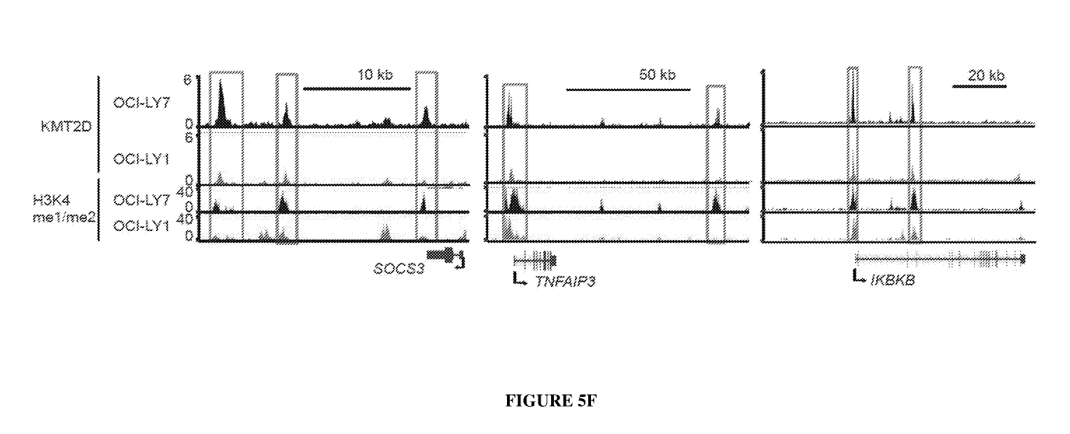

For H3K4me1 and H3K4me2 ChIP data from OCI-LY1 and OCI-LY7 cell lines, candidate peaks were the union of the peaks called from two OCI-LY7 replicates (KMT2D WT) with ChIPseeqer. Promoter and enhancer peaks were determined by the same method described above for mouse B220 H3K4me1-H3K4me2 ChIPseq. In addition all enhancer peaks were overlapped with annotated enhancers previously determined in OCI-LY7. Human_H3K4^{me1/me2} LOSS50 were genes with H3K4me1 and H3K4me2 depletion (>50% read density loss, n=4416) in OCI-LY1 versus OCI-LY7 (FIG. 5b).

KMT2D peaks from KMT2D ChIP-seq data were called using ChIPseeqer. Human_H3K4^{me1/me2}_Loss50_KMT2D were genes with H3K4me1-H3K4me2 loss peaks (>50% read density loss and overlapped with KMT2D peaks, n=1,248, FIG. 5d). We chose 1,248 genes as leading-edge genes (ranked by H3K4me1-H3K4me2 loss from OCI-LY1 and OCI-LY7 ChIP-seq).

Gene Ontology (GO) Analysis with iPAGE.

The GO analyses were performed with iPAGE⁴¹. The concept of mutual information (MI)⁴² to directly quantify the dependency between expression and known pathways in MsigDB⁴³ or in the lymphoid signature database from the Staudt Lab⁴⁴ are used in iPAGE. Nonparametric statistical tests are then used to determine whether a pathway is significantly informative about the observed expression measurements. An iPAGE input file is defined across around 24,000 genes from Refseq genes, where each gene is associated with a unique expression status in our analysis. Meanwhile, each gene can be associated with a subset of M known pathways (for example, from the Gene Ontology annotations). For each pathway, the pathway profile is defined as binary vector with N elements, one for each gene. “1” indicates that the gene belongs to the pathway and “0” indicates that it does not.

Given a pathway profile and an expression file with Nₑ groups, iPAGE creates a table C of dimensions 2×Nₑ, in which C(1,j) represents the number of genes that are contained in the j^{th} expression group and are also present in the given pathway. C(2,j) contains the number of genes that are in the j^{th} expression group but not assigned to the pathway. Given this table, we calculate the empirical mutual information (MI) as follows:

$$I ( candidate pathway ; expression ) = {\sum }_{i = 1}^{2} {\sum }_{j = 1}^{{N}_{e}} P ( i , j ) log \frac{P ( i , j )}{P ( i ) P ( j )}$$

$$where$$

$$P ( i , j ) = C ( i , j ) / {N}_{e} , \\ P ( i ) = {\sum }_{j = 1}^{{N}_{e}} P ( i , j ) and P ( j ) = {\sum }_{i = 1}^{2} P ( i , j )$$

To assess the statistical significance of the calculated MI values, we used a nonparametric randomized-based statistical test. Given I as the real MI value and keeping the pathway profile unaltered, the expression file is shuffled 10,000 times and the corresponding MI values I_{random} are calculated. A pathway is accepted only if I is larger than (1-max_p) of the I_{random} values (max_p is set to 0.005). This corresponds to a P<0.005. In iPAGE, pathways are first sorted by information (from informative to noninformative). Starting from the most informative pathways, the statistical test described above is applied to each pathway, and pathways that pass the test are returned. When 20 contiguous pathways in the sorted list do not pass the test, the procedure is stopped.

Highly statistically significant mutual information is explained by combination of over-representation and under-representation in specific expression groups. To quantify the level of over- and under-representation, the hypergeometric distribution is used to calculate two distinct P values:

### For Over-Representation:

$${P}_{over} ( X \geq x ) = {\sum }_{i = x}^{N} \frac{( \begin{matrix}m \\ i\end{matrix} ) ( \begin{matrix}n - m \\ n - i\end{matrix} )}{( \begin{matrix}N \\ n\end{matrix} )}$$

### For Under-Representation:

$${P}_{under} ( X \leq x ) = {\sum }_{i = 0}^{N} \frac{( \begin{matrix}m \\ i\end{matrix} ) ( \begin{matrix}N - m \\ n - i\end{matrix} )}{( \begin{matrix}N \\ n\end{matrix} )}$$

where x equals the number of genes in the given expression group that are also assigned to the give pathway, m is the number of genes assigned to the pathway (foreground), n is the number of genes in the expression group and N is the total number of genes (background). If Pₒᵥₑᵣ<P_{under}, we consider the pathway to be over-represented in the expression cluster, otherwise it is under-represented. In the heat map, the red color indicates (in log₁₀) the over-represented P values and the blue shows under-representation.

Gene Set Enrichment (GSEA) Analysis.

All the GSEA analysis results in this manuscript were generated from GSEA preranked mode^{43,45}. There were two kinds of input files: (i) FL subjects: gene expression level log FC (nonsense KMT2D mutation versus WT) and (ii) B220: gene expression level log FC (shKMT2D versus MLS). In those input files, we chose the minimum log FC when a gene had multi-transcripts. All the gene sets used in GSEA were described in the Computational Methods section.

Human Cell Lines.

The lymphoma cell lines HT, DoHH2, SU-DHL4, Toledo, Karpas-442, OCI-LY8, NU-DUL1 and SU-DHL10 were maintained in RPMI 1640 with 10% FBS, 1% L-Glutamine and 1% penicillin-streptomycin. OCI-LY7, OCI-LY1 and OCI-LY18 cells were cultured with IMDM media (GIBCO) with 15% FBS, 1% L-Glutamine and 1% penicillin-streptomycin. When indicated OCI-LY7 or SU-DHL4 lymphoma cells were transduced with lentiviruses expressing empty vector (pLKO.1) or shRNA against KMT2D (pLKO.1; Sigma, shKMT2D #1: TRCN0000013140; shKMT2D #2: TRCN0000013142; shKMT2D #3: TRCN0000235742). Source of cell lines are as follows: OCI-LY7, OCI-LY1 and OCI-LY18 from OCI (Ontario Cancer Institute); HT (ATCC® CRL2260™) from ATCC (American Type Culture Collection); SU-DHL4 and NU-DUL1 from DSMZ (Deutsche Sammlung von Mikroorganismen and Zellkulturen GmbH). Cell lines were authenticated by STR DNA profiling by biosynthesis (http://www.biosyn.com/celllinetesting.aspx). Mycoplasma contamination is routinely tested with Universal Mycoplasma detection kit ATCC (http://www.atcc.org/products/all/30-1012K.aspx).

Proliferation assays in lentiviral-transduced OCI-LY7 cells were performed using Viacount assay from Guava Technologies performed as reported⁴⁶. 5×10⁵ cells were seeded in 2 ml into a single well of a 6-well dish. Each experiment was done in triplicate.

For the IL-21 stimulation assay, OCI-LY7 cells transduced with lentiviruses with vector or shRNA against KMT2D were seeded and recombinant human IL-21 (PeproTech #200-21) was added to a 10 ng/ml final concentration; cells were collected after 48 h and whole cell lysates were prepared.

For CD40-IgM stimulation assays, DLBCL cells were seeded at 2.5×10⁵ cells in 500 ul into a single well of a 12-well plate and cultured with anti-CD40 (2.5 ug/ml; RD Systems #AF632) alone or in combination with anti-IgM (10 ug/ml; Jackson ImmunoResearch #109-006-129) for 1, 2 or 4 d. After 1 or 2 d, cells were collected for RNA isolation. After 4 d, cell death was measured using annexin-V and DAPI staining.

Histone Extraction and Quantitative Mass Spectrometry Analysis.

Nuclei were isolated and histone proteins were extracted as described previously with minor modifications⁴⁷. Briefly, histones were acid-extracted from nuclei with 0.2 M H₂SO₄ for 2 h and precipitated with 25% trichloroacetic acid (TCA) overnight. Protein pellets were redissolved in 100 mM NH₄HCO₃ and the protein concentration was measured by Bradford assay. Histone proteins were derivatized by propionic anhydride and digested with trypsin for about 6 h (ref. 47). Peptides were also derivatized by propionic anhydride and desalted by C₁₈ Stage-tips. Histone peptides were loaded to a 75 μm inner diameter (I.D.)×15 cm fused silica capillary column packed with Reprosil-Pur C₁₈-AQ resin (3 μm; Dr. Maisch GmbH, Germany) using an EASY-nLC 1000 HPLC system (Thermo Scientific, Odense, Denmark). The HPLC gradient was 2-35% solvent B (A=0.1% formic acid in water; B=0.1% formic acid in acetonitrile) in 40 min and from 35% to 98% solvent B in 20 min at a flow rate of 300 nl/min. HPLC was coupled to an LTQ-Orbitrap Elite (Thermo Fisher Scientific, Bremen, Germany). Full MS spectrum (m/z 290-1400) was performed in the Orbitrap with a resolution of 60,000 (at 400 m/z), and the 10 most intense ions were selected for tandem mass spectrometry (MS/MS) performed with collision-induced dissociation (CID) with normalized collision energy of 35 in the ion trap. Automatic gain control (AGC) targets of full MS and MS/MS scans are 1×10⁶ and 1×10⁴, respectively. Precursor ion charge state screening was enabled and all unassigned charge states as well as singly-charged species were rejected. The dynamic exclusion list was restricted to a maximum of 500 entries with a maximum retention period of 30 s. Lock mass calibration in full MS scan is implemented using polysiloxane ion 371.10123. Histone peptide abundances were calculated from the acquired raw data by EpiProfile program⁴⁸.

Immunoblot Analysis.

PBS lysis buffer (1% Triton X-100, 1 mM DTT, in PBS) followed by 0.2 N HCl solution was used to prepare lysates for histone fraction of lymphoma (B220⁺) cells. RIPA buffer (Boston Bioproducts) was used to prepare whole-cell lysates of OCI-LY7 cells. Immunoblot analyses were performed according to standard procedures. Membranes were probed with the indicated primary antibodies to: H3K4^{me1} (Abcam, #ab8895), H3K4^{me2} (Millipore #07-030), H3K4^{me3} (Millipore #07-473), total H3 (abcam #ab1791), p-Tyr705-STAT3 (Cell Signaling #9145), total-STAT3 (Cell Signaling #12640) and SOCS3 (Cell Signaling #2932). Enhanced chemiluminescence was used for detection (ECL, Amersham).

Validation of KMT2D Targets by Quantitative Real Time PCR Analyses.

Total RNA from cells was extracted using TRIzol (Invitrogen). Reverse transcription was performed using random primers and SuperScript III First Strand (Invitrogen #18080-400). Quantitative real time-PCR was performed using TaqMan Universal Master Mix (Applied Biosystems) in a 7900 HT Fast Real Time thermocycler (Applied Biosystem). The housekeeping gene used for input normalization of all the qRT-PCR data is β-actin. Taqman gene expression assays used: Kmt2d (Mm02600438_m1), Actb (encoding β-actin) (#4352663), Socs3 (Mm00545913), Dusp1 (Mm00457274), Tnfaip3 (Mm00437121), Arid1a (Mm00473838), Fos (Mm00487425), Ikbkb (Mm01222247), Tnfrsf14 (Mm00619239), KMT2D (Hs00231606), SOCS3 (Hs02330328), TNFRSF14 (Hs00998604), TNFAIP3 (Hs00234713), ARID1A (Hs00195664), DUSP1 (Hs00610256), TRAF3 (Hs00936781), NR4A1 (Hs00374226), IKBKB (Hs00233287), DNMT3A (Hs01027166), ASXL1 (Hs00392415), ARID3B (Hs00356736), MAP3K8 (Hs00178297) and ACTB (#4352667).

Statistical Methods.

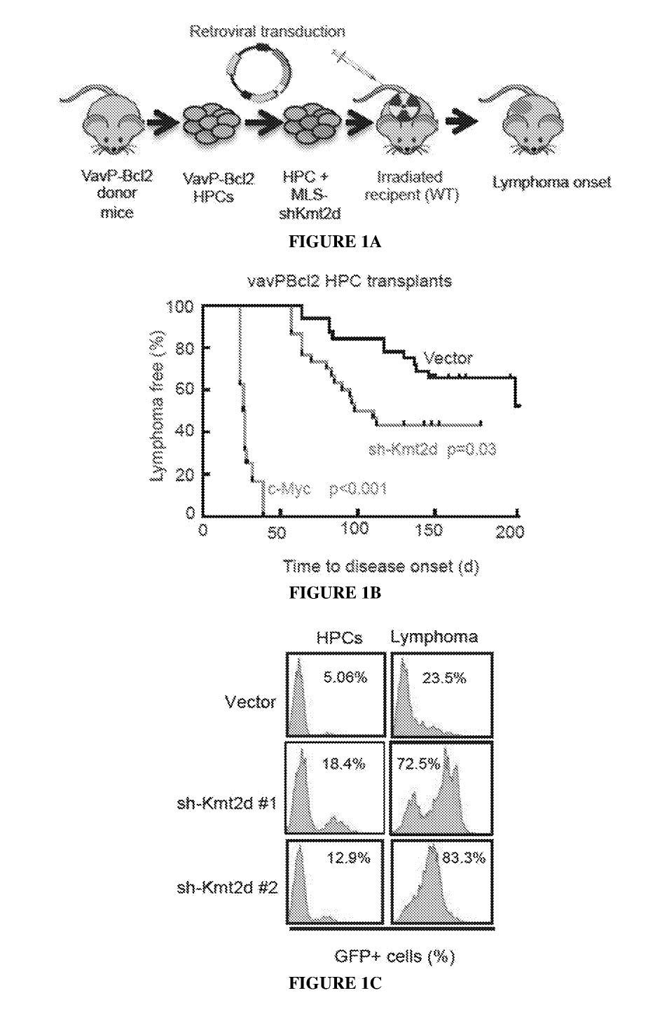

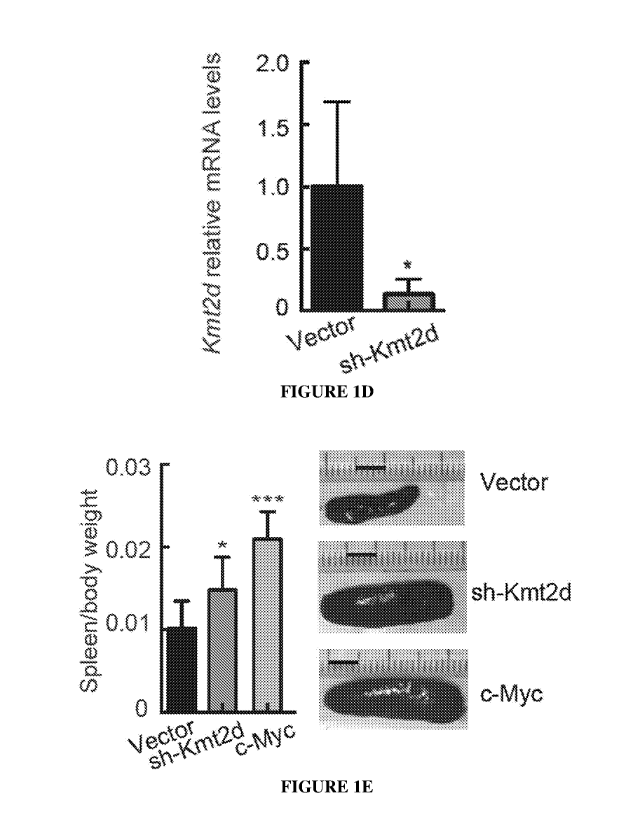

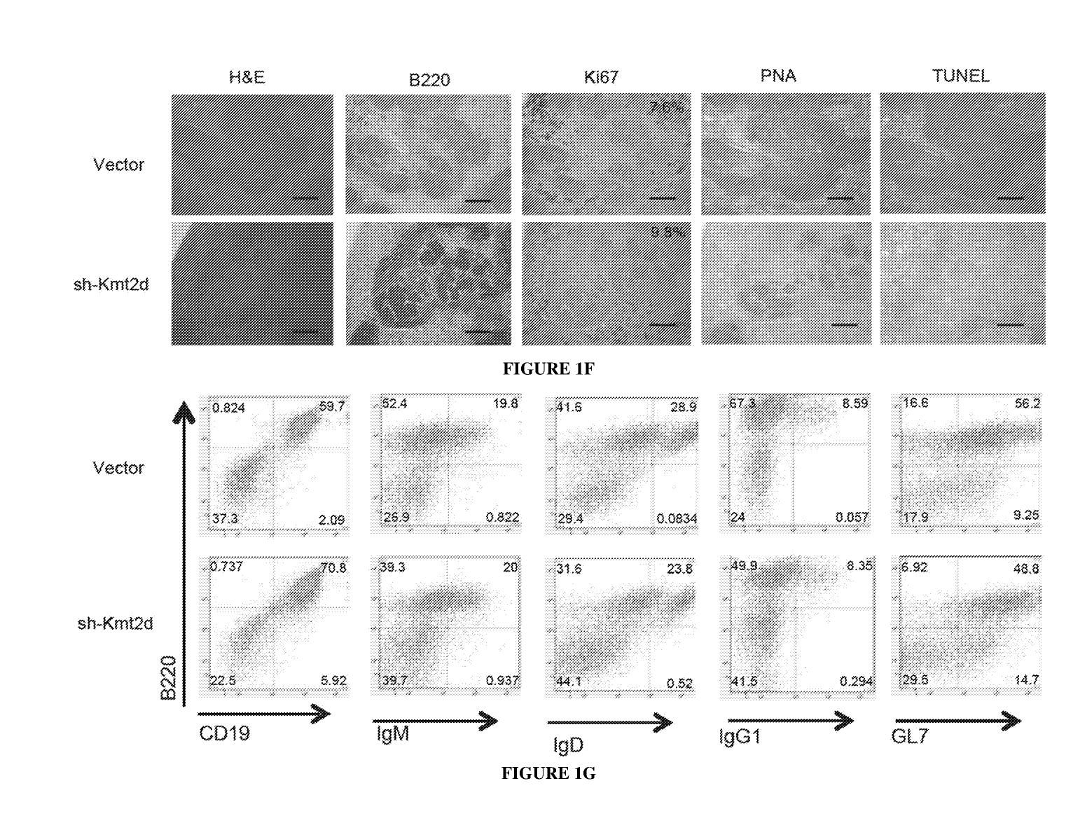

Sample sizes for comparisons between cell types or between mouse genotypes followed Mead's recommendations⁴⁹. Samples were allocated to their experimental groups according to their predetermined type (i.e., mouse genotype) and, therefore, there was no randomization. Investigators were not blinded to the experimental groups unless indicated. In the case in FIG. 1b, only mice that developed lymphomas were considered; mice that didn't develop lymphomas were censored and indicated with ticks in the Kaplan-Meier curves. Quantitative PCR data were obtained from independent biological replicates (n values indicated in the corresponding figure legends). Normal distribution and equal variance was confirmed in the large majority of data and, therefore, we assumed normality and equal variance for all samples. On this basis we used the Student's t-test (two-tailed, unpaired) to estimate statistical significance. Survival in mouse experiments was represented with Kaplan-Meier curves, and significance was estimated with the log-rank test. For contingency analysis (proportion of H3K4me1-H3K4me2 peaks) we used the chi-squared exact test.

Accession Codes.

The Gene Expression Omnibus accession codes for the data in this manuscript are: GES67291 (mouse B220+ lymphoma H3K4me1 and H3K4me2 ChIPseq and RNAseq), GES67314 (KMT2D ChIPseq in OCI-LY7 lymhoma cells), GES67381 (H3K4me1 and H3K4me2 ChIPseq in OCI-LY7 and OCI-LY1 lymphoma cells), SRP056293 (FL samples RNA-seq), SRP056292 (targeted resequencing in FL samples) and SRP056291 (exome sequencing in FL samples).

### Example 2. KMT2D Deficiency Promotes Lymphoma Development In Vivo

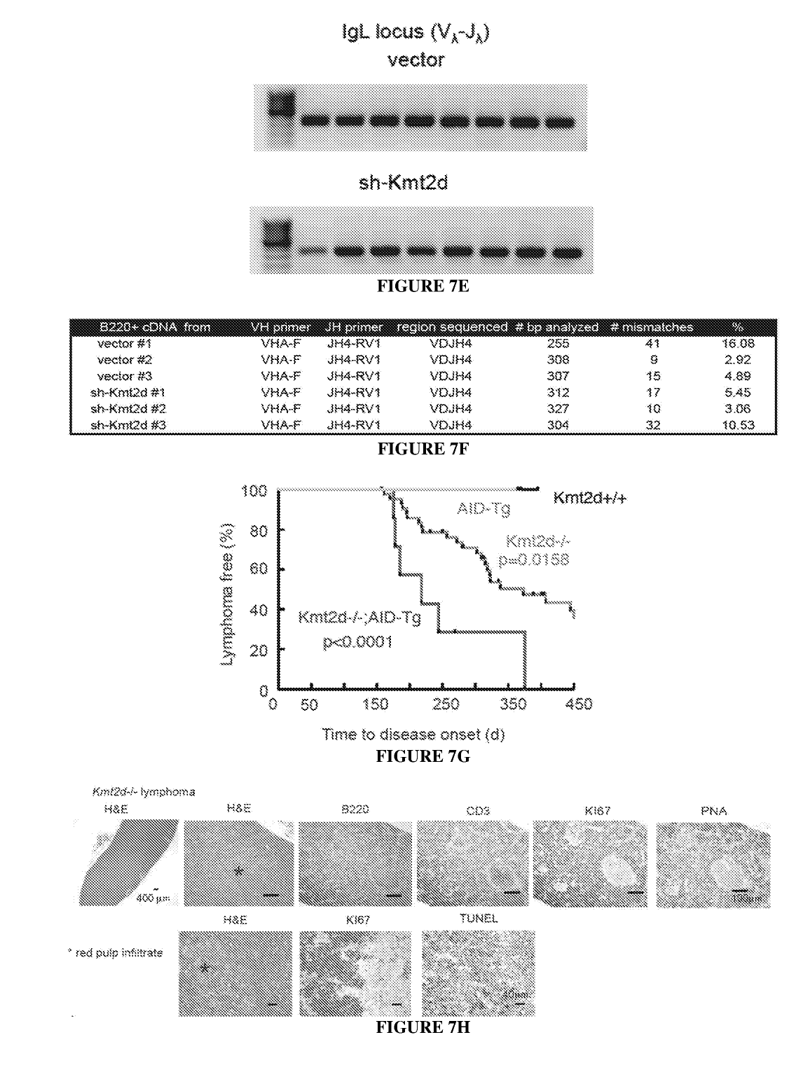

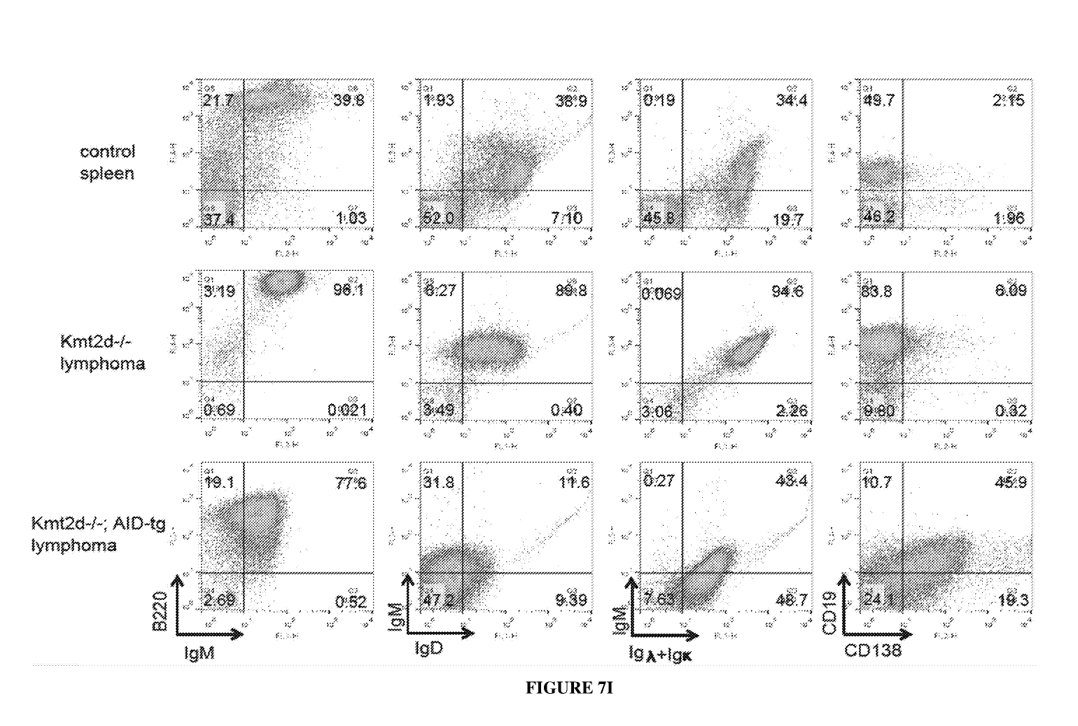

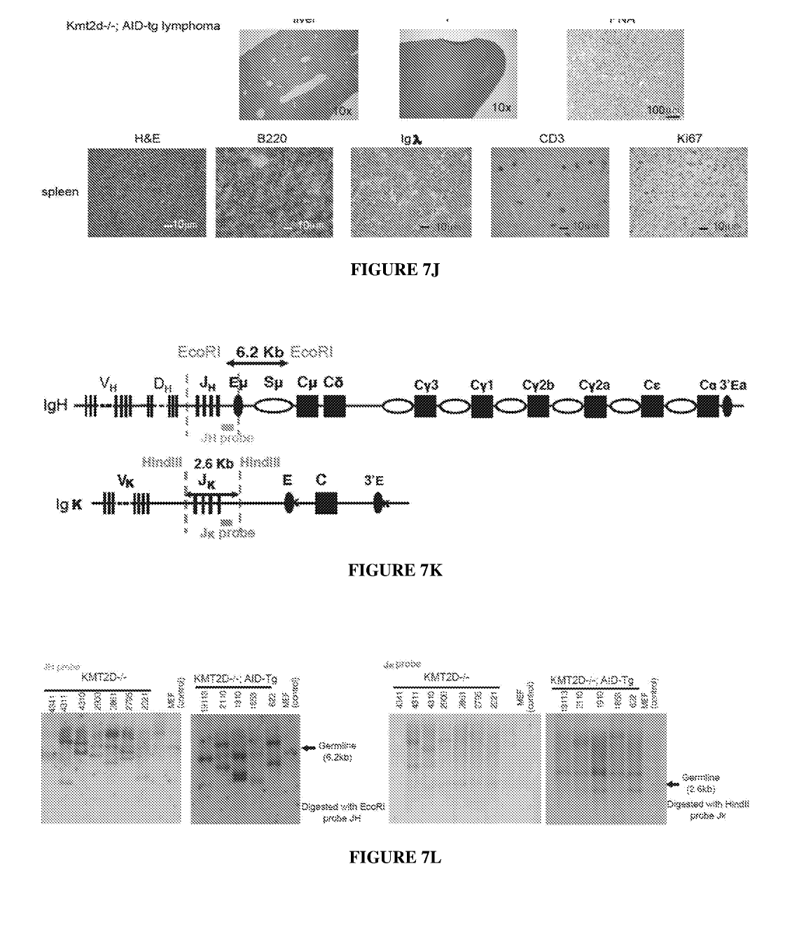

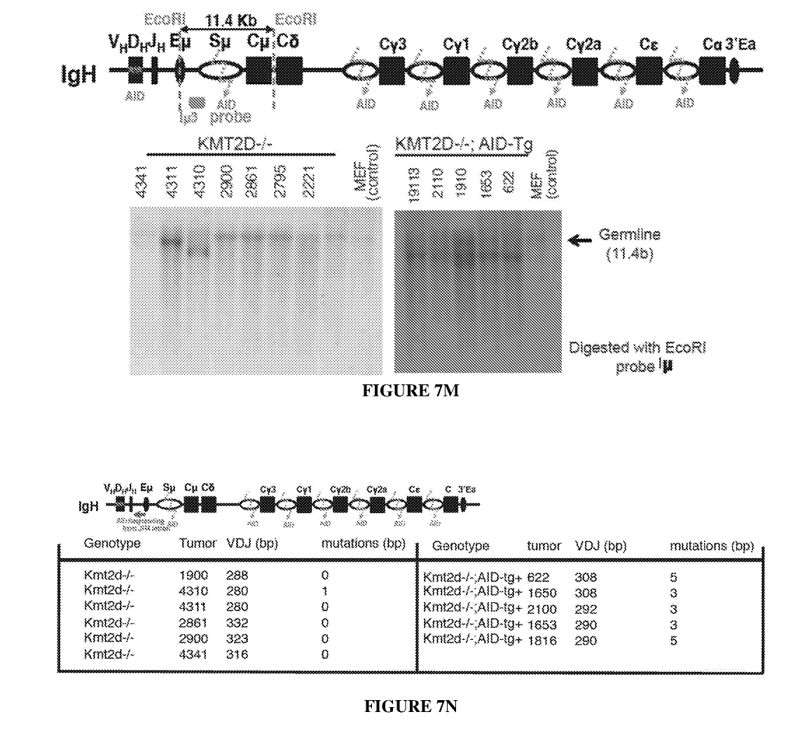

To directly test the effect of KMT2D deficiency in the development of GC-derived lymphoma, we used the VavP-Bcl2 mouse model. In this model, the Vav promoter drives expression of the Bcl2 oncogene in all hematopoietic lineages, and this results in the development of B cell lymphomas that recapitulate key aspects of the genetics, pathology and GC origin of human FLs⁹⁻¹¹. To knock down Kmt2d we transduced unselected VavP-Bcl2 (C57BL/6) transgenic fetal liver cells (embryonic day (ED) 14.5, which are a rich source of hematopoietic progenitor cells (HPCs), with MSCV (Murine Stem Cell Virus) retroviruses that encoded a GFP reporter and either short hairpin RNAs targeting Kmt2d (shKmt2d; n=30), an empty vector (vector; n=37) or the Myc oncogene as a positive control for lymphomagenesis (c-Myc; n=16). We injected an unsorted mix of transduced and untransduced HPCs into syngeneic (C57BL/6) wild-type (WT), lethally irradiated female mice and monitored the recipients for 200 d by peripheral blood smears for the emergence of lymphomas (FIG. 1a). Knockdown of Kmt2d caused a marked acceleration of lymphomagenesis and an increase in FL penetrance from 30% to 60% (FIG. 1b). The lymphomas expressing the Kmt2d-specific shRNA displayed a substantial enrichment of cells that were transduced with two different shRNAs to Kmt2d tethered to GFP as compared to the unsorted HPCs they were derived from and to the HPCs transduced with empty retrovirus (FIG. 1c). We confirmed reduction of Kmt2d mRNA levels in mouse B cells expressing the Kmt2d-specific shRNA constructs (FIG. 1d and FIG. 7a).

The mice transplanted with the VavP-Bcl2-shKmt2d HPCs showed significant splenomegaly and the lymphomas were marked by pathognomonic follicular expansion of neoplastic B220⁺ B lymphocytes that showed positive staining with peanut agglutinin (PNA) and had low Ki67 staining indicating slow proliferation like human FLs (FIG. 13). The PNA-positive staining of and the localization within follicular structures of the B cells are indicative of their germinal center origin (FIG. 1f). Compared to the lymphomas arising in control animals (recipients of VavP-Bcl2 HPCs expressing the empty vector), the Kmt2d-deficient tumors revealed a greater expansion of neoplastic B220⁺PNA⁺ B cells and an advanced destruction of the underlying splenic architecture with invasion of the red pulp in nodular, and sometimes diffuse, patterns (FIG. 1f). Kmt2d-deficient tumors were composed of a greater number of larger, centroblast-like B cells (FIG. 7c), and had more prominent extranodal infiltration into the lung, liver and kidneys (FIG. 7d). Immunophenotyping showed a similar composition of cells in control and Kmt2d-deficient lymphomas, with neoplastic B cells expressing B220, CD19, IgM, IgD and the GC marker GL7 (FIG. 1g and FIG. 7b) and Table 1). PCR analysis of the immunoglobulin light chain (IgL) locus indicated clonal disease (FIG. 7e), and sequence analysis of the VDJH4 variable region showed evidence of SHM (FIG. 7f). Hence, Kmt2d deficiency cooperates with Bcl2 to promote the development of high-grade, GC-derived FLs.

Next we analyzed the potential tumor suppressor function of KMT2D in the absence of any cooperating genetic lesions. We crossed Kmt2d conditional knockout mice (Kmt2d^{fl/fl})⁷ with a CD19-Cre strain to induce Kmt2d deletion in CD19⁺ early B cells. The majority (58%) of the Kmt2d^{fl/fl}×CD19-Cre mice (herein referred to as Kmt2d^{-/-}) became moribund with a survival of 338 d (FIG. 7g). Pathology indicated that the Kmt2d^{-/-} B cell lymphomas in spleens and lymph nodes arose from a pre-GC B cell and were composed of monotonous, atypical B lymphocytes with a high proliferative index (>90% Ki67⁺) and abundant numbers of apoptotic cells, as assayed by TUNEL staining (FIG. 7h). Flow cytometry analysis of these tumors revealed the presence of CD19⁺B220⁺IgM⁺ B cells that often express immunoglobulin kappa (Igκ) or lambda (Igλ) light chains and that have variable expression of IgD and the plasmacytic marker CD138 (FIG. 7i) (Table 1). Genomic analyses of the immunoglobulin locus in these lymphoma cells indicated an oligoclonal origin from cells that had undergone V(D)J recombination at the immunoglobulin heavy chain (IgH) and Igκ loci (FIG. 7k,l). However, the lymphoma cells did not undergo class switch recombination (CSR), as they retained the unrearranged IgH constant region (FIG. 7m). They also showed no evidence of SHM at the IgH locus (FIG. 7n) and lacked expression of markers for mouse GC B cells (PNA; (FIG. 7h). Although the mouse tumors may not directly resemble human lymphomas, these results indicate that Kmt2d acts as a tumor suppressor in B lymphocytes and that this contrasts with its oncogenic function in the myeloid lineage¹².

KMT2D mutations are typically seen in lymphomas that originate from GC B cells that are exposed to the genotoxic activity of the GC-specific enzyme activation-induced cytidine deaminase (AID). Therefore we tested whether the genomic instability caused by AID would synergize with the Kmt2d deficiency to promote lymphoma development in vivo. We crossed the Kmt2d^{-/-} mice to animals overexpressing AID (encoded by Aicda; referred to here as ‘AID-Tg’ mice) and observed a further acceleration of lymphoma onset (FIG. 7g). The Kmt2d^{-/-}×AID-Tg tumors were more aggressive than Kmt2d^{-/-} tumors and showed extensive dissemination into solid organs and complete effacement of the splenic architecture by diffuse proliferation of large atypical B220⁺ B cells with monotypic expression of IgL light chain and very high proliferative fraction (Ki67 positivity >90%). Neoplastic cells were focally positive for CD138 and had intracytoplasmic accumulation of immunoglobulins, suggesting plasmacytic differentiation (FIG. 7i,j). These tumors were oligoclonal and, contrary to the tumors arising in Kmt2d^{-/-} mice, showed AID-induced CSR and SHM and were PNA⁻ (FIG. 7k-n). Hence, AID-induced genomic instability, a hallmark feature of the mutagenic GC environment, cooperates with Kmt2d deficiency in lymphomagenesis.

### Example 3. KMT2D Deficiency Affects Physiological B Cell Behavior

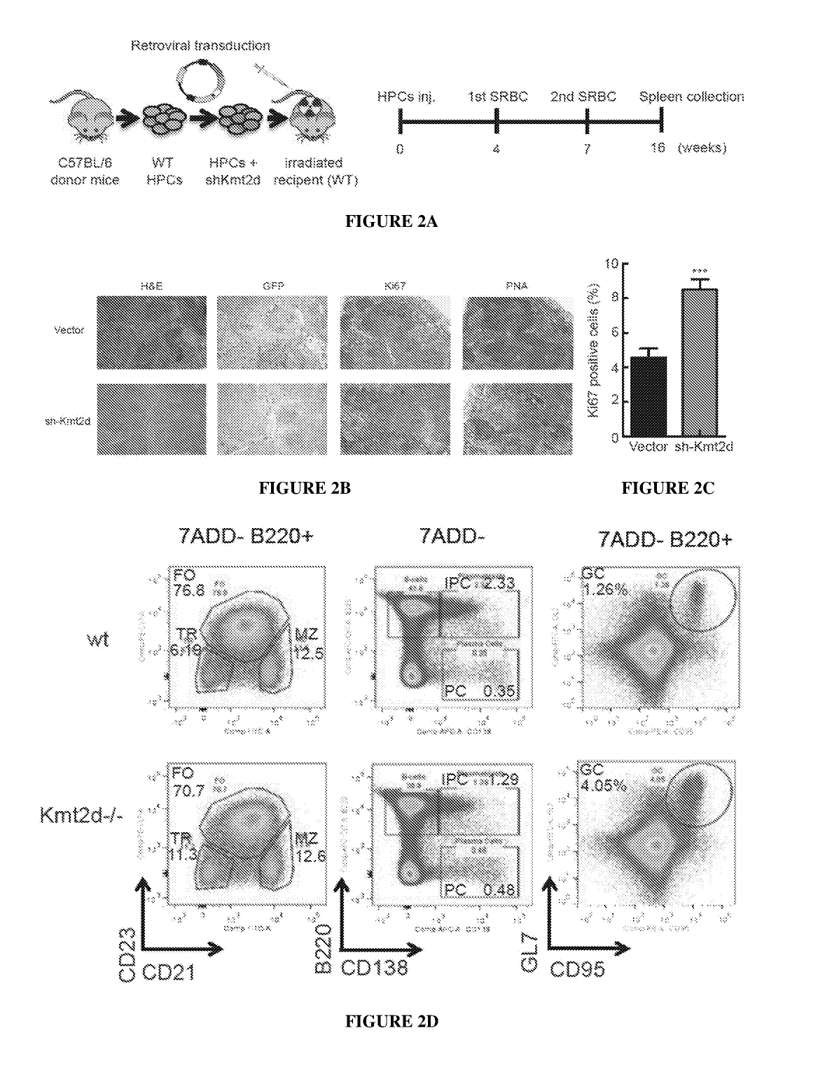

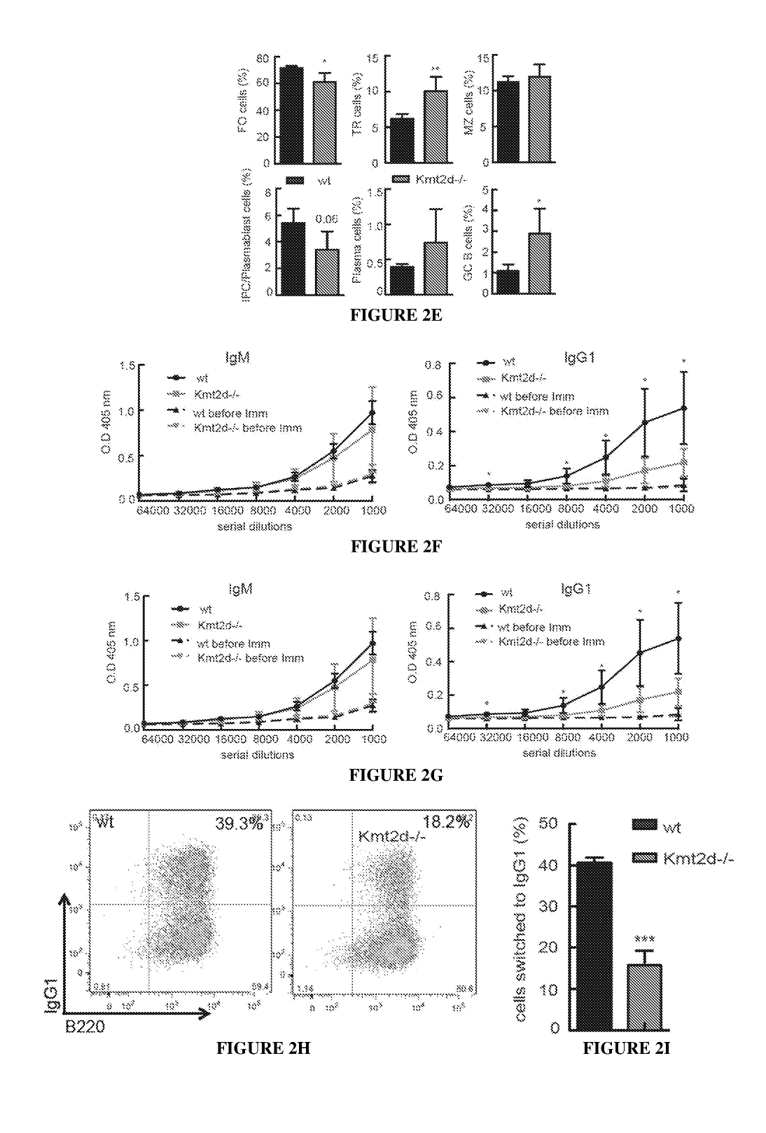

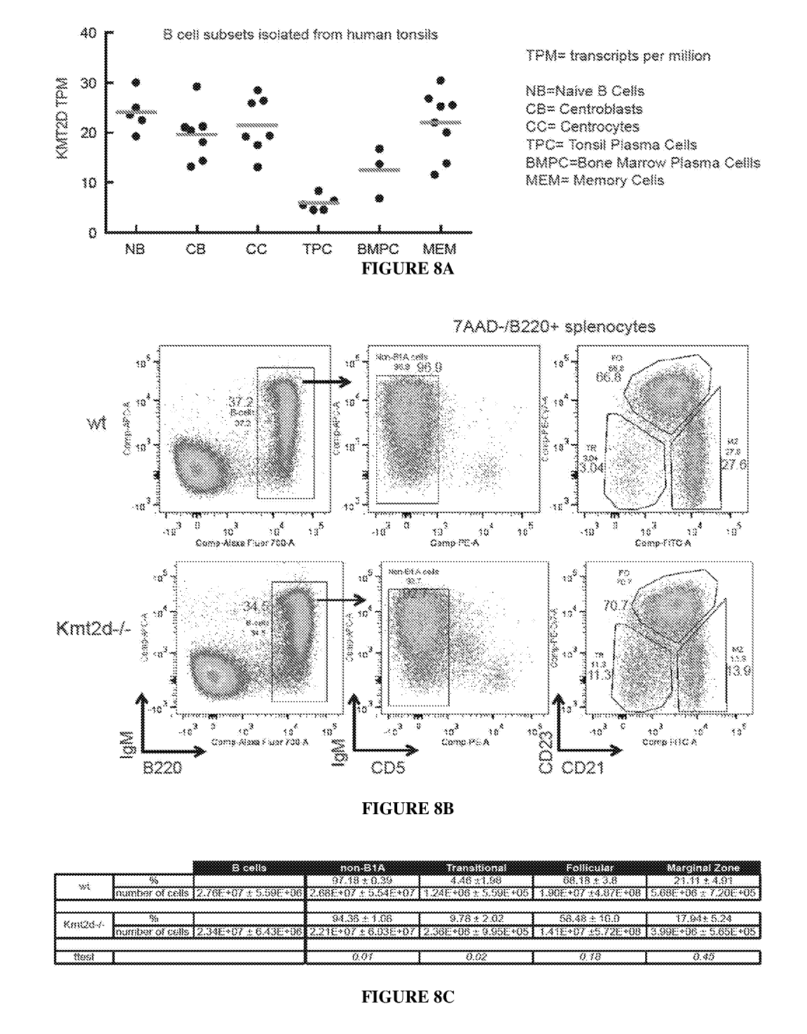

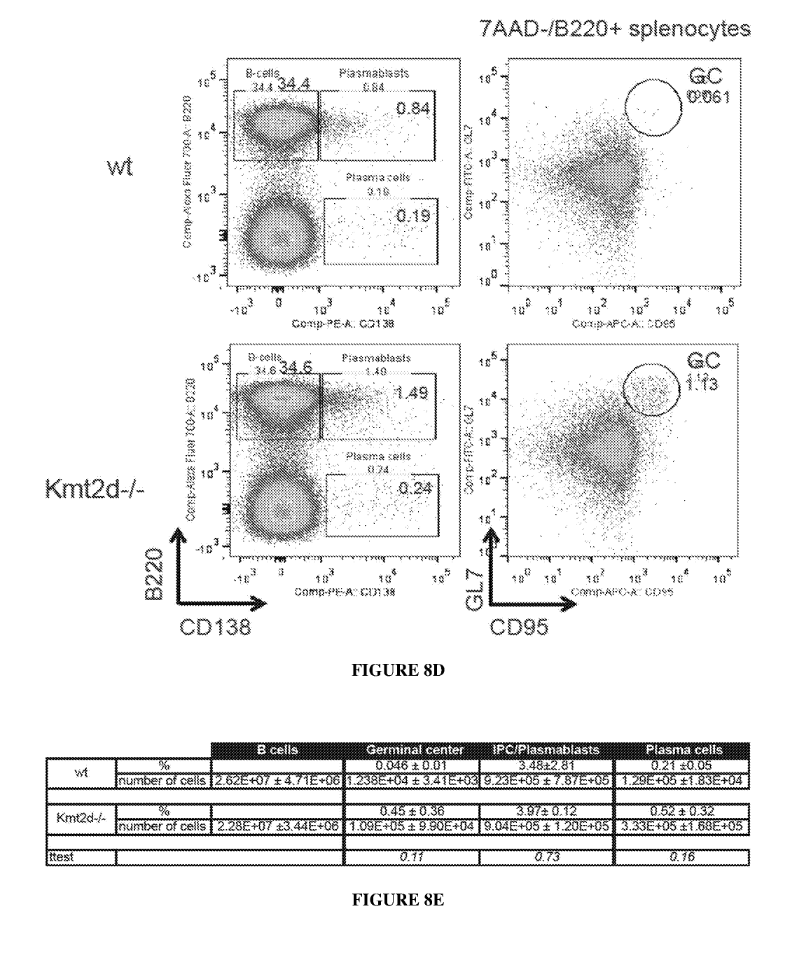

Heritable nonsense mutations in KMT2D are a major cause of the rare congenital Kabuki syndrome (also known as Kabuki makeup or Niikawa-Kuroki syndrome). The syndrome is named for its typical facial features and often comprises a mild immune defect with decreased production of class-switched antibodies and a propensity for ear infections, although a link to tumor development has not been clearly established¹³. We wanted to examine how KMT2D deficiency affects normal B cells. First we analyzed KMT2D expression using RNA-seq in purified mature B cell subsets isolated from human tonsils. KMT2D expression levels were similar in naive, centroblast, centrocyte and memory B cells, whereas it was reduced in plasma B cells, suggesting a functional role for KMT2D before terminal B cell differentiation (FIG. 8a). Next we examined the effect of KMT2D knock down on GC formation using a transplantation model with WT HPCs transduced with retroviruses containing either empty vector (as a control) or Kmt2d-specific shRNA, followed by immunization with sheep red blood cells (SRBC) (FIG. 2a). In control mice, all of the GCs resolved by week 16, as indicated by loss of PNA and Ki67 staining. In contrast, Kmt2d-knockdown mice showed persistent GCs beyond week 16 that consisted of B cells with high PNA and Ki67 staining (FIG. 2b,c). To determine how complete genetic Kmt2d inactivation affects mature B cell populations, we examined unimmunized 4- to 5-month-old Kmt2d^{-/-} mice (before lymphoma onset). Flow cytometric analysis of splenocytes harvested from WT and Kmt2d^{-/-} mice indicated there were equal numbers of total B220⁺ B cells, intermediate plasmablasts (IPCs; B220⁺CD138⁺) and plasma cells (B220⁻CD138⁺) in both sets of mice (FIG. 8b,c). We observed two-fold increase in the number of transitional B cells (B220⁺CD21⁻CD23⁻) and a trend toward elevated numbers of GC B cells (B220⁺GL7⁺CD95⁺) in the Kmt2d^{-/-} mice (FIG. 8b-e). To determine the impact of Kmt2d deletion on GC formation and differentiation, we immunized mice with SRBC, to induce germinal center (GC) formation, and analyzed splenocytes harvested from WT and Kmt2d^{-/-} mice 6 d after immunization. Flow cytometric analysis indicated a modest decrease in follicular B cells (FO; B220⁺CD23⁺CD21^{lo}), a trend toward decreased numbers of plasmablasts and increased numbers of transitional B cells (TR) and, most notably, a significant three-fold increase in the number of GC B cells in Kmt2d^{-/-} splenocytes, as compared to those in splenocytes from WT mice (FIG. 2d,e). These results indicate that Kmt2d loss results in an expansion of GC B cells (which represent the cell type from which DLBCLs and FLs arise in humans) after immunization.

To determine whether Kmt2d loss affects B cell antibody production, we measured serum IgM and IgG1 levels by ELISA in WT and Kmt2d^{-/-} mice. Results showed that IgM antibody levels were similar for both groups of mice under basal conditions, and although the wild-type mice showed the expected increase in IgG1 levels following NP-CGG (Chicken Gamma Globulin) immunization, Kmt2d^{-/-} mice had decreased IgG1 levels, indicating a class switch defect (FIG. 2f). Consistent with these in vivo findings, we also observed a defect in CSR to IgG1 in Kmt2d-deficient B cells after in vitro stimulation with lipopolysaccharide (LPS), CD80-specific antibody and interleukin-4 (IL-4), as indicated by reduced surface IgG1 expression on the Kmt2d^{-/-} B cells (FIG. 2g-i). Hence, KMT2D loss affects B cell differentiation and impedes the B cell immune response in a manner consistent with the mild immune defect associated with Kabuki syndrome.

### Example 4. Consequences of KMT2D Mutations in Human Lymphomas

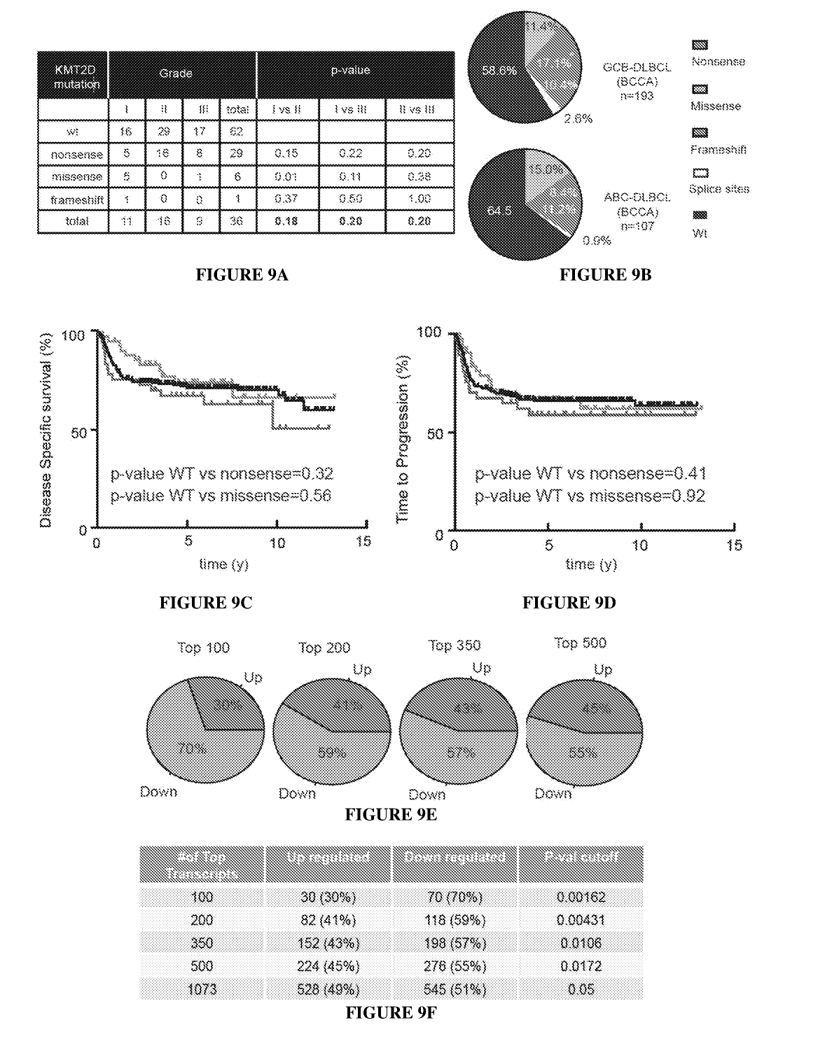

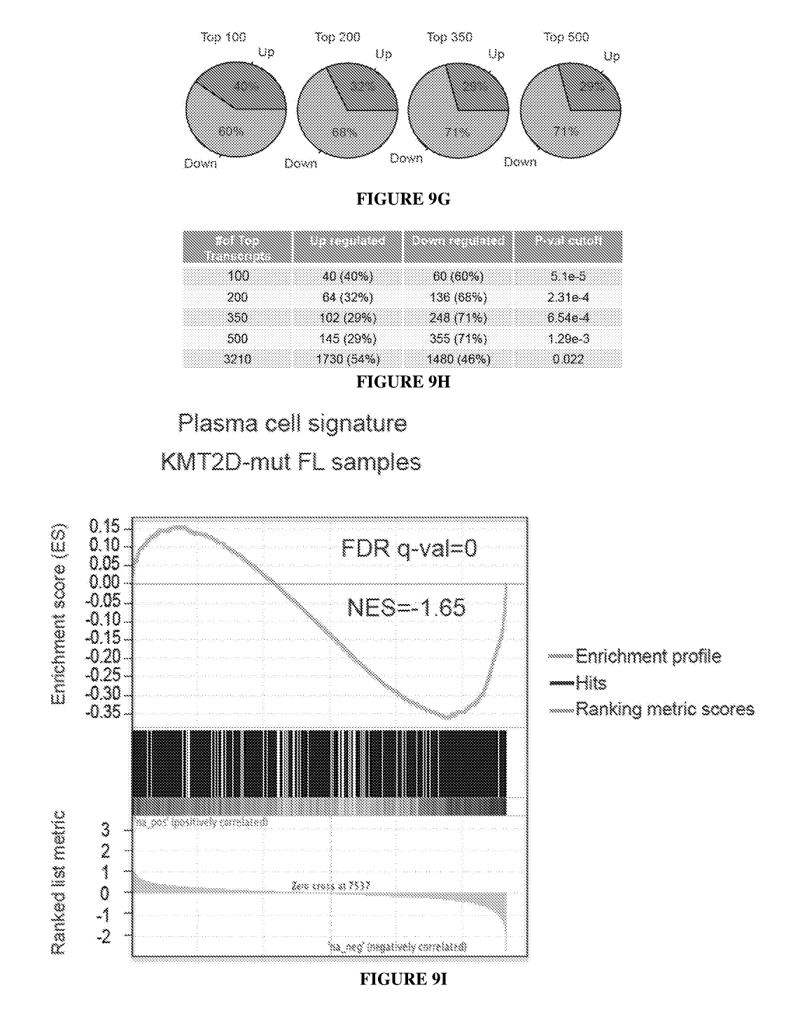

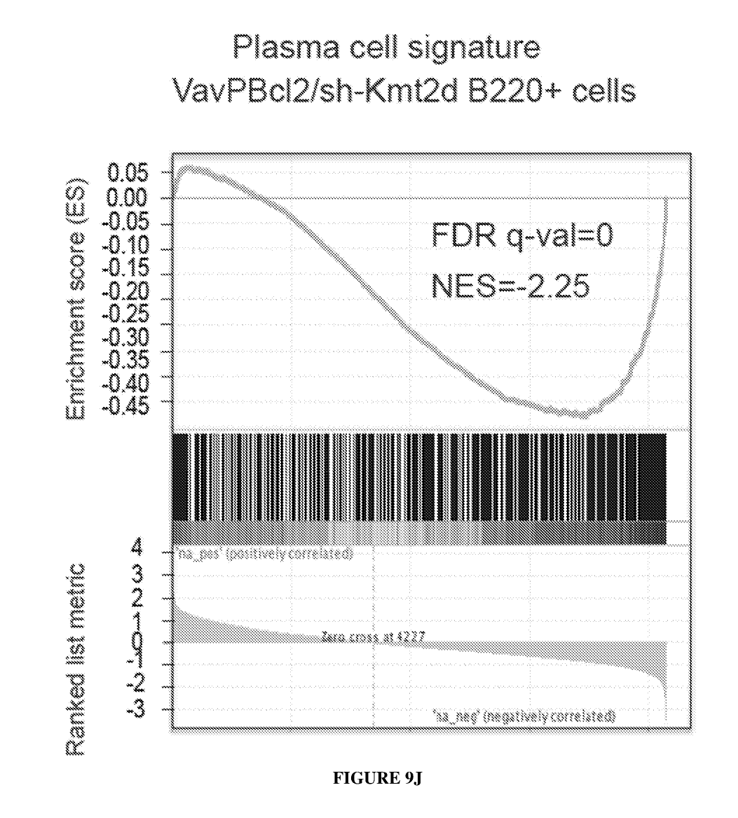

To explore the effects of KMT2D mutations on clinical behavior, we established the KMT2D mutation status in a cohort of 104 human FL specimens. We detected KMT2D mutations in nearly 40% of samples but did not find an apparent hotspot (FIG. 3a,b) (Table 2). In these FLs, 38 of the 104 samples had KMT2D mutations, with four being homozygous. Of a total of 49 KMT2D mutations, 36 were nonsense, 12 were missense and one was a frameshift mutation. KMT2D mutations in FL were not significantly associated with FL grade (FIG. 9a).

Next we analyzed KMT2D status in a cohort of 347 newly diagnosed, clinically annotated DLBCL cases that were all treated with rituximab (R) plus a combination of cyclophosphamide, vincristine, doxorubicin and prednisone (CHOP)—referred to here as R-CHOP—at the BC Cancer Agency (Vancouver) and that were classified as GC B cell (GCB) or activated B cell (ABC) subtype by gene expression profiling. The cases were selected on the basis of the following criteria: individuals were 16 years of age or older with histologically confirmed de novo DLBCL according to the 2008 World Health Organization (WHO) classification, and DNA extracted from fresh-frozen biopsy material (tumor content >30%) was available. The overall mutation frequency was similar to our FL cohort, however we noticed a higher prevalence of nonsense mutations in the GCB subtype (17.6%) than in the ABC subtype (8.4%) (FIG. 9b). KMT2D mutations were not significantly linked to overall survival (OS), progression-free survival (PFS), disease-specific survival (DSS) or time to progression (TTP) (FIG. 3c,d and FIG. 9c,d) Table 2). The lack of correlation may indicate no effect of this specific treatment, or it may reflect alternate changes in tumors with wild-type KMT2D that equally affect outcomes.

### Example 5. KMT2D Controls a Common Set of Genes in Mouse and Human FLs

Next we investigated the transcriptional changes related to KMT2D mutation status by RNA-seq on seven human FL specimens with KMT2D nonsense mutations and 12 with wild-type KMT2D. As expected, the most differentially expressed genes in FLs with nonsense mutation-containing KMT2D were skewed toward gene downregulation, such that among the top 100 genes 70% were decreased, whereas that fraction decreased to 55% when 500 genes were included (FIG. 3e and FIG. 9e,f). Similarly, RNA-seq on magnetic cell sorting (MACS)-purified mouse B220⁺ cells from Kmt2d-knockdown (n=5) and control lymphomas (n=4) revealed that differentially expressed genes in Kmt2d-deficient lymphomas were skewed toward gene downregulation, further supporting the established role of Kmt2d as an activator of gene expression (FIG. 3f and FIG. 9g,h). Moreover, genes that were downregulated in the mouse Kmt2d-deficient lymphomas were highly enriched among genes that were downregulated in human KMT2D mutant specimens and vice versa (FIG. 3g,h; Table 3). By contrast, there was no enrichment among the upregulated genes. This suggests the downregulation of a conserved gene expression signature in human and mouse KMT2D-deficient lymphomas. To further explore these signatures, we examined the leading-edge genes that drive this reciprocal relationship for potential B cell functions that are perturbed by KMT2D inactivating mutations (Table 4). The analysis revealed an enrichment for genes implicated in the immediate early response to antigen/growth factor stimulation, IL-6, IL-10, RAS and tumor necrosis factor (TNF) signaling pathways (FIG. 3i and Table 4) and for plasma cell differentiation-related genes (FIG. 9i,j). Hence in human and mouse FLs, KMT2D controls a common set of genes related to immune signaling and B cell differentiation pathways

To assess how KMT2D depletion contributes to transcriptional regulation, we measured H3K4 mono- and dimethylation (H3K4me1 and H3K4me2, respectively) in Kmt2d-deficient and control lymphomas. Using an antibody that specifically recognizes H3K4me1 and H3K4me2 on DNA, we performed ChIP-seq on purified B220⁺ mouse lymphoma cells (n=3 for both empty vector-containing and shKmt2d-containing VavP-Bcl2 cells). First, analysis of ChIP-seq data for H3K4me1 and H3K4me2 abundance did not reveal a global loss of the marks genome wide (FIG. 4a). We confirmed this observation with immunoblots for H3K4me1, H3K4me2 and trimethylated H3K4 (H3K4me3) on lysates from sorted B220⁺ mouse Kmt2d-knockdown lymphoma cells (FIG. 10a,b) and nonmalignant B220⁺ cells from WT and Kmt2d^{-/-} mice (FIG. 10c,d). By contrast, we observed focal depletion of the H3K4me1 and H3K4me2 marks at a subset of genomic sites in the mouse VavP-Bcl2-shKmt2d lymphomas. Specifically, H3K4me1 and H3K4me2 depletion was significantly more pronounced at putative enhancers as compared to that in promoter elements (FIG. 4b). Using gene set enrichment analyses (GSEA) we found that the genes associated with significant H3K4me1 and H3K4me2 depletion in enhancers and promoters (≥25% read density reduction) were enriched among downregulated genes in both mouse and human KMT2D-deficient lymphomas (FIG. 4c,d and FIG. 10e,f). The leading-edge genes driving this association were enriched for target genes induced by CD40, NF-κB, IL-6, IL-10, LPS, TGF-τ and TNF-α (FIG. 4e, FIG. 10g). Notably, among the genes that showed depletion of the H3K4me1 and H3K4me2 marks at enhancers with concurrent changes in gene expression were tumor suppressor genes such as Tnfaip3 (A20) (ref. 14), Socs3 (ref. 15), Tnfrsf14 (Hvem)¹⁶, Asxl1 and Arid1A (FIG. 4f and FIG. 4h).

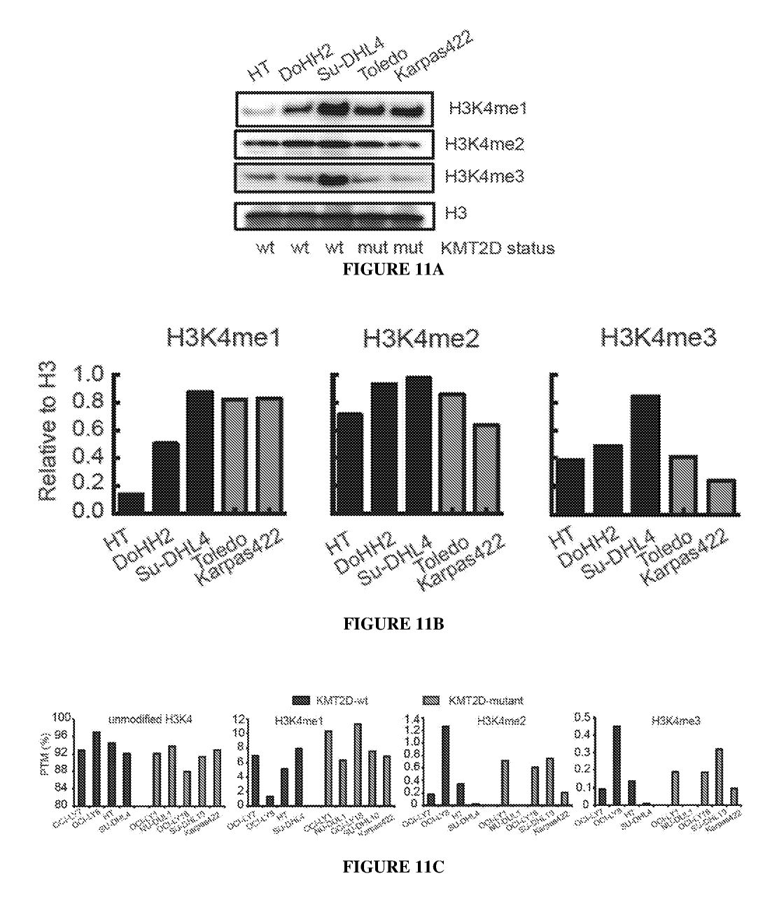

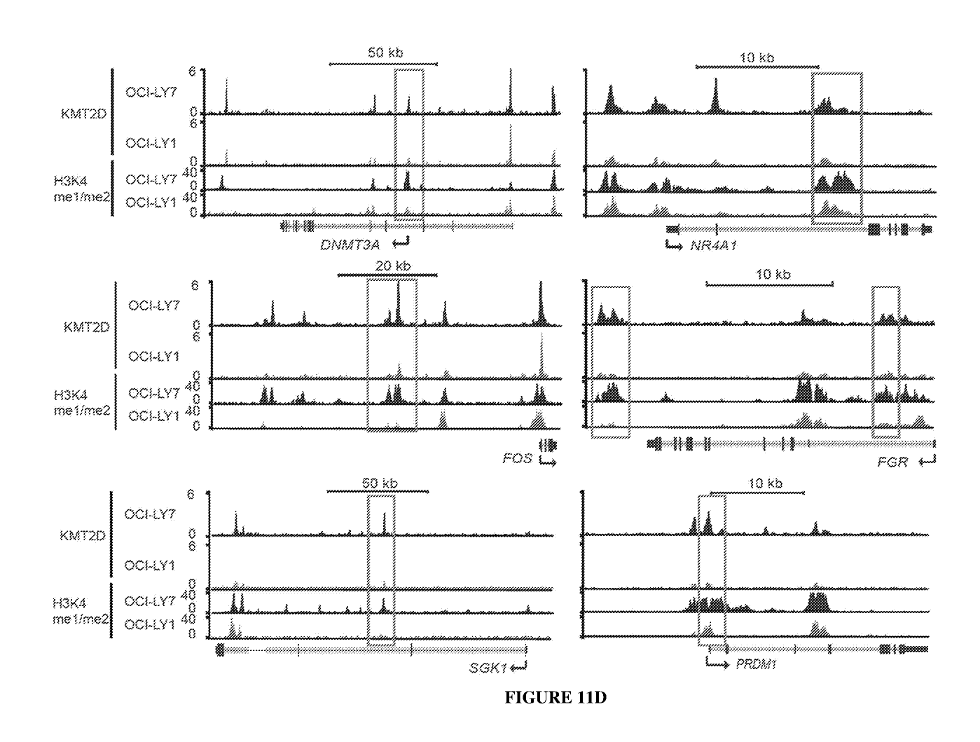

Next we analyzed H3K4me1 and H3K4me2 abundance in human lymphoma cells lines that were either wild type (OCI-LY7, HT, DOHH2 and SU-DHL4) or mutant (OCI-LY1, OCI-LY18, Toledo and Karpas422) for KMT2D. As in the mouse lymphomas, measurements of global H3K4 methylation by immunoblotting and mass spectrometry showed no differences between the lymphoma lines with WT and mutant KMT2D (FIG. 11a-c). Consistent with the results in the mouse lymphomas, H3K4me1 and H3K4me2 ChIP-Seq on human lymphoma cells containing either WT (OCI-LY7) or mutant (OCI-LY1) KMT2D showed a focal defect that was limited to a subset of H3K4me1 and H3K4me2 sites, and ranking based on the extent of H3K4me1 and H3K4me2 depletion confirmed a predominant effect on enhancers similar to those observed in the experiments in mouse lymphoma cells (FIG. 5a). However, initial analyses for the enrichment of the loss of H3K4me1 and H3K4me2 among genes that were downregulated in human FLs with mutant KMT2D did not show the expected level of enrichment (FIG. 5b). This indicated that we needed to more accurately define the direct KMT2D target genes in human lymphoma cells. To measure KMT2D binding directly we performed ChIP-seq in OCI-LY7 and OCI-LY1 cells using a validated antibody against KMT2D. Similarly to previous studies, we identified ˜24,000 KMT2D-binding sites; 32% were associated within transcriptional start sites (TSS) and the others were distributed to locations that were intragenic or upstream or downstream of genes in the OCI-LY7 cells (FIG. 5c)⁵. Of note, genes in OCI-LY7 cells that were bound directly by KMT2D and that had a loss of H3K4me1 and H3K4me2 were also highly enriched among the downregulated genes that were identified in human FL subjects with KMT2D mutations (FIG. 5d). Once again these KMT2D target genes were associated with immune signaling pathways including those involving CD40, IL-6, IL-10, NF-κB, IRF4 and others (FIG. 5e). Consistent with the analysis of the mouse lymphomas, these genes included the lymphoid tumor suppressors TNFAIP3 (A20) and SOCS3, which showed consistent changes in KMT2D binding and H3K4 methylation in cells with WT (OCI-LY7) and mutant (OCI-LY1) KMT2D (FIG. 5f and FIG. 11d).|

### Example 6. Functional Validation of Selected KMT2D Target Genes

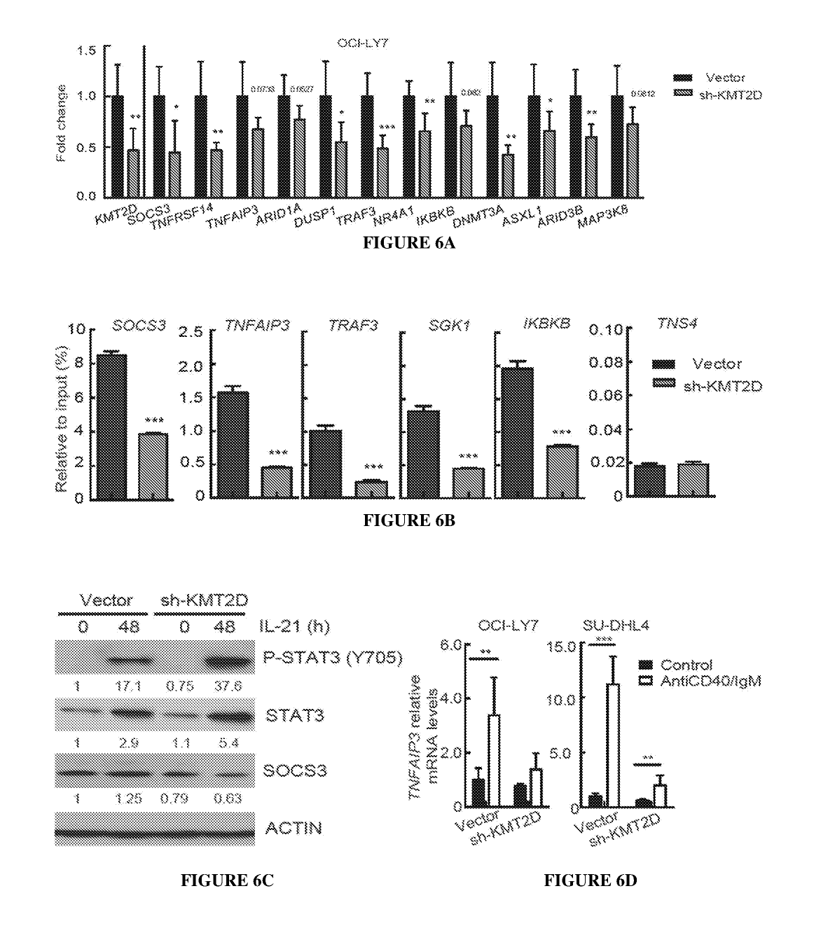

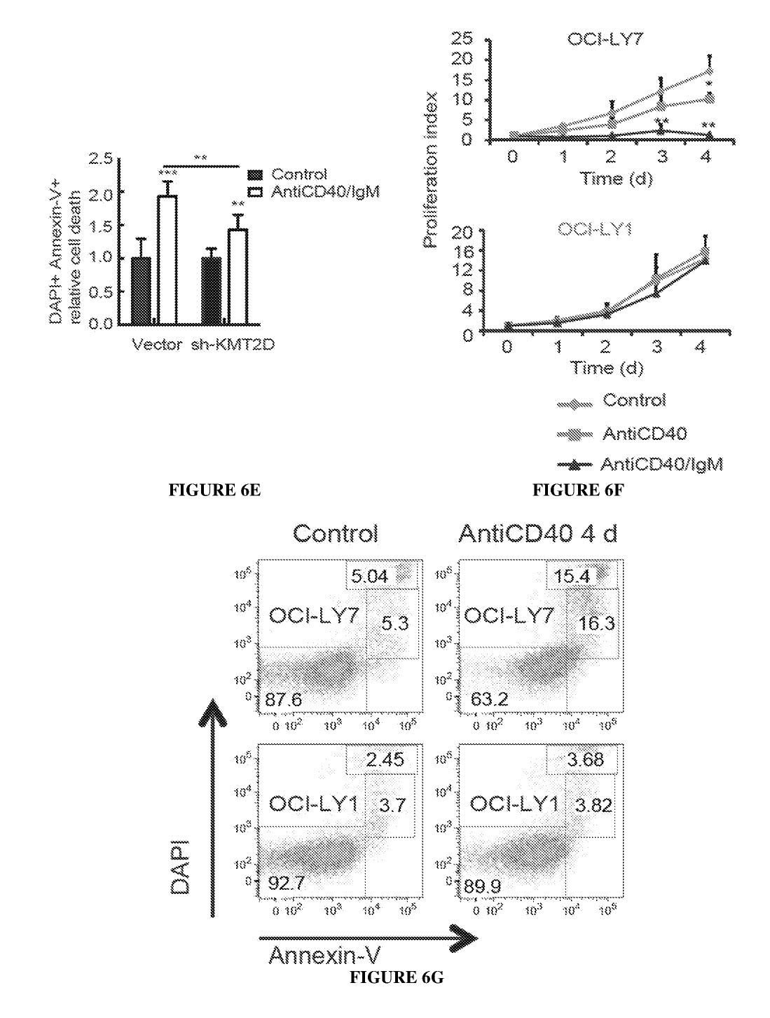

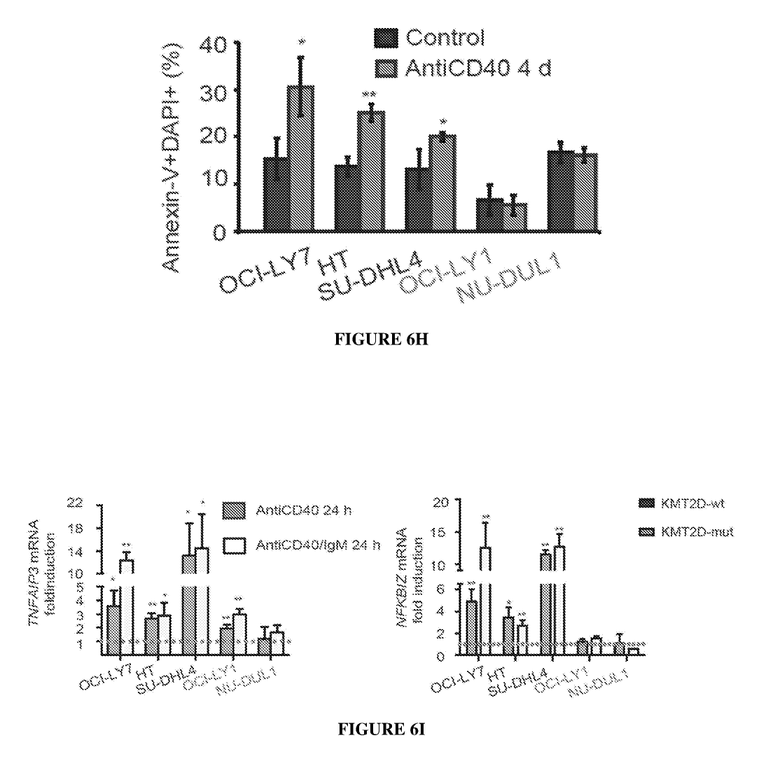

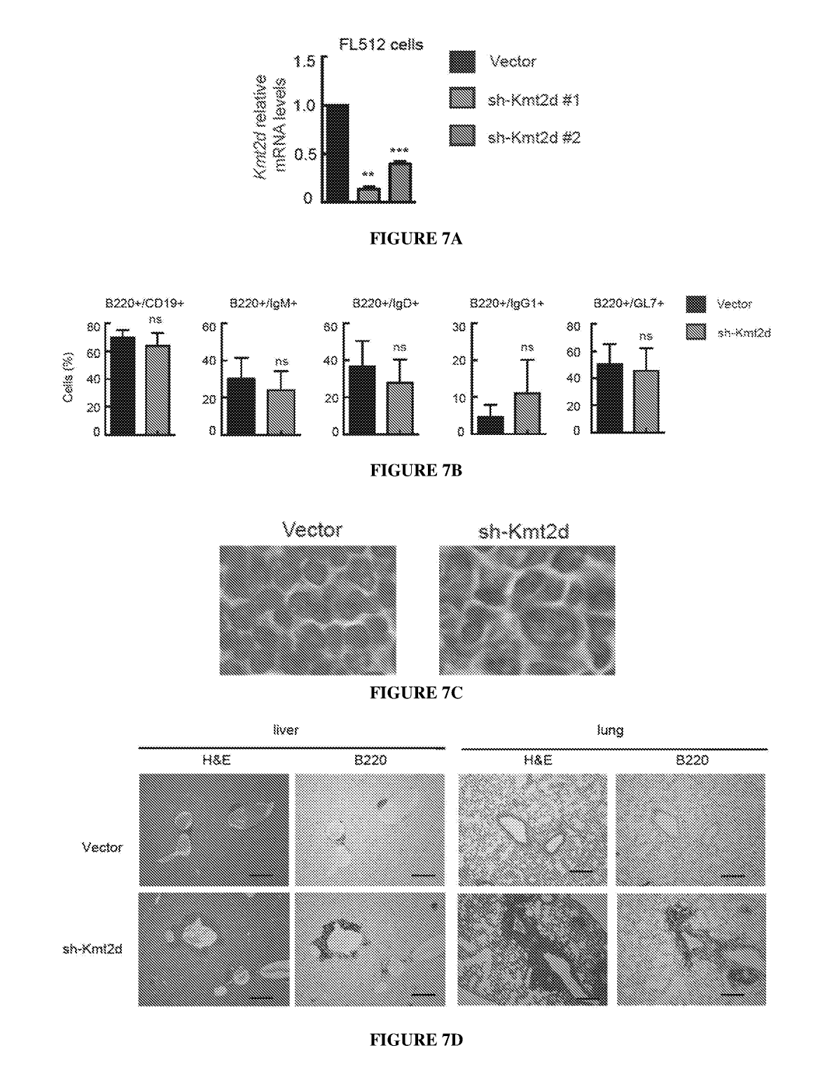

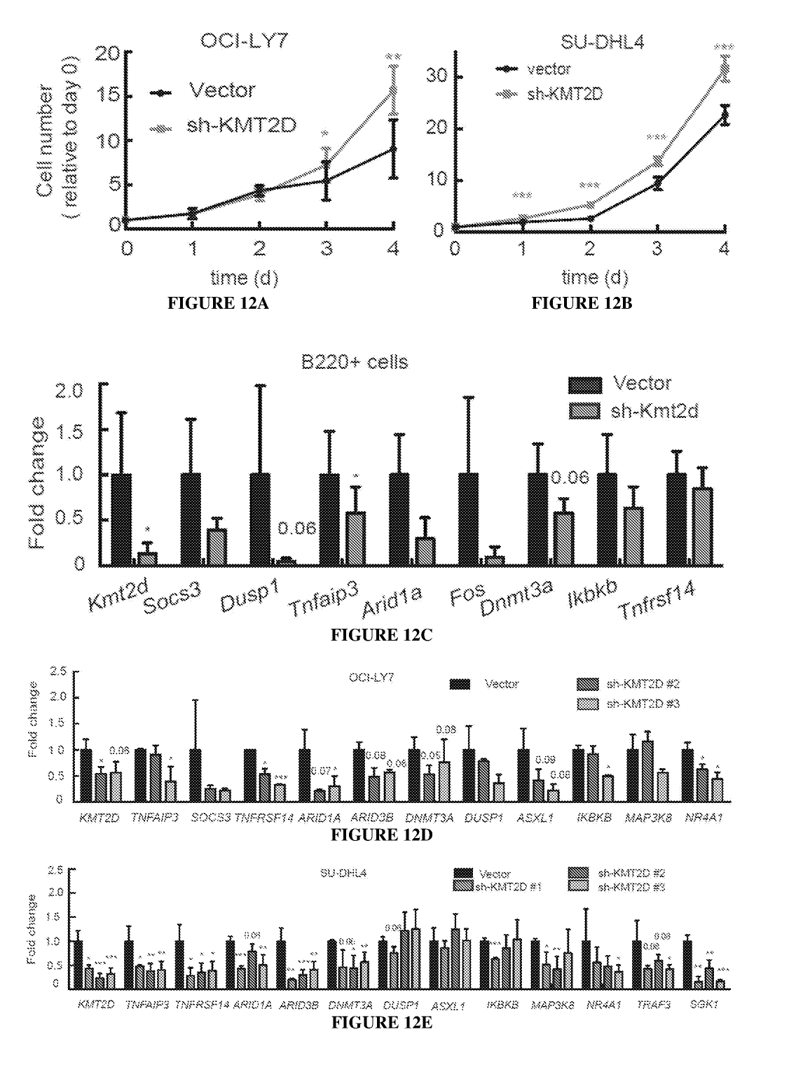

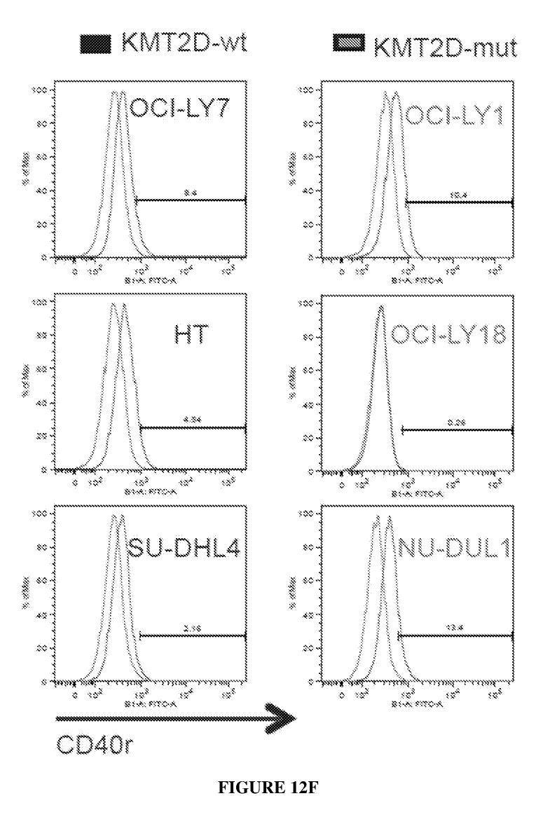

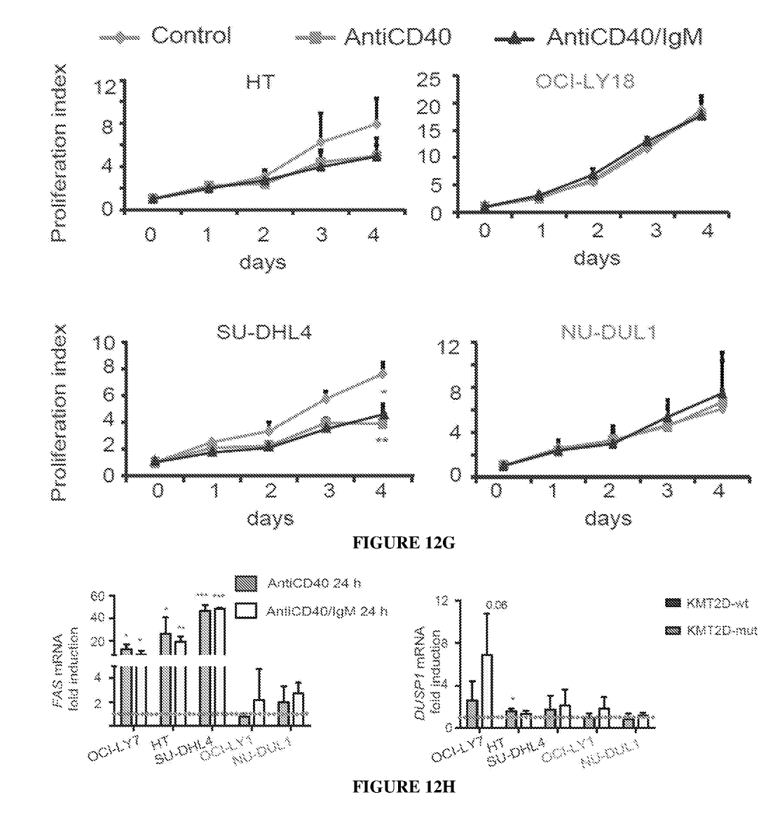

On the basis of concordant changes in expression, H3K4me1 and H3K4me2 depletion and KMT2D binding, we selected several candidate KMT2D targets for further validation (SOCS3, TNFSRF14, TNFAIP3, ARID1A, DUSP1, TRAF3, NR4A1, IKBKB, DNMT3A, ASXL1, ARID3B, MAP3K8 and SGK1). First we generated isogenic pairs of parental and KMT2D-knockdown human lymphoma cells using the wild-type KMT2D-containing lines OCI-LY7 and SU-DHL4. Unlike in certain solid tumor cells¹⁷, KMT2D-deficient lymphoma cells were more proliferative in vitro than their KMT2D-proficient parental counterparts (FIG. 12a,b). Next we tested three additional shRNAs for KMT2D knockdown (shKMT2D #1-3) and used qRT-PCR to measure effects on the expression of candidate target genes. We found a marked loss of expression for the indicated KMT2D targets in both isogenic, paired human cell lines and also in purified mouse lymphoma B220⁺ B cells (FIG. 6a and FIG. 12c-e). For further confirmation of KMT2D-mediated H3K4me1 and H3K4me2 methylation at enhancer regions, we performed H3K4me1 and H3K4me2 quantitative ChIP (qChIP) on the isogenic pairs of KMT2D-knockdown and parental OCI-LY7 cells. We observed substantial loss of H3K4me1 and H3K4me2 in the enhancer regions of SOCS3, TNFAIP3, TRAF3, SGK1 and IKBKB, as compared to their levels in TNS4, which is not a KMT2D target gene (FIG. 6b). Hence, KMT2D targets the regulatory regions of several tumor suppressor genes that control B cell signaling pathways.

Next we probed how KMT2D loss in human lymphoma cells affected the specific functions of key KMT2D targets. We generated isogenic pairs of KMT2D-proficient and KMT2D-deficient human lymphoma cell lines using shRNA knockdown. We identified SOCS3, a negative regulator of STAT3 signaling, as a KMT2D target. Accordingly, we found a reduction of SOCS3 protein levels and an augmentation in the JAK-STAT response to IL-21 stimulation in the KMT2D-deficient cells as compared to those in the isogenic control OCI-LY7 cells (FIG. 6c). Among the KMT2D target genes we also identified key signaling molecules involved in the CD40, B cell receptor (BCR) and Toll-like receptor (TLR) pathways (such as TRAF3, TNFAIP3, MAPK3K8 and DUSP1). Transcriptional expression of many of these target genes, including TNFAIP3, is dependent on CD40 and BCR signal activation (refs. 18,19). Therefore, we tested whether loss of KMT2D in the wild-type KMT2D-containing cell lines OCI-LY7 and SU-DHL4 affected the induction of KMT2D target genes when the cells were stimulated with antibodies to CD40 and IgM. Analysis by qRT-PCR showed that the induction of TNFAIP3 was greatly diminished in both cell lines after KMT2D knockdown (FIG. 6d). CD40 signaling has also been shown to be pro-apoptotic in a panel of DLBCL cell lines²⁰. Therefore we tested if KMT2D knockdown could protect OCI-LY7 cells from apoptosis induced by CD40 signaling and found that, after treatment with antibodies to CD40 and IgM, OCI-LY7 cells harboring the KMT2D-specific shRNA showed reduced cell death induction, as measured by annexin V and DAPI staining (FIG. 6e). We made analogous observations when comparing panels of lymphoma cell lines with WT (OCI-LY7, HT, SU-DHL4) or mutant (OCI-LY1, OCI-LY18, NU-DUL1) KMT2D. For example, OCI-LY7, HT and SU-DHL4 cells (which contain wild-type KMT2D) showed greater growth inhibition than cell lines OCI-LY1, OCI-LY18 and NU-DUL1 (which contain mutant KMT2D) upon treatment with antibody to CD40 alone or in combination with that to IgM (FIG. 6f and FIG. 12g). Similarly, viability assays showed that cells with wild-type KMT2D were more sensitive than cells with mutant KMT2D to CD40 stimulation and had increased levels of apoptosis, as measured by annexin V and DAPI staining (FIG. 6g,h). These differences were not caused by differential CD40 receptor expression, as only OCI-LY18 does not express the CD40 receptor and was not affected by treatment with the CD40-specific antibody (FIG. 12f). Analysis of target gene expression showed that in KMT2D-mutant cell lines there was an overall attenuated transcriptional response for important KMT2D targets such as the tumor suppressor genes TNFAIP3 (A20), NFKBIZ, FAS and DUSP1 (FIG. 6i and FIG. 12h). Hence, KMT2D deficiency affects key effects of BCR, CD40 and JAK-STAT signaling in lymphoma B cells.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

The following tables are included herein:
Table 1. Surface marker analysis in murine lymphomas
Table 2. FL and DLBCL subject features and KMT2D mutation
Table 3. Downregulated genes in FL subjects with KMT2D mutations and B220+ lymphoma cells from vav-BCL2 tumors
Table 4. Leading edge genes from GSEA and genes associated with pathways.

### LITERATURE CITED

1. De Silva, N. S. & Klein, U. Dynamics of B cells in germinal centres. Nat. Rev. Immunol. 15, 137-148 (2015).
2. Kridel, R., Sehn, L. H. & Gascoyne, R. D. Pathogenesis of follicular lymphoma. J. Clin. Invest. 122, 3424-3431 (2012).
3. Morin, R. D. et al. Frequent mutation of histone-modifying genes in non-Hodgkin lymphoma. Nature 476, 298-303 (2011).
4. Pasqualucci, L. et al. Inactivating mutations of acetyltransferase genes in B cell lymphoma. Nature 471, 189-195 (2011).
5. Hu, D. et al. The MLL3-MLL4 branches of the COMPASS family function as major histone H3K4 monomethylases at enhancers. Mol. Cell. Biol. 33, 4745-4754 (2013).
6. Herz, H. M. et al. Enhancer-associated H3K4 monomethylation by Trithorax-related, the Drosophila homolog of mammalian Mll3/Mll4. Genes Dev. 26, 2604-2620 (2012).
7. Lee, J. E., et al. H3K4 mono- and dimethyltransferase MLL4 is required for enhancer activation during cell differentiation. eLife 2:e01503 (2013).</unknown>
8. Herz, H. M., Hu, D. & Shilatifard, A. Enhancer malfunction in cancer. Mol. Cell 53, 859-866 (2014).
9. Egle, A. VavP-Bcl2 transgenic mice develop follicular lymphoma preceded by germinal center hyperplasia. Blood 103, 2276-2283 (2004).
10. Oricchio, E. et al. The Eph-receptor A7 is a soluble tumor suppressor for follicular lymphoma. Cell 147, 554-564 (2011).
11. Stadtfeld, M. & Graf, T. Assessing the role of hematopoietic plasticity for endothelial and hepatocyte development by noninvasive lineage tracing. Development 132, 203-213 (2005).
12. Santos, M. A. et al. DNA damage-induced differentiation of leukemic cells as an anti-cancer barrier. Nature 514, 107-111 (2014).
13. Hoffman, J. D. et al. Immune abnormalities are a frequent manifestation of Kabuki syndrome. Am. J. Med. Genet. A. 135, 278-281 (2005).
14. Compagno, M. et al. Mutations of multiple genes cause deregulation of NF-□B in diffuse large B cell lymphoma. Nature 459, 717-721 (2009).
15. Molavi, O. et al. Gene methylation and silencing of SOCS3 in mantle cell lymphoma. Br. J. Haematol. 161, 348-356 (2013).
16. Cheung, K. J. et al. Acquired TNFRSF14 mutations in follicular lymphoma are associated with worse prognosis. Cancer Res. 70, 9166-9174 (2010).
17. Guo, C. et al. KMT2D maintains neoplastic cell proliferation and global histone H3 lysine 4 monomethylation. Oncotarget 4, 2144-2153 (2013).
18. Sarma, V. et al. Activation of the B cell surface receptor CD40 induces A20, a novel zinc-finger protein that inhibits apoptosis. J. Biol. Chem. 270, 12343-12346 (1995).
19. Hsing, Y., Hostager, B. S. & Bishop, G. A. Characterization of CD40 signaling determinants regulating nuclear factor-kappa B activation in B lymphocytes. J. Immunol. 159, 4898-4906 (1997).
20. Hollmann, C. A., Owens, T., Nalbantoglu, J., Hudson, T. J. & Sladek, R. Constitutive activation of extracellular signal-regulated kinase predisposes diffuse large B cell lymphoma cell lines to CD40-mediated cell death. Cancer Res. 66, 3550-3557 (2006).
21. Bjornsson, H. T., et al. Histone deacetylase inhibition rescues structural and functional brain deficits in a mouse model of Kabuki syndrome. Sci. Transl. Med. 6:256ra135 (2014).</unknown>
22. Højfeldt, J. W., Agger, K. & Helin, K. Histone lysine demethylases as targets for anticancer therapy. Nat. Rev. Drug Discov. 12, 917-930 (2013).23.
23. Li, H. & Durbin, R. Fast and accurate short-read alignment with Burrows-Wheeler transform. Bioinformatics 25, 1754-1760 (2009).
24. Jiang, Y., Soong, T. D., Wang, L., Melnick, A. M. & Elemento, O. Genome-wide detection of genes targeted by non-Ig somatic hypermutation in lymphoma. PLoS ONE 7, e40332 (2012).
25. Wacker, S. A., Houghtaling, B. R., Elemento, O. & Kapoor, T. M. Using transcriptome sequencing to identify mechanisms of drug action and resistance. Nat. Chem. Biol. 8, 235-237 (2012).
26. Rajadhyaksha, A. M. et al. Mutations in FLVCR1 cause posterior column ataxia and retinitis pigmentosa. Am. J. Hum. Genet. 87, 643-654 (2010).
27. Scott, D. W. et al. Determining cell-of-origin subtypes of diffuse large B cell lymphoma using gene expression in formalin-fixed paraffin-embedded tissue. Blood 123, 1214-1217 (2014).
28. Hans, C. P. et al. Confirmation of the molecular classification of diffuse large B cell lymphoma by immunohistochemistry using a tissue microarray. Blood 103, 275-282 (2004).
29. Wendel, H. G. et al. Survival signalling by Akt and eIF4E in oncogenesis and cancer therapy. Nature 428, 332-337 (2004).
30. Dickins, R. A. et al. Probing tumor phenotypes using stable and regulated synthetic microRNA precursors. Nat. Genet. 37, 1289-1295 (2005).
31. Béguelin, W. et al. EZH2 is required for germinal center formation and somatic EZH2 mutations promote lymphoid transformation. Cancer Cell 23, 677-692 (2013).
32. Mavrakis, K. J. et al. Tumorigenic activity and therapeutic inhibition of Rheb GTPase. Genes Dev. 22, 2178-2188 (2008).
33. Hanna, J. et al. Direct reprogramming of terminally differentiated mature B lymphocytes to pluripotency. Cell 133, 250-264 (2008).
34. Ehlich, A., Martin, V., Muller, W. & Rajewsky, K. Analysis of the B cell progenitor compartment at the level of single cells. Curr. Biol. 4, 573-583 (1994).
35. Gostissa, M. et al. Conditional inactivation of p53 in mature B cells promotes generation of nongerminal center-derived B cell lymphomas. Proc. Natl. Acad. Sci. USA 110, 2934-2939 (2013).
36. Brochet, X., Lefranc, M. P. & Giudicelli, V. IMGT/V-QUEST: the highly customized and integrated system for IG and TR standardized V-J and V-D-J sequence analysis. Nucleic Acids Res. 36, W503-W508 (2008).
37. Robbiani, D. F. et al. AID is required for the chromosomal breaks in c-myc that lead to c-myc/IgH translocations. Cell 135, 1028-1038 (2008).
38. Ci, W. et al. The BCL6 transcriptional program features repression of multiple oncogenes in primary B cells and is deregulated in DLBCL. Blood 113, 5536-5548 (2009).
39. Lee, T. I., Johnstone, S. E. & Young, R. A. Chromatin immunoprecipitation and microarray-based analysis of protein location. Nat. Protoc. 1, 729-748 (2006).
40. Goodarzi, H., Elemento, O. & Tavazoie, S. Revealing global regulatory perturbations across human cancers. Mol. Cell 36, 900-911 (2009).
41. Cover, T. M. & Thomas, J. A. Elements of Information Theory (Wiley-Interscience, Hoboken, N.J., 2006).
42. Subramanian, A. et al. Gene set enrichment analysis: a knowledge-based approach for interpreting genome-wide expression profiles. Proc. Natl. Acad. Sci. USA 102, 15545-15550 (2005).
43. Shaffer, A. L. et al. A library of gene expression signatures to illuminate normal and pathological lymphoid biology. Immunol. Rev. 210, 67-85 (2006).
44. Mootha, V. K. et al. PGC-1α-responsive genes involved in oxidative phosphorylation are coordinately downregulated in human diabetes. Nat. Genet. 34, 267-273 (2003).
45. Mavrakis, K. J. & Wendel, H. G. Translational control and cancer therapy. Cell Cycle 7, 2791-2794 (2008).
46. Lin, S. & Garcia, B. A. Examining histone posttranslational modification patterns by high-resolution mass spectrometry. Methods Enzymol. 512, 3-28 (2012).
47. Yuan, Z. F. et al. EpiProfile quantifies histone peptides with modifications by extracting retention time and intensity in high-resolution mass spectra. Mol. Cell. Proteomics 14, 1696-1707 (2015).
48. Festing, M. F. W. & Laboratory Animals Ltd. The Design of Animal Experiments: Reducing the Use of Animals in Research Through Better Experimental Design (Royal Society of Medicine, London, 2002).

TABLE 1
Surface marker analysis of murine vavP-Bcl2 lymphomas

| | B220+ | | | | | | | | |
|---|---|---|---|---|---|---|---|---|---|
| | CD19+ | IgM+ | IgD+ | IgG1+ | GL7+ | Thy1+ | CD4+ | CD8+ | Sca-1 |
| VavPBcl2-v1 | 72.6 | 19.8 | 28.9 | 8.59 | 53.2 | 16.3 | 15.1 | 4.03 | 87.3 |
| VavPBcl2-v2 | 70.4 | 20.9 | 21.5 | 2.55 | 62.8 | 26.2 | 16.2 | 6.19 | 90.3 |
| VavPBcl2-v3 | 62 | 41.7 | 50.7 | 1.51 | 29.5 | 31.9 | 14.9 | 13.5 | 83.4 |
| VavPBcl2-v4 | 73.8 | 38.2 | 45.4 | 5.76 | 56.2 | 19.1 | 10.9 | 7.44 | 85.4 |
| VavPBcl2-shKmt2d-1 | 56.6 | 24.4 | 39.2 | 3.43 | 33.3 | 20.4 | 14.8 | 7.86 | 69.9 |
| VavPBcl2-shKmt2d-2 | 62.5 | 19.9 | 36.4 | 8.18 | 32 | 17.9 | 15.1 | 7.51 | 70.3 |
| VavPBcl2-shKmt2d-3 | 77 | 16.4 | 11.7 | 24.2 | 67.6 | 12.9 | 17.7 | 2.59 | 91.5 |
| VavPBcl2-shKmt2d-4 | 58.9 | 20 | 23.8 | 8.35 | 48.8 | 15.8 | 16.4 | 4.47 | 74.1 |

Surface markers analysis in Kmt2d−/− lymphomas

| | | IgM | IgD | IgL | CD19 | B220 | CD138 | HSA | CD43 | CD11b | CD4 | CD8 | CD3 |
|---|---|---|---|---|---|---|---|---|---|---|---|---|---|
| Kmt2d−/− | 1558 | − | − | − | + | − | +/− | + | int | nd | − | − | − |
| Kmt2d−/− | 2221 | + | − | + | + | low | low | + | low | nd | − | − | − |
| Kmt2d−/− | 4311 | low | low | + | low | low | +/− | + | + | nd | − | − | − |
| Kmt2d−/− | 2861 | low | − | low | + | + | + | + | + | nd | − | − | − |
| Kmt2d−/− | 2900 | low | − | − | low | − | +/− | + | + | nd | − | − | − |
| Kmt2d−/− | 4341 | + | low | + | low | + | +/− | + | low | nd | − | − | − |
| Kmt2d−/− | 2119 | + | − | + | low | low | − | + | low | nd | − | − | − |
| Kmt2d−/− | 4390 | + | − | + | + | low | + | + | int | − | − | − | − |
| Kmt2d−/− | 4383 | low | − | + | + | low | +/− | + | int | − | − | − | − |
| Kmt2d−/− | 1643 | low | low | + | + | + | − | + | low int | − | − | − | − |
| Kmt2d−/− | 4812 | + | + | + | − | + | low | + | int | − | − | − | − |
| Kmt2d−/− | 1651 | − | − | − | low | − | +/− | + | int | − | − | − | − |
| Kmt2d−/− | 4380 | + | + | + | + | + | − | + | + | − | − | − | − |
| Kmt2d−/− | 4982 | low | − | + | − | + | + | + | int | − | − | − | − |
| Kmt2d−/− | 5020 | − | − | − | low | + | − | + | low | − | − | − | − |
| Kmt2d−/− | 4737 | low | − | low | + | + | +/− | − | + | − | − | − | − |
| Kmt2d−/− | 1673 | − | nd | − | + | low | − | + | low | − | − | − | − |
| Kmt2d−/− | 4378 | + | + | + | + | + | − | + | low | − | − | − | − |
| Kmt2d−/− | 2795 | low | − | + | + | low | − | + | int | nd | − | − | − |
| Kmt2d−/− | 4384 | + | low | + | + | + | − | + | low | − | − | − | − |
| Kmt2d−/− | 1719 | − | − | − | + | − | − | + | int | − | − | − | − |
| Kmt2d−/−; AID-tg | 1816 | low | − | low | low | + | + | + | low | nd | − | − | − |
| Kmt2d−/−; AID-tg | 622 | − | − | − | − | + | + | + | + | nd | − | − | − |
| Kmt2d−/−; AID-tg | 1910 | + | − | + | + | + | +/− | + | int | nd | − | − | − |
| Kmt2d−/−; AID-tg | 2110 | + | low | + | low | + | + | + | low | nd | − | − | − |
| Kmt2d−/−; AID-tg | 19113 | − | − | − | + | + | low | + | + | nd | − | − | − |

−, negative
+, positive
low, low positive
int, intermedia
nd, not determined

SUPPL TABLE 3
Downregulated genes in FL subjects with KMT2D mutations and
B220+ lymphoma cells from vav-BCL2 mice with Kmt2d knockdown
Downreuglated genes in FL subjects with KMT2D mutations

| hgnc_symbol | ensembl | pvalue | log2FoldChange | baseMean |
|---|---|---|---|---|
| HOOK1 | ENSG00000134709 | 6.28E−08 | −2.606026655 | 706.7347038 |
| KLF11 | ENSG00000172059 | 9.53E−07 | −2.370164524 | 909.6064188 |
| HSPA1A | ENSG00000204389 | 4.20E−06 | −2.401910925 | 5976.573103 |
| GATA3 | ENSG00000107485 | 5.72E−06 | −2.242672978 | 56.00309097 |
| HSPA1B | ENSG00000204388 | 5.84E−06 | −2.217373949 | 22492.80402 |
| RARG | ENSG00000172819 | 1.18E−05 | −1.311486444 | 271.3112 |
| PMEPA1 | ENSG00000124225 | 1.68E−05 | −2.129472956 | 653.6239975 |
| LDOC1L | ENSG00000188636 | 2.29E−05 | −2.02552378 | 117.4697088 |
| GAS7 | ENSG00000007237 | 2.57E−05 | −1.966175677 | 227.0592444 |
| SLC12A7 | ENSG00000113504 | 3.52E−05 | −1.894448891 | 352.2864915 |
| HSF5 | ENSG00000176160 | 4.15E−05 | −1.982160205 | 199.2051362 |
| | ENSG00000244620 | 6.98E−05 | −2.074107287 | 73.26705493 |
| FBLN2 | ENSG00000163520 | 7.03E−05 | −1.938154167 | 152.8460752 |
| DNAJB1 | ENSG00000132002 | 7.44E−05 | −1.725961423 | 84153.89323 |
| FSCN1 | ENSG00000075618 | 0.000139244 | −1.492263287 | 648.6603338 |
| LGMN | ENSG00000100600 | 0.000141125 | −1.851330386 | 598.6973801 |
| ZBTB32 | ENSG00000011590 | 0.000160118 | −1.653053877 | 191.3119358 |
| CADM1 | ENSG00000182985 | 0.000165012 | −1.937632766 | 280.200564 |
| INSR | ENSG00000171105 | 0.00016601 | −1.775591642 | 122.2444057 |
| TOX2 | ENSG00000124191 | 0.000175472 | −1.935813853 | 280.5918922 |
| EPHA4 | ENSG00000116106 | 0.000258582 | −1.855175751 | 109.5839998 |
| JUP | ENSG00000173801 | 0.000268424 | −1.486993433 | 2295.612852 |
| DIP2B | ENSG00000066084 | 0.000312881 | −0.709884398 | 4475.311588 |
| DFNB31 | ENSG00000095397 | 0.000317896 | −1.469110018 | 169.2763435 |
| TNRC6C | ENSG00000078687 | 0.00031964 | −1.485288921 | 931.1163257 |
| KLF3 | ENSG00000109787 | 0.000340936 | −1.664604344 | 730.5146987 |
| IGHV1-24 | ENSG00000211950 | 0.000366089 | −1.848397032 | 202.089348 |
| CCR7 | ENSG00000126353 | 0.000373795 | −1.658934272 | 1849.86227 |
| SELF | ENSG00000174175 | 0.000392464 | −1.640301592 | 112.0636809 |
| IGHV5-51 | ENSG00000211966 | 0.000447991 | −1.757680571 | 630.4949681 |
| CSF1 | ENSG00000184371 | 0.000487984 | −1.732989273 | 92.5838862 |
| C10orf128 | ENSG00000204161 | 0.000489472 | −1.599042647 | 684.3551232 |
| CELF2-AS1 | ENSG00000181800 | 0.000553244 | −1.404190481 | 76.7017806 |
| MB21D1 | ENSG00000164430 | 0.000588397 | −0.867517431 | 1248.833364 |
| PLBD1 | ENSG00000121316 | 0.000610453 | −1.691797845 | 73.5238732 |
| PRKAG2 | ENSG00000106617 | 0.000614037 | −1.542477676 | 370.9465921 |
| BTBD19 | ENSG00000222009 | 0.000614118 | −1.281490271 | 238.8069845 |
| MYO15B | ENSG00000266714 | 0.000629424 | −1.581909602 | 5175.793929 |
| SERPINB9 | ENSG00000170542 | 0.000674352 | −1.058396243 | 4643.215211 |
| SELL | ENSG00000188404 | 0.000702888 | −1.195693157 | 15709.3632 |
| GGT7 | ENSG00000131067 | 0.000715818 | −1.605322203 | 409.1227928 |
| PTGR1 | ENSG00000106853 | 0.000718652 | −1.717406138 | 38.73941355 |
| HSPA6 | ENSG00000173110 | 0.000733966 | −1.781914772 | 5397.603654 |
| CTSW | ENSG00000172543 | 0.000751669 | −1.188254629 | 74.82128206 |
| NRROS | ENSG00000174004 | 0.000756384 | −1.55748082 | 423.7121647 |
| KLHL29 | ENSG00000119771 | 0.000756525 | −1.418125515 | 406.3435128 |
| PDCD1 | ENSG00000188389 | 0.000766331 | −1.622870799 | 61.43230126 |
| LTBP3 | ENSG00000168056 | 0.000767411 | −1.231417808 | 1416.607314 |
| SNX9 | ENSG00000130340 | 0.000769685 | −1.487982352 | 1176.107246 |
| CLNK | ENSG00000109684 | 0.000802396 | −1.7229022 | 1927.054381 |
| HS3ST1 | ENSG00000002587 | 0.000822461 | −1.610432857 | 573.5306956 |
| MVB12B | ENSG00000196814 | 0.000857942 | −1.53314016 | 193.7879193 |
| SDK2 | ENSG00000069188 | 0.000915363 | −1.660615152 | 881.4188107 |
| HSPA7 | ENSG00000225217 | 0.000920588 | −1.696494557 | 586.6180212 |
| IGHG3 | ENSG00000211897 | 0.000951943 | −1.494874772 | 2906.414024 |
| NA | ENSG00000182909 | 0.00111216 | −1.032777566 | 80.71002825 |
| MED13L | ENSG00000123066 | 0.001188338 | −0.658233094 | 6649.698973 |
| ZC3HAV1 | ENSG00000105939 | 0.001240794 | −0.726893259 | 13880.41689 |
| CDYL | ENSG00000153046 | 0.001283033 | −0.683945294 | 1556.132604 |
| ERRFI1 | ENSG00000116285 | 0.001290286 | −1.296969393 | 79.33536462 |
| PREX1 | ENSG00000124126 | 0.001342103 | −1.588815424 | 2323.837504 |
| SOCS3 | ENSG00000184557 | 0.001353499 | −1.341546299 | 2872.511726 |
| COL9A3 | ENSG00000092758 | 0.001360053 | −1.629672474 | 382.5054064 |
| IL7R | ENSG00000168685 | 0.001420076 | −1.482575543 | 49.01257605 |
| TNFRSF1B | ENSG00000028137 | 0.001452244 | −1.49147337 | 961.3391172 |
| NA | ENSG00000197701 | 0.001484156 | −1.607546481 | 939.2321272 |
| PLCH2 | ENSG00000149527 | 0.001509958 | −1.382678088 | 537.582655 |
| SLC37A2 | ENSG00000134955 | 0.001555573 | −1.528370771 | 96.52147424 |
| CALHM2 | ENSG00000138172 | 0.0015569 | −1.248271638 | 431.2546323 |
| ARID3A | ENSG00000116017 | 0.001558929 | −1.347961842 | 541.910903 |
| NLRP7 | ENSG00000167634 | 0.001562051 | −1.600107604 | 104.7830301 |
| DOK2 | ENSG00000147443 | 0.001575767 | −1.616930201 | 236.5509308 |
| ZNF433 | ENSG00000197647 | 0.001607769 | −1.005493262 | 207.8991903 |
| AHDC1 | ENSG00000126705 | 0.001646654 | −1.275105058 | 510.3285749 |
| SNAI1 | ENSG00000124216 | 0.001742307 | −1.221692456 | 869.9016096 |
| KLF4 | ENSG00000136826 | 0.00174801 | −1.478876785 | 2119.99914 |
| IGHV3-11 | ENSG00000211941 | 0.001783889 | −1.594447265 | 170.8703115 |
| LRRC56 | ENSG00000161328 | 0.001798764 | −1.057658253 | 359.108889 |
| EGR3 | ENSG00000179388 | 0.001804274 | −1.43261652 | 5883.571467 |
| | ENSG00000264781 | 0.001826948 | −1.649283356 | 170.1717937 |
| MTMR12 | ENSG00000150712 | 0.001842663 | −0.656343035 | 5496.286669 |
| | ENSG00000212371 | 0.001954581 | −1.379710068 | 178.3533953 |
| IGKVI-9 | ENSG00000241755 | 0.002107246 | −1.539916047 | 62.00709916 |
| HLX | ENSG00000136630 | 0.002123136 | −1.522513001 | 125.2251883 |
| GPR132 | ENSG00000183484 | 0.002136375 | −1.333499997 | 1024.258188 |
| KAZALD1 | ENSG00000107821 | 0.002335779 | −1.30357649 | 59.17193049 |
| IGKV3-11 | ENSG00000241351 | 0.002343156 | −1.477833276 | 306.3712762 |
| PLK3 | ENSG00000173846 | 0.002350355 | −0.726482468 | 3365.839368 |
| | ENSG00000233874 | 0.00237089 | −0.555137641 | 91.38616381 |
| IGKVI-16 | ENSG00000240864 | 0.002430561 | −1.569526749 | 104.1648362 |
| AXIN2 | ENSG00000168646 | 0.002485382 | −1.567904975 | 156.6789534 |
| ARRDC4 | ENSG00000140450 | 0.002504073 | −1.559261551 | 496.355505 |
| IFITM2 | ENSG00000185201 | 0.002665645 | −0.93144556 | 1518.133196 |
| ZMYND11 | ENSG00000015171 | 0.002692508 | −0.847938071 | 2071.931908 |
| C1orf115 | ENSG00000162817 | 0.0027265 | −1.577717726 | 302.0243169 |
| BAG3 | ENSG00000151929 | 0.002741882 | −1.331103564 | 3203.394329 |
| LINC00963 | ENSG00000204054 | 0.002761605 | −1.425780833 | 81.76453826 |
| NMT2 | ENSG00000152465 | 0.00278311 | −0.791688205 | 212.9669998 |
| ARHGEF5 | ENSG00000050327 | 0.00279243 | −1.567143123 | 45.57794457 |
| | ENSG00000268015 | 0.002927409 | −0.958910746 | 48.45373821 |
| CACNA1A | ENSG00000141837 | 0.002955696 | −1.304419472 | 288.897533 |
| CHORDC1 | ENSG00000110172 | 0.002962833 | −0.985695148 | 9769.159506 |
| CD274 | ENSG00000120217 | 0.003021799 | −1.162072546 | 101.4330369 |
| RN7SK | ENSG00000202198 | 0.003047549 | −1.226467906 | 1893.629558 |
| HMOX1 | ENSG00000100292 | 0.003249469 | −1.262052669 | 705.3677841 |
| RAB34 | ENSG00000109113 | 0.003362995 | −1.456135048 | 372.0656921 |
| IGHV3-49 | ENSG00000211965 | 0.003409312 | −1.499872487 | 154.1956644 |
| | ENSG00000260077 | 0.003412572 | −1.518147581 | 50.06376417 |
| IL27RA | ENSG00000104998 | 0.003479438 | −0.951994149 | 1270.998213 |
| SQSTM1 | ENSG00000161011 | 0.003515455 | −0.52215924 | 5545.646248 |
| CLCN7 | ENSG00000103249 | 0.003584541 | −0.672514959 | 6413.100849 |
| JAM3 | ENSG00000166086 | 0.00361224 | −1.283885068 | 429.7201065 |
| SYCE2 | ENSG00000161860 | 0.003695917 | −0.646980558 | 86.46650367 |
| PARP14 | ENSG00000173193 | 0.003720935 | −0.896192383 | 15218.57649 |
| PATL2 | ENSG00000229474 | 0.003819702 | −0.907410449 | 1715.947542 |
| SIK3 | ENSG00000160584 | 0.003872304 | −0.531970885 | 5416.305576 |
| PELI3 | ENSG00000174516 | 0.003885214 | −0.74178842 | 185.2758224 |
| RNF130 | ENSG00000113269 | 0.003918125 | −1.331353855 | 341.1860825 |
| SUFU | ENSG00000107882 | 0.004023752 | −0.541831939 | 1059.354877 |
| FBXW4 | ENSG00000107829 | 0.004249071 | −0.475495941 | 4387.838616 |
| FAM43A | ENSG00000185112 | 0.004346381 | −1.044728963 | 2305.832956 |
| CAMKK1 | ENSG00000004660 | 0.004375984 | −1.052154902 | 293.0369706 |
| SPG20 | ENSG00000133104 | 0.004413395 | −1.467249269 | 560.3494788 |
| PRDM1 | ENSG00000057657 | 0.004443008 | −1.374809739 | 739.5559853 |
| | ENSG00000203362 | 0.004568927 | −0.843026285 | 46.85485639 |
| SESTD1 | ENSG00000187231 | 0.004633775 | −0.820078329 | 2728.772328 |
| IGHV1-2 | ENSG00000211934 | 0.004641079 | −1.43870881 | 298.5982172 |
| FGR | ENSG00000000938 | 0.004657393 | −1.390733538 | 1256.254801 |
| SNORD3A | ENSG00000263934 | 0.004887424 | −1.397721152 | 178.4536263 |
| C110rf85 | ENSG00000168070 | 0.004903994 | −1.480483334 | 65.77374793 |
| NABP1 | ENSG00000173559 | 0.004978121 | −0.976766388 | 2382.127912 |
| | ENSG00000263606 | 0.004998783 | −0.650360155 | 519.9027944 |
| | ENSG00000267216 | 0.00500429 | −0.678572327 | 51.57936185 |
| PKD1 | ENSG00000008710 | 0.005009649 | −0.487782388 | 3879.783724 |
| PLAUR | ENSG00000011422 | 0.005024536 | −1.196117419 | 53.72817587 |
| RYKP1 | ENSG00000263219 | 0.005094108 | −0.824682147 | 45.57856763 |
| PIEZO1 | ENSG00000103335 | 0.005101844 | −1.364610047 | 1469.392301 |
| RILPL2 | ENSG00000150977 | 0.005106538 | −0.76331206 | 1848.624506 |
| GPX1 | ENSG00000233276 | 0.005196913 | −0.851218057 | 2568.928957 |
| TCF7 | ENSG00000081059 | 0.005236157 | −1.067454918 | 406.4773948 |
| SMG1P3 | ENSG00000180747 | 0.00525927 | −0.678554114 | 645.4986796 |
| NAALAD2 | ENSG00000077616 | 0.005356353 | −0.821532871 | 269.397953 |
| CD7 | ENSG00000173762 | 0.005444962 | −1.408884263 | 43.76833236 |
| HPS1 | ENSG00000107521 | 0.005557405 | −0.387824182 | 10154.93153 |
| LY6E | ENSG00000160932 | 0.005655379 | −1.376047788 | 961.3049692 |
| IGLV1-40 | ENSG00000211653 | 0.005660673 | −1.214471175 | 166.1517526 |
| CDC42EP4 | ENSG00000179604 | 0.005801027 | −1.188779076 | 52.43855765 |
| ACSS2 | ENSG00000131069 | 0.005807684 | −1.19132702 | 102.8866055 |
| CD72 | ENSG00000137101 | 0.005834777 | −0.959280191 | 14157.69454 |
| GDF11 | ENSG00000135414 | 0.005876632 | −0.898791037 | 275.7392487 |
| ITGA5 | ENSG00000161638 | 0.005912479 | −1.388670037 | 91.36828002 |
| XAB2 | ENSG00000076924 | 0.005961575 | −0.296193042 | 5034.424651 |
| TNFRSF13B | ENSG00000240505 | 0.006175912 | −1.388276609 | 1211.91348 |
| | ENSG00000265517 | 0.00621176 | −1.372646918 | 223.6550471 |
| CCND1 | ENSG00000110092 | 0.006298205 | −0.994997881 | 231.3684014 |
| GRK6P1 | ENSG00000215571 | 0.006379697 | −0.595153742 | 98.31827799 |
| TLE1P1 | ENSG00000228158 | 0.006431417 | −1.398792453 | 137.465281 |
| PTPRK | ENSG00000152894 | 0.006442566 | −1.370200204 | 570.8393436 |
| IGHV3-21 | ENSG00000211947 | 0.006499833 | −1.357188874 | 264.2493277 |
| SERPINB6 | ENSG00000124570 | 0.006546525 | −1.313945181 | 603.0642676 |
| RNF125 | ENSG00000101695 | 0.006701709 | −1.357384629 | 198.4843949 |
| UST | ENSG00000111962 | 0.006816397 | −1.299257377 | 360.1798649 |
| ZNF492 | ENSG00000229676 | 0.006833281 | −1.428684519 | 47.4562949 |
| TECR | ENSG00000099797 | 0.006833706 | −0.828520855 | 2142.475421 |
| ARIDSA | ENSG00000196843 | 0.006839714 | −0.783748744 | 3670.977783 |
| RNF43 | ENSG00000108375 | 0.006882135 | −1.165735136 | 114.2890116 |
| TBXAS1 | ENSG00000059377 | 0.007065814 | −1.316926216 | 157.1527074 |
| GPAT2 | ENSG00000186281 | 0.007110011 | −1.38613601 | 49.83170129 |
| DUSP6 | ENSG00000139318 | 0.007126128 | −0.887185222 | 4858.862542 |
| TNFSF12 | ENSG00000239697 | 0.007145773 | −1.121247896 | 473.6690034 |
| | ENSG00000237938 | 0.007215338 | −0.850937628 | 69.13874639 |
| SCML2 | ENSG00000102098 | 0.007224749 | −1.415022461 | 57.49995465 |
| IL6 | ENSG00000136244 | 0.007334792 | −1.205049967 | 1759.424118 |
| ARNTL | ENSG00000133794 | 0.007378966 | −0.969262593 | 1525.822384 |
| | ENSG00000245017 | 0.007407381 | −0.853435557 | 44.25613498 |
| NA | ENSG00000251606 | 0.007422564 | −1.283536115 | 76.81784924 |
| IRAK2 | ENSG00000134070 | 0.007680771 | −0.927445761 | 1868.408049 |
| | ENSG00000263751 | 0.007703752 | −1.404398383 | 171.9567079 |
| ADAT3 | ENSG00000213638 | 0.007712794 | −0.614234878 | 40.82795355 |
| NA | ENSG00000174194 | 0.007729464 | −0.712977978 | 138.7163078 |
| PARP15 | ENSG00000173200 | 0.007811301 | −1.174499508 | 9328.897754 |
| THRA | ENSG00000126351 | 0.007897511 | −0.865052788 | 296.1160371 |
| ST6GALNAC3 | ENSG00000184005 | 0.007909181 | −1.358396716 | 42.35874597 |
| HHEX | ENSG00000152804 | 0.007916049 | −0.836242478 | 6494.896917 |
| JUNB | ENSG00000171223 | 0.007985014 | −0.749184467 | 62245.27361 |
| ESAM | ENSG00000149564 | 0.008104033 | −1.15466855 | 306.7436799 |
| | ENSG00000230076 | 0.008137058 | −0.629438838 | 442.5234279 |
| NDRG2 | ENSG00000165795 | 0.008173915 | −1.236563446 | 98.69135497 |
| | ENSG00000261207 | 0.008227809 | −0.922712128 | 106.6976694 |
| SLCO4A1 | ENSG00000101187 | 0.008472558 | −1.322724118 | 143.3655277 |
| NAB2 | ENSG00000166886 | 0.008502231 | −0.766644034 | 1608.845189 |
| MYRIP | ENSG00000170011 | 0.008800867 | −1.38450835 | 40.53691189 |
| MLF1 | ENSG00000178053 | 0.009016763 | −1.366785254 | 77.73280177 |
| IGKV1-8 | ENSG00000240671 | 0.009100048 | −1.2828098 | 69.70373344 |
| IL15RA | ENSG00000134470 | 0.009238207 | −0.936494606 | 214.7324397 |
| C10orf32 | ENSG00000166275 | 0.009274214 | −0.488659858 | 929.204537 |
| HSPG2 | ENSG00000142798 | 0.009498091 | −1.307391609 | 67.64690145 |
| | ENSG00000260521 | 0.009553421 | −0.515066012 | 1924.570674 |
| RPL19P21 | ENSG00000230508 | 0.009626061 | −0.858190511 | 205.8398479 |
| KLF9 | ENSG00000119138 | 0.009834837 | −1.088107119 | 1673.680535 |
| CSF1R | ENSG00000182578 | 0.009881442 | −0.781550933 | 91.52630848 |
| HES6 | ENSG00000144485 | 0.00993007 | −1.139381237 | 176.4842882 |
| HSPE1 | ENSG00000115541 | 0.010061733 | −0.83502009 | 2773.999223 |
| | ENSG00000217801 | 0.010142748 | −1.201413301 | 162.9180178 |
| KRT8P50 | ENSG00000260799 | 0.010200039 | −0.745914217 | 40.24249239 |
| HSP90AA1 | ENSG00000080824 | 0.010324636 | −0.810978127 | 166583.0975 |
| ZNF677 | ENSG00000197928 | 0.010552071 | −1.327290514 | 86.69821157 |
| TCP1 | ENSG00000120438 | 0.010648019 | −0.585688181 | 11059.68746 |
| HSP90AB2P | ENSG00000205940 | 0.010693579 | −0.646087199 | 555.9836404 |
| NOXA1 | ENSG00000188747 | 0.010804546 | −1.144873547 | 570.2339145 |
| GDPGP1 | ENSG00000183208 | 0.010844745 | −0.481764667 | 130.1386326 |
| IGLV7-43 | ENSG00000211652 | 0.010853388 | −1.164768667 | 41.44311137 |
| MORN1 | ENSG00000116151 | 0.010935368 | −0.865462547 | 166.7716055 |
| | ENSG00000266706 | 0.010954896 | −1.34572516 | 133.5281503 |
| IGLV2-14 | ENSG00000211666 | 0.011049827 | −1.159262864 | 147.920407 |
| RNY1P16 | ENSG00000199933 | 0.011113381 | −0.993415692 | 53.30064432 |
| MAN2A2 | ENSG00000196547 | 0.011123908 | −0.67989203 | 6355.79058 |
| | ENSG00000234750 | 0.011200454 | −0.966241286 | 41.04084603 |
| FRAT1 | ENSG00000165879 | 0.011370555 | −0.501404353 | 1228.096356 |
| CCDC113 | ENSG00000103021 | 0.011493114 | −1.051968416 | 80.64716993 |
| CRAT | ENSG00000095321 | 0.011494857 | −1.263462911 | 193.767124 |
| PI4K2A | ENSG00000155252 | 0.011521642 | −0.631058792 | 1899.479914 |
| IGHV3-13 | ENSG00000211942 | 0.011539003 | −1.30098874 | 53.93412894 |
| CHPT1 | ENSG00000111666 | 0.011550456 | −0.618869384 | 2254.916593 |
| APOD | ENSG00000189058 | 0.011572134 | −1.232696043 | 195.6866898 |
| TRPM2 | ENSG00000142185 | 0.011642077 | −1.279229136 | 350.1801288 |
| ATN1 | ENSG00000111676 | 0.011763253 | −0.959922214 | 4620.846114 |
| CUBN | ENSG00000107611 | 0.011839579 | −1.038946745 | 337.6392211 |
| CLEC17A | ENSG00000187912 | 0.011850053 | −1.065950296 | 4117.56126 |
| | ENSG00000226915 | 0.011950717 | −0.717010124 | 106.8592618 |
| ANXA4 | ENSG00000196975 | 0.012046453 | −1.222438866 | 671.3293009 |
| BCAS1 | ENSG00000064787 | 0.012100929 | −1.11821548 | 318.6914879 |
| CRTC3 | ENSG00000140577 | 0.012135728 | −0.796529357 | 2857.465993 |
| TLE1 | ENSG00000196781 | 0.012156023 | −1.315249851 | 951.6040937 |
| SNORD64 | ENSG00000270704 | 0.012340205 | −0.976689672 | 46.5993745 |
| MRPL18 | ENSG00000112110 | 0.012391051 | −0.531437021 | 2477.98452 |
| CD5 | ENSG00000110448 | 0.012428311 | −1.207098153 | 114.9373747 |
| HSPA2 | ENSG00000126803 | 0.012555351 | −1.184832125 | 573.2886948 |
| SLC25A28 | ENSG00000155287 | 0.012587065 | −0.540945388 | 4196.766933 |
| EFCAB12 | ENSG00000172771 | 0.012696544 | −0.944380622 | 874.7597314 |
| ELL | ENSG00000105656 | 0.012718151 | −0.47608329 | 1528.60021 |
| | ENSG00000266408 | 0.012749697 | −1.255563897 | 163.6684072 |
| PDLIM1P1 | ENSG00000270788 | 0.012768889 | −1.111097815 | 40.22381041 |
| SIRPB1 | ENSG00000101307 | 0.012827767 | −1.285673684 | 257.1286648 |
| TMPPE | ENSG00000188167 | 0.01293819 | −0.594812226 | 227.9648767 |
| FSIP2 | ENSG00000188738 | 0.013106601 | −1.261574775 | 182.1930909 |
| | ENSG00000259363 | 0.013293063 | −1.089505882 | 73.1916989 |
| NA | ENSG00000271738 | 0.013337744 | −1.010575242 | 105.2468375 |
| IGLV2-11 | ENSG00000211668 | 0.013360593 | −1.261924075 | 93.33711656 |
| RPS6KL1 | ENSG00000198208 | 0.013384424 | −1.017349154 | 232.6989824 |
| | ENSG00000260051 | 0.013395157 | −0.856098969 | 173.7458631 |
| C17orf51 | ENSG00000212719 | 0.013501487 | −1.033206873 | 117.9556452 |
| FLNA | ENSG00000196924 | 0.013528771 | −0.80118106 | 12729.56122 |
| APCDD1 | ENSG00000154856 | 0.013768831 | −1.204136495 | 48.51628979 |
| ZNF57 | ENSG00000171970 | 0.013890307 | −0.904575927 | 160.6008636 |
| NFATC3 | ENSG00000072736 | 0.013952989 | −0.592538936 | 5030.012426 |
| HSPD1 | ENSG00000144381 | 0.014079291 | −0.834656504 | 16542.34288 |
| RGMB | ENSG00000174136 | 0.014121423 | −0.969031231 | 816.7904819 |
| | ENSG00000265612 | 0.01424417 | −1.273655611 | 181.195624 |
| HSP90AA2P | ENSG00000224411 | 0.014273528 | −0.809563115 | 9517.453407 |
| ANXA1 | ENSG00000135046 | 0.014359378 | −1.186019182 | 66.98079353 |
| | ENSG00000231434 | 0.014402849 | −0.820539972 | 703.996571 |
| PDCD11 | ENSG00000148843 | 0.014512013 | −0.393152452 | 4084.309393 |
| ERICH6-AS1 | ENSG00000240137 | 0.014533272 | −0.858461816 | 77.02174085 |
| MAPK8IP3 | ENSG00000138834 | 0.014598676 | −0.678645373 | 21935.24166 |
| CHL1 | ENSG00000134121 | 0.014607331 | −1.256049708 | 2873.305448 |
| DOPEY2 | ENSG00000142197 | 0.014615774 | −0.812167024 | 3098.701088 |
| DEDD2 | ENSG00000160570 | 0.014620194 | −0.755969098 | 8743.643667 |
| RP9P | ENSG00000205763 | 0.014658921 | −1.20680086 | 60.63961666 |
| NA | ENSG00000248835 | 0.014718481 | −0.462671362 | 319.4248328 |
| SIPA1 | ENSG00000213445 | 0.014762347 | −0.533353162 | 15191.76327 |
| UTRN | ENSG00000152818 | 0.014801752 | −0.847065271 | 6079.33611 |
| FHL1 | ENSG00000022267 | 0.014896032 | −1.097678174 | 65.31473023 |
| LDOC1 | ENSG00000182195 | 0.014902289 | −1.284149894 | 182.101876 |
| TMEM173 | ENSG00000184584 | 0.015038176 | −1.131433361 | 47.81961483 |
| IGHV4-59 | ENSG00000224373 | 0.015071556 | −1.159388204 | 141.0175931 |
| PDLIM7 | ENSG00000196923 | 0.015131305 | −0.732939801 | 1290.000033 |
| ZKSCAN3 | ENSG00000189298 | 0.015291931 | −0.361461407 | 620.9615611 |
| STARD5 | ENSG00000172345 | 0.015316097 | −0.619180438 | 1471.911204 |
| EPHB1 | ENSG00000154928 | 0.015562346 | −1.261887238 | 1051.26415 |
| PRR5 | ENSG00000186654 | 0.015724868 | −1.267072882 | 50.94493953 |
| FBXO6 | ENSG00000116663 | 0.015924967 | −0.810615078 | 444.2864148 |
| IGKV1-12 | ENSG00000243290 | 0.015947631 | −1.148121859 | 41.73155506 |
| IFFO2 | ENSG00000169991 | 0.016114377 | −1.019836834 | 1365.794226 |
| ZNF703 | ENSG00000183779 | 0.016228734 | −1.114062663 | 208.5500355 |
| TBC1D27 | ENSG00000128438 | 0.016513844 | −1.22934711 | 1401.307749 |
| TCTEX1D4 | ENSG00000188396 | 0.016652488 | −0.938040149 | 41.17765798 |
| MSX1 | ENSG00000163132 | 0.016718997 | −1.251394405 | 156.5782407 |
| NLRP6 | ENSG00000174885 | 0.016726927 | −1.098990167 | 54.41473605 |
| IGHV1-46 | ENSG00000211962 | 0.016729207 | −1.188249692 | 92.1533394 |
| CERS6 | ENSG00000172292 | 0.016774947 | −1.223816963 | 194.38565 |
| TMEM8B | ENSG00000137103 | 0.017062675 | −0.533310732 | 1237.689541 |
| SNORA20 | ENSG00000207392 | 0.017068628 | −0.886575436 | 59.40897376 |
| NA | ENSG00000174111 | 0.017122423 | −0.638358286 | 321.2116424 |
| TLR4 | ENSG00000136869 | 0.017140445 | −1.201233838 | 518.7400472 |
| SCML1 | ENSG00000047634 | 0.017243146 | −1.250568353 | 435.2201969 |
| AMOT | ENSG00000126016 | 0.017253164 | −1.13474413 | 273.9477046 |
| SH3RF3 | ENSG00000172985 | 0.017309234 | −1.185269628 | 54.67603133 |
| CHN2 | ENSG00000106069 | 0.017501618 | −1.253695566 | 691.5220056 |
| RRAGD | ENSG00000025039 | 0.017838901 | −1.030968477 | 193.3427167 |
| GNE | ENSG00000159921 | 0.017928242 | −0.419064032 | 2274.374385 |
| CD19 | ENSG00000177455 | 0.018308888 | −0.509899627 | 33891.93637 |
| TNNT3 | ENSG00000130595 | 0.01850668 | −0.936756888 | 98.14925769 |
| ITGB7 | ENSG00000139626 | 0.018815613 | −0.966572825 | 5215.626484 |
| CIB1 | ENSG00000185043 | 0.018830561 | −0.479005766 | 6788.374752 |
| | ENSG00000264469 | 0.018884791 | −0.69481884 | 236.3138535 |
| GPD1 | ENSG00000167588 | 0.018936182 | −0.643883077 | 98.13013357 |
| GRAP2 | ENSG00000100351 | 0.019209399 | −1.194004512 | 71.49612787 |
| SLC12A8 | ENSG00000221955 | 0.019220746 | −1.005503819 | 96.32113805 |
| TOR4A | ENSG00000198113 | 0.01929589 | −0.808531582 | 665.5792394 |
| | ENSG00000228143 | 0.019532879 | −0.853784472 | 41.80405943 |
| IGKV1-27 | ENSG00000244575 | 0.019621737 | −1.165439195 | 85.18834223 |
| MAP1A | ENSG00000166963 | 0.01975222 | −1.022125996 | 423.4757321 |
| IRF1 | ENSG00000125347 | 0.020115773 | −0.668801439 | 10626.68444 |
| TGM2 | ENSG00000198959 | 0.020407851 | −1.133547932 | 258.9611168 |
| DCBLD2 | ENSG00000057019 | 0.020448429 | −1.216961212 | 46.60052254 |
| BANK1 | ENSG00000153064 | 0.020618461 | −0.511113901 | 35631.35192 |
| TCFL5 | ENSG00000101190 | 0.020743562 | −0.636180545 | 742.9839979 |
| PNMAL1 | ENSG00000182013 | 0.020787187 | −1.161353538 | 48.04038577 |
| TYMP | ENSG00000025708 | 0.020939726 | −0.811615728 | 1226.980696 |
| | ENSG00000227176 | 0.02098221 | −0.911402431 | 53.9165327 |
| FAM90A1 | ENSG00000171847 | 0.02099384 | −0.983626669 | 56.16716739 |
| CDH13 | ENSG00000140945 | 0.021029805 | −1.136767259 | 40.29529628 |
| CARD9 | ENSG00000187796 | 0.021117524 | −0.995073783 | 217.5525363 |
| | ENSG00000227359 | 0.021229996 | −1.126016678 | 45.80711325 |
| | ENSG00000215154 | 0.021424781 | −0.682289565 | 85.62623181 |
| MTND2P28 | ENSG00000225630 | 0.021531275 | −1.075526849 | 288.2213016 |
| BTBD3 | ENSG00000132640 | 0.021580741 | −1.18368154 | 71.15113667 |
| FAM46C | ENSG00000183508 | 0.021625756 | −0.771754908 | 18720.49254 |
| ARRDC5 | ENSG00000205784 | 0.021800878 | −1.016204994 | 72.36016585 |
| SPATA6 | ENSG00000132122 | 0.021823805 | −1.214065055 | 62.45963769 |
| ATP2B4 | ENSG00000058668 | 0.022049182 | −1.140421472 | 444.0579454 |
| KIAA0125 | ENSG00000226777 | 0.022107671 | −1.15660731 | 661.657367 |
| MYBPC2 | ENSG00000086967 | 0.022111096 | −0.967889346 | 393.5371292 |
| C21orf140 | ENSG00000222018 | 0.022148374 | −0.828459219 | 47.59763205 |
| SYNPO | ENSG00000171992 | 0.022195358 | −1.090259999 | 813.8920424 |
| CA11 | ENSG00000063180 | 0.022222987 | −0.873093888 | 272.5468224 |
| CNKSR2 | ENSG00000149970 | 0.022242361 | −1.071050097 | 996.362537 |
| NINJ1 | ENSG00000131669 | 0.022262221 | −1.014629363 | 922.4266601 |
| PIK3R4 | ENSG00000196455 | 0.022351509 | −0.369837388 | 2652.545308 |
| SNORD14E | ENSG00000200879 | 0.022388624 | −0.759407038 | 462.5671564 |
| PKN3 | ENSG00000160447 | 0.022424372 | −0.896253018 | 506.0252494 |
| STK10 | ENSG00000072786 | 0.02244301 | −0.398798107 | 12049.32294 |
| FAM213A | ENSG00000122378 | 0.022836876 | −0.757713935 | 506.3941194 |
| IKZF1 | ENSG00000185811 | 0.022841952 | −0.412822476 | 23021.15871 |
| PDGFRB | ENSG00000113721 | 0.022870116 | −1.065747545 | 61.41169631 |
| GTPBP1 | ENSG00000100226 | 0.022882485 | −0.347002526 | 5962.246064 |
| SCARNA21 | ENSG00000252835 | 0.02301958 | −0.979527529 | 54.59687538 |
| BATF2 | ENSG00000168062 | 0.023059478 | −1.097151916 | 90.58658659 |
| CD48 | ENSG00000117091 | 0.023196459 | −0.76188096 | 8329.524048 |
| IGLV2-23 | ENSG00000211660 | 0.023311434 | −1.118587022 | 139.7149559 |
| HSPB1 | ENSG00000106211 | 0.023530229 | −1.13167149 | 2233.454021 |
| PODXL2 | ENSG00000114631 | 0.023645662 | −1.035746757 | 648.2074529 |
| LDB1 | ENSG00000198728 | 0.023656708 | −0.434590018 | 7288.490979 |
| SLC18B1 | ENSG00000146409 | 0.023699892 | −0.760350324 | 855.7405937 |
| PLXND1 | ENSG00000004399 | 0.023890109 | −0.974891276 | 864.1082873 |
| SDC4 | ENSG00000124145 | 0.023935312 | −1.155656524 | 484.0291519 |
| TBX21 | ENSG00000073861 | 0.023971334 | −0.979969137 | 244.8584004 |
| IGHV3-15 | ENSG00000211943 | 0.024006111 | −1.073020891 | 159.2467412 |
| KCNG1 | ENSG00000026559 | 0.024079196 | −1.175195855 | 71.21895559 |
| IFIT2 | ENSG00000119922 | 0.024080414 | −0.79047955 | 3508.199757 |
| PLCB2 | ENSG00000137841 | 0.024134149 | −0.647318237 | 8241.957018 |
| FAR2P2 | ENSG00000178162 | 0.024243235 | −1.083516578 | 332.3887502 |
| KHDRBS2 | ENSG00000112232 | 0.024245578 | −1.183892663 | 120.1829082 |
| METRN | ENSG00000103260 | 0.024364815 | −0.700828677 | 226.6386241 |
| DUSP3 | ENSG00000108861 | 0.024505009 | −0.714683152 | 964.0530077 |
| DDAH2 | ENSG00000213722 | 0.024520179 | −0.72889651 | 1379.478662 |
| RBM17P4 | ENSG00000259585 | 0.024594336 | −0.351613097 | 159.5923786 |
| ZNF267 | ENSG00000185947 | 0.024634397 | −0.501381917 | 2596.404339 |
| IGHV3-48 | ENSG00000211964 | 0.024725303 | −1.143943773 | 171.5860897 |
| ZNF597 | ENSG00000167981 | 0.02473649 | −0.957524502 | 542.7136742 |
| PHF20 | ENSG00000025293 | 0.024809101 | −0.252735525 | 4441.204806 |
| KLHL25 | ENSG00000183655 | 0.024899764 | −0.541812203 | 173.0940416 |
| MYOM1 | ENSG00000101605 | 0.024935569 | −0.743892948 | 659.6661694 |
| RABEP2 | ENSG00000177548 | 0.025051723 | −0.466575911 | 8983.660561 |
| NUMBL | ENSG00000105245 | 0.025063835 | −0.512635726 | 695.6593648 |
| DSE | ENSG00000111817 | 0.025097389 | −0.91952286 | 625.4506096 |
| IGHV2-26 | ENSG00000211951 | 0.025118403 | −0.98558931 | 50.00836084 |
| NFRKB | ENSG00000170322 | 0.02514458 | −0.285763143 | 3387.403527 |
| PTCHD2 | ENSG00000204624 | 0.025304818 | −1.100355729 | 62.4863163 |
| RILP | ENSG00000167705 | 0.025416055 | −0.600578964 | 109.3833789 |
| | ENSG00000269896 | 0.025440795 | −0.877356158 | 177.7866638 |
| ALOX15 | ENSG00000161905 | 0.02552522 | −1.102847588 | 122.0581344 |
| DKK1 | ENSG00000107984 | 0.025619909 | −1.181142246 | 41.77606574 |
| | ENSG00000233597 | 0.025835654 | −0.750499917 | 144.0436501 |
| KLF2 | ENSG00000127528 | 0.025871361 | −0.677178429 | 22016.7369 |
| SIK1 | ENSG00000142178 | 0.025945818 | −0.906324575 | 68402.83126 |
| NEIL2 | ENSG00000154328 | 0.026041419 | −0.678441789 | 701.1145322 |
| | ENSG00000233028 | 0.026047896 | −0.747664693 | 165.6573677 |
| MYO1F | ENSG00000142347 | 0.026107467 | −1.102330974 | 474.2062267 |
| MYO5B | ENSG00000167306 | 0.02612698 | −1.086714941 | 57.69740462 |
| FAM129C | ENSG00000167483 | 0.026440782 | −0.713921141 | 21002.88114 |
| FAM69B | ENSG00000165716 | 0.026458923 | −0.807096159 | 152.8128786 |
| CPM | ENSG00000135678 | 0.026459213 | −1.045619258 | 125.5256103 |
| SKI | ENSG00000157933 | 0.026694256 | −0.784390029 | 1493.096729 |
| CLMN | ENSG00000165959 | 0.026778966 | −1.022945471 | 763.1112578 |
| | ENSG00000258733 | 0.026903358 | −0.963759035 | 88.5244254 |
| LGALS3BP | ENSG00000108679 | 0.026903661 | −1.03448771 | 407.4829786 |
| PLTP | ENSG00000100979 | 0.027089295 | −0.86262273 | 160.7073169 |
| ALDOC | ENSG00000109107 | 0.027407162 | −0.705658636 | 1369.69727 |
| WDR81 | ENSG00000167716 | 0.027461234 | −0.580517147 | 4867.117434 |
| | ENSG00000244480 | 0.027508668 | −0.619413673 | 108.922964 |
| SYNGR1 | ENSG00000100321 | 0.027565078 | −0.919114562 | 1413.994046 |
| NKG7 | ENSG00000105374 | 0.02770669 | −0.915811135 | 38.48480891 |
| SH3BGR | ENSG00000185437 | 0.027900178 | −0.625091 | 47.53933385 |
| | ENSG00000270442 | 0.027942198 | −0.558009866 | 58.26058264 |
| CACFD1 | ENSG00000160325 | 0.028100598 | −0.611212486 | 943.356209 |
| NEAT1 | ENSG00000245532 | 0.028125571 | −0.664301549 | 21998.08567 |
| ZSCAN31 | ENSG00000235109 | 0.02824106 | −0.700936189 | 61.94573732 |
| WDFY1 | ENSG00000085449 | 0.028318021 | −0.45272126 | 4749.323481 |
| ARSD | ENSG00000006756 | 0.028371393 | −1.078338413 | 79.69941964 |
| LINC00884 | ENSG00000233058 | 0.028655818 | −0.779689819 | 41.77406133 |
| CYB5RL | ENSG00000215883 | 0.028796359 | −0.480385523 | 326.0419821 |
| IFI30 | ENSG00000216490 | 0.028889607 | −0.861232978 | 586.8956869 |
| RPS15AP40 | ENSG00000233921 | 0.029184009 | −0.657117578 | 71.10198154 |
| NRARP | ENSG00000198435 | 0.029202336 | −0.944582958 | 904.0486298 |
| CD2 | ENSG00000116824 | 0.02951878 | −1.148674702 | 52.69369994 |
| AFF1 | ENSG00000172493 | 0.029522721 | −0.820964101 | 2053.189617 |
| CKAP4 | ENSG00000136026 | 0.029890309 | −0.998370867 | 482.1952569 |
| CELSR3 | ENSG00000008300 | 0.030320353 | −0.943170934 | 567.8092691 |
| CD69 | ENSG00000110848 | 0.030352174 | −0.824052158 | 125525.2147 |
| KREMEN2 | ENSG00000131650 | 0.03093099 | −1.040851881 | 303.5627628 |
| GLTSCR2 | ENSG00000105373 | 0.031020656 | −0.428803354 | 17703.00275 |
| | ENSG00000182574 | 0.03106349 | −0.760489093 | 48.42077363 |
| WIPF2 | ENSG00000171475 | 0.031219271 | −0.345258424 | 5873.987233 |
| ZFYVE27 | ENSG00000155256 | 0.031224253 | −0.322528945 | 5618.218826 |
| IGHV1-3 | ENSG00000211935 | 0.03132182 | −1.050139337 | 84.73989246 |
| DPP7 | ENSG00000176978 | 0.031365163 | −0.416176546 | 10119.77943 |
| | ENSG00000265714 | 0.031388897 | −1.110696926 | 178.2894238 |
| CSGALNACT1 | ENSG00000147408 | 0.031490456 | −1.092553024 | 181.502698 |
| FBXL15 | ENSG00000107872 | 0.031877287 | −0.427693864 | 660.2233671 |
| ADAP2 | ENSG00000184060 | 0.031942642 | −0.670802085 | 777.6831187 |
| CCDC144NL-AS1 | ENSG00000233098 | 0.031965708 | −1.131294411 | 104.9278031 |
| VDR | ENSG00000111424 | 0.032062003 | −1.116989092 | 764.438711 |
| HSPH1 | ENSG00000120694 | 0.032417519 | −0.836036183 | 37131.89193 |
| LAMP3 | ENSG00000078081 | 0.03243215 | −1.002698583 | 401.7316037 |
| ADAM8 | ENSG00000151651 | 0.032453368 | −0.77466405 | 3725.033253 |
| VWA7 | ENSG00000204396 | 0.03251523 | −0.933578055 | 48.74599777 |
| PLEKHG4 | ENSG00000196155 | 0.032659111 | −0.924447286 | 86.042865 |
| PGAP3 | ENSG00000161395 | 0.032668618 | −0.424053351 | 1319.056036 |
| HSP90AB1 | ENSG00000096384 | 0.032752592 | −0.557921944 | 106947.8432 |
| OAT | ENSG00000065154 | 0.03279342 | −0.707895618 | 3547.950083 |
| C10orf76 | ENSG00000120029 | 0.032824503 | −0.415153356 | 4550.945215 |
| BPGM | ENSG00000172331 | 0.032955289 | −0.683577542 | 1577.295769 |
| NR4A2 | ENSG00000153234 | 0.032985333 | −0.931532486 | 31536.93308 |
| PHTF1 | ENSG00000116793 | 0.033376285 | −0.483419061 | 1633.776673 |
| SEMA3B | ENSG00000012171 | 0.033394342 | −0.885056535 | 90.80456692 |
| TRPV3 | ENSG00000167723 | 0.033458754 | −1.040270095 | 249.3318062 |
| TGIF1 | ENSG00000177426 | 0.033498727 | −1.023714282 | 3928.642239 |
| RAB20 | ENSG00000139832 | 0.033651418 | −1.111274053 | 50.53392861 |
| | ENSG00000237989 | 0.034529095 | −1.027494773 | 1108.405343 |
| DNAJA1 | ENSG00000086061 | 0.03462001 | −0.653604105 | 48893.6446 |
| SLC16A5 | ENSG00000170190 | 0.03470936 | −1.059949347 | 145.7113355 |
| EEF1A1P13 | ENSG00000250182 | 0.034785565 | −0.491604877 | 6606.241509 |
| SLA2 | ENSG00000101082 | 0.034898423 | −1.000028585 | 97.17387756 |
| IGHV3-7 | ENSG00000211938 | 0.034980627 | −0.714466274 | 322.799965 |
| AASS | ENSG00000008311 | 0.035134334 | −1.054848039 | 191.4758339 |
| MMP17 | ENSG00000198598 | 0.035311394 | −1.044396632 | 358.6460142 |
| | ENSG00000260461 | 0.035351393 | −0.591219971 | 125.5073405 |
| POLL | ENSG00000166169 | 0.03571765 | −0.309837119 | 1887.888278 |
| CCDC102B | ENSG00000150636 | 0.035892782 | −0.679114211 | 55.27593255 |
| FBXO24 | ENSG00000106336 | 0.035899011 | −0.686220382 | 48.04625965 |
| CD44 | ENSG00000026508 | 0.035999501 | −0.761054415 | 11474.22524 |
| RAP2B | ENSG00000181467 | 0.036391863 | −0.420652378 | 2221.716265 |
| TJP3 | ENSG00000105289 | 0.036449432 | −0.803301813 | 63.07905413 |
| CRB2 | ENSG00000148204 | 0.03647401 | −0.761576483 | 356.5059343 |
| PAQR7 | ENSG00000182749 | 0.036503249 | −0.750270482 | 139.3848283 |
| FAM150B | ENSG00000189292 | 0.036647813 | −0.951977341 | 51.31512636 |
| MIR25 | ENSG00000207547 | 0.036676996 | −0.662859949 | 82.3998348 |
| RNF149 | ENSG00000163162 | 0.036737914 | −0.411117723 | 2220.106209 |
| SLC25A30 | ENSG00000174032 | 0.036770547 | −0.571260187 | 1024.53341 |
| TBKBP1 | ENSG00000198933 | 0.036786213 | −1.031552962 | 598.3495595 |
| DNAJA1P3 | ENSG00000215007 | 0.036797241 | −0.667026797 | 821.4598011 |
| HERC1 | ENSG00000103657 | 0.037076957 | −0.403058937 | 8732.618435 |
| NTN1 | ENSG00000065320 | 0.037085164 | −0.924474319 | 85.40276875 |
| SERPINE1 | ENSG00000106366 | 0.037273577 | −1.055427988 | 66.8821918 |
| PLD4 | ENSG00000166428 | 0.03729425 | −1.019128192 | 1806.498631 |
| TRGV4 | ENSG00000211698 | 0.037313512 | −0.776234876 | 104.3255648 |
| FURIN | ENSG00000140564 | 0.037342723 | −0.783918495 | 6054.720588 |
| FAM65A | ENSG00000039523 | 0.037579158 | −0.74582307 | 5572.927206 |
| SPHK1 | ENSG00000176170 | 0.037735568 | −0.871859787 | 65.54749775 |
| LDLRAP1 | ENSG00000157978 | 0.038061603 | −0.909970091 | 117.7267336 |
| ZNF473 | ENSG00000142528 | 0.038233551 | −0.394956481 | 1187.617972 |
| | ENSG00000225637 | 0.038349242 | −1.017413232 | 88.46721738 |
| IL17RA | ENSG00000177663 | 0.038952707 | −0.480252097 | 2007.789839 |
| PGF | ENSG00000119630 | 0.039060982 | −0.803900141 | 70.27778726 |
| FPGT-TNNI3K | ENSG00000259030 | 0.039095308 | −0.934364666 | 78.14747369 |
| IGHJ4 | ENSG00000240041 | 0.039453436 | −1.083658923 | 68.67369071 |
| PAFAH2 | ENSG00000158006 | 0.039631827 | −0.336538914 | 840.1578067 |
| RARA | ENSG00000131759 | 0.039874593 | −0.723110759 | 1419.615028 |
| TNFRSF12A | ENSG00000006327 | 0.039963907 | −0.81536304 | 65.12605032 |
| MTMR3 | ENSG00000100330 | 0.040114051 | −0.25893991 | 5906.828642 |
| NAV2 | ENSG00000166833 | 0.040197798 | −1.030034484 | 73.78288523 |
| COL5A2 | ENSG00000204262 | 0.040249337 | −0.7596624 | 140.940164 |
| TMEM184A | ENSG00000164855 | 0.040323086 | −0.776172325 | 38.78938458 |
| ITPRIP | ENSG00000148841 | 0.040567224 | −0.508058835 | 4532.6864 |
| TELO2 | ENSG00000100726 | 0.040730743 | −0.404082659 | 3714.008493 |
| KLRF1 | ENSG00000150045 | 0.040747352 | −1.066119307 | 97.28848873 |
| | ENSG00000173727 | 0.040759747 | −0.837550411 | 211.6706785 |
| ABLIM2 | ENSG00000163995 | 0.040992961 | −1.035135847 | 67.87364371 |
| FOXN3P1 | ENSG00000176318 | 0.041038045 | −0.606526518 | 62.8319276 |
| ZNF442 | ENSG00000198342 | 0.041067379 | −0.703614518 | 310.356268 |
| ADCY7 | ENSG00000121281 | 0.041262726 | −0.418980171 | 3909.215889 |
| LFNG | ENSG00000106003 | 0.041577258 | −0.753545533 | 974.983984 |
| CTRC | ENSG00000162438 | 0.041988855 | −0.780673286 | 37.71496075 |
| PLAU | ENSG00000122861 | 0.042150006 | −0.891823079 | 42.02881402 |
| FMNL3 | ENSG00000161791 | 0.042164533 | −0.433624987 | 17082.6478 |
| | ENSG00000257924 | 0.042506737 | −1.061502409 | 83.47856494 |
| RASA3 | ENSG00000185989 | 0.042545385 | −0.846872986 | 2470.783708 |
| LINC00996 | ENSG00000242258 | 0.042572771 | −1.035550976 | 116.4294968 |
| IGLV8-61 | ENSG00000211638 | 0.042738443 | −1.000732377 | 99.88302369 |
| PRR5L | ENSG00000135362 | 0.042846835 | −1.029804623 | 74.30085527 |
| CHD7 | ENSG00000171316 | 0.043039725 | −0.382248282 | 10036.08605 |
| RASD1 | ENSG00000108551 | 0.043371868 | −0.729662829 | 100.5060963 |
| ALPL | ENSG00000162551 | 0.043546151 | −1.045384778 | 746.2978616 |
| ZNF14 | ENSG00000105708 | 0.043628376 | −0.548291616 | 938.46441 |
| HERC3 | ENSG00000138641 | 0.043709401 | −0.55529592 | 5095.814568 |
| ALPK2 | ENSG00000198796 | 0.043727694 | −1.032704778 | 43.62858665 |
| C19orf71 | ENSG00000183397 | 0.043838796 | −0.46492666 | 237.8674143 |
| SAFB2 | ENSG00000130254 | 0.043868439 | −0.234300485 | 7831.469912 |
| SLC4A3 | ENSG00000114923 | 0.044069583 | −1.01434518 | 60.38008962 |
| NOTCH1 | ENSG00000148400 | 0.044083672 | −0.644278717 | 4679.644712 |
| HSP90AB3P | ENSG00000183199 | 0.044148842 | −0.51016921 | 10744.85819 |
| RAB24 | ENSG00000169228 | 0.044247504 | −0.540769751 | 306.0746047 |
| IFNAR2 | ENSG00000159110 | 0.044249509 | −0.57905528 | 1277.573042 |
| PHLDB3 | ENSG00000176531 | 0.044278656 | −0.525056477 | 580.3355019 |
| RAB11FIP5 | ENSG00000135631 | 0.044331533 | −0.887300755 | 160.2423971 |
| CD3E | ENSG00000198851 | 0.044355198 | −0.98148352 | 81.81750275 |
| NFKBIZ | ENSG00000144802 | 0.0444216 | −0.612527615 | 15426.39283 |
| CRY1 | ENSG00000008405 | 0.044587701 | −1.03683301 | 59.53112408 |
| UACA | ENSG00000137831 | 0.044626096 | −1.054907938 | 57.94598744 |
| HSPA1L | ENSG00000204390 | 0.044830773 | −0.97272724 | 100.8703761 |
| NA | ENSG00000265150 | 0.044866356 | −0.840325803 | 2852.430192 |
| EPHB6 | ENSG00000106123 | 0.044912739 | −0.888922528 | 741.568143 |
| VASH1 | ENSG00000071246 | 0.044969159 | −0.810827913 | 114.9492951 |
| HEXIM1 | ENSG00000186834 | 0.045075983 | −0.709136234 | 12457.39581 |
| DOK3 | ENSG00000146094 | 0.045190441 | −0.508914662 | 9843.265333 |
| CYSLTR1 | ENSG00000173198 | 0.045294395 | −0.811705205 | 568.8399641 |
| FAM132A | ENSG00000184163 | 0.045296189 | −0.721194648 | 40.87301843 |
| THEMIS2 | ENSG00000130775 | 0.045303311 | −0.830338957 | 1596.292919 |
| APOL3 | ENSG00000128284 | 0.045618869 | −0.840043487 | 1123.986606 |
| HSP90AA4P | ENSG00000205100 | 0.045767914 | −0.674038632 | 124.3280168 |
| COL8A2 | ENSG00000171812 | 0.046004526 | −0.791211594 | 43.2095101 |
| C1R | ENSG00000159403 | 0.046079859 | −0.727901518 | 58.15374921 |
| NA | ENSG00000211939 | 0.04668379 | −1.04207084 | 147.5792767 |
| PARP9 | ENSG00000138496 | 0.04699084 | −0.57515454 | 2703.29381 |
| | ENSG00000250155 | 0.04702265 | −0.604993632 | 289.3496425 |
| CD6 | ENSG00000013725 | 0.047067069 | −0.983694236 | 371.5244664 |
| REPS1 | ENSG00000135597 | 0.047139536 | −0.430978364 | 4074.643438 |
| RGS12 | ENSG00000159788 | 0.047263148 | −0.736972691 | 166.2071664 |
| MFSD1P1 | ENSG00000261868 | 0.047283726 | −0.823923501 | 43.61282406 |
| SIRT1 | ENSG00000096717 | 0.047448513 | −0.582715163 | 7653.279866 |
| RENBP | ENSG00000102032 | 0.047486318 | −0.860780685 | 485.363642 |
| NA | ENSG00000198374 | 0.047529469 | −0.837507141 | 121.7619742 |
| TERF1 | ENSG00000147601 | 0.047580439 | −0.251681982 | 1050.44628 |
| SLC12A6 | ENSG00000140199 | 0.047726963 | −0.762319861 | 1847.516403 |
| SLC2A3P4 | ENSG00000254088 | 0.047864413 | −0.826336595 | 51.67196752 |
| PITPNM3 | ENSG00000091622 | 0.048024601 | −0.753384842 | 51.49363126 |
| WWP2 | ENSG00000198373 | 0.048163726 | −0.298834405 | 6857.522287 |
| ABCD1 | ENSG00000101986 | 0.048378578 | −0.497404841 | 836.1464356 |
| SIGIRR | ENSG00000185187 | 0.04838764 | −0.68612648 | 666.7086672 |
| RNASET2 | ENSG00000026297 | 0.0485324 | −0.570442924 | 5617.148898 |
| | ENSG00000223821 | 0.04860157 | −0.83215865 | 52.43949408 |
| TPM2 | ENSG00000198467 | 0.048744832 | −0.961209154 | 499.096811 |
| APOBR | ENSG00000184730 | 0.048874739 | −0.780604669 | 231.9644076 |
| MPP1 | ENSG00000130830 | 0.04896819 | −0.925063824 | 481.2364463 |
| RIN3 | ENSG00000100599 | 0.048970369 | −0.981704754 | 913.562486 |
| CDKN1A | ENSG00000124762 | 0.049076486 | −0.766899361 | 21148.91718 |
| CDIP1 | ENSG00000089486 | 0.049319306 | −0.818503091 | 456.1616353 |
| FMNL2 | ENSG00000157827 | 0.049491502 | −0.91603724 | 554.9980333 |
| ENO2 | ENSG00000111674 | 0.049505784 | −0.691625271 | 5171.433409 |
| CDKN2D | ENSG00000129355 | 0.049522938 | −0.528084154 | 2363.226701 |
| EFHD2 | ENSG00000142634 | 0.04968295 | −0.472111822 | 7065.929214 |
| | ENSG00000260279 | 0.049795021 | −0.663479043 | 51.31535361 |
| APOL1 | ENSG00000100342 | 0.049815844 | −0.870335664 | 764.5184107 |

Genes downregulated in B220 positive lymphoma cells from VavPBcl2 sh-Kmt2d tumors vs VavPBcl2-vector tumors

| gene symbol | ensembl | pvalue | padj | log2FoldChange | baseMean | hgnc_symbol |
|---|---|---|---|---|---|---|
| Lpl | ENSMUSG00000015568 | 2.06E−09 | 5.00E−06 | −2.66849639 | 49.48262037 | LPL |
| Rgs1 | ENSMUSG00000026358 | 1.00E−08 | 1.46E−05 | −2.816396672 | 1073.995217 | RGS1 |
| Adrbk2 | ENSMUSG00000042249 | 2.55E−08 | 2.89E−05 | −2.316244258 | 55.76356027 | ADRBK2 |
| Dusp1 | ENSMUSG00000024190 | 2.58E−08 | 2.89E−05 | −2.999263903 | 2217.251525 | DUSP1 |
| Klf4 | ENSMUSG00000003032 | 3.02E−08 | 3.15E−05 | −3.287908872 | 299.2336182 | KLF4 |
| Plk2 | ENSMUSG00000021701 | 4.82E−08 | 4.39E−05 | −2.74328799 | 217.2694707 | PLK2 |
| Rasgrp4 | ENSMUSG00000030589 | 1.88E−07 | 0.000124988 | −1.032147316 | 92.64153278 | RASGRP4 |
| Fosb | ENSMUSG00000003545 | 3.20E−07 | 0.000180082 | −3.477771152 | 4934.293017 | FOSB |
| Pbx1 | ENSMUSG00000052534 | 3.21E−07 | 0.000180082 | −1.610571041 | 165.32453 | PBX1 |
| Asph | ENSMUSG00000028207 | 5.71E−07 | 0.000297659 | −1.477735757 | 27.67753747 | ASPH |
| Wbscr17 | ENSMUSG00000034040 | 1.07E−06 | 0.000490279 | −1.769389326 | 48.282298 | WBSCR17 |
| Scn8a | ENSMUSG00000023033 | 1.38E−06 | 0.00059403 | −1.909292146 | 204.8407371 | SCN8A |
| Clu | ENSMUSG00000022037 | 1.95E−06 | 0.000749744 | −2.124981508 | 28.97217448 | CLU |
| Grin3a | ENSMUSG00000039579 | 2.74E−06 | 0.001001185 | −3.456710374 | 4.798432678 | GRIN3A |
| Marco | ENSMUSG00000026390 | 2.86E−06 | 0.001019084 | −2.757499587 | 38.88665003 | MARCO |
| Adcy6 | ENSMUSG00000022994 | 2.97E−06 | 0.001032043 | −3.225511666 | 7.175592488 | ADCY6 |
| Lilrb4 | ENSMUSG00000062593 | 3.23E−06 | 0.001096783 | −1.307407974 | 109.1638048 | LILRB4 |
| Egr1 | ENSMUSG00000038418 | 3.87E−06 | 0.00120682 | −2.660218304 | 4879.586708 | EGR1 |
| Ppp1r15a | ENSMUSG00000040435 | 3.89E−06 | 0.00120682 | −2.439855766 | 3085.260647 | PPP1R15A |
| Apobr | ENSMUSG00000042759 | 4.44E−06 | 0.001351529 | −1.538778953 | 37.28736229 | APOBR |
| Tns1 | ENSMUSG00000055322 | 4.77E−06 | 0.001392569 | −1.912291852 | 38.50870665 | TNS1 |
| Gramd1b | ENSMUSG00000040111 | 7.65E−06 | 0.002068562 | −1.743407429 | 141.2322456 | GRAMD1B |
| Vwf | ENSMUSG00000001930 | 1.03E−05 | 0.002633483 | −2.382697447 | 6.738996347 | VWF |
| Nod2 | ENSMUSG00000055994 | 1.27E−05 | 0.003096494 | −0.922291646 | 221.1208057 | NOD2 |
| Siglec1 | ENSMUSG00000027322 | 1.32E−05 | 0.003136553 | −2.726949868 | 15.52301448 | SIGLEC1 |
| Itga2b | ENSMUSG00000034664 | 1.39E−05 | 0.003145765 | −1.456121576 | 28.09287216 | ITGA2B |
| Psd2 | ENSMUSG00000024347 | 1.54E−05 | 0.003304846 | −2.125792023 | 16.96418361 | PSD2 |
| Ptp4a1 | ENSMUSG00000026064 | 1.55E−05 | 0.003304846 | −1.976484282 | 134.8184132 | PTP4A1 |
| Pld2 | ENSMUSG00000020828 | 1.58E−05 | 0.003304846 | −1.378714908 | 94.14783773 | PLD2 |
| Slc22a23 | ENSMUSG00000038267 | 1.76E−05 | 0.003574348 | −1.491328389 | 21.37201334 | SLC22A23 |
| Star | ENSMUSG00000031574 | 2.19E−05 | 0.004142547 | −1.653300584 | 56.74425576 | STAR |
| Dock5 | ENSMUSG00000044447 | 2.47E−05 | 0.004561579 | −1.896753351 | 17.43941047 | DOCK5 |
| Ptplad2 | ENSMUSG00000028497 | 2.64E−05 | 0.004641388 | −2.061147475 | 12.38206067 | PTPLAD2 |
| Psd3 | ENSMUSG00000030465 | 3.42E−05 | 0.005605076 | −2.04565605 | 78.56759814 | PSD3 |
| Bgn | ENSMUSG00000031375 | 4.13E−05 | 0.006613811 | −2.297184737 | 10.41904097 | BGN |
| Slc8a1 | ENSMUSG00000054640 | 4.24E−05 | 0.006613811 | −2.446393473 | 8.712193186 | SLC8A1 |
| Nlrp1b | ENSMUSG00000070390 | 4.43E−05 | 0.006739063 | −1.371451662 | 45.31224194 | NLRP1 |
| Csf2rb2 | ENSMUSG00000071714 | 4.71E−05 | 0.007089168 | −1.805202078 | 44.47042122 | CSF2RB |
| Cd69 | ENSMUSG00000030156 | 4.83E−05 | 0.007114717 | −1.697054756 | 1499.601504 | CD69 |
| Sspo | ENSMUSG00000029797 | 5.13E−05 | 0.007352169 | −1.962387251 | 14.73690907 | SSPO |
| Dock4 | ENSMUSG00000035954 | 5.28E−05 | 0.007408941 | −2.088623557 | 14.33005442 | DOCK4 |
| Atf3 | ENSMUSG00000026628 | 5.99E−05 | 0.007872282 | −2.272845585 | 57.89351011 | ATF3 |
| 6430548M08Rik | ENSMUSG00000031824 | 6.00E−05 | 0.007872282 | −1.689481524 | 34.80936177 | KIAA0513 |
| Fyco1 | ENSMUSG00000025241 | 6.09E−05 | 0.007872282 | −1.775754791 | 567.4333134 | FYCO1 |
| Gm684 | ENSMUSG00000079559 | 6.22E−05 | 0.007958499 | −2.629265541 | 30.80289861 | COLCA2 |
| Nlgn3 | ENSMUSG00000031302 | 6.81E−05 | 0.008316188 | −1.836724839 | 13.12952716 | NLGN3 |
| Lrp1 | ENSMUSG00000040249 | 6.84E−05 | 0.008316188 | −2.22911854 | 128.5806173 | LRP1 |
| Lrp4 | ENSMUSG00000027253 | 6.90E−05 | 0.008316188 | −2.099484255 | 8.345478819 | LRP4 |
| Havcr2 | ENSMUSG00000020399 | 7.63E−05 | 0.008987505 | −1.899705598 | 12.39990071 | HAVCR2 |
| Nfkbiz | ENSMUSG00000035356 | 7.81E−05 | 0.00910765 | −1.506989112 | 638.1804961 | NFKBIZ |
| Trim15 | ENSMUSG00000050747 | 7.87E−05 | 0.00910765 | −2.277779784 | 5.122292611 | TRIM15 |
| 6330403A02Rik | ENSMUSG00000053963 | 8.46E−05 | 0.009642784 | −1.471477346 | 13.86003662 | C1orf95 |
| Slc16a10 | ENSMUSG00000019838 | 9.11E−05 | 0.010001108 | −1.480688129 | 64.54262 | SLC16A10 |
| Padi2 | ENSMUSG00000028927 | 9.58E−05 | 0.010353618 | −1.068143616 | 260.4942761 | PADI2 |
| Frmd4a | ENSMUSG00000026657 | 0.00010485 | 0.011253612 | −1.72228982 | 23.05872621 | FRMD4A |
| Tead2 | ENSMUSG00000030796 | 0.000110389 | 0.011761699 | −1.268349705 | 46.63791248 | TEAD2 |
| Fam169a | ENSMUSG00000041817 | 0.000121609 | 0.012589575 | −1.710241457 | 9.253708334 | FAM169A |
| Vasn | ENSMUSG00000039646 | 0.000131586 | 0.013402871 | −2.076497998 | 139.5475629 | VASN |
| Gp49a | ENSMUSG00000089672 | 0.000133357 | 0.013402871 | −1.615020534 | 23.39187201 | LILRB4 |
| Tbkbp1 | ENSMUSG00000038517 | 0.000134056 | 0.013402871 | −0.997421145 | 59.47426537 | TBKBP1 |
| Sort1 | ENSMUSG00000068747 | 0.000153184 | 0.014116364 | −1.619502059 | 39.84809268 | SORT1 |
| Itgb1bp2 | ENSMUSG00000031312 | 0.000153449 | 0.014116364 | −2.050345677 | 8.191181455 | ITGB1BP2 |
| Arhgap32 | ENSMUSG00000041444 | 0.000154309 | 0.014116364 | −1.900267823 | 11.00374662 | ARHGAP32 |
| Nrg4 | ENSMUSG00000032311 | 0.000154588 | 0.014116364 | −2.020306571 | 20.88599242 | NRG4 |
| Tspan9 | ENSMUSG00000030352 | 0.000155496 | 0.014116364 | −2.191326532 | 4.29913557 | TSPAN9 |
| Zbtb37 | ENSMUSG00000043467 | 0.000158556 | 0.014116364 | −1.446576548 | 258.6765671 | ZBTB37 |
| Zcchc14 | ENSMUSG00000061410 | 0.000176414 | 0.015328068 | −2.166427137 | 10.70475468 | ZCCHC14 |
| Pde4c | ENSMUSG00000031842 | 0.0001794 | 0.015413323 | −1.302281586 | 138.4238558 | PDE4C |
| Ttbk1 | ENSMUSG00000015599 | 0.000183056 | 0.015413323 | −1.390377996 | 66.39089031 | TTBK1 |
| Dnhd1 | ENSMUSG00000030882 | 0.000183285 | 0.015413323 | −0.926979245 | 204.6546441 | DNHD1 |
| Ankrd16 | ENSMUSG00000047909 | 0.000185197 | 0.015413323 | −0.998792317 | 857.1580809 | ANKRD16 |
| Ankrd61 | ENSMUSG00000029607 | 0.000186213 | 0.015413323 | −2.046083793 | 16.98421956 | ANKRD61 |
| Etohil | ENSMUSG00000074519 | 0.000187954 | 0.015413323 | −1.217110782 | 68.53278883 | ZNF442 |
| Zfp36 | ENSMUSG00000044786 | 0.000190474 | 0.015532684 | −1.456474181 | 8404.428092 | ZFP36 |
| Bbs2 | ENSMUSG00000031755 | 0.000193814 | 0.01568575 | −0.787575799 | 740.2933078 | BBS2 |
| Runx2 | ENSMUSG00000039153 | 0.0001945 | 0.01568575 | −1.904864387 | 26.56547154 | RUNX2 |
| Slc26a1 | ENSMUSG00000046959 | 0.000199211 | 0.015900571 | −1.561801487 | 11.30257131 | SLC26A1 |
| Elk4 | ENSMUSG00000026436 | 0.000206469 | 0.016011688 | −1.624547145 | 950.3110468 | ELK4 |
| Ptk6 | ENSMUSG00000038751 | 0.00020723 | 0.016011688 | −1.850532537 | 10.82785673 | PTK6 |
| Spred3 | ENSMUSG00000037239 | 0.000208979 | 0.016011688 | −1.416959185 | 31.21992833 | SPRED3 |
| Hpn | ENSMUSG00000001249 | 0.000210983 | 0.016011688 | −2.171439542 | 6.446607001 | HPN |
| Tbc1d8 | ENSMUSG00000003134 | 0.000214106 | 0.016011688 | −1.470253374 | 39.44898738 | TBC1D8 |
| Carns1 | ENSMUSG00000075289 | 0.000219911 | 0.016294608 | −1.41037029 | 590.4947326 | CARNS1 |
| Stard9 | ENSMUSG00000033705 | 0.000237108 | 0.017219261 | −1.822797935 | 115.7567616 | STARD9 |
| Gpr182 | ENSMUSG00000058396 | 0.000242295 | 0.017422573 | −1.713383849 | 10.58878259 | GPR182 |
| Mfsd2b | ENSMUSG00000037336 | 0.000252729 | 0.017829658 | −2.138048658 | 14.80263876 | MFSD2B |
| Fgd4 | ENSMUSG00000022788 | 0.000254064 | 0.017829658 | −2.159733108 | 4.616439369 | FGD4 |
| Dchs1 | ENSMUSG00000036862 | 0.000261269 | 0.018028809 | −1.621875815 | 17.92123837 | DCHS1 |
| Cd4 | ENSMUSG00000023274 | 0.000262997 | 0.018028809 | −1.295262339 | 134.1673578 | CD4 |
| Rims3 | ENSMUSG00000032890 | 0.000270585 | 0.018029762 | −1.950090439 | 22.81460888 | RIMS3 |
| Deptor | ENSMUSG00000022419 | 0.000274332 | 0.018029762 | −1.772251398 | 10.1358429 | DEPTOR |
| Zc3h6 | ENSMUSG00000042851 | 0.000276376 | 0.018029762 | −1.316978163 | 340.8142937 | ZC3H6 |
| Fbxl20 | ENSMUSG00000020883 | 0.000276461 | 0.018029762 | −1.400047212 | 822.1908281 | FBXL20 |
| Kcna2 | ENSMUSG00000040724 | 0.000279612 | 0.018029762 | −2.672876169 | 5.873194136 | KCNA2 |
| Chadl | ENSMUSG00000063765 | 0.000285076 | 0.018067559 | −2.330056283 | 6.658916452 | CHADL |
| Itgad | ENSMUSG00000070369 | 0.000285107 | 0.018067559 | −1.501323109 | 137.956606 | ITGAD |
| Pde1b | ENSMUSG00000022489 | 0.000285922 | 0.018067559 | −1.27673416 | 390.0942618 | PDE1B |
| Ceacam16 | ENSMUSG00000014686 | 0.00029084 | 0.018226519 | −1.682749383 | 227.6048411 | CEACAM16 |
| Zfp287 | ENSMUSG00000005267 | 0.000296237 | 0.018255867 | −0.965142445 | 281.7186824 | ZNF287 |
| Klf6 | ENSMUSG00000000078 | 0.000298436 | 0.018255867 | −1.821621138 | 3288.546144 | KLF6 |
| Egr2 | ENSMUSG00000037868 | 0.000298907 | 0.018255867 | −1.393708127 | 703.293856 | EGR2 |
| Gpr35 | ENSMUSG00000026271 | 0.000302486 | 0.01832109 | −1.521787015 | 16.29382022 | GPR35 |
| Clec9a | ENSMUSG00000046080 | 0.000319253 | 0.019162219 | −1.723879784 | 13.51143487 | CLEC9A |
| Rhob | ENSMUSG00000054364 | 0.000324982 | 0.019162219 | −1.748356408 | 1486.356103 | RHOB |
| Cacnb4 | ENSMUSG00000017412 | 0.000325562 | 0.019162219 | −1.041322794 | 32.76046261 | CACNB4 |
| Adam23 | ENSMUSG00000025964 | 0.000330081 | 0.019350193 | −2.224862791 | 15.68728041 | ADAM23 |
| Arl4c | ENSMUSG00000049866 | 0.000334635 | 0.019438666 | −1.411187557 | 49.70635225 | ARL4C |
| Npff | ENSMUSG00000023052 | 0.000336917 | 0.019438666 | −1.286345909 | 19.73125903 | NPFF |
| Pdzd4 | ENSMUSG00000002006 | 0.000338417 | 0.019448302 | −2.0564967 | 17.71933299 | PDZD4 |
| Tifab | ENSMUSG00000049625 | 0.000354388 | 0.02012841 | −1.132022255 | 29.51786383 | TIFAB |
| Dnajb9 | ENSMUSG00000014905 | 0.000361291 | 0.020326045 | −1.139495968 | 1169.569645 | DNAJB9 |
| Lrrc39 | ENSMUSG00000027961 | 0.000364435 | 0.020326045 | −1.545482427 | 23.0552134 | LRRC39 |
| Tnnt3 | ENSMUSG00000061723 | 0.000386286 | 0.021118403 | −0.987352253 | 43.77323144 | TNNT3 |
| Hspg2 | ENSMUSG00000028763 | 0.000402718 | 0.021691792 | −1.679153333 | 53.47278252 | HSPG2 |
| Zfyve9 | ENSMUSG00000034557 | 0.000418847 | 0.021992476 | −1.764697161 | 11.76562696 | ZFYVE9 |
| Nr4a1 | ENSMUSG00000023034 | 0.000431055 | 0.022312433 | −1.673712587 | 562.962306 | NR4A1 |
| Peli2 | ENSMUSG00000021846 | 0.000435928 | 0.022402604 | −2.422663668 | 3.880881547 | PELI2 |
| Erp27 | ENSMUSG00000030219 | 0.000438927 | 0.022402604 | −1.426292137 | 51.65998631 | ERP27 |
| Zscan30 | ENSMUSG00000024274 | 0.000441607 | 0.022460416 | −1.322286164 | 24.89340076 | ZSCAN30 |
| Fos | ENSMUSG00000021250 | 0.000464525 | 0.023301257 | −2.357949096 | 13275.11206 | FOS |
| Ccnd1 | ENSMUSG00000070348 | 0.000466927 | 0.023341567 | −1.17070802 | 31.02358047 | CCND1 |
| Ahnak | ENSMUSG00000069833 | 0.000470683 | 0.023358764 | −2.062416424 | 1680.173855 | AHNAK |
| Tet2 | ENSMUSG00000040943 | 0.000474655 | 0.023358764 | −1.897938543 | 284.6863129 | TET2 |
| Socs3 | ENSMUSG00000053113 | 0.000482331 | 0.023456565 | −0.778914185 | 381.5964304 | SOCS3 |
| Fam196b | ENSMUSG00000069911 | 0.000490387 | 0.023702587 | −2.514870474 | 4.595410993 | FAM196B |
| Zfp369 | ENSMUSG00000021514 | 0.000494442 | 0.023748617 | −1.665629112 | 159.9610263 | ZNF274 |
| Dgkh | ENSMUSG00000034731 | 0.000497279 | 0.023799292 | −1.547762715 | 38.8310628 | DGKH |
| Tgm2 | ENSMUSG00000037820 | 0.000512176 | 0.024153243 | −1.448477355 | 67.85696055 | TGM2 |
| Pde8b | ENSMUSG00000021684 | 0.000517871 | 0.024153243 | −1.570338766 | 9.885745994 | PDE8B |
| Ttc39b | ENSMUSG00000038172 | 0.000521879 | 0.024153243 | −1.252097216 | 1288.028067 | TTC39B |
| Sgpp2 | ENSMUSG00000032908 | 0.000522421 | 0.024153243 | −1.317481065 | 36.71535726 | SGPP2 |
| Usp35 | ENSMUSG00000035713 | 0.000531228 | 0.024384685 | −1.082047744 | 115.7539621 | USP35 |
| Parvb | ENSMUSG00000022438 | 0.000533758 | 0.024424045 | −1.639534795 | 16.94850224 | PARVB |
| Gpr152 | ENSMUSG00000044724 | 0.000579798 | 0.025802775 | −1.686187576 | 7.310734836 | GPR152 |
| Pear1 | ENSMUSG00000028073 | 0.000588984 | 0.025914239 | −0.962898932 | 752.752543 | PEAR1 |
| Ube2i | ENSMUSG00000015120 | 0.000596984 | 0.025981678 | −1.295610489 | 954.5547059 | UBE2I |
| Tmcc2 | ENSMUSG00000042066 | 0.000598016 | 0.025981678 | −2.279008363 | 52.12486744 | TMCC2 |
| Sez6l2 | ENSMUSG00000030683 | 0.000607528 | 0.026082627 | −1.174744011 | 45.06918512 | SEZ6L2 |
| Rab11fip2 | ENSMUSG00000040022 | 0.000617794 | 0.026368242 | −1.375870651 | 262.6668625 | RAB11FIP2 |
| Dpm1 | ENSMUSG00000078919 | 0.000619853 | 0.026378988 | −1.454695115 | 116.1047827 | DPM1 |
| Mecp2 | ENSMUSG00000031393 | 0.000638248 | 0.026848711 | −1.062638145 | 1280.061831 | MECP2 |
| Acp2 | ENSMUSG00000002103 | 0.000653192 | 0.027218676 | −1.111920414 | 157.0098306 | ACP2 |
| Samd8 | ENSMUSG00000021770 | 0.000682225 | 0.027894796 | −1.162239057 | 193.3696486 | SAMD8 |
| Iqce | ENSMUSG00000036555 | 0.000699693 | 0.028292003 | −1.013854798 | 552.3055062 | IQCE |
| Chrna2 | ENSMUSG00000022041 | 0.000724236 | 0.028853706 | −1.880406556 | 23.03180046 | CHRNA2 |
| Brat | ENSMUSG00000002413 | 0.000732744 | 0.029064841 | −1.645744132 | 482.0690938 | BRAF |
| Adam22 | ENSMUSG00000040537 | 0.000736352 | 0.029128807 | −1.467519536 | 21.67314744 | ADAM22 |
| Alpk3 | ENSMUSG00000038763 | 0.000738987 | 0.029154048 | −2.047155916 | 4.816961555 | ALPK3 |
| Zc3hav1l | ENSMUSG00000047749 | 0.000753422 | 0.029643379 | −1.618197185 | 30.70909319 | ZC3HAV1L |
| Clcn2 | ENSMUSG00000022843 | 0.000763569 | 0.029877171 | −1.191921359 | 43.40641403 | CLCN2 |
| Slc38a6 | ENSMUSG00000044712 | 0.000776559 | 0.03008192 | −1.048071927 | 142.5907025 | SLC38A6 |
| Tbc1d23 | ENSMUSG00000022749 | 0.000777335 | 0.03008192 | −0.999479142 | 424.229294 | TBC1D23 |
| Ptpdc1 | ENSMUSG00000038042 | 0.000790761 | 0.030329028 | −1.484380314 | 29.03252488 | PTPDC1 |
| Tctn1 | ENSMUSG00000038593 | 0.000795347 | 0.030329028 | −0.622094666 | 294.8930879 | TCTN1 |
| Itpripl2 | ENSMUSG00000073859 | 0.000795977 | 0.030329028 | −1.552002946 | 8.816377642 | ITPRIPL2 |
| Jun | ENSMUSG00000052684 | 0.000802027 | 0.030329028 | −2.160247006 | 8020.746127 | JUN |
| Metap1d | ENSMUSG00000041921 | 0.000802449 | 0.030329028 | −0.609973578 | 606.0931886 | METAP1D |
| Obscn | ENSMUSG00000061462 | 0.000803165 | 0.030329028 | −2.183798912 | 24.35334393 | OBSCN |
| Nr4a2 | ENSMUSG00000026826 | 0.000803594 | 0.030329028 | −1.989067864 | 25.0441257 | NR4A2 |
| Axl | ENSMUSG00000002602 | 0.000817943 | 0.03052382 | −1.530728281 | 359.039184 | AXL |
| Cln8 | ENSMUSG00000026317 | 0.000819236 | 0.03052382 | −1.34287642 | 46.28912776 | CLN8 |
| Klf7 | ENSMUSG00000025959 | 0.000819712 | 0.03052382 | −1.223255019 | 260.61713 | KLF7 |
| Ccnl1 | ENSMUSG00000027829 | 0.000822814 | 0.030561357 | −1.11646967 | 4010.711626 | CCNL1 |
| Plp1 | ENSMUSG00000031425 | 0.000837012 | 0.030853205 | −1.401889911 | 55.30656797 | PLP1 |
| Vamp2 | ENSMUSG00000020894 | 0.000840388 | 0.030899599 | −1.12310418 | 1037.633535 | VAMP2 |
| Zfyve28 | ENSMUSG00000037224 | 0.0008486 | 0.031022264 | −0.772489103 | 57.0132211 | ZFYVE28 |
| Lcp2 | ENSMUSG00000002699 | 0.000875632 | 0.031327442 | −0.862374515 | 510.7997865 | LCP2 |
| Gabbr1 | ENSMUSG00000024462 | 0.000890466 | 0.031740688 | −1.346141353 | 680.5141611 | GABBR1 |
| Zfp174 | ENSMUSG00000054939 | 0.000898687 | 0.031840142 | −0.967707083 | 28.98228042 | ZNF174 |
| Thbs3 | ENSMUSG00000028047 | 0.000905663 | 0.032009602 | −1.546344617 | 12.52096958 | THBS3 |
| Fgr | ENSMUSG00000028874 | 0.000912921 | 0.032188174 | −0.614812973 | 556.771912 | FGR |
| Ptprm | ENSMUSG00000033278 | 0.000923418 | 0.03235568 | −2.176539279 | 10.76546163 | PTPRM |
| Ccr2 | ENSMUSG00000049103 | 0.000957734 | 0.033085629 | −1.546823902 | 12.46833031 | CCR2 |
| Adam11 | ENSMUSG00000020926 | 0.000960738 | 0.033085629 | −2.090077764 | 35.19529734 | ADAM11 |
| Hip1 | ENSMUSG00000039959 | 0.000966141 | 0.033085629 | −1.330696206 | 182.2892978 | HIP1 |
| Sox5 | ENSMUSG00000041540 | 0.000974751 | 0.03318395 | −1.330513151 | 75.21368604 | SOX5 |
| Synpo | ENSMUSG00000043079 | 0.000990985 | 0.033407396 | −1.438892605 | 15.99326057 | SYNPO |
| Gm608 | ENSMUSG00000068284 | 0.001001189 | 0.03367364 | −1.739947459 | 1983.226642 | KIAA2018 |
| Pou2f1 | ENSMUSG00000026565 | 0.001007623 | 0.033812121 | −1.607513731 | 939.7315583 | POU2F1 |
| 1700028K03Rik | ENSMUSG00000089798 | 0.001010216 | 0.033821385 | −1.971705771 | 10.69377346 | C1orf146 |
| Nfat5 | ENSMUSG00000003847 | 0.001018325 | 0.033871836 | −1.821256847 | 1631.378438 | NFAT5 |
| Carf | ENSMUSG00000026017 | 0.001021005 | 0.033871836 | −1.32669663 | 45.79511781 | CARF |
| Gm614 | ENSMUSG00000090141 | 0.001027951 | 0.034009558 | −1.856503549 | 9.752766971 | CXorf65 |
| Gfra2 | ENSMUSG00000022103 | 0.001037691 | 0.034009558 | −1.972942717 | 30.54222793 | GFRA2 |
| Cttnbp2nl | ENSMUSG00000062127 | 0.001039136 | 0.034009558 | −1.727450521 | 13.58433501 | CTTNBP2NL |
| Rad9b | ENSMUSG00000038569 | 0.001045166 | 0.034076491 | −1.12502387 | 67.51132281 | RAD9B |
| Rab6b | ENSMUSG00000032549 | 0.001049312 | 0.034113143 | −0.777837523 | 231.3277775 | RAB6B |
| Mybpc2 | ENSMUSG00000038670 | 0.001068908 | 0.034524263 | −0.787990887 | 194.7536505 | MYBPC2 |
| Atxn1 | ENSMUSG00000046876 | 0.00106988 | 0.034524263 | −1.825378503 | 76.01911992 | ATXN1 |
| Pik3r6 | ENSMUSG00000046207 | 0.001073783 | 0.034524263 | −1.191103345 | 28.26961953 | PIK3R6 |
| Farp2 | ENSMUSG00000034066 | 0.001109036 | 0.035225629 | −0.971150366 | 75.08956959 | FARP2 |
| Itgam | ENSMUSG00000030786 | 0.00111149 | 0.035225629 | −1.278158644 | 109.7427865 | ITGAM |
| Fhdc1 | ENSMUSG00000041842 | 0.001112406 | 0.035225629 | −1.992900235 | 11.75848047 | FHDC1 |
| Gpr157 | ENSMUSG00000047875 | 0.001117337 | 0.035225629 | −1.186495937 | 51.21112294 | GPR157 |
| Sema5a | ENSMUSG00000022231 | 0.00111973 | 0.035225629 | −1.594643342 | 57.4750412 | SEMA5A |
| Tdo2 | ENSMUSG00000028011 | 0.001138023 | 0.035647475 | −1.479747742 | 12.53458474 | TDO2 |
| Iqgap2 | ENSMUSG00000021676 | 0.001149215 | 0.035742861 | −1.258050011 | 29.08656655 | IQGAP2 |
| Gda | ENSMUSG00000058624 | 0.001150863 | 0.035742861 | −1.910003382 | 32.44621308 | GDA |
| Dusp6 | ENSMUSG00000019960 | 0.001155247 | 0.035802853 | −1.191094253 | 906.6456664 | DUSP6 |
| Il1b | ENSMUSG00000027398 | 0.001169181 | 0.03615792 | −1.89775988 | 47.08800643 | IL1B |
| Wdfy1 | ENSMUSG00000073643 | 0.001172593 | 0.036186753 | −1.255042649 | 371.5815498 | WDFY1 |
| Itgax | ENSMUSG00000030789 | 0.001184169 | 0.036237565 | −1.247330309 | 172.4690963 | ITGAX |
| Zc3h12c | ENSMUSG00000035164 | 0.001186863 | 0.036244013 | −1.999811212 | 145.8967019 | ZC3H12C |
| Fut1 | ENSMUSG00000008461 | 0.001196734 | 0.036359068 | −1.215891369 | 22.88567235 | FUT1 |
| Sspn | ENSMUSG00000030255 | 0.001202941 | 0.036359068 | −1.77726208 | 143.8983592 | SSPN |
| Agap1 | ENSMUSG00000055013 | 0.001205321 | 0.036359068 | −1.827005908 | 9.997808786 | AGAP1 |
| Slc20a1 | ENSMUSG00000027397 | 0.001205576 | 0.036359068 | −0.99645932 | 915.8355255 | SLC20A1 |
| Cd5l | ENSMUSG00000015854 | 0.001211302 | 0.036383132 | −1.673802969 | 305.6733454 | CD5L |
| Slc38a5 | ENSMUSG00000031170 | 0.001211359 | 0.036383132 | −2.451657428 | 7.827237746 | SLC38A5 |
| Tulp2 | ENSMUSG00000023467 | 0.001227043 | 0.036597733 | −1.630866903 | 65.52661594 | TULP2 |
| Cd300ld | ENSMUSG00000034641 | 0.001228533 | 0.036597733 | −1.633135674 | 17.06044102 | CD300LD |
| Dse | ENSMUSG00000039497 | 0.001242309 | 0.036857689 | −1.383628182 | 43.67319584 | DSE |
| Man1c1 | ENSMUSG00000037306 | 0.001247163 | 0.036926658 | −1.018797877 | 43.36779018 | MAN1C1 |
| Slc38a9 | ENSMUSG00000047789 | 0.001251777 | 0.036972668 | −0.676897822 | 589.701862 | SLC38A9 |
| Armc9 | ENSMUSG00000062590 | 0.001253783 | 0.036972668 | −0.757768677 | 151.331344 | ARMC9 |
| Dgke | ENSMUSG00000000276 | 0.001281769 | 0.037565847 | −1.04217362 | 382.7516859 | DGKE |
| Tagln | ENSMUSG00000032085 | 0.001305948 | 0.037631548 | −0.799451295 | 64.02575513 | TAGLN |
| Impg2 | ENSMUSG00000035270 | 0.001306141 | 0.037631548 | −1.277898096 | 62.07141876 | IMPG2 |
| Cyp2u1 | ENSMUSG00000027983 | 0.001307063 | 0.037631548 | −1.625322473 | 10.96789881 | CYP2U1 |
| Per1 | ENSMUSG00000020893 | 0.001329551 | 0.037848569 | −1.493817053 | 1026.104012 | PER1 |
| Wdr78 | ENSMUSG00000035126 | 0.001329929 | 0.037848569 | −1.105139407 | 80.6910908 | WDR78 |
| Piwil2 | ENSMUSG00000033644 | 0.00133989 | 0.037848569 | −1.057180547 | 16.83165035 | PIWIL2 |
| Lcor | ENSMUSG00000025019 | 0.00134053 | 0.037848569 | −1.802566928 | 257.9653543 | LCOR |
| Fam126b | ENSMUSG00000038174 | 0.001346831 | 0.037879955 | −1.514600603 | 207.8671293 | FAM126B |
| Ar19 | ENSMUSG00000063820 | 0.001361604 | 0.038010233 | −2.007697083 | 3.970194176 | ARL9 |
| Mfsd4 | ENSMUSG00000059149 | 0.001403555 | 0.038772731 | −1.937948775 | 444.739803 | MFSD4 |
| Gem | ENSMUSG00000028214 | 0.001407793 | 0.038772731 | −1.030813296 | 735.252472 | GEM |
| Nrp1 | ENSMUSG00000025810 | 0.0014233 | 0.039126003 | −1.026079656 | 41.49195176 | NRP1 |
| Loxl3 | ENSMUSG00000000693 | 0.001433139 | 0.039253589 | −1.091331274 | 15.85034464 | LOXL3 |
| Ctnnd1 | ENSMUSG00000034101 | 0.001433319 | 0.039253589 | −1.456561402 | 48.63625709 | CTNND1 |
| Mtx3 | ENSMUSG00000021704 | 0.001440455 | 0.03932394 | −1.555572943 | 256.3570554 | MTX3 |
| Srgap3 | ENSMUSG00000030257 | 0.00144281 | 0.03932394 | −1.287860548 | 102.5694615 | SRGAP3 |
| Ppp1r3f | ENSMUSG00000039556 | 0.001456575 | 0.039593351 | −0.748642694 | 101.5104631 | PPP1R3F |
| Specc1 | ENSMUSG00000042331 | 0.001491099 | 0.04005965 | −1.522339041 | 38.14391744 | SPECC1 |
| Pak1 | ENSMUSG00000030774 | 0.001504271 | 0.040127081 | −1.324409193 | 15.64719258 | PAK1 |
| Tlr8 | ENSMUSG00000040522 | 0.001507355 | 0.040127081 | −1.98114572 | 13.91611408 | TLR8 |
| Aoc2 | ENSMUSG00000078651 | 0.001509198 | 0.040127081 | −1.189031157 | 87.54427606 | AOC2 |
| Ifih1 | ENSMUSG00000026896 | 0.001530799 | 0.040392874 | −0.853168096 | 77.83204665 | IFIH1 |
| Col20a1 | ENSMUSG00000016356 | 0.001534908 | 0.040392874 | −1.195109289 | 35.60469803 | COL20A1 |
| Zfand5 | ENSMUSG00000024750 | 0.001550711 | 0.04063866 | −0.587413401 | 1199.813172 | ZFAND5 |
| Dqx1 | ENSMUSG00000009145 | 0.001567283 | 0.040865398 | −0.944582107 | 96.17668182 | DQX1 |
| Impact | ENSMUSG00000024423 | 0.001582996 | 0.040897325 | −1.262143225 | 352.4035286 | IMPACT |
| Guf1 | ENSMUSG00000029208 | 0.001612152 | 0.040959899 | −0.785685742 | 529.2192097 | GUF1 |
| Lilra5 | ENSMUSG00000070873 | 0.001616853 | 0.040959899 | −1.856486125 | 13.04937651 | LILRA5 |
| St8sia1 | ENSMUSG00000030283 | 0.001617552 | 0.040959899 | −0.923887131 | 30.47780576 | ST8SIA1 |
| Evi5 | ENSMUSG00000011831 | 0.00161909 | 0.040959899 | −1.35473236 | 73.63568417 | EVI5 |
| Ptpre | ENSMUSG00000041836 | 0.001629267 | 0.041008996 | −1.022144533 | 284.6119241 | PTPRE |
| Vcam1 | ENSMUSG00000027962 | 0.001654649 | 0.04135772 | −1.512930989 | 463.4984908 | VCAM1 |
| Afap1 | ENSMUSG00000029094 | 0.001676451 | 0.041687418 | −1.511150161 | 26.15138084 | AFAP1 |
| Kcna3 | ENSMUSG00000047959 | 0.001680231 | 0.041687418 | −1.421594373 | 270.7066815 | KCNA3 |
| B4galnt2 | ENSMUSG00000013418 | 0.001682119 | 0.041687418 | −1.207778662 | 11.55931098 | B4GALNT2 |
| Mical3 | ENSMUSG00000003178 | 0.001690196 | 0.041816605 | −1.076535212 | 95.4659356 | MICAL3 |
| Sh3bp4 | ENSMUSG00000036206 | 0.001719547 | 0.042185262 | −1.680178506 | 25.78754028 | SH3BP4 |
| P2rx7 | ENSMUSG00000029468 | 0.001728037 | 0.042322411 | −1.011597402 | 43.62655299 | P2RX7 |
| Scai | ENSMUSG00000035236 | 0.001734236 | 0.042349233 | −1.617574656 | 1233.894058 | SCAI |
| Ubn2 | ENSMUSG00000038538 | 0.001737836 | 0.042349233 | −1.796709477 | 791.2081954 | UBN2 |
| Ino80d | ENSMUSG00000040865 | 0.001747516 | 0.042360628 | −1.839055403 | 1154.013149 | INO80D |
| Sox6 | ENSMUSG00000051910 | 0.001749583 | 0.042360628 | −2.37272878 | 7.592385073 | SOX6 |
| Daam2 | ENSMUSG00000040260 | 0.001759771 | 0.042528781 | −1.79754816 | 5.46460294 | DAAM2 |
| Adamdec1 | ENSMUSG00000022057 | 0.001776703 | 0.042701049 | −1.3876336 | 77.10787512 | ADAMDEC1 |
| Cd46 | ENSMUSG00000016493 | 0.001794176 | 0.043004253 | −1.007818003 | 41.30256018 | CD46 |
| Stx17 | ENSMUSG00000061455 | 0.001798121 | 0.043028149 | −1.118731586 | 435.4264888 | STX17 |
| Ppia | ENSMUSG00000071866 | 0.001813502 | 0.043134194 | −0.83904338 | 194.8989037 | PPIA |
| Trpm2 | ENSMUSG00000009292 | 0.00182745 | 0.043192322 | −1.699378652 | 112.6912182 | TRPM2 |
| Hipk2 | ENSMUSG00000061436 | 0.001829821 | 0.043192322 | −1.014916571 | 297.0900864 | HIPK2 |
| Slc14a1 | ENSMUSG00000059336 | 0.001835899 | 0.043192322 | −0.678347814 | 481.6850918 | SLC14A1 |
| Yod1 | ENSMUSG00000046404 | 0.00183753 | 0.043192322 | −1.406278097 | 173.53393 | YOD1 |
| Baz2b | ENSMUSG00000026987 | 0.001846712 | 0.043243687 | −1.409386369 | 2296.598948 | BAZ2B |
| Phlda1 | ENSMUSG00000020205 | 0.001849472 | 0.043243687 | −1.136949209 | 94.65351752 | PHLDA1 |
| Taok1 | ENSMUSG00000017291 | 0.001856046 | 0.043243687 | −1.602859887 | 1238.630797 | TAOK1 |
| Ccdc38 | ENSMUSG00000036168 | 0.0018582 | 0.043243687 | −1.410899128 | 32.88986262 | CCDC38 |
| Nrip2 | ENSMUSG00000001520 | 0.001860453 | 0.043243687 | −1.800337069 | 28.44699656 | NRIP2 |
| Ankrd23 | ENSMUSG00000067653 | 0.001914453 | 0.043976315 | −0.941425775 | 160.7948308 | ANKRD23 |
| Neb | ENSMUSG00000026950 | 0.001925784 | 0.044129772 | −1.54919577 | 17.68960529 | NEB |
| Guca1b | ENSMUSG00000023979 | 0.001937385 | 0.04423654 | −1.088884026 | 42.83725195 | GUCA1B |
| Plcb4 | ENSMUSG00000039943 | 0.001941167 | 0.04423654 | −1.375639815 | 19.44996019 | PLCB4 |
| Kctd18 | ENSMUSG00000054770 | 0.001942565 | 0.04423654 | −1.083525904 | 347.4675541 | KCTD18 |
| Kcnip4 | ENSMUSG00000029088 | 0.001960055 | 0.044339257 | −1.772663601 | 8.05695919 | KCNIP4 |
| Ptar1 | ENSMUSG00000074925 | 0.001977223 | 0.044339257 | −1.062707492 | 123.9886755 | PTAR1 |
| Gramd1a | ENSMUSG00000001248 | 0.001979745 | 0.044339257 | −0.424852529 | 3045.373649 | GRAMD1A |
| Ttn | ENSMUSG00000051747 | 0.001980412 | 0.044339257 | −1.667179394 | 75.16634357 | TTN |
| Phactr2 | ENSMUSG00000062866 | 0.001983526 | 0.044339257 | −1.025550939 | 27.5924515 | PHACTR2 |
| Rad54l2 | ENSMUSG00000040661 | 0.001993949 | 0.044436148 | −1.525655164 | 416.5510434 | RAD54L2 |
| Tsc22d1 | ENSMUSG00000022010 | 0.00205227 | 0.045320697 | −0.987807941 | 98.18193001 | TSC22D1 |
| Ubc | ENSMUSG00000008348 | 0.00206653 | 0.045566668 | −0.97134897 | 2816.026898 | UBC |
| Nav1 | ENSMUSG00000009418 | 0.002092325 | 0.045858368 | −1.905821597 | 18.05060464 | NAV1 |
| Myh10 | ENSMUSG00000020900 | 0.002129334 | 0.046249219 | −1.540387918 | 16.85034676 | MYH10 |
| Ache | ENSMUSG00000023328 | 0.002135986 | 0.046249219 | −1.552066261 | 25.655598 | ACHE |
| Tsnaxip1 | ENSMUSG00000031893 | 0.002140448 | 0.046249219 | −1.057077659 | 69.78482173 | TSNAXIP1 |
| Nfix | ENSMUSG00000001911 | 0.002147465 | 0.046249219 | −0.97261198 | 51.35588561 | NFIX |
| Alkbh1 | ENSMUSG00000079036 | 0.002153497 | 0.046249219 | −1.010881702 | 51.29066794 | ALKBH1 |
| Katnal1 | ENSMUSG00000041298 | 0.002154394 | 0.046249219 | −0.804276845 | 223.280228 | KATNAL1 |
| Bmp8a | ENSMUSG00000032726 | 0.002166725 | 0.046426588 | −1.004131251 | 18.41173419 | BMP8A |
| Lats1 | ENSMUSG00000040021 | 0.002174684 | 0.046426588 | −1.376277679 | 1162.252141 | LATS1 |
| Clec4n | ENSMUSG00000023349 | 0.002183619 | 0.046426588 | −1.386549259 | 81.89343304 | CLEC6A |
| Efcab5 | ENSMUSG00000050944 | 0.002189126 | 0.046426588 | −1.489437872 | 9.929930977 | EFCAB5 |
| Sparc | ENSMUSG00000018593 | 0.002192419 | 0.046426588 | −1.975327488 | 9.129997659 | SPARC |
| Cbl | ENSMUSG00000034342 | 0.00220791 | 0.046426588 | −1.744766638 | 782.9917131 | CBL |
| Klhl11 | ENSMUSG00000048732 | 0.002213644 | 0.046426588 | −1.343955816 | 146.3406721 | KLHL11 |
| Enpp4 | ENSMUSG00000023961 | 0.002214191 | 0.046426588 | −1.321217721 | 22.62334124 | ENPP4 |
| Plekhn1 | ENSMUSG00000078485 | 0.00221426 | 0.046426588 | −1.136355252 | 138.9971781 | PLEKHN1 |
| Ppp1r32 | ENSMUSG00000035179 | 0.00221962 | 0.046426588 | −1.523298784 | 9.419578344 | PPP1R32 |
| Mtss1l | ENSMUSG00000033763 | 0.002221923 | 0.046426588 | −1.174775994 | 14.1449068 | MTSS1L |
| Galnt3 | ENSMUSG00000026994 | 0.002236168 | 0.046426588 | −1.762087884 | 18.76526829 | GALNT3 |
| Il9r | ENSMUSG00000020279 | 0.002236372 | 0.046426588 | −0.97765408 | 3809.992506 | IL9R |
| Tbc1d2b | ENSMUSG00000037410 | 0.002267764 | 0.046696408 | −0.999270003 | 85.55462826 | TBC1D2B |
| Trip11 | ENSMUSG00000021188 | 0.0022702 | 0.046696408 | −1.535176925 | 800.9216782 | TRIP11 |
| Tmem86b | ENSMUSG00000045282 | 0.002286901 | 0.046884685 | −1.047217658 | 63.04238682 | TMEM86B |
| Syne1 | ENSMUSG00000019769 | 0.0023038 | 0.047058371 | −1.877779368 | 3279.656252 | SYNE1 |
| Gp1ba | ENSMUSG00000050675 | 0.002311492 | 0.047058371 | −1.356583323 | 31.53577933 | GP1BA |
| Pfkfb2 | ENSMUSG00000026409 | 0.002322318 | 0.047212926 | −1.497045407 | 294.8531523 | PFKFB2 |
| Cbx7 | ENSMUSG00000053411 | 0.002328259 | 0.047267858 | −0.715779175 | 3459.102659 | CBX7 |
| Al607873 | ENSMUSG00000073490 | 0.00234156 | 0.047429523 | −1.296738804 | 35.49601509 | IFI16 |
| Map3k12 | ENSMUSG00000023050 | 0.002355717 | 0.047429523 | −0.829678314 | 102.446822 | MAP3K12 |
| Zbtb20 | ENSMUSG00000022708 | 0.002379116 | 0.04770324 | −1.820714241 | 2455.246872 | ZBTB20 |
| Dennd2a | ENSMUSG00000038456 | 0.0024103 | 0.048185748 | −1.350111632 | 15.06832752 | DENND2A |
| Cd300e | ENSMUSG00000048498 | 0.002414153 | 0.048185748 | −1.729362091 | 29.660255 | CD300E |
| Mycbp2 | ENSMUSG00000033004 | 0.002439526 | 0.048361253 | −1.568007187 | 5286.770923 | MYCBP2 |
| Tnrc6b | ENSMUSG00000047888 | 0.002447265 | 0.048361253 | −1.560737971 | 1599.751295 | TNRC6B |
| Shank1 | ENSMUSG00000038738 | 0.002448377 | 0.048361253 | −1.643087277 | 209.4328718 | SHANK1 |
| Sntb2 | ENSMUSG00000041308 | 0.002486274 | 0.048911235 | −1.090574093 | 73.33470884 | SNTB2 |
| Dbndd2 | ENSMUSG00000017734 | 0.002535013 | 0.049135724 | −1.73560582 | 4.677556244 | DBNDD2 |
| Cd300lf | ENSMUSG00000047798 | 0.002553957 | 0.049144066 | −1.322632383 | 273.8252648 | CD300LF |
| Crb2 | ENSMUSG00000035403 | 0.002555343 | 0.049144066 | −0.947061108 | 29.09071441 | CRB2 |
| Krba1 | ENSMUSG00000042810 | 0.002567868 | 0.049255149 | −0.83320778 | 579.0831227 | KRBA1 |
| Kel | ENSMUSG00000029866 | 0.002579825 | 0.049307603 | −2.261612038 | 11.44607619 | KEL |
| Orai2 | ENSMUSG00000039747 | 0.002580736 | 0.049307603 | −0.822809874 | 729.947539 | ORAI2 |
| Rsad2 | ENSMUSG00000020641 | 0.002600861 | 0.049562356 | −1.537633979 | 50.73956779 | RSAD2 |
| Rpl9 | ENSMUSG00000047215 | 0.002641511 | 0.050271371 | −0.858074342 | 387.871764 | RPL9 |
| Map3k8 | ENSMUSG00000024235 | 0.002670136 | 0.050679991 | −0.975127519 | 333.0591158 | MAP3K8 |
| Ccdc15 | ENSMUSG00000034303 | 0.002675095 | 0.050679991 | −1.302502076 | 98.21644668 | CCDC15 |
| Leng8 | ENSMUSG00000035545 | 0.002699137 | 0.050964335 | −1.467377723 | 2103.885735 | LENG8 |
| Mdn1 | ENSMUSG00000058006 | 0.002712939 | 0.050964335 | −1.7505979 | 1735.486177 | MDN1 |
| Dclre1b | ENSMUSG00000027845 | 0.002724063 | 0.050964335 | −0.57992325 | 219.655892 | DCLRE1B |
| Sowaha | ENSMUSG00000044352 | 0.002733984 | 0.050964335 | −2.238144676 | 3.917222135 | SOWAHA |
| 2410089E03Rik | ENSMUSG00000039801 | 0.002769457 | 0.051366919 | −1.348311345 | 390.3736865 | C5orf42 |
| Rasgrf1 | ENSMUSG00000032356 | 0.002787622 | 0.051507493 | −2.052113626 | 4.694585864 | RASGRF1 |
| Sh2d4b | ENSMUSG00000037833 | 0.002802791 | 0.051670273 | −1.537446362 | 9.694337764 | SH2D4B |
| Zfp398 | ENSMUSG00000062519 | 0.002803511 | 0.051670273 | −0.915779997 | 299.5144412 | ZNF398 |
| Col27a1 | ENSMUSG00000045672 | 0.0028163 | 0.051783881 | −1.845653573 | 81.36372086 | COL27A1 |
| Myo5a | ENSMUSG00000034593 | 0.002822162 | 0.051792157 | −1.396819539 | 2909.691163 | MYO5A |
| Abl2 | ENSMUSG00000026596 | 0.002838045 | 0.051848492 | −1.628702314 | 1225.082798 | ABL2 |
| Krt80 | ENSMUSG00000037185 | 0.002841812 | 0.051852421 | −1.746138151 | 6.231696218 | KRT80 |
| Tnn | ENSMUSG00000026725 | 0.002875676 | 0.052209263 | −1.959886816 | 5.971039031 | TNN |
| Thbs1 | ENSMUSG00000040152 | 0.002887544 | 0.052294633 | −1.298747578 | 8.562818138 | THBS1 |
| 1700094D03Rik | ENSMUSG00000078667 | 0.002929464 | 0.052651874 | −0.732196825 | 209.5151333 | C1orf189 |
| Ash1l | ENSMUSG00000028053 | 0.002932519 | 0.052651874 | −1.754682273 | 1970.209966 | ASH1L |
| Tmc4 | ENSMUSG00000019734 | 0.002960344 | 0.052858346 | −1.004546281 | 54.44834478 | TMC4 |
| Hp | ENSMUSG00000031722 | 0.002960995 | 0.052858346 | −1.83794817 | 8.653475049 | HP |
| Nav2 | ENSMUSG00000052512 | 0.002963538 | 0.052858346 | −1.782120759 | 107.5243491 | NAV2 |
| Catsperg1 | ENSMUSG00000049676 | 0.002968173 | 0.052858346 | −1.508112659 | 12.13922305 | CATSPERG |
| Sec61a2 | ENSMUSG00000025816 | 0.002969651 | 0.052858346 | −0.877853229 | 93.98347743 | SEC61A2 |
| Prdm9 | ENSMUSG00000051977 | 0.002972988 | 0.052858346 | −1.429579504 | 115.538749 | PRDM9 |
| Cd300a | ENSMUSG00000034652 | 0.002998384 | 0.053245034 | −1.403402141 | 77.70668999 | CD300A |
| Slc25a37 | ENSMUSG00000034248 | 0.003008499 | 0.053294975 | −1.134730368 | 1650.989541 | SLC25A37 |
| Tnfaip2 | ENSMUSG00000021281 | 0.003039429 | 0.053612831 | −1.775717837 | 45.89237934 | TNFAIP2 |
| Chd2 | ENSMUSG00000078671 | 0.003041133 | 0.053612831 | −1.591895817 | 2542.436522 | CHD2 |
| Ikzf2 | ENSMUSG00000025997 | 0.003048044 | 0.053669846 | −1.404534203 | 24.58346807 | IKZF2 |
| Fam43a | ENSMUSG00000046546 | 0.003078616 | 0.053934152 | −1.417878054 | 907.7240983 | FAM43A |
| Akap7 | ENSMUSG00000039166 | 0.00309197 | 0.053934152 | −0.655605359 | 145.4381737 | AKAP7 |
| Bhlhe40 | ENSMUSG00000030103 | 0.003129818 | 0.05432337 | −1.04247025 | 667.5769426 | BHLHE40 |
| Stac3 | ENSMUSG00000040287 | 0.003133567 | 0.054323852 | −1.266363504 | 20.95984489 | STAC3 |
| Dact1 | ENSMUSG00000044548 | 0.003185813 | 0.054870363 | −1.342059338 | 11.41799361 | DACT1 |
| Fyb | ENSMUSG00000022148 | 0.003191156 | 0.054870363 | −1.610030752 | 122.6887965 | FYB |
| Avil | ENSMUSG00000025432 | 0.003214187 | 0.054947866 | −1.255659772 | 16.17126052 | AVIL |
| Kcnd1 | ENSMUSG00000009731 | 0.003247193 | 0.055250963 | −1.599185958 | 6.01168036 | KCND1 |
| Csf3r | ENSMUSG00000028859 | 0.003255178 | 0.055250963 | −1.642389834 | 33.57414213 | CSF3R |
| Rmnd1 | ENSMUSG00000019763 | 0.003321531 | 0.056009456 | −0.48995226 | 276.3258512 | RMND1 |
| Setd1a | ENSMUSG00000042308 | 0.003327595 | 0.056009456 | −1.041558973 | 418.5694017 | SETD1A |
| Gan | ENSMUSG00000052557 | 0.003330562 | 0.056009456 | −1.473204946 | 43.50547852 | GAN |
| Zc3h12b | ENSMUSG00000035045 | 0.003337328 | 0.056058662 | −1.896993441 | 7.052766727 | ZC3H12B |
| Zbtb34 | ENSMUSG00000068966 | 0.00334372 | 0.056101467 | −1.427308398 | 133.0693166 | ZBTB34 |
| Cdh1 | ENSMUSG00000000303 | 0.003348595 | 0.056118755 | −1.881882209 | 7.820055577 | CDH1 |
| Ypel4 | ENSMUSG00000034059 | 0.003358768 | 0.05612327 | −2.11772407 | 7.526145089 | YPEL4 |
| Tubb4a | ENSMUSG00000062591 | 0.003361102 | 0.05612327 | −0.786324181 | 49.46057703 | TUBB4A |
| Ffar3 | ENSMUSG00000019429 | 0.003375778 | 0.05612327 | −1.202308212 | 17.75717605 | FFAR3 |
| B3gntl1 | ENSMUSG00000046605 | 0.003386681 | 0.056162854 | −0.771750202 | 178.7966126 | B3GNTL1 |
| Syngap1 | ENSMUSG00000067629 | 0.003410452 | 0.056288681 | −1.215855871 | 13.03974214 | SYNGAP1 |
| Nsd1 | ENSMUSG00000021488 | 0.003413082 | 0.056288681 | −1.375376804 | 2148.679253 | NSD1 |
| Brwd1 | ENSMUSG00000022914 | 0.003416577 | 0.056288681 | −1.336369197 | 2984.392424 | BRWD1 |
| Abcc10 | ENSMUSG00000032842 | 0.003447156 | 0.056728455 | −0.92578508 | 166.5399318 | ABCC10 |
| Sgk1 | ENSMUSG00000019970 | 0.003472636 | 0.056928135 | −1.217964374 | 242.1021591 | SGK1 |
| Sept8 | ENSMUSG00000018398 | 0.003473557 | 0.056928135 | −1.662776306 | 15.29154322 | SEPT8 |
| Il18rap | ENSMUSG00000026068 | 0.003478685 | 0.056928135 | −1.294182699 | 18.67261236 | IL18RAP |
| Pak6 | ENSMUSG00000074923 | 0.003484399 | 0.056928135 | −1.38425969 | 28.95921849 | PAK6 |
| Cxcr2 | ENSMUSG00000026180 | 0.003496867 | 0.056928135 | −1.643901634 | 6.206052059 | CXCR2 |
| Arrb1 | ENSMUSG00000018909 | 0.00349829 | 0.056928135 | −1.261598395 | 460.854442 | ARRB1 |
| Golgb1 | ENSMUSG00000034243 | 0.003536364 | 0.057322531 | −1.586091342 | 1426.180884 | GOLGB1 |
| Znrf3 | ENSMUSG00000041961 | 0.003552849 | 0.057358555 | −1.289816816 | 115.5558828 | ZNRF3 |
| Herc1 | ENSMUSG00000038664 | 0.003553369 | 0.057358555 | −1.600993273 | 2288.188266 | HERC1 |
| Rfx3 | ENSMUSG00000040929 | 0.00364492 | 0.05799888 | −1.44710635 | 277.553011 | RFX3 |
| Kcnc3 | ENSMUSG00000062785 | 0.003666679 | 0.05799888 | −1.108328538 | 54.16139083 | KCNC3 |
| Card9 | ENSMUSG00000026928 | 0.003673456 | 0.05799888 | −1.389264569 | 8.917365199 | CARD9 |
| Srcap | ENSMUSG00000090663 | 0.003676912 | 0.05799888 | −1.153580425 | 748.3731953 | SRCAP |
| Nova1 | ENSMUSG00000021047 | 0.003680797 | 0.05799888 | −1.007380636 | 59.97185208 | NOVA1 |
| Nid2 | ENSMUSG00000021806 | 0.00369383 | 0.05799888 | −1.610131061 | 4.342383157 | NID2 |
| Coro2b | ENSMUSG00000041729 | 0.003696141 | 0.05799888 | −0.936981347 | 219.8276205 | CORO2B |
| Ccdc88c | ENSMUSG00000021182 | 0.003699182 | 0.05799888 | −1.40760636 | 2689.728726 | CCDC88C |
| Rnasel | ENSMUSG00000066800 | 0.003716039 | 0.058030887 | −1.222592056 | 150.2687524 | RNASEL |
| Maml3 | ENSMUSG00000061143 | 0.003767585 | 0.058640834 | −1.36668595 | 50.91665869 | MAML3 |
| Rnf170 | ENSMUSG00000013878 | 0.003769616 | 0.058640834 | −0.820828806 | 111.3265901 | RNF170 |
| Pigm | ENSMUSG00000050229 | 0.003819737 | 0.058964109 | −1.080536143 | 886.5961741 | PIGM |
| Srcap | ENSMUSG00000053877 | 0.003827437 | 0.058964109 | −1.52836921 | 708.9162389 | SRCAP |
| Dennd1c | ENSMUSG00000002668 | 0.003846367 | 0.058964109 | −0.471981213 | 1600.046754 | DENND1C |
| Hebp1 | ENSMUSG00000042770 | 0.003846644 | 0.058964109 | −1.488000117 | 56.87657606 | HEBP1 |
| Tnfsf15 | ENSMUSG00000050395 | 0.003858959 | 0.059045314 | −1.441145642 | 7.753118656 | TNFSF15 |
| Abca1 | ENSMUSG00000015243 | 0.003867151 | 0.059083883 | −1.52438333 | 1690.872149 | ABCA1 |
| Arih2 | ENSMUSG00000064145 | 0.003869712 | 0.059083883 | −0.563328206 | 727.5401647 | ARIH2 |
| Wfikkn1 | ENSMUSG00000071192 | 0.003885635 | 0.059083883 | −1.257147849 | 12.30750834 | WFIKKN1 |
| Slc45a3 | ENSMUSG00000026435 | 0.003888102 | 0.059083883 | −1.434128841 | 17.82713476 | SLC45A3 |
| Trip6 | ENSMUSG00000023348 | 0.003894598 | 0.059095054 | −0.966567622 | 56.87380867 | TRIP6 |
| Dstyk | ENSMUSG00000042046 | 0.003906042 | 0.059185894 | −1.553150494 | 129.1257716 | DSTYK |
| Arhgap39 | ENSMUSG00000033697 | 0.00392035 | 0.059185894 | −0.928186181 | 161.7130493 | ARHGAP39 |
| Bmp2k | ENSMUSG00000034663 | 0.003927368 | 0.059185894 | −1.120089834 | 2711.929603 | BMP2K |
| Cep250 | ENSMUSG00000038241 | 0.003934201 | 0.059185894 | −1.460020135 | 1147.850975 | CEP250 |
| Far2 | ENSMUSG00000030303 | 0.00397624 | 0.059346804 | −1.622020245 | 48.45500845 | FAR2 |
| Sowahc | ENSMUSG00000071286 | 0.003990287 | 0.059392796 | −1.093748254 | 268.1302328 | SOWAHC |
| Spata1 | ENSMUSG00000028188 | 0.003997948 | 0.059392796 | −0.860112342 | 58.1100795 | SPATA1 |
| Arhgap29 | ENSMUSG00000039831 | 0.00400681 | 0.059438427 | −1.570536108 | 19.1194466 | ARHGAP29 |
| Mxd1 | ENSMUSG00000001156 | 0.004018874 | 0.059515288 | −0.856241302 | 320.1744354 | MXD1 |
| Aasdh | ENSMUSG00000055923 | 0.004028825 | 0.059539384 | −0.759002725 | 331.7909899 | AASDH |
| Tmem8b | ENSMUSG00000078716 | 0.004029932 | 0.059539384 | −0.995315434 | 23.33478386 | TMEM8B |
| Mdm4 | ENSMUSG00000054387 | 0.004063883 | 0.059841595 | −1.596188925 | 1628.120635 | MDM4 |
| Myo1h | ENSMUSG00000066952 | 0.004086459 | 0.059841595 | −1.177975603 | 48.93474814 | MYO1H |
| Mapk8ip3 | ENSMUSG00000024163 | 0.004103724 | 0.059841595 | −1.170440197 | 1821.199673 | MAPK8IP3 |
| Zbtb46 | ENSMUSG00000027583 | 0.004124179 | 0.059841595 | −1.126693849 | 33.20585388 | ZBTB46 |
| Szt2 | ENSMUSG00000033253 | 0.004138726 | 0.059993027 | −1.12068361 | 1435.76862 | SZT2 |
| Arhgap33 | ENSMUSG00000036882 | 0.004205858 | 0.060304885 | −1.015205327 | 132.308769 | ARHGAP33 |
| Adora3 | ENSMUSG00000000562 | 0.00425102 | 0.060680686 | −1.725183182 | 4.984216455 | ADORA3 |
| Tor1aIp2 | ENSMUSG00000050565 | 0.004283291 | 0.060938789 | −1.338929927 | 560.3701064 | TOR1AIP2 |
| Etv6 | ENSMUSG00000030199 | 0.004318738 | 0.061026738 | −0.818437178 | 629.1260978 | ETV6 |
| Cpeb4 | ENSMUSG00000020300 | 0.004334407 | 0.061070791 | −1.467384446 | 502.19185 | CPEB4 |
| 1810046K07Rik | ENSMUSG00000036027 | 0.004342074 | 0.061092939 | −1.60859306 | 24.11001019 | C11orf53 |
| Phc3 | ENSMUSG00000037652 | 0.00439176 | 0.061308249 | −1.556736277 | 1067.209404 | PHC3 |
| Slco2a1 | ENSMUSG00000032548 | 0.004433474 | 0.06162591 | −1.054883975 | 71.23045085 | SLCO2A1 |
| Plekhm3 | ENSMUSG00000051344 | 0.004439294 | 0.06162591 | −1.255686597 | 1386.353413 | PLEKHM3 |
| 4833420G17Rik | ENSMUSG00000062822 | 0.004468578 | 0.06171035 | −0.536805313 | 1272.52028 | C5orf34 |
| Igfbp7 | ENSMUSG00000036256 | 0.004485698 | 0.061728754 | −1.739114272 | 11.93676987 | IGFBP7 |
| Ffar1 | ENSMUSG00000044453 | 0.004486827 | 0.061728754 | −1.423557065 | 294.0493983 | FFAR1 |
| Fbxo24 | ENSMUSG00000089984 | 0.004535313 | 0.06191093 | −1.163643833 | 15.0020521 | FBXO24 |
| Papss2 | ENSMUSG00000024899 | 0.00453824 | 0.06191093 | −1.517502056 | 8.874841112 | PAPSS2 |
| Pkd1l3 | ENSMUSG00000048827 | 0.004546556 | 0.061966459 | −1.21493691 | 38.22903908 | PKD1L3 |
| Hemgn | ENSMUSG00000028332 | 0.004579932 | 0.062117596 | −1.325076701 | 182.0621619 | HEMGN |
| Wwc2 | ENSMUSG00000031563 | 0.004601686 | 0.062117596 | −1.45414707 | 22.74419776 | WWC2 |
| Rnd3 | ENSMUSG00000017144 | 0.004616673 | 0.062117596 | −1.233769836 | 12.48476459 | RND3 |
| Snai1 | ENSMUSG00000042821 | 0.004633021 | 0.062117596 | −1.105681494 | 12.17247913 | SNAI1 |
| Sdc3 | ENSMUSG00000025743 | 0.004667927 | 0.062340096 | −1.293709622 | 349.3145776 | SDC3 |
| Ptprf | ENSMUSG00000033295 | 0.004715146 | 0.062573088 | −2.05447342 | 9.076929938 | PTPRF |
| Myo10 | ENSMUSG00000022272 | 0.004716172 | 0.062573088 | −1.168366523 | 63.26967784 | MYO10 |
| Tep1 | ENSMUSG00000006281 | 0.004787927 | 0.063161972 | −0.95245391 | 981.7871334 | TEP1 |
| Zfp112 | ENSMUSG00000052675 | 0.004829136 | 0.063163419 | −1.041550117 | 19.37446062 | ZNF112 |
| Zmym3 | ENSMUSG00000031310 | 0.004843402 | 0.063194562 | −0.541459127 | 745.7185446 | ZMYM3 |
| Csrnp1 | ENSMUSG00000032515 | 0.004844469 | 0.063194562 | −0.636381524 | 2137.416521 | CSRNP1 |
| Rnf213 | ENSMUSG00000070327 | 0.004854846 | 0.063273376 | −1.534003425 | 3460.74652 | RNF213 |
| Pik3c2b | ENSMUSG00000026447 | 0.004872695 | 0.063400617 | −1.281902831 | 3027.686018 | PIK3C2B |
| AW554918 | ENSMUSG00000033632 | 0.004923976 | 0.063400617 | −0.665426781 | 264.6898486 | KIAA1328 |
| Junb | ENSMUSG00000052837 | 0.004964373 | 0.063400617 | −1.077389476 | 5217.437044 | JUNB |
| Mgat4a | ENSMUSG00000026110 | 0.004972945 | 0.063400617 | −1.18800224 | 987.5953597 | MGAT4A |
| Cd302 | ENSMUSG00000060703 | 0.004975971 | 0.063400617 | −1.188706479 | 38.06684966 | CD302 |
| Tmcc3 | ENSMUSG00000020023 | 0.004979606 | 0.063400617 | −1.258450169 | 151.4550485 | TMCC3 |
| Kcng1 | ENSMUSG00000074575 | 0.004989246 | 0.063400617 | −1.206992014 | 51.83194036 | KCNG1 |
| Mtcp1 | ENSMUSG00000031200 | 0.004997812 | 0.063400617 | −1.27792211 | 138.7569717 | CMC4 |
| Amigo3 | ENSMUSG00000032593 | 0.005013296 | 0.063400617 | −1.231008729 | 98.18197283 | AMIGO3 |
| Map3k9 | ENSMUSG00000042724 | 0.00502935 | 0.063400617 | −1.29755695 | 800.0182799 | MAP3K9 |
| Insr | ENSMUSG00000005534 | 0.005031478 | 0.063400617 | −1.401230214 | 410.6007626 | INSR |
| Hfe | ENSMUSG00000006611 | 0.005034001 | 0.063400617 | −1.420298017 | 41.01244102 | HFE |
| Tnfaip3 | ENSMUSG00000019850 | 0.005048695 | 0.063476148 | −1.293504858 | 1834.228509 | TNFAIP3 |
| Tmem170b | ENSMUSG00000087370 | 0.005100505 | 0.06396329 | −1.242339415 | 281.2235186 | TMEM170B |
| St6galnac2 | ENSMUSG00000057286 | 0.005162675 | 0.064301438 | −1.296597821 | 13.47907473 | ST6GALNAC2 |
| Adamtsl4 | ENSMUSG00000015850 | 0.00518308 | 0.064301438 | −1.039438821 | 106.148533 | ADAMTSL4 |
| Plxnb3 | ENSMUSG00000031385 | 0.005185852 | 0.064301438 | −1.383598251 | 11.54105353 | PLXNB3 |
| 1700029J07Rik | ENSMUSG00000071103 | 0.005189223 | 0.064301438 | −1.006833881 | 124.0464484 | C4orf47 |
| Ghrl | ENSMUSG00000064177 | 0.005282908 | 0.064484775 | −1.945347672 | 6.752720904 | GHRL |
| Zfp831 | ENSMUSG00000050600 | 0.005322418 | 0.064745525 | −1.442124786 | 738.7168355 | ZNF831 |
| Ankrd11 | ENSMUSG00000035569 | 0.005327079 | 0.064745525 | −1.481449147 | 3727.235855 | ANKRD11 |
| Ikbke | ENSMUSG00000042349 | 0.005392208 | 0.065103445 | −0.804442999 | 270.451603 | IKBKE |
| Hspa1a | ENSMUSG00000091971 | 0.00540187 | 0.065107898 | −2.01272039 | 1638.470421 | HSPA1B |
| Hspa1a | ENSMUSG00000091971 | 0.00540187 | 0.065107898 | −2.01272039 | 1638.470421 | HSPA1A |
| Smg1 | ENSMUSG00000030655 | 0.005414353 | 0.065107898 | −1.620661319 | 5537.654304 | SMG1 |
| Paqr9 | ENSMUSG00000064225 | 0.005415298 | 0.065107898 | −1.43889666 | 11.23505493 | PAQR9 |
| Huwe1 | ENSMUSG00000025261 | 0.005430988 | 0.065158666 | −1.474874189 | 6709.960857 | HUWE1 |
| Atp8b4 | ENSMUSG00000060131 | 0.005432493 | 0.065158666 | −1.153326875 | 29.11909392 | ATP8B4 |
| Trim7 | ENSMUSG00000040350 | 0.005460034 | 0.065221046 | −0.749729901 | 2125.564446 | TRIM7 |
| Iffo1 | ENSMUSG00000038271 | 0.005469791 | 0.065284165 | −0.723614942 | 782.5437224 | IFFO1 |
| Lrrc16b | ENSMUSG00000022211 | 0.005517392 | 0.065322115 | −0.916604133 | 48.51566458 | LRRC16B |
| Zfp113 | ENSMUSG00000037007 | 0.005517721 | 0.065322115 | −1.073910455 | 182.7190222 | ZNF3 |
| Slc4a8 | ENSMUSG00000023032 | 0.005534959 | 0.065394022 | −1.021457519 | 698.6968473 | SLC4A8 |
| Kcnb1 | ENSMUSG00000050556 | 0.005542382 | 0.065401897 | −0.987740681 | 109.5872635 | KCNB1 |
| Wdr13 | ENSMUSG00000031166 | 0.005596886 | 0.065779187 | −1.161926916 | 368.6716062 | WDR13 |
| Mob1b | ENSMUSG00000006262 | 0.005616374 | 0.065955123 | −0.977642742 | 507.2650533 | MOB1B |
| Zcchc7 | ENSMUSG00000035649 | 0.005624965 | 0.065991755 | −1.033788412 | 1941.581156 | ZCCHC7 |
| Col7a1 | ENSMUSG00000025650 | 0.005628536 | 0.065991755 | −1.147532895 | 27.14193201 | COL7A1 |
| Mybpc3 | ENSMUSG00000002100 | 0.005654101 | 0.066079188 | −1.16002188 | 11.44074656 | MYBPC3 |
| Zzef1 | ENSMUSG00000055670 | 0.005690898 | 0.066188857 | −1.275593674 | 1810.175411 | ZZEF1 |
| Isl1 | ENSMUSG00000042258 | 0.005724826 | 0.066188857 | −1.037685718 | 45.06655869 | ISL1 |
| Slc9a9 | ENSMUSG00000031129 | 0.005757394 | 0.066188857 | −0.832841552 | 71.06300195 | SLC9A9 |
| Klf11 | ENSMUSG00000020653 | 0.005771183 | 0.066188857 | −1.355579779 | 165.3404477 | KLF11 |
| Ccl3 | ENSMUSG00000000982 | 0.005780896 | 0.066188857 | −1.443307871 | 25.28154734 | CCL3L3 |
| Fat1 | ENSMUSG00000070047 | 0.005782589 | 0.066188857 | −1.967272625 | 6.060135749 | FAT1 |
| Lrch4 | ENSMUSG00000029720 | 0.005809925 | 0.066188857 | −1.075722367 | 65.25377889 | LRCH4 |
| Herc2 | ENSMUSG00000030451 | 0.005815333 | 0.066188857 | −1.532534847 | 2148.837442 | HERC2 |
| Palm3 | ENSMUSG00000047986 | 0.005820557 | 0.066188857 | −1.191443456 | 19.84764565 | PALM3 |
| Dsel | ENSMUSG00000038702 | 0.005831258 | 0.066188857 | −0.98287202 | 15.73048342 | DSEL |
| Med1 | ENSMUSG00000018160 | 0.005848133 | 0.06627733 | −1.105994554 | 1269.517001 | MED1 |
| Rnf150 | ENSMUSG00000047747 | 0.005902488 | 0.0665206 | −1.54731947 | 4.722111847 | RNF150 |
| Ccnj | ENSMUSG00000025010 | 0.005906056 | 0.0665206 | −1.151212748 | 200.6602739 | CCNJ |
| Dbndd1 | ENSMUSG00000031970 | 0.005929244 | 0.066627542 | −1.670026792 | 8.639444548 | DBNDD1 |
| Plxdc1 | ENSMUSG00000017417 | 0.005952523 | 0.066628684 | −1.234409063 | 12.45504943 | PLXDC1 |
| Ppm1k | ENSMUSG00000037826 | 0.005953266 | 0.066628684 | −1.286787958 | 503.49303 | PPM1K |
| Hsd3b7 | ENSMUSG00000042289 | 0.005963327 | 0.066628684 | −0.66988067 | 47.16164 | HSD3B7 |
| Rxra | ENSMUSG00000015846 | 0.006014065 | 0.066693009 | −1.275539069 | 62.88204094 | RXRA |
| Snapc4 | ENSMUSG00000036281 | 0.006027291 | 0.066693009 | −0.977672401 | 567.576063 | SNAPC4 |
| Eng | ENSMUSG00000026814 | 0.00604389 | 0.066693009 | −1.507521038 | 9.067408936 | ENG |
| Mafg | ENSMUSG00000051510 | 0.006059564 | 0.066693009 | −0.987908116 | 354.4876684 | MAFG |
| Aak1 | ENSMUSG00000057230 | 0.006125685 | 0.067058591 | −1.28357627 | 209.4298147 | AAK1 |
| Hdgfrp3 | ENSMUSG00000025104 | 0.006130934 | 0.067058591 | −1.617522948 | 35.61538229 | HDGFRP3 |
| Ppl | ENSMUSG00000039457 | 0.006132988 | 0.067058591 | −1.420542331 | 105.313222 | PPL |
| F8 | ENSMUSG00000031196 | 0.006140491 | 0.067064609 | −0.949792321 | 28.91801433 | F8 |
| Sfxn5 | ENSMUSG00000033720 | 0.006174661 | 0.067162096 | −0.664024562 | 121.0857564 | SFXN5 |
| Zfp329 | ENSMUSG00000057894 | 0.006205406 | 0.067267616 | −1.098171615 | 350.5182412 | ZNF329 |
| Fan1 | ENSMUSG00000033458 | 0.006207404 | 0.067267616 | −1.28583474 | 73.11320238 | FAN1 |
| Slc38a9 | ENSMUSG00000069056 | 0.006217953 | 0.067282035 | −1.317676608 | 79.57055357 | SLC38A9 |
| Setbp1 | ENSMUSG00000024548 | 0.006263451 | 0.067508043 | −1.467343435 | 923.3840662 | SETBP1 |
| Fry | ENSMUSG00000056602 | 0.006273479 | 0.067508043 | −1.438069053 | 454.1576082 | FRY |
| Lrp5 | ENSMUSG00000024913 | 0.006281916 | 0.067508043 | −1.200802026 | 20.17448104 | LRP5 |
| Ampd3 | ENSMUSG00000005686 | 0.006285088 | 0.067508043 | −1.177240612 | 101.6812504 | AMPD3 |
| Heatr5b | ENSMUSG00000073113 | 0.006299001 | 0.067607739 | −1.000644902 | 21.57806596 | HEATR5B |
| Rbbp6 | ENSMUSG00000030779 | 0.006352633 | 0.067912648 | −1.111321921 | 2263.888205 | RBBP6 |
| Mfsd9 | ENSMUSG00000041945 | 0.006376618 | 0.068040562 | −1.268604805 | 11.31954383 | MFSD9 |
| Megf8 | ENSMUSG00000045039 | 0.006424186 | 0.068296032 | −1.535851825 | 17.50301396 | MEGF8 |
| Ccr3 | ENSMUSG00000035448 | 0.006454714 | 0.068402961 | −1.338024426 | 54.02627742 | CCR3 |
| Rbm15 | ENSMUSG00000048109 | 0.006487665 | 0.068547606 | −1.007967982 | 676.3291177 | RBM15 |
| Rab3il1 | ENSMUSG00000024663 | 0.006500087 | 0.068547606 | −1.417229715 | 36.77181948 | RAB3IL1 |
| Cep97 | ENSMUSG00000022604 | 0.006571376 | 0.068860286 | −0.799332426 | 327.8690795 | CEP97 |
| Sned1 | ENSMUSG00000047793 | 0.006588593 | 0.068891979 | −1.500202894 | 10.86745422 | SNED1 |
| Ephb2 | ENSMUSG00000028664 | 0.006634926 | 0.068891979 | −1.289117984 | 84.99084886 | EPHB2 |
| Capn3 | ENSMUSG00000079110 | 0.006644226 | 0.068891979 | −1.107875414 | 22.61978555 | CAPN3 |
| Prkdc | ENSMUSG00000022672 | 0.006676123 | 0.06891893 | −1.322132873 | 932.1180528 | PRKDC |
| Dcaf10 | ENSMUSG00000035572 | 0.006684921 | 0.068960984 | −0.58808453 | 444.4298506 | DCAF10 |
| Igsf6 | ENSMUSG00000035004 | 0.006744162 | 0.06909071 | −1.282504898 | 54.58029474 | IGSF6 |
| Dnmt3a | ENSMUSG00000020661 | 0.006794 | 0.069153049 | −0.997039468 | 510.6695913 | DNMT3A |
| Rapgef4 | ENSMUSG00000049044 | 0.006797874 | 0.069153049 | −0.776932861 | 325.4108559 | RAPGEF4 |
| Prkab2 | ENSMUSG00000038205 | 0.006802918 | 0.069153049 | −1.197868527 | 188.1813361 | PRKAB2 |
| Nfic | ENSMUSG00000055053 | 0.006819277 | 0.069221825 | −0.96858722 | 255.2294523 | NFIC |
| Pou6f1 | ENSMUSG00000009739 | 0.006861902 | 0.069439372 | −1.068296249 | 871.4861743 | POU6F1 |
| Dopey2 | ENSMUSG00000022946 | 0.006887921 | 0.069464463 | −1.061419275 | 373.3857369 | DOPEY2 |
| Pcf11 | ENSMUSG00000041328 | 0.006917224 | 0.069484715 | −1.222381213 | 1799.227623 | PCF11 |
| Trim36 | ENSMUSG00000033949 | 0.006921338 | 0.069484715 | −1.252366973 | 72.35444993 | TRIM36 |
| Klhdc10 | ENSMUSG00000029775 | 0.006994734 | 0.069932964 | −0.665516207 | 379.1279401 | KLHDC10 |
| Clcn1 | ENSMUSG00000029862 | 0.007005496 | 0.069944749 | −1.570622082 | 4.617537397 | CLCN1 |
| Ric8b | ENSMUSG00000035620 | 0.007029323 | 0.070065212 | −0.631125899 | 295.3321598 | RIC8B |
| Siglece | ENSMUSG00000030474 | 0.007046835 | 0.070108842 | −1.470618851 | 41.54527022 | SIGLEC9 |
| Zfand1 | ENSMUSG00000039795 | 0.007094405 | 0.070113266 | −0.637343811 | 164.3488286 | ZFAND1 |
| Adam8 | ENSMUSG00000025473 | 0.007099643 | 0.07011738 | −1.036707183 | 24.71823835 | ADAM8 |
| Bod1l | ENSMUSG00000061755 | 0.007106209 | 0.070134776 | −1.543825167 | 1261.686746 | BOD1L1 |
| 8030462N17Rik | ENSMUSG00000047466 | 0.007145753 | 0.070287439 | −0.922145578 | 338.3174376 | C1801125 |
| Gtpbp2 | ENSMUSG00000023952 | 0.007159196 | 0.070309167 | −0.772834724 | 1803.078417 | GTPBP2 |
| Lair1 | ENSMUSG00000055541 | 0.007249811 | 0.070348737 | −1.037738249 | 105.3515399 | LAIR1 |
| P2rx1 | ENSMUSG00000020787 | 0.007254594 | 0.070348737 | −1.254284652 | 12.15216666 | P2RX1 |
| C4a | ENSMUSG00000015451 | 0.007287478 | 0.070348737 | −1.01503145 | 16.26541338 | C4B |
| C4a | ENSMUSG00000015451 | 0.007287478 | 0.070348737 | −1.01503145 | 16.26541338 | C4A |
| Hoxb4 | ENSMUSG00000038692 | 0.007287846 | 0.070348737 | −1.209095074 | 14.45189147 | HOXB4 |
| Gas7 | ENSMUSG00000033066 | 0.007392635 | 0.070621924 | −1.009367658 | 81.37762259 | GAS7 |
| Brca2 | ENSMUSG00000041147 | 0.007484544 | 0.070998401 | −1.191456499 | 238.2777251 | BRCA2 |
| Nbeal2 | ENSMUSG00000056724 | 0.007531899 | 0.071206686 | −1.126827729 | 808.7592777 | NBEAL2 |
| Kif19a | ENSMUSG00000010021 | 0.007622393 | 0.071736989 | −0.734737348 | 91.5843586 | KIF19 |
| Ica1l | ENSMUSG00000026018 | 0.007690092 | 0.071775223 | −1.443336665 | 7.367650444 | ICA1L |
| Ttc23 | ENSMUSG00000030555 | 0.007702962 | 0.071775223 | −0.812200801 | 21.91071957 | TTC23 |
| Atxn7l1 | ENSMUSG00000020564 | 0.007705129 | 0.071775223 | −0.824102575 | 989.933163 | ATXN7L1 |
| Cul9 | ENSMUSG00000040327 | 0.007788677 | 0.072118409 | −0.864679679 | 308.5469277 | CUL9 |
| Plec | ENSMUSG00000022565 | 0.007804307 | 0.072165478 | −1.54174215 | 1574.721059 | PLEC |
| Kcnj16 | ENSMUSG00000051497 | 0.007835819 | 0.072220199 | −1.511399235 | 17.29287385 | KCNJ16 |
| Slc3a1 | ENSMUSG00000024131 | 0.007848542 | 0.072220199 | −1.383078768 | 7.33339704 | SLC3A1 |
| Eif4ebp3 | ENSMUSG00000090264 | 0.007885379 | 0.072283221 | −1.776557782 | 5.771300248 | EIF4EBP3 |
| Mks1 | ENSMUSG00000034121 | 0.007891176 | 0.072283221 | −0.550475874 | 233.977386 | MKS1 |
| Fblim1 | ENSMUSG00000006219 | 0.007933775 | 0.072341363 | −0.81082868 | 29.76432556 | FBLIM1 |
| Itga4 | ENSMUSG00000027009 | 0.007967186 | 0.072341363 | −1.413970532 | 1931.831585 | ITGA4 |
| Ikbkb | ENSMUSG00000031537 | 0.007968547 | 0.072341363 | −0.89207925 | 2706.709153 | IKBKB |
| Rc3h2 | ENSMUSG00000075376 | 0.008032308 | 0.072378521 | −1.364570284 | 932.1174907 | RC3H2 |
| Haus5 | ENSMUSG00000078762 | 0.008062662 | 0.072438104 | −0.283835278 | 679.5890562 | HAUS5 |
| Ptpn23 | ENSMUSG00000036057 | 0.008064117 | 0.072438104 | −0.994782459 | 739.6890278 | PTPN23 |
| Myl9 | ENSMUSG00000067818 | 0.008082098 | 0.072510375 | −1.549232468 | 5.941545471 | MYL9 |
| Glcci1 | ENSMUSG00000029638 | 0.00810744 | 0.072555875 | −1.6279329 | 50.39073291 | GLCCI1 |
| Kifc2 | ENSMUSG00000004187 | 0.008121568 | 0.072596773 | −0.774165418 | 71.08273593 | KIFC2 |
| Synj1 | ENSMUSG00000022973 | 0.008164294 | 0.072836589 | −1.161059001 | 317.353507 | SYNJ1 |
| Nlrc4 | ENSMUSG00000039193 | 0.008185729 | 0.072836589 | −0.735662725 | 598.0362046 | NLRC4 |
| Itpr1 | ENSMUSG00000030102 | 0.008211332 | 0.072836589 | −1.433819177 | 1622.512534 | ITPR1 |
| Pgap1 | ENSMUSG00000073678 | 0.008212343 | 0.072836589 | −1.295297968 | 243.338791 | PGAP1 |
| Ttc28 | ENSMUSG00000033209 | 0.008245609 | 0.072969953 | −1.297327488 | 106.70871 | TTC28 |
| Map4k2 | ENSMUSG00000024948 | 0.00827772 | 0.072969953 | −0.78559624 | 5121.273547 | MAP4K2 |
| Slc9a8 | ENSMUSG00000039463 | 0.008297202 | 0.073048404 | −0.652089377 | 1216.717822 | SLC9A8 |
| Map3k2 | ENSMUSG00000024383 | 0.008348944 | 0.073238905 | −1.350346656 | 682.1872842 | MAP3K2 |
| Tnfrsf19 | ENSMUSG00000060548 | 0.008421582 | 0.073566153 | −1.41626026 | 8.20855857 | TNFRSF19 |
| Plxna1 | ENSMUSG00000030084 | 0.008431607 | 0.073566153 | −1.2286356 | 94.76484065 | PLXNA1 |
| Wipi2 | ENSMUSG00000029578 | 0.008456703 | 0.073653039 | −0.487224804 | 1735.024181 | WIPI2 |
| Tcte1 | ENSMUSG00000023949 | 0.008632849 | 0.074454297 | −1.202709829 | 18.31170416 | TCTE1 |
| Tnik | ENSMUSG00000027692 | 0.008652909 | 0.074454297 | −1.252380548 | 62.58573792 | TNIK |
| B4galt6 | ENSMUSG00000056124 | 0.00868173 | 0.074543057 | −1.262365127 | 7.834040838 | B4GALT6 |
| Kcnc1 | ENSMUSG00000058975 | 0.008684849 | 0.074543057 | −0.915850033 | 30.18553447 | KCNC1 |
| 4932438A13Rik | ENSMUSG00000037270 | 0.008686562 | 0.074543057 | −1.437686908 | 2636.784272 | KIAA1109 |
| Plcxd2 | ENSMUSG00000087141 | 0.00873337 | 0.074762504 | −1.415657244 | 155.538752 | PLCXD2 |
| Cubn | ENSMUSG00000026726 | 0.008737753 | 0.074762504 | −1.327862452 | 5.270437768 | CUBN |
| Sfi1 | ENSMUSG00000023764 | 0.008803498 | 0.075095961 | −0.828576189 | 2164.403671 | SFI1 |
| Dusp18 | ENSMUSG00000047205 | 0.008807583 | 0.075095961 | −1.363665468 | 5.859329124 | DUSP18 |
| Tulp4 | ENSMUSG00000034377 | 0.008910063 | 0.075366556 | −1.029548616 | 374.1201536 | TULP4 |
| Zfp609 | ENSMUSG00000040524 | 0.008929624 | 0.075452185 | −1.397384853 | 292.4276051 | ZNF609 |
| Aire | ENSMUSG00000000731 | 0.008963416 | 0.075452185 | −1.188740601 | 288.4261911 | AIRE |
| Epb4.1l1 | ENSMUSG00000027624 | 0.008989707 | 0.075452185 | −1.504385872 | 7.357302465 | EPB41L1 |
| Slc24a5 | ENSMUSG00000035183 | 0.009004574 | 0.075466731 | −1.398389371 | 11.00741238 | SLC24A5 |
| Dmxl2 | ENSMUSG00000041268 | 0.009006422 | 0.075466731 | −1.38499073 | 102.8793973 | DMXL2 |
| Egf | ENSMUSG00000028017 | 0.009030382 | 0.075466731 | −0.839725579 | 34.96998486 | EGF |
| Itgb8 | ENSMUSG00000025321 | 0.009035378 | 0.075466731 | −1.424100187 | 29.70300082 | ITGB8 |
| Ttll3 | ENSMUSG00000030276 | 0.009060147 | 0.075466731 | −0.897196027 | 229.134985 | TTLL3 |
| Ulk3 | ENSMUSG00000032308 | 0.009080264 | 0.075566723 | −0.59051229 | 709.6822763 | ULK3 |
| Il18r1 | ENSMUSG00000026070 | 0.009097507 | 0.075624321 | −1.391598706 | 128.4917149 | IL18R1 |
| Prr22 | ENSMUSG00000090273 | 0.009104819 | 0.075642027 | −0.819095831 | 19.44019122 | PRR22 |
| Btbd11 | ENSMUSG00000020042 | 0.009127177 | 0.075735185 | −1.425794149 | 5.042056587 | BTBD11 |
| Zfp318 | ENSMUSG00000015597 | 0.009176156 | 0.075735185 | −1.395553179 | 1929.947983 | ZNF318 |
| Klf12 | ENSMUSG00000072294 | 0.009189901 | 0.075735185 | −1.374393318 | 52.04201957 | KLF12 |
| Timd4 | ENSMUSG00000055546 | 0.009204937 | 0.07574096 | −1.223804755 | 26.80434413 | TIMD4 |
| Atf7 | ENSMUSG00000052414 | 0.009224568 | 0.075774346 | −1.13733044 | 330.7587183 | ATF7 |
| Pggt1b | ENSMUSG00000024477 | 0.009273798 | 0.076135901 | −0.794339849 | 438.3002968 | PGGT1B |
| 6330408A02Rik | ENSMUSG00000070814 | 0.009349864 | 0.076301544 | −0.83965435 | 124.1229997 | C19orf68 |
| Slc7a8 | ENSMUSG00000022180 | 0.009400941 | 0.076362011 | −1.362461261 | 18.84625293 | SLC7A8 |
| Oaf | ENSMUSG00000032014 | 0.009474784 | 0.076482045 | −1.338191086 | 5.323340694 | OAF |
| Epb4.1l3 | ENSMUSG00000024044 | 0.009497623 | 0.076510375 | −1.390452016 | 35.21219197 | EPB41L3 |
| Cpd | ENSMUSG00000020841 | 0.009579655 | 0.076743728 | −1.214763362 | 66.18727319 | CPD |
| Zbtb43 | ENSMUSG00000026788 | 0.009590203 | 0.076743728 | −1.179724555 | 482.4853546 | ZBTB43 |
| Pf4 | ENSMUSG00000029373 | 0.009606944 | 0.076762804 | −1.282712847 | 7.78264168 | PF4 |
| Fbxl18 | ENSMUSG00000066640 | 0.009632796 | 0.076794062 | −0.932708501 | 208.3937876 | FBXL18 |
| Cd8a | ENSMUSG00000053977 | 0.009660648 | 0.076796357 | −1.199732644 | 21.17237337 | CD8A |
| Shb | ENSMUSG00000044813 | 0.009664651 | 0.076796357 | −1.007689943 | 62.78622235 | SHB |
| Mast1 | ENSMUSG00000053693 | 0.009670486 | 0.076800919 | −1.235703388 | 25.47046984 | MAST1 |
| Rccd1 | ENSMUSG00000038930 | 0.009700371 | 0.076870965 | −0.614437766 | 330.4262645 | RCCD1 |
| BC017158 | ENSMUSG00000030780 | 0.009734412 | 0.077098865 | −0.447126887 | 279.8311681 | C16orf58 |
| Bcl9l | ENSMUSG00000063382 | 0.009745742 | 0.07711483 | −1.331878174 | 643.554208 | BCL9L |
| Cd300lb | ENSMUSG00000063193 | 0.009751711 | 0.07711483 | −1.516559706 | 8.868505377 | CD300LB |
| Zfp867 | ENSMUSG00000054519 | 0.009773899 | 0.077160413 | −0.70745998 | 99.61684205 | ZNF627 |
| Eml5 | ENSMUSG00000051166 | 0.009804541 | 0.077208873 | −1.250789071 | 536.441661 | EML5 |
| Zcchc4 | ENSMUSG00000029179 | 0.009823956 | 0.077221476 | −0.653739629 | 367.9298302 | ZCCHC4 |
| Fam179a | ENSMUSG00000045761 | 0.009868742 | 0.077240875 | −1.256245883 | 11.75812909 | FAM179A |
| Apc | ENSMUSG00000005871 | 0.009971268 | 0.077461736 | −1.512694223 | 1193.695641 | APC |
| Zufsp | ENSMUSG00000039531 | 0.009979342 | 0.077467882 | −0.571475058 | 872.6293115 | ZUFSP |
| Mitf | ENSMUSG00000035158 | 0.010003561 | 0.077523519 | −1.377312097 | 9.762648442 | MITF |
| Cabin1 | ENSMUSG00000020196 | 0.010014623 | 0.077550903 | −0.705389929 | 1908.191728 | CABIN1 |
| Itpripl2 | ENSMUSG00000073858 | 0.010034802 | 0.077638083 | −1.549402052 | 7.266360753 | ITPRIPL2 |
| Atf7ip | ENSMUSG00000053935 | 0.010036519 | 0.077638083 | −1.249684979 | 473.0102725 | ATF7IP |
| Tnrc6c | ENSMUSG00000025571 | 0.010098179 | 0.077738507 | −1.370300193 | 1068.118831 | TNRC6C |
| Bace1 | ENSMUSG00000032086 | 0.010100092 | 0.077738507 | −1.581333569 | 107.0940562 | BACE1 |
| Arhgap31 | ENSMUSG00000022799 | 0.010102722 | 0.077738507 | −1.070499824 | 351.5413968 | ARHGAP31 |
| Camk2g | ENSMUSG00000021820 | 0.010114604 | 0.077738507 | −0.69358034 | 359.6236645 | CAMK2G |
| Abcc5 | ENSMUSG00000022822 | 0.010148019 | 0.077738507 | −1.037009776 | 283.9034448 | ABCC5 |
| Ankdd1b | ENSMUSG00000047117 | 0.010197876 | 0.077805875 | −0.690139797 | 28.30465108 | ANKDD1B |
| Rel | ENSMUSG00000020275 | 0.010217197 | 0.077805875 | −1.24474685 | 751.2718915 | REL |
| Coro7 | ENSMUSG00000039637 | 0.01021946 | 0.077805875 | −0.679054753 | 2271.570983 | COR07-PAM16 |
| Coro7 | ENSMUSG00000039637 | 0.01021946 | 0.077805875 | −0.679054753 | 2271.570983 | COR07 |
| Fam84b | ENSMUSG00000072568 | 0.010235383 | 0.077805875 | −1.279368329 | 153.003237 | FAM84B |
| Dock7 | ENSMUSG00000028556 | 0.01026318 | 0.077824228 | −1.325236649 | 13.18297993 | DOCK7 |
| Scn1b | ENSMUSG00000019194 | 0.010279127 | 0.077889678 | −1.507647476 | 10.25864626 | SCN1B |
| Coro2a | ENSMUSG00000028337 | 0.010282483 | 0.077889678 | −0.766701661 | 1175.534362 | CORO2A |
| Smarcad1 | ENSMUSG00000029920 | 0.010298272 | 0.07790701 | −0.512818993 | 650.0448339 | SMARCAD1 |
| Ep400 | ENSMUSG00000029505 | 0.010310769 | 0.077942155 | −1.227772446 | 3221.936449 | EP400 |
| Pcyt1a | ENSMUSG00000005615 | 0.010338473 | 0.077952492 | −0.92222021 | 219.1119949 | PCYT1A |
| Sytl2 | ENSMUSG00000030616 | 0.010343618 | 0.077952492 | −1.498680012 | 3.879270156 | SYTL2 |
| Pigl | ENSMUSG00000014245 | 0.010379841 | 0.077952492 | −0.538816063 | 163.6964408 | PIGL |
| Crybg3 | ENSMUSG00000022723 | 0.010402189 | 0.077952492 | −1.181733286 | 272.8233253 | CRYBG3 |
| Tgm1 | ENSMUSG00000022218 | 0.010429239 | 0.078036842 | −1.362663195 | 37.77807492 | TGM1 |
| Fanca | ENSMUSG00000032815 | 0.010436785 | 0.078045976 | −0.530541536 | 402.1041096 | FANCA |
| Teti | ENSMUSG00000047146 | 0.010465681 | 0.078181957 | −1.565179149 | 9.24043738 | TETI |
| Rbak | ENSMUSG00000061898 | 0.010480995 | 0.078256307 | −1.096973558 | 178.9377105 | RBAK |
| Trp53inp2 | ENSMUSG00000038375 | 0.010567025 | 0.078315781 | −1.030868272 | 237.5116097 | TP53INP2 |
| Traf3 | ENSMUSG00000021277 | 0.010581861 | 0.078315781 | −0.981581757 | 1147.414028 | TRAF3 |
| Asxl2 | ENSMUSG00000037486 | 0.010604351 | 0.078375553 | −1.203026061 | 1459.529668 | ASXL2 |
| Tomt | ENSMUSG00000078630 | 0.010631728 | 0.078458711 | −1.320509765 | 9.686753967 | LRTOMT |
| Fzd7 | ENSMUSG00000041075 | 0.010656151 | 0.078534581 | −1.553523229 | 4.871059402 | FZD7 |
| Gm15800 | ENSMUSG00000042744 | 0.010658149 | 0.078534581 | −1.282111988 | 1435.465857 | HECTD4 |
| Dcdc2b | ENSMUSG00000078552 | 0.010711017 | 0.07878532 | −0.847482737 | 19.73892051 | DCDC2B |
| Vamp1 | ENSMUSG00000030337 | 0.01075619 | 0.078842585 | −1.118476656 | 590.3626967 | VAMP1 |
| Lpp | ENSMUSG00000033306 | 0.010770895 | 0.078842585 | −1.35364225 | 1178.236921 | LPP |
| Adamts10 | ENSMUSG00000024299 | 0.01078637 | 0.078842585 | −0.803103708 | 555.7565365 | ADAMTS10 |
| Arid3b | ENSMUSG00000004661 | 0.010833864 | 0.078976016 | −0.883450121 | 1073.825873 | ARID3B |
| Tnfrsf14 | ENSMUSG00000042333 | 0.010856609 | 0.078976016 | −0.764972942 | 211.1197315 | TNFRSF14 |
| Slc9a5 | ENSMUSG00000014786 | 0.010920563 | 0.078998504 | −0.920059587 | 86.75320744 | SLC9A5 |
| Armcx5 | ENSMUSG00000072969 | 0.010925785 | 0.078998504 | −0.848281897 | 211.1113973 | ARMCX5 |
| Pde7a | ENSMUSG00000069094 | 0.010961749 | 0.079068331 | −0.777034393 | 3063.094707 | PDE7A |
| Atp6v0a1 | ENSMUSG00000019302 | 0.01097445 | 0.079068331 | −0.681684194 | 1341.480343 | ATP6V0A1 |
| Nlrp3 | ENSMUSG00000032691 | 0.011010566 | 0.079162474 | −1.360081646 | 6.66949537 | NLRP3 |
| Parp4 | ENSMUSG00000054509 | 0.011025369 | 0.079162474 | −1.162848635 | 1349.639882 | PARP4 |
| Acvr2a | ENSMUSG00000052155 | 0.011078839 | 0.079429181 | −1.410795042 | 39.41078536 | ACVR2A |
| Klhl15 | ENSMUSG00000043929 | 0.01110063 | 0.079546341 | −0.722058534 | 130.8752231 | KLHL15 |
| H6pd | ENSMUSG00000028980 | 0.011139486 | 0.079629323 | −0.945303747 | 159.2618312 | H6PD |
| Ahnak2 | ENSMUSG00000072812 | 0.011193122 | 0.07977832 | −1.598257354 | 5.196868755 | AHNAK2 |
| Vps13d | ENSMUSG00000020220 | 0.011247414 | 0.079906123 | −1.395797153 | 1714.340931 | VPS13D |
| Pparg | ENSMUSG00000000440 | 0.011266344 | 0.079906123 | −1.268339931 | 23.51793828 | PPARG |
| Rdh5 | ENSMUSG00000025350 | 0.011310794 | 0.079906123 | −0.66050462 | 53.16966775 | RDH5 |
| Elac1 | ENSMUSG00000036941 | 0.011319719 | 0.079906123 | −0.99352373 | 254.0938282 | ELAC1 |
| Snx21 | ENSMUSG00000050373 | 0.01132781 | 0.079906123 | −0.99227817 | 56.81944821 | SNX21 |
| Atrx | ENSMUSG00000031229 | 0.011369959 | 0.080094606 | −1.477889154 | 3213.381127 | ATRX |
| Cflar | ENSMUSG00000026031 | 0.011382236 | 0.080094606 | −0.840014556 | 932.5102315 | CFLAR |
| Fpr2 | ENSMUSG00000052270 | 0.011398353 | 0.080094606 | −1.583510094 | 11.91190566 | FPR2 |
| Gatsl2 | ENSMUSG00000015944 | 0.011413786 | 0.080094606 | −1.011859696 | 45.13997458 | GATSL2 |
| Zfp182 | ENSMUSG00000054737 | 0.011418571 | 0.080094606 | −0.934727361 | 355.9913983 | ZNF182 |
| Vps13b | ENSMUSG00000037646 | 0.01152131 | 0.080416747 | −1.219340813 | 1775.383479 | VPS13B |
| Zfc3h1 | ENSMUSG00000034163 | 0.011529173 | 0.080416747 | −1.153676097 | 1066.170397 | ZFC3H1 |
| Scn11a | ENSMUSG00000034115 | 0.011568523 | 0.080448967 | −1.399713114 | 25.44866353 | SCN11A |
| Slc37a1 | ENSMUSG00000024036 | 0.011589979 | 0.080448967 | −0.4685812 | 419.8661639 | SLC37A1 |
| K1h117 | ENSMUSG00000078484 | 0.011607689 | 0.080452452 | −0.724141288 | 487.3366677 | KLHL17 |
| Mink1 | ENSMUSG00000020827 | 0.011612887 | 0.080452452 | −1.080707027 | 962.9203679 | MINK1 |
| Fam160a2 | ENSMUSG00000044465 | 0.011633901 | 0.080479239 | −0.701047218 | 425.3063312 | FAM160A2 |
| Iqsec1 | ENSMUSG00000034312 | 0.011687417 | 0.080554359 | −1.093656066 | 1786.461153 | IQSEC1 |
| Dcaf17 | ENSMUSG00000041966 | 0.011714786 | 0.080554359 | −0.547531481 | 492.8370775 | DCAF17 |
| Fgfr1op | ENSMUSG00000069135 | 0.011722399 | 0.080561139 | −0.763547185 | 905.6041286 | FGFR1OP |
| Ccdc157 | ENSMUSG00000051427 | 0.011789868 | 0.080777152 | −1.029000742 | 183.4357355 | CCDC157 |
| Clip2 | ENSMUSG00000063146 | 0.011826074 | 0.080777152 | −1.122569614 | 1329.764484 | CLIP2 |
| Itsn1 | ENSMUSG00000022957 | 0.01182741 | 0.080777152 | −0.903866867 | 255.9923569 | ITSN1 |
| Asb1 | ENSMUSG00000026311 | 0.011842372 | 0.080777152 | −0.831421912 | 462.6032187 | ASB1 |
| Mlh3 | ENSMUSG00000021245 | 0.011856197 | 0.080833677 | −1.04460159 | 209.1006906 | MLH3 |
| Zfp799 | ENSMUSG00000059000 | 0.011910005 | 0.080951538 | −0.732542317 | 65.27742718 | ZNF799 |
| Zfp169 | ENSMUSG00000050954 | 0.011926204 | 0.081003752 | −1.294908135 | 167.1144371 | ZNF169 |
| Cbfa2t2 | ENSMUSG00000038533 | 0.011962036 | 0.081070464 | −0.908767566 | 371.3575282 | CBFA2T2 |
| Chd9 | ENSMUSG00000056608 | 0.011994045 | 0.081166932 | −1.100159791 | 1491.218452 | CHD9 |
| Irf2bp2 | ENSMUSG00000051495 | 0.012021745 | 0.081235296 | −0.76236622 | 756.3026508 | IRF2BP2 |
| Cds2 | ENSMUSG00000058793 | 0.012049733 | 0.081235296 | −1.025521462 | 2172.610644 | CDS2 |
| Fgfr1 | ENSMUSG00000031565 | 0.012051033 | 0.081235296 | −1.376954383 | 6.561949327 | FGFR1 |
| Kdm6b | ENSMUSG00000018476 | 0.012147736 | 0.081414373 | −1.225070515 | 476.2268269 | KDM6B |
| Eml6 | ENSMUSG00000044072 | 0.012185013 | 0.081574279 | −0.935140648 | 158.0163731 | EML6 |
| Zfp652 | ENSMUSG00000075595 | 0.012191332 | 0.081574279 | −1.149662359 | 838.1985096 | ZNF652 |
| Ern1 | ENSMUSG00000020715 | 0.012193949 | 0.081574279 | −1.144574032 | 333.6826332 | ERN1 |
| Pikfyve | ENSMUSG00000025949 | 0.012204559 | 0.081601543 | −1.234326087 | 653.0891598 | PIKFYVE |
| Arap2 | ENSMUSG00000037999 | 0.012237526 | 0.081613544 | −1.326495466 | 850.5387718 | ARAP2 |
| Notch2 | ENSMUSG00000027878 | 0.012261467 | 0.081613544 | −1.347836903 | 2849.787421 | NOTCH2 |
| Pilra | ENSMUSG00000046245 | 0.012305428 | 0.081677278 | −1.264267285 | 54.74976696 | PILRA |
| Ypel1 | ENSMUSG00000022773 | 0.012380591 | 0.081884681 | −1.274817096 | 20.16524856 | YPEL1 |
| Zfp619 | ENSMUSG00000068959 | 0.012408281 | 0.081997141 | −1.16471656 | 105.1300748 | ZNF208 |
| Mapkbp1 | ENSMUSG00000033902 | 0.012434665 | 0.082041416 | −1.318094576 | 171.109109 | MAPKBP1 |
| 2310035C23Rik | ENSMUSG00000026319 | 0.012438058 | 0.082041416 | −1.048083287 | 948.2303659 | KIAA1468 |
| Fam175a | ENSMUSG00000035234 | 0.012458381 | 0.082101576 | −0.322958493 | 357.1688676 | FAM175A |
| Rreb1 | ENSMUSG00000039087 | 0.012473416 | 0.082106217 | −1.258615152 | 566.2203327 | RREB1 |
| Vps13c | ENSMUSG00000035284 | 0.012502494 | 0.082132721 | −1.449884329 | 636.8272029 | VPS13C |
| Dicer1 | ENSMUSG00000041415 | 0.012563814 | 0.082248674 | −1.179366227 | 1209.609337 | DICER1 |
| Ypel2 | ENSMUSG00000018427 | 0.012566472 | 0.082248674 | −0.949394041 | 90.30261257 | YPEL2 |
| Glb1l | ENSMUSG00000026200 | 0.012600324 | 0.082248674 | −0.594557335 | 103.5500513 | GLB1L |
| Foxj1 | ENSMUSG00000034227 | 0.012604363 | 0.082248674 | −1.193647 | 7.897467497 | FOXJ1 |
| Mybpc1 | ENSMUSG00000020061 | 0.012640075 | 0.082270966 | −1.105863237 | 23.95869301 | MYBPC1 |
| Ccr5 | ENSMUSG00000079227 | 0.01265449 | 0.082279553 | −0.838907149 | 45.72863491 | CCR5 |
| Abca5 | ENSMUSG00000018800 | 0.012711996 | 0.08247724 | −1.137525942 | 54.20326943 | ABCA5 |
| Rpl5 | ENSMUSG00000058558 | 0.01273057 | 0.082477951 | −0.719350178 | 185.1102909 | RPL5 |
| Phlpp2 | ENSMUSG00000031732 | 0.012742223 | 0.082477951 | −1.296775998 | 529.3274256 | PHLPP2 |
| Gpr137c | ENSMUSG00000049092 | 0.012747347 | 0.082477951 | −0.968774283 | 32.38544774 | GPR137C |
| Agbl3 | ENSMUSG00000038836 | 0.01281255 | 0.082552489 | −0.712128 | 57.57861492 | AGBL3 |
| Rnft2 | ENSMUSG00000032850 | 0.012878863 | 0.082639568 | −1.138329132 | 13.45292751 | RNFT2 |
| Chd7 | ENSMUSG00000041235 | 0.012879702 | 0.082639568 | −1.344358345 | 1174.750646 | CHD7 |
| Snx30 | ENSMUSG00000028385 | 0.012915274 | 0.082679318 | −1.254863809 | 2511.189704 | SNX30 |
| Cry2 | ENSMUSG00000068742 | 0.012933438 | 0.082693557 | −0.780392708 | 237.0308156 | CRY2 |
| Bcl11a | ENSMUSG00000000861 | 0.013059492 | 0.08313169 | −0.846871521 | 2381.390264 | BCL11A |
| Zfp266 | ENSMUSG00000060510 | 0.013064256 | 0.08313169 | −0.836201437 | 421.7016832 | ZNF266 |
| Setd2 | ENSMUSG00000044791 | 0.013076919 | 0.083153903 | −1.233858658 | 3109.883982 | SETD2 |
| Golga4 | ENSMUSG00000038708 | 0.013085396 | 0.08315521 | −1.410611746 | 946.3615855 | GOLGA4 |
| Pkd1 | ENSMUSG00000032855 | 0.013107031 | 0.083256453 | −1.25706393 | 1450.558608 | PKD1 |
| Tom1l2 | ENSMUSG00000000538 | 0.013160293 | 0.083473397 | −0.872553987 | 354.9400208 | TOM1L2 |
| Olfr164 | ENSMUSG00000050742 | 0.013169777 | 0.083473397 | −1.769577951 | 4.603460573 | OR2M3 |
| Ylpm1 | ENSMUSG00000021244 | 0.013238012 | 0.083666662 | −1.269094094 | 1085.088728 | YLPM1 |
| Rnf144b | ENSMUSG00000038068 | 0.01324156 | 0.083666662 | −1.409513394 | 16.36125811 | RNF144B |
| Pygl | ENSMUSG00000021069 | 0.01324386 | 0.083666662 | −1.263482657 | 30.1429258 | PYGL |
| Zbtb10 | ENSMUSG00000069114 | 0.013246181 | 0.083666662 | −1.307274071 | 163.1503619 | ZBTB10 |
| Tnni3 | ENSMUSG00000035458 | 0.013262794 | 0.083668691 | −1.494042954 | 9.45094598 | TNNI3 |
| Slc16a6 | ENSMUSG00000041920 | 0.01328088 | 0.083717371 | −1.006810443 | 392.8612392 | SLC16A6 |
| Trim39 | ENSMUSG00000045409 | 0.013354128 | 0.083895144 | −1.074887341 | 1079.819369 | TRIM39 |
| Plekhg3 | ENSMUSG00000052609 | 0.01338217 | 0.083903509 | −1.296103299 | 162.5257675 | PLEKHG3 |
| Ccdc93 | ENSMUSG00000026339 | 0.013414866 | 0.083933475 | −0.965654671 | 690.6223703 | CCDC93 |
| Srsf6 | ENSMUSG00000016921 | 0.013446932 | 0.083987327 | −0.54523613 | 4162.449902 | SRSF6 |
| Srms | ENSMUSG00000027579 | 0.013467864 | 0.084043482 | −0.728866239 | 107.6238678 | SRMS |
| Ttll4 | ENSMUSG00000033257 | 0.013496395 | 0.084072805 | −1.124863854 | 699.3602601 | TTLL4 |
| Stx16 | ENSMUSG00000027522 | 0.013507237 | 0.084072805 | −1.128009415 | 1169.25115 | STX16 |
| Nnat | ENSMUSG00000067786 | 0.013531746 | 0.084072805 | −1.725508992 | 5.25931015 | NNAT |
| Fcamr | ENSMUSG00000026415 | 0.01355892 | 0.084072805 | −1.362688019 | 54.51808086 | FCAMR |
| Lrrk2 | ENSMUSG00000036273 | 0.013590515 | 0.084072805 | −1.296594474 | 3758.29599 | LRRK2 |
| Pik3r5 | ENSMUSG00000020901 | 0.013594177 | 0.084072805 | −0.891695424 | 285.3127537 | PIK3R5 |
| Chst10 | ENSMUSG00000026080 | 0.01360608 | 0.084072805 | −0.75860699 | 352.0285415 | CHST10 |
| Mest | ENSMUSG00000051855 | 0.013606933 | 0.084072805 | −1.445806419 | 7.063098048 | MEST |
| Sec31b | ENSMUSG00000051984 | 0.013611219 | 0.084072805 | −0.845463405 | 52.73258518 | SEC31B |
| Ppm1h | ENSMUSG00000034613 | 0.013615582 | 0.084072805 | −0.908904339 | 90.97169596 | PPM1H |
| Zfp319 | ENSMUSG00000074140 | 0.013773578 | 0.084511524 | −0.90977173 | 91.47128561 | ZNF319 |
| Rapgef6 | ENSMUSG00000037533 | 0.013813357 | 0.084622765 | −1.264839477 | 2122.483734 | RAPGEF6 |
| Ift172 | ENSMUSG00000038564 | 0.013839401 | 0.084622765 | −0.906813775 | 494.1281579 | IFT172 |
| Vwa3b | ENSMUSG00000026115 | 0.013850167 | 0.084622765 | −0.895667969 | 42.76212315 | VWA3B |
| Cacna1e | ENSMUSG00000004110 | 0.013858321 | 0.084622765 | −1.556859069 | 1292.314025 | CACNA1E |
| Aff1 | ENSMUSG00000029313 | 0.013875677 | 0.084622765 | −1.302883707 | 1175.504973 | AFF1 |
| Ermap | ENSMUSG00000028644 | 0.013892969 | 0.084622765 | −1.450463901 | 46.88382186 | ERMAP |
| Tmem184c | ENSMUSG00000031617 | 0.013930664 | 0.084622765 | −0.525068253 | 989.7359675 | TMEM184C |
| Rhbdf1 | ENSMUSG00000020282 | 0.013938767 | 0.084622765 | −0.516294435 | 838.4283363 | RHBDF1 |
| Soat2 | ENSMUSG00000023045 | 0.013943717 | 0.084622765 | −0.737847006 | 100.0823366 | SOAT2 |
| Gng7 | ENSMUSG00000048240 | 0.01395281 | 0.084622765 | −1.175175213 | 9.126091088 | GNG7 |
| Lmbrd2 | ENSMUSG00000039704 | 0.013971423 | 0.084622765 | −1.334315393 | 161.8316829 | LMBRD2 |
| Lpin1 | ENSMUSG00000020593 | 0.014021343 | 0.08479343 | −0.897112933 | 158.3380693 | LPIN1 |
| Slfn4 | ENSMUSG00000000204 | 0.014055661 | 0.084909809 | −1.37454427 | 8.686481829 | SLFN12L |
| Ralgapa1 | ENSMUSG00000021027 | 0.014059533 | 0.084909809 | −1.035979226 | 1359.770867 | RALGAPA1 |
| Hbs1l | ENSMUSG00000019977 | 0.014137838 | 0.085171286 | −0.383636946 | 594.4111461 | HBS1L |
| Ubr4 | ENSMUSG00000066036 | 0.014165681 | 0.08529108 | −1.367407789 | 4783.041973 | UBR4 |
| Plcg1 | ENSMUSG00000016933 | 0.014169409 | 0.08529108 | −1.05144565 | 716.5135272 | PLCG1 |
| Hivep3 | ENSMUSG00000028634 | 0.014221665 | 0.085500479 | −1.366544196 | 437.8539865 | HIVEP3 |
| Fbxo33 | ENSMUSG00000035329 | 0.014226749 | 0.085500479 | −0.922857492 | 595.0925604 | FBXO33 |
| Atp7a | ENSMUSG00000033792 | 0.014235662 | 0.085500479 | −1.338673753 | 139.417406 | ATP7A |
| Gm5595 | ENSMUSG00000069727 | 0.0142382 | 0.085500479 | −0.773170744 | 25.05705696 | ZNF14 |
| Phyhd1 | ENSMUSG00000079484 | 0.014249014 | 0.085515489 | −1.22606884 | 8.545978464 | PHYHD1 |
| Pdzd3 | ENSMUSG00000032105 | 0.014256958 | 0.085515489 | −1.096249586 | 18.00905285 | PDZD3 |
| Hpgd | ENSMUSG00000031613 | 0.014263393 | 0.085515489 | −1.168539353 | 58.67471523 | HPGD |
| Camsap1 | ENSMUSG00000026933 | 0.01426552 | 0.085515489 | −1.034453308 | 295.3556777 | CAMSAP1 |
| Clk4 | ENSMUSG00000020385 | 0.014292701 | 0.085577969 | −0.746533133 | 2293.58935 | CLK4 |
| Zrsr2 | ENSMUSG00000031370 | 0.014333022 | 0.085587696 | −0.795569435 | 613.161291 | ZRSR2 |
| Tiam1 | ENSMUSG00000002489 | 0.014335062 | 0.085587696 | −1.161710618 | 18.69859792 | TIAM1 |
| Arsg | ENSMUSG00000020604 | 0.014362218 | 0.085587696 | −0.611249494 | 66.61526984 | ARSG |
| Col4a1 | ENSMUSG00000031502 | 0.014362343 | 0.085587696 | −1.736436393 | 4.319012166 | COL4A1 |
| Htt | ENSMUSG00000029104 | 0.014447773 | 0.085712485 | −1.182975753 | 1374.807682 | HTT |
| Nbeal1 | ENSMUSG00000073664 | 0.014576816 | 0.086106778 | −1.42251323 | 516.0124635 | NBEAL1 |
| Dntt | ENSMUSG00000025014 | 0.014586083 | 0.086106778 | −1.485311762 | 10.27976592 | DNTT |
| Mysm1 | ENSMUSG00000062627 | 0.014625077 | 0.086118908 | −1.309020275 | 899.5386502 | MYSM1 |
| Ier2 | ENSMUSG00000053560 | 0.014631424 | 0.086118908 | −1.061424662 | 1466.65366 | IER2 |
| Thsd7a | ENSMUSG00000032625 | 0.014651373 | 0.086187473 | −1.51658018 | 130.2103467 | THSD7A |
| Gabpb2 | ENSMUSG00000038766 | 0.014654882 | 0.086187473 | −1.368904628 | 2568.323307 | GABPB2 |
| Cx3cr1 | ENSMUSG00000052336 | 0.014748951 | 0.086577436 | −1.234893752 | 24.71223945 | CX3CR1 |
| Hmbox1 | ENSMUSG00000021972 | 0.014790464 | 0.086590072 | −1.100806882 | 411.2616329 | HMBOX1 |
| Tmem116 | ENSMUSG00000029452 | 0.014794522 | 0.086590072 | −0.829693167 | 27.65454245 | TMEM116 |
| Nktr | ENSMUSG00000032525 | 0.014816785 | 0.086685615 | −1.276541199 | 2126.128977 | NKTR |
| Son | ENSMUSG00000022961 | 0.014939539 | 0.086916083 | −1.250812194 | 10173.49236 | SON |
| Luc7l2 | ENSMUSG00000029823 | 0.01501031 | 0.087035524 | −1.216686276 | 3120.446062 | LUC7L2 |
| Mga | ENSMUSG00000033943 | 0.015021126 | 0.087035524 | −1.422140515 | 1856.862273 | MGA |
| Mtf1 | ENSMUSG00000028890 | 0.015051469 | 0.087035524 | −0.614696943 | 565.2191351 | MTF1 |
| Osgin1 | ENSMUSG00000074063 | 0.015074657 | 0.087035524 | −0.934577205 | 35.34599529 | OSGIN1 |
| Prrc2b | ENSMUSG00000039262 | 0.015085901 | 0.087035524 | −1.315815004 | 3211.520645 | PRRC2B |
| Atf7ip | ENSMUSG00000030213 | 0.015094996 | 0.087035524 | −1.157296426 | 1295.961609 | ATF7IP |
| Tubb1 | ENSMUSG00000016255 | 0.015121462 | 0.087043584 | −1.469723141 | 4.437466774 | TUBB1 |
| Bambi | ENSMUSG00000024232 | 0.015157437 | 0.087057911 | −1.148265183 | 26.42514752 | BAMBI |
| Bmpr2 | ENSMUSG00000067336 | 0.015172738 | 0.087057911 | −1.334507897 | 7.503170042 | BMPR2 |
| Gpr126 | ENSMUSG00000039116 | 0.015173335 | 0.087057911 | −1.803152709 | 4.140667144 | GPR126 |
| Pofut1 | ENSMUSG00000046020 | 0.01517908 | 0.087057911 | −0.733564734 | 681.5215003 | POFUT1 |
| Camk1 | ENSMUSG00000030272 | 0.015187628 | 0.087057911 | −0.884412759 | 33.3091197 | CAMK1 |
| Zscan18 | ENSMUSG00000070822 | 0.015190072 | 0.087057911 | −0.936068641 | 41.92761479 | ZSCAN18 |
| Cpt1b | ENSMUSG00000078937 | 0.015296491 | 0.087236616 | −1.388248338 | 4.656045908 | CPT1B |
| Clec4a1 | ENSMUSG00000049037 | 0.015321287 | 0.087284863 | −1.260313227 | 43.50034413 | ZNF705A |
| Ankzf1 | ENSMUSG00000026199 | 0.015360904 | 0.087284863 | −0.627372186 | 644.9610673 | ANKZF1 |
| Tbc1d9 | ENSMUSG00000031709 | 0.015366445 | 0.087284863 | −1.229353144 | 120.5297296 | TBC1D9 |
| Akap13 | ENSMUSG00000066406 | 0.015446813 | 0.087284863 | −1.36765445 | 6065.538078 | AKAP13 |
| Large | ENSMUSG00000004383 | 0.015469655 | 0.087284863 | −1.718597325 | 4.55211812 | LARGE |
| Zfp451 | ENSMUSG00000042197 | 0.015500985 | 0.087284863 | −1.071814361 | 670.2461643 | ZNF451 |
| Snrnp48 | ENSMUSG00000021431 | 0.015501114 | 0.087284863 | −0.461869294 | 620.0762651 | SNRNP48 |
| Ccnt2 | ENSMUSG00000026349 | 0.015517392 | 0.087284863 | −1.128650326 | 1592.840299 | CCNT2 |
| Per3 | ENSMUSG00000028957 | 0.015537494 | 0.087284863 | −1.230573666 | 84.97264151 | PER3 |
| Zc3h12a | ENSMUSG00000042677 | 0.015622475 | 0.087554701 | −0.746839415 | 635.7939315 | ZC3H12A |
| Slc26a2 | ENSMUSG00000034320 | 0.015638796 | 0.087554701 | −1.165021166 | 396.2704163 | SLC26A2 |
| Bdp1 | ENSMUSG00000049658 | 0.015639697 | 0.087554701 | −1.40316165 | 977.7803975 | BDP1 |
| Nap1l5 | ENSMUSG00000055430 | 0.015643123 | 0.087554701 | −1.59336728 | 4.272282326 | NAP1L5 |
| Prpf40b | ENSMUSG00000023007 | 0.015662169 | 0.087611499 | −0.863146261 | 181.1449725 | PRPF40B |
| BC049715 | ENSMUSG00000047515 | 0.015665274 | 0.087611499 | −0.969711821 | 14.44523504 | C12orf60 |
| Hlcs | ENSMUSG00000040820 | 0.015685998 | 0.087660225 | −0.599258484 | 657.5224388 | HLCS |
| Phf20l1 | ENSMUSG00000072501 | 0.015806079 | 0.087879521 | −1.309698817 | 825.0755001 | PHF20L1 |
| Inpp4a | ENSMUSG00000026113 | 0.015954269 | 0.088257431 | −1.137529888 | 422.0426148 | INPP4A |
| Gspt2 | ENSMUSG00000071723 | 0.016016795 | 0.088395311 | −1.130172901 | 8.448835997 | GSPT2 |
| Lnpep | ENSMUSG00000023845 | 0.016056573 | 0.088395311 | −1.423623155 | 2012.995619 | LNPEP |
| Snca | ENSMUSG00000025889 | 0.016066477 | 0.088395311 | −1.653914076 | 37.47042992 | SNCA |
| BC030499 | ENSMUSG00000037593 | 0.016066595 | 0.088395311 | −1.318292994 | 6.051871419 | SGK494 |
| Adap2 | ENSMUSG00000020709 | 0.016077509 | 0.088395311 | −1.448725563 | 17.46968355 | ADAP2 |
| Trim40 | ENSMUSG00000073399 | 0.016091193 | 0.088395311 | −1.023776522 | 12.88183169 | TRIM40 |
| Sesn3 | ENSMUSG00000032009 | 0.016091853 | 0.088395311 | −0.784184409 | 1054.326844 | SESN3 |
| Cebpa | ENSMUSG00000034957 | 0.016140471 | 0.088456158 | −0.981001607 | 26.20247198 | CEBPA |
| Wdr11 | ENSMUSG00000042055 | 0.016149472 | 0.088456158 | −0.887442614 | 816.802302 | WDR11 |
| Usp48 | ENSMUSG00000043411 | 0.016225097 | 0.08863688 | −1.077064654 | 1800.697166 | USP48 |
| Znf512b | ENSMUSG00000000823 | 0.016235577 | 0.088658351 | −1.183329772 | 740.0764865 | ZNF512B |
| Disc1 | ENSMUSG00000043051 | 0.016278169 | 0.088787979 | −1.195411885 | 13.24347935 | DISC1 |
| Rai1 | ENSMUSG00000062115 | 0.01629877 | 0.08883986 | −1.003850007 | 559.4775749 | RAI1 |
| Crtc1 | ENSMUSG00000003575 | 0.016331268 | 0.08898377 | −0.657622848 | 378.3122648 | CRTC1 |
| Rgs2 | ENSMUSG00000026360 | 0.016353982 | 0.089007859 | −0.615116525 | 3983.63183 | RGS2 |
| Ino80 | ENSMUSG00000034154 | 0.016449247 | 0.089260097 | −1.068757463 | 1052.95832 | INO80 |
| Ndor1 | ENSMUSG00000006471 | 0.016465402 | 0.089276715 | −0.63707781 | 1204.967915 | NDOR1 |
| Rnf157 | ENSMUSG00000052949 | 0.016476324 | 0.089276715 | −0.417063462 | 1203.793529 | RNF157 |
| Ercc6 | ENSMUSG00000054051 | 0.016488115 | 0.089276715 | −1.248311611 | 630.521908 | ERCC6 |
| Zfp445 | ENSMUSG00000047036 | 0.016497604 | 0.089276715 | −1.374387942 | 794.9400438 | ZNF445 |
| Herc6 | ENSMUSG00000029798 | 0.01651996 | 0.089278731 | −0.479168344 | 499.7955735 | HERC6 |
| Kif21b | ENSMUSG00000041642 | 0.016550411 | 0.089335086 | −1.313960897 | 3951.091126 | KIF21B |
| Plcb2 | ENSMUSG00000040061 | 0.016741841 | 0.089978148 | −1.166546653 | 799.7453429 | PLCB2 |
| Mafb | ENSMUSG00000074622 | 0.016817768 | 0.090238122 | −1.232904092 | 78.41605515 | MAFB |
| Tet3 | ENSMUSG00000034832 | 0.016843747 | 0.090244986 | −1.412408945 | 950.5493543 | TET3 |
| Dak | ENSMUSG00000034371 | 0.016849619 | 0.090244986 | −0.54048695 | 234.7445952 | DAK |
| B3gnt5 | ENSMUSG00000022686 | 0.016860797 | 0.090244986 | −0.893470582 | 3041.540574 | B3GNT5 |
| Gpd1 | ENSMUSG00000023019 | 0.016864678 | 0.090244986 | −1.123377461 | 12.24390341 | GPD1 |
| Ipcef1 | ENSMUSG00000064065 | 0.01693754 | 0.090563104 | −1.197339847 | 223.1098492 | IPCEF1 |
| Cdk12 | ENSMUSG00000003119 | 0.016948738 | 0.090583774 | −1.114775744 | 1261.43648 | CDK12 |
| Fam46a | ENSMUSG00000032265 | 0.017075474 | 0.090867918 | −1.241760066 | 178.0250189 | FAM46A |
| Cep63 | ENSMUSG00000032534 | 0.01709408 | 0.09089013 | −0.643345706 | 721.3101443 | CEP63 |
| Brwd3 | ENSMUSG00000063663 | 0.017104058 | 0.09089013 | −1.288878033 | 414.7772581 | BRWD3 |
| Dnajc27 | ENSMUSG00000020657 | 0.017132721 | 0.09089013 | −0.916556136 | 365.4529266 | DNAJC27 |
| Megf11 | ENSMUSG00000036466 | 0.017144438 | 0.090903508 | −1.397054429 | 5.992120988 | MEGF11 |
| Sidt2 | ENSMUSG00000034908 | 0.01717632 | 0.090973418 | −0.30389852 | 3650.980474 | SIDT2 |
| Bptf | ENSMUSG00000040481 | 0.017219244 | 0.091068588 | −1.255484699 | 5645.938968 | BPTF |
| Scml4 | ENSMUSG00000044770 | 0.017282247 | 0.091147173 | −1.058845613 | 558.2429221 | SCML4 |
| Dync1h1 | ENSMUSG00000018707 | 0.017314966 | 0.091244245 | −1.31189975 | 4297.71315 | DYNC1H1 |
| Grap2 | ENSMUSG00000042351 | 0.017337264 | 0.091326307 | −0.966980345 | 688.6004098 | GRAP2 |
| Gpr68 | ENSMUSG00000047415 | 0.017346335 | 0.091326307 | −0.827817603 | 14.37827547 | GPR68 |
| Safb2 | ENSMUSG00000042625 | 0.01736713 | 0.091326307 | −1.081222905 | 2419.542369 | SAFB2 |
| Ubxn7 | ENSMUSG00000053774 | 0.017377945 | 0.091337443 | −1.161631386 | 1066.259893 | UBXN7 |
| Chd6 | ENSMUSG00000057133 | 0.017400551 | 0.091337443 | −1.275910886 | 1739.67433 | CHD6 |
| Unc13b | ENSMUSG00000028456 | 0.017403684 | 0.091337443 | −1.123222103 | 20.84620494 | UNC13B |
| Ccdc77 | ENSMUSG00000030177 | 0.017441783 | 0.091450324 | −0.711506154 | 294.3748403 | CCDC77 |
| Tub | ENSMUSG00000031028 | 0.01748396 | 0.091539944 | −1.462061832 | 31.63584374 | TUB |
| Cstf3 | ENSMUSG00000027176 | 0.017507937 | 0.091569224 | −0.331073953 | 737.3833886 | CSTF3 |
| Clk2 | ENSMUSG00000068917 | 0.017577028 | 0.091569224 | −0.5001039 | 1412.030818 | CLK2 |
| Ccnl2 | ENSMUSG00000029068 | 0.017584162 | 0.091569224 | −0.780939107 | 3762.265218 | CCNL2 |
| Dip2b | ENSMUSG00000023026 | 0.01763293 | 0.091655615 | −1.133991122 | 1036.281289 | DIP2B |
| Tbl1xr1 | ENSMUSG00000027630 | 0.017654385 | 0.091688755 | −0.965538897 | 1524.808036 | TBL1XR1 |
| Jup | ENSMUSG00000001552 | 0.017742308 | 0.09180532 | −0.986127033 | 49.20842536 | JUP |
| Asxl1 | ENSMUSG00000042548 | 0.017747737 | 0.09180532 | −1.040607547 | 1579.330115 | ASXL1 |
| Drp2 | ENSMUSG00000000223 | 0.017792355 | 0.091869475 | −0.983140086 | 8.632973327 | DRP2 |
| Mgat5 | ENSMUSG00000036155 | 0.017882428 | 0.092106492 | −1.144317333 | 152.6544653 | MGAT5 |
| Cdo1 | ENSMUSG00000033022 | 0.017923746 | 0.092230527 | −0.930566557 | 11.92817261 | CDO1 |
| Nup210l | ENSMUSG00000027939 | 0.01795143 | 0.092230527 | −1.064090562 | 41.08688347 | NUP210L |
| Nsun6 | ENSMUSG00000026707 | 0.017983684 | 0.092230527 | −0.525898694 | 208.924779 | NSUN6 |
| Acsl1 | ENSMUSG00000018796 | 0.017991026 | 0.092230527 | −0.755538669 | 337.0473674 | ACSL1 |
| Epha2 | ENSMUSG00000006445 | 0.018014672 | 0.092230527 | −1.430145009 | 8.540709877 | EPHA2 |
| Ddi2 | ENSMUSG00000078515 | 0.018021228 | 0.092230527 | −1.154007887 | 920.2798774 | DDI2 |
| Hoxb6 | ENSMUSG00000000690 | 0.01803016 | 0.092230527 | −1.408633271 | 4.529366864 | HOXB6 |
| Fbxo48 | ENSMUSG00000044966 | 0.018033016 | 0.092230527 | −0.740128736 | 46.60243776 | FBXO48 |
| Cdon | ENSMUSG00000038119 | 0.018037027 | 0.092230527 | −0.882313552 | 240.5108328 | CDON |
| Cd274 | ENSMUSG00000016496 | 0.018056972 | 0.092230527 | −1.07871155 | 562.3129892 | CD274 |
| Uevld | ENSMUSG00000043262 | 0.018087926 | 0.092230527 | −0.817079183 | 66.60563383 | UEVLD |
| Zfp612 | ENSMUSG00000044676 | 0.018090738 | 0.092230527 | −1.152059327 | 9.674349939 | ZNF23 |
| Klhl28 | ENSMUSG00000020948 | 0.018123377 | 0.09224091 | −0.91884418 | 288.5210522 | KLHL28 |
| Fam89a | ENSMUSG00000043068 | 0.018168698 | 0.092310644 | −0.798230277 | 26.10340703 | FAM89A |
| Zbtb49 | ENSMUSG00000029127 | 0.018232097 | 0.092568319 | −0.476637852 | 284.1677482 | ZBTB49 |
| Ttbk2 | ENSMUSG00000090100 | 0.018295275 | 0.092695635 | −1.391336151 | 87.55931245 | TTBK2 |
| Tgfbi | ENSMUSG00000035493 | 0.018379081 | 0.092830343 | −1.072885574 | 115.2375347 | TGFBI |
| Zfp26 | ENSMUSG00000063108 | 0.018421429 | 0.092915547 | −1.084262276 | 560.8614655 | ZNF778 |
| Lime1 | ENSMUSG00000090077 | 0.018570164 | 0.09343974 | −1.225126372 | 12.31650091 | LIME1 |
| Myo1f | ENSMUSG00000024300 | 0.018662534 | 0.093807512 | −0.882847964 | 458.6474595 | MYO1F |
| Prrc2c | ENSMUSG00000040225 | 0.018715482 | 0.093894237 | −1.47120844 | 3170.162399 | PRRC2C |
| Wdr60 | ENSMUSG00000042050 | 0.018729727 | 0.093894237 | −0.906283249 | 14.95670664 | WDR60 |
| Btbd8 | ENSMUSG00000070632 | 0.018749861 | 0.093933419 | −0.870852395 | 17.29502599 | BTBD8 |
| Ddx19b | ENSMUSG00000033658 | 0.018764804 | 0.093933419 | −0.836449429 | 484.5248653 | DDX19B |
| Gm13139 | ENSMUSG00000067916 | 0.018790437 | 0.094029487 | −1.086064455 | 10.94732811 | ZNF616 |
| Spen | ENSMUSG00000040761 | 0.018829033 | 0.094096986 | −1.381698329 | 1027.433588 | SPEN |
| Col11a2 | ENSMUSG00000024330 | 0.018829367 | 0.094096986 | −0.834142813 | 190.2070782 | COL11A2 |
| Zfp628 | ENSMUSG00000074406 | 0.01883451 | 0.094096986 | −0.542042243 | 397.8378315 | ZNF628 |
| Tas1r3 | ENSMUSG00000029072 | 0.018884467 | 0.094096986 | −0.568213464 | 51.20226003 | TAS1R3 |
| Cep350 | ENSMUSG00000033671 | 0.018926406 | 0.094096986 | −1.377044351 | 1466.110192 | CEP350 |
| Plk3 | ENSMUSG00000028680 | 0.018987117 | 0.094198506 | −0.70968689 | 229.0856183 | PLK3 |
| Serac1 | ENSMUSG00000015659 | 0.019011358 | 0.094198506 | −1.053993423 | 73.33354374 | SERAC1 |
| Hap1 | ENSMUSG00000006930 | 0.019065758 | 0.094403443 | −0.996510611 | 69.59838136 | HAP1 |
| Ccdc9 | ENSMUSG00000041375 | 0.01908246 | 0.094403443 | −0.702718121 | 1162.527853 | CCDC9 |
| Acot11 | ENSMUSG00000034853 | 0.019145482 | 0.094542043 | −1.135314984 | 30.51018249 | ACOT11 |
| Myo9a | ENSMUSG00000039585 | 0.019250347 | 0.094664867 | −1.088410817 | 303.0822416 | MYO9A |
| Aff4 | ENSMUSG00000049470 | 0.019289776 | 0.094741879 | −1.258925338 | 3470.402189 | AFF4 |
| Gpm6a | ENSMUSG00000031517 | 0.019373053 | 0.094801018 | −1.162763364 | 247.9406564 | GPM6A |
| Zmiz1 | ENSMUSG00000007817 | 0.019376935 | 0.094801018 | −1.3866755 | 931.2942481 | ZMIZ1 |
| Dnaic2 | ENSMUSG00000034706 | 0.019413302 | 0.094832313 | −0.986996817 | 13.02632144 | DNAI2 |
| Eme2 | ENSMUSG00000073436 | 0.019415967 | 0.094832313 | −0.729757875 | 282.211322 | EME2 |
| Rgp1 | ENSMUSG00000028468 | 0.019460355 | 0.094941037 | −1.028395859 | 844.9241291 | RGP1 |
| Zkscan1 | ENSMUSG00000029729 | 0.019481088 | 0.094978437 | −1.085946236 | 335.651014 | ZKSCAN1 |
| Ncor2 | ENSMUSG00000029478 | 0.019530941 | 0.095189696 | −1.184467949 | 1277.810028 | NCOR2 |
| Ranbp10 | ENSMUSG00000037415 | 0.019629871 | 0.095462941 | −0.869754355 | 767.6113446 | RANBP10 |
| Nin | ENSMUSG00000021068 | 0.019648724 | 0.095462941 | −1.308382086 | 1346.623763 | NIN |
| Pdp2 | ENSMUSG00000048371 | 0.019723627 | 0.095703519 | −0.849250329 | 140.40094 | PDP2 |
| Adam19 | ENSMUSG00000011256 | 0.01976855 | 0.095715263 | −1.126960649 | 634.7672398 | ADAM19 |
| Rnf214 | ENSMUSG00000042790 | 0.019811125 | 0.095715263 | −0.84177797 | 309.0177764 | RNF214 |
| Lpgat1 | ENSMUSG00000026623 | 0.01981555 | 0.095715263 | −0.879803263 | 2137.753401 | LPGAT1 |
| Acap3 | ENSMUSG00000029033 | 0.019821331 | 0.095715263 | −0.625280884 | 392.831564 | ACAP3 |
| Ppip5k1 | ENSMUSG00000033526 | 0.019822628 | 0.095715263 | −0.668430693 | 365.8628434 | PPIP5K1 |
| Zyg11b | ENSMUSG00000034636 | 0.019826354 | 0.095715263 | −1.139551873 | 915.9211197 | ZYG11B |
| Atad2b | ENSMUSG00000052812 | 0.019844406 | 0.095715263 | −1.330003003 | 552.766295 | ATAD2B |
| Mical3 | ENSMUSG00000051586 | 0.019965345 | 0.095846214 | −0.992577279 | 224.4029859 | MICAL3 |
| Clec4b1 | ENSMUSG00000030147 | 0.019965902 | 0.095846214 | −1.533481839 | 4.42757991 | CLEC4C |
| Degs2 | ENSMUSG00000021263 | 0.020009357 | 0.096014656 | −1.045449322 | 249.6306372 | DEGS2 |
| Hif3a | ENSMUSG00000004328 | 0.020027935 | 0.096040658 | −1.062633445 | 8.487928197 | HIF3A |
| Gp9 | ENSMUSG00000030054 | 0.020049221 | 0.09611116 | −1.433603363 | 3.975682944 | GP9 |
| Med13 | ENSMUSG00000034297 | 0.020097096 | 0.096182726 | −1.253932367 | 3615.226429 | MED13 |
| Fcgr4 | ENSMUSG00000059089 | 0.020143715 | 0.096255477 | −1.160522854 | 84.67335768 | FCGR3A |
| Fcgr4 | ENSMUSG00000059089 | 0.020143715 | 0.096255477 | −1.160522854 | 84.67335768 | FCGR3B |
| Shisa3 | ENSMUSG00000050010 | 0.020148657 | 0.096255477 | −1.354883089 | 7.396057646 | SHISA3 |
| Card14 | ENSMUSG00000013483 | 0.020151863 | 0.096255477 | −1.488784593 | 4.134855448 | CARD14 |
| Pgam2 | ENSMUSG00000020475 | 0.020208691 | 0.096453838 | −0.874254092 | 42.29415437 | PGAM2 |
| Serping1 | ENSMUSG00000023224 | 0.020257234 | 0.096600732 | −1.46851951 | 4.460761253 | SERPING1 |
| Nipa1 | ENSMUSG00000047037 | 0.020305754 | 0.096733808 | −1.355631035 | 6.049910928 | NIPA1 |
| Acrbp | ENSMUSG00000072770 | 0.020379924 | 0.096995679 | −0.665882973 | 127.5221364 | ACRBP |
| Usp34 | ENSMUSG00000056342 | 0.020394315 | 0.097000917 | −1.245592918 | 2387.682289 | USP34 |
| Rictor | ENSMUSG00000050310 | 0.020473487 | 0.097282386 | −1.340904671 | 1042.8486 | RICTOR |
| Atxn1l | ENSMUSG00000069895 | 0.020532234 | 0.097316188 | −1.167706306 | 824.1639304 | ATXN1L |
| Kcnh7 | ENSMUSG00000059742 | 0.020552572 | 0.097338954 | −1.421541182 | 20.19665951 | KCNH7 |
| Tmem194b | ENSMUSG00000043015 | 0.020588529 | 0.097384643 | −0.702580895 | 1251.883956 | TMEM194B |
| Atxn7 | ENSMUSG00000021738 | 0.02065255 | 0.097401325 | −1.255286468 | 773.0175735 | ATXN7 |
| Pitpnm1 | ENSMUSG00000024851 | 0.020670104 | 0.097401325 | −0.685730815 | 1893.409241 | PITPNM1 |
| Abca7 | ENSMUSG00000035722 | 0.020672008 | 0.097401325 | −0.896529318 | 1678.793683 | ABCA7 |
| Chd8 | ENSMUSG00000053754 | 0.02073566 | 0.097497474 | −1.108100007 | 2582.749866 | CHD8 |
| Dcbld2 | ENSMUSG00000035107 | 0.020739169 | 0.097497474 | −0.896278859 | 41.47789801 | DCBLD2 |
| Atr | ENSMUSG00000032409 | 0.020773627 | 0.097619935 | −1.045071667 | 730.5907763 | ATR |
| Arc | ENSMUSG00000022602 | 0.020785282 | 0.097619935 | −1.046036651 | 21.25006924 | ARC |
| Tmem81 | ENSMUSG00000048174 | 0.020817663 | 0.097662613 | −1.066412805 | 101.5300744 | TMEM81 |
| Trim56 | ENSMUSG00000043279 | 0.020833411 | 0.097662613 | −1.347669212 | 1567.961398 | TRIM56 |
| Otud4 | ENSMUSG00000036990 | 0.020887229 | 0.097846879 | −1.312902713 | 1718.933544 | OTUD4 |
| Trerf1 | ENSMUSG00000064043 | 0.020954295 | 0.098007493 | −0.954539436 | 436.5849234 | TRERF1 |
| Rc3h1 | ENSMUSG00000040423 | 0.0209618 | 0.098007493 | −1.098873456 | 1021.721514 | RC3H1 |
| Pyroxd2 | ENSMUSG00000060224 | 0.021008992 | 0.09813384 | −1.101720545 | 12.90247692 | PYROXD2 |
| Fnbp4 | ENSMUSG00000008200 | 0.021025321 | 0.098178699 | −1.083836292 | 2906.519261 | FNBP4 |
| C8g | ENSMUSG00000015083 | 0.021093421 | 0.098276624 | −0.710398943 | 42.09913467 | C8G |
| A230050P20Rik | ENSMUSG00000038884 | 0.021163595 | 0.098310039 | −0.735455602 | 413.8436488 | C19orf66 |
| A830010M20Rik | ENSMUSG00000044060 | 0.021166803 | 0.098310039 | −1.062248038 | 169.4242824 | KIAA1107 |
| Arid1a | ENSMUSG00000007880 | 0.021174661 | 0.098310039 | −1.267323856 | 3807.911199 | ARID1A |
| Pigo | ENSMUSG00000028454 | 0.02117672 | 0.098310039 | −0.574199906 | 731.2224965 | PIGO |
| Tmem87b | ENSMUSG00000014353 | 0.021181412 | 0.098310039 | −0.950106858 | 453.9553155 | TMEM87B |
| Ralgapa2 | ENSMUSG00000037110 | 0.021355006 | 0.098707733 | −1.119998066 | 366.6264567 | RALGAPA2 |
| Atm | ENSMUSG00000034218 | 0.02140639 | 0.098766949 | −1.356995482 | 1085.266522 | ATM |
| Ccdc114 | ENSMUSG00000040189 | 0.021437127 | 0.098868165 | −0.749043204 | 49.0581057 | CCDC114 |
| Mphosph9 | ENSMUSG00000038126 | 0.021455559 | 0.098921921 | −0.986818653 | 509.2254015 | MPHOSPH9 |
| Rab12 | ENSMUSG00000023460 | 0.021525336 | 0.099149677 | −0.473015915 | 261.6254043 | RAB12 |
| Birc6 | ENSMUSG00000024073 | 0.021554805 | 0.099191515 | −1.30548857 | 4536.630806 | BIRC6 |
| Cdh5 | ENSMUSG00000031871 | 0.021609473 | 0.099349126 | −0.816789219 | 22.856724 | CDH5 |
| Lyst | ENSMUSG00000019726 | 0.021649841 | 0.099444322 | −1.41308572 | 2164.991428 | LYST |
| Ppargc1b | ENSMUSG00000033871 | 0.021758245 | 0.09962519 | −1.002746533 | 110.3899446 | PPARGC1B |
| Fem1c | ENSMUSG00000033319 | 0.021809067 | 0.099732756 | −0.743798118 | 607.1405448 | FEM1C |
| Upf3b | ENSMUSG00000036572 | 0.021853625 | 0.099805941 | −0.985226886 | 762.2502718 | UPF3B |
| Atp2a1 | ENSMUSG00000030730 | 0.021854223 | 0.099805941 | −1.213156411 | 22.42177377 | ATP2A1 |
| Slc4a7 | ENSMUSG00000021733 | 0.021906324 | 0.099895848 | −1.209137871 | 1840.303917 | SLC4A7 |
| Tsc1 | ENSMUSG00000026812 | 0.021915951 | 0.099908536 | −1.144463056 | 1042.615413 | TSC1 |
| Gfod1 | ENSMUSG00000051335 | 0.021929835 | 0.099938159 | −1.016421256 | 167.1676106 | GFOD1 |
| Proz | ENSMUSG00000031445 | 0.021970318 | 0.099938159 | −1.073825063 | 42.30681128 | PROZ |

TABLE 4
Leading edge genes from GSEA and genes associated with pathways
Leading edge genes from FIGS. 3g and 3h
400 leading edge genes determined by GSEA. Downregulated genes ranked by log2-fold
change and determined by RNAseq were used for analysis
(B220 gene set q-value <0.1 and FL gene set p-value <0.05

| B220+ VavPBcl2-shKmt2d | KMT2D nonsense mutant FL |
|---|---|
| ACHE | ABCD1 |
| ADAM8 | ADAM8 |
| ADAMTSL4 | ADAP2 |
| ADAP2 | ADCY7 |
| ADRBK2 | AFF1 |
| AFF1 | AHDC1 |
| AHNAK | ALPK2 |
| AHNAK2 | ALPL |
| APC | AMOT |
| APOBR | APOBR |
| ARAP2 | ARID3A |
| ARHGAP31 | ARID5A |
| ATP6V0A1 | ARRDC4 |
| BAMBI | ATN1 |
| BCL9L | BANK1 |
| BHLHE40 | C10orf128 |
| BOD1L1 | C10orf76 |
| CAPN3 | C19orf71 |
| CARD14 | C1R |
| CARD9 | CACNA1A |
| CARNS1 | CADM1 |
| CCDC9 | CAMKK1 |
| CCND1 | CARD9 |
| CCR2 | CCND1 |
| CD274 | CD274 |
| CD300A | CD44 |
| CD4 | CD69 |
| CD69 | CDC42EP4 |
| CDH1 | CDYL |
| CHD7 | CHD7 |
| CLIP2 | CHN2 |
| CLN8 | CKAP4 |
| CLU | CLCN7 |
| CRB2 | COL9A3 |
| CSRNP1 | CRB2 |
| CUBN | CRTC3 |
| CX3CR1 | CUBN |
| DACT1 | CYB5RL |
| DBNDD1 | DCBLD2 |
| DCBLD2 | DFNB31 |
| DEGS2 | DIP2B |
| DIP2B | DNAJA1 |
| DOPEY2 | DNAJB1 |
| DSE | DOK2 |
| DUSP1 | DOPEY2 |
| DUSP6 | DSE |
| EGR1 | DUSP3 |
| EGR2 | DUSP6 |
| EML5 | EGR3 |
| ENG | ELL |
| EPHA2 | EPHB6 |
| FAM43A | ERRFI1 |
| FAM46A | ESAM |
| FAN1 | FAM129C |
| FAR2 | FAM43A |
| FARP2 | FAM46C |
| FBXL20 | FAM65A |
| FBXO24 | FBXO24 |
| FFAR1 | FGR |
| FGR | FLNA |
| FOS | FMNL3 |
| FOSB | FSCN1 |
| FOXJ1 | FURIN |
| FRMD4A | GAS7 |
| FYB | GATA3 |
| GALNT3 | GDF11 |
| GAS7 | GPD1 |
| GHRL | GPR132 |
| GPD1 | GRAP2 |
| GPR157 | GTPBP1 |
| GRAP2 | HERC1 |
| HAVCR2 | HERC3 |
| HEBP1 | HEXIM1 |
| HERC1 | HHEX |
| HFE | HMOX1 |
| HIPK2 | HOOK1 |
| HMBOX1 | HSP90AA1 |
| HSD3B7 | HSPA1A |
| HSPA1A | HSPA1B |
| HSPA1B | HSPA1L |
| HSPG2 | HSPG2 |
| HTT | HSPH1 |
| IER2 | IFFO2 |
| IL18R1 | IFIT2 |
| IL1B | IFITM2 |
| INSR | IKZF1 |
| IQSEC1 | IL17RA |
| ITGAM | INSR |
| JUNB | IRAK2 |
| JUP | ITGA5 |
| KCNG1 | ITGB7 |
| KDM6B | ITPRIP |
| KIAA2018 | JAM3 |
| KIF21B | JUNB |
| KLF11 | JUP |
| KLF4 | KCNG1 |
| LAIR1 | KLF11 |
| LILRB4 | KLF2 |
| LOXL3 | KLF3 |
| LRCH4 | KLF4 |
| LRP1 | KLF9 |
| LRRC16B | LAMP3 |
| MAPK8IP3 | LDLRAP1 |
| MDN1 | LFNG |
| MEGF8 | LRRC56 |
| MICAL3 | LTBP3 |
| MTSS1L | MAN2A2 |
| MYBPC2 | MAPK8IP3 |
| MYBPC3 | MED13L |
| MYO1F | MTMR12 |
| MYO5A | MTMR3 |
| NAV2 | MYBPC2 |
| NCOR2 | MYO1F |
| NEB | MYO5B |
| NFKBIZ | NAV2 |
| NOTCH2 | NFATC3 |
| NR4A1 | NFKBIZ |
| NR4A2 | NFRKB |
| NRP1 | NMT2 |
| NUP210L | NOTCH1 |
| OSGIN1 | NR4A2 |
| P2RX1 | NRARP |
| PAK1 | NTN1 |
| PARVB | PAFAH2 |
| PDE4C | PARP14 |
| PELI2 | PDCD11 |
| PER1 | PELI3 |
| PER3 | PHF20 |
| PHACTR2 | PHLDB3 |
| PIK3C2B | PHTF1 |
| PIK3R5 | PI4K2A |
| PKD1 | PIK3R4 |
| PLCB2 | PKD1 |
| PLEC | PKN3 |
| PLEKHM3 | PLAUR |
| PLK2 | PLCB2 |
| PLK3 | PLK3 |
| PTK6 | PLXND1 |
| PTPDC1 | PRDM1 |
| PTPRE | PREX1 |
| PYROXD2 | PRR5L |
| RAB6B | PTPRK |
| RAI1 | RAB11FIP5 |
| RDH5 | RABEP2 |
| RNF213 | RAP2B |
| RREB1 | RARA |
| SCML4 | RARG |
| SERPING1 | RASA3 |
| SESN3 | RGMB |
| SGK1 | RGS12 |
| SIDT2 | RIN3 |
| SLFN12L | RNF149 |
| SNAI1 | RNF43 |
| SOCS3 | SAFB2 |
| SPARC | SDC4 |
| SPECC1 | SELP |
| SPEN | SERPINE1 |
| SRGAP3 | SIK3 |
| STAC3 | SIRT1 |
| STARD9 | SKI |
| SYNGAP1 | SLC12A6 |
| SYNPO | SLC12A7 |
| TAGLN | SLC16A5 |
| TBC1D8 | SLC25A30 |
| TBC1D9 | SLC4A3 |
| TBKBP1 | SNAI1 |
| TGFBI | SNX9 |
| TGM1 | SOCS3 |
| TGM2 | SPATA6 |
| TIAM1 | ST6GALNAC3 |
| TLR8 | STK10 |
| TMC4 | SUFU |
| TMEM8B | SYNPO |
| TNFRSF14 | TBKBP1 |
| TNNT3 | TBXAS1 |
| TNRC6B | TELO2 |
| TNRC6C | TERF1 |
| TRIM56 | TGM2 |
| TRPM2 | THRA |
| TSC1 | TLR4 |
| TTC39B | TMEM8B |
| TTN | TNFRSF1B |
| VASN | TNNT3 |
| VCAM1 | TNRC6C |
| VPS13C | TRPM2 |
| VPS13D | UST |
| WDFY1 | UTRN |
| ZBTB20 | WDFY1 |
| ZBTB43 | WDR81 |
| ZC3H12A | WIPF2 |
| ZC3H12B | ZBTB32 |
| ZFP36 | ZC3HAV1 |
| ZMIZ1 | ZFYVE27 |
| ZNF14 | ZKSCAN3 |
| ZNF208 | ZMYND11 |
| ZNF23 | ZNF14 |
| ZNF3 | ZNF267 |
| ZNF398 | ZNF442 |
| ZNF442 | ZNF473 |
| ZNF628 | ZNF597 |

Genes associated with significantly enriched pathways in FIG. 3i

| Lymphochip database | | | | | | | | |
|---|---|---|---|---|---|---|---|---|
| pathways (http:// | | | | | | | | |
| lymphochip.nih.gov/ | | | | | | | | |
| signaturedb/index.html) | | | | | | | | |
| Immediate Early genes = | | EGR1 | FOS | FOSB | ZFP36 | JUNB | DUSP1 | |
| Immediate_early | | | | | | | | |
| IL6 induced genes = | | EGR2 | ZFP36 | SGK1 | JUNB | SOCS3 | ZBTB20 | |
| IL6 LY10 Up all | | | | | | | | |
| IL10 induced genes = | | CD274 | BANK1 | HMOX1 | ZFP36 | SGK1 | SESN3 | |
| IL10_OCILy3 Up | | SNX9 | JUNB | IFITM2 | BCL9L | SOCS3 | PRDM1 | |
| | | DUSP1 | | | | | | |
| HRAS target genes = | | NFKBIZ | EPHA2 | EGR1 | FOS | IL1B | ADAM8 | |
| HRAS_overexpression | | CD274 | ZFP36 | SDC4 | IER2 | JUNB | PLAUR | |
| 2x up | | PTPRE | DUSP6 | DUSP1 | | | | |
| KRAS target genes = | | TAGLN | ADCY7 | HSPG2 | NLRP1 | SPARC | SNAI1 | |
| KRAS_Up.txt | | SGK1 | TGFBI | SERPINE1 | JUNB | CADM1 | | |
| HRAS target genes = | | EPHA2 | EGR1 | FOS | IL1B | ADAM8 | ZFP36 | |
| HRAS_overexpression_ | | IER2 | JUNB | PLAUR | DUSP6 | DUSP1 | | |
| 4x_up | | | | | | | | |
| IL10 induced genes = | | CD274 | BANK1 | HMOX1 | ZFP36 | SGK1 | SESN3 | |
| 1L10_OCILy3_Up | | SNX9 | JUNB | IFITM2 | BCL9L | SOCS3 | PRDM1 | |
| | | DUSP1 | | | | | | |
| IL6 induced genes = | | EGR2 | ZFP36 | SGK1 | JUNB | SOCS3 | ZBTB20 | |
| IL6_Ly10_Up_all | | | | | | | | |
| Immediate Early genes = | | EGR1 | FOS | FOSB | ZFP36 | JUNB | DUSP1 | |
| Immediate_early | | | | | | | | |
| JAK_IL10_Ly10_Up | | ZFP36 | JUNB | IFITM2 | PRDM1 | | | |
| KRAS target genes = | | TAGLN | ADCY7 | HSPG2 | SPARC | SNAI1 | SGK1 | |
| KRAS_Up | | TGFBI | SERPINE1 | JUNB | CADM1 | | | |
| Broad institute Molecular | | | | | | | | |
| signatures Database | | | | | | | | |
| (http://www. | | | | | | | | |
| broadinstitute.org/ | | | | | | | | |
| gsea/msigdb/index.jsp) | ID | | | | | | | |
| HRAS Oncogenic | msig_1335 | NFKBIZ | EPHA2 | EGR1 | FOS | IL1B | ADAM8 | CD274 |
| Signature = BILD | | ZFP36 | SDC4 | KDM6B | IER2 | JUNB | PLAUR | PTPRE |
| HRAS Oncogenic | | ITPRIP | DUSP6 | DUSP1 | AHNAK2 | | | |
| EGF signaling target | msig_308 | EPHA2 | EGR2 | EGR3 | EGR1 | FOS | FOSB | DNAJB1 |
| genes = NAGASHIMA_ | | ZFP36 | NR4A2 | NR4A1 | BHLHE40 | KDM6B | IER2 | JUNB |
| EGF_SIG | | DUSP1 | | | | | | |
| TGFB1 induced genes = | msig_2312 | NOTCH2 | LRP1 | HSPG2 | APC | JUP | SPARC | SERPINE1 |
| VERRECCHIA_ | | CD44 | PAK1 | | | | | |
| EARLY_RES | | | | | | | | |
| Serum Response genes = | msig_977 | ZC3H12A | EGR3 | EGR1 | NR4A2 | PLK2 | BHLHE40 | SGK1 |
| AMIT_SERUM_ | | IER2 | | | | | | |
| RESPONSI | | | | | | | | |
| LPS (TLR4) induced | msig_1707 | NFKBIZ | ZC3H12A | EGR2 | EGR3 | EGR1 | VCAM1 | PLK2 |
| genes = SEKI_INFLAM- | | PTPRE | CD44 | | | | | |
| MATORY | | | | | | | | |
| TNF induced genes = | msig_994 | CDC42EP4 | NFKBIZ | ZC3H12A | DSE | IL1B | SDC4 | SGK1 |
| ZHANG_RESPONSE_ | | IRAK2 | SNX9 | PLAUR | SOCS3 | GPR132 | | |
| TO_IKK_ | | | | | | | | |
| CROONQUIST_NRAS_ | msig_1832 | CX3CR1 | CCR2 | TBC1D9 | DUSP6 | | | |
| SIGNALING_UP | | | | | | | | |
| CHIARADONNA_ | msig_398 | NOTCH1 | SLC4A3 | FOS | KLF2 | STK10 | JUP | PER1 |
| NEOPLASTIC_ | | RAB11FIP5 | SOCS3 | DUSP1 | | | | |
| TRANSFORMATIO | | | | | | | | |
| PEREZ_TP53_AND_ | msig_556 | ADAP2 | NTN1 | COL9A3 | EGR2 | EGR1 | NRARP | BAMBI |
| TP63_TARGETS | | INSR | DFNB31 | FAM46C | TNRC6C | FAM43A | VASN | CAPN3 |

| Leading Edge Genes from B220 ChlPseq | | Leading Edge Genes from B220 ChlPseq | |
|---|---|---|---|
| Enhancer FIGS. 4c, d 400 leading edge | | promoter from Suppl FIGS. 4e, f 400 leading | |
| genes determined by GSEA. | | edge genes determined by GSEA. | |
| B220+ | KMT2D | B220+ | KMT2D |
| VavPBcl2-5hKmt2d | nonsense mutant FL | VavPBcl2-shKmt2d | nonsense mutant FL |
| ABR | ACAD9 | ABR | ABAT |
| ACSL1 | ADAMTSL4 | ACACB | ABCB6 |
| ACVR1B | ADCK5 | ACHE | ACACB |
| ACVRL1 | ADORA2A | ADAR | ACHE |
| ADAMTSL4 | ADRBK1 | AEBP2 | ACOT7 |
| ADCY9 | AGPAT4 | AGRN | ADCK5 |
| AFF3 | AHCYL2 | AIM2 | ADORA2A |
| AHCYL2 | AKAP2 | AIRE | AGRN |
| AKAP2 | ALPK2 | AKT3 | ALDH7A1 |
| ANKRD11 | ANKRD11 | ALDH1L2 | ALKBH7 |
| ANKRD44 | AP1S3 | APP | ANKRD9 |
| APOBEC2 | ARHGAP22 | ARHGAP23 | APP |
| ARHGAP26 | ARHGAP26 | ARHGAP29 | ARHGEF40 |
| ARHGAP29 | ARID3A | ARHGAP6 | ARID2 |
| ARHGAP32 | ARID5A | ARID1A | ARMC5 |
| ARID3B | AXIN1 | ARID2 | ATE1 |
| ARMC9 | AZIN1 | ARID5B | B3GAT2 |
| ASAP1 | BCAR3 | ATXN1 | BAHD1 |
| ASXL1 | BCL9L | B3GAT2 | BCAP31 |
| ATP11B | BFSP2 | BAHD1 | BCL3 |
| ATP8B4 | BTG1 | BCR | BMP1 |
| ATXN7L1 | C100rf32 | BRD3 | C19orf66 |
| B4GALT5 | C1orf95 | C19orf66 | C1orf95 |
| BATF3 | C9orf85 | C1orf95 | CASZ1 |
| BCL9 | CAPN10 | C3orf70 | CBX6 |
| BCL9L | CCRN4L | CACNA1H | CCDC64 |
| BEGAIN | CD69 | CAMK2A | CCDC88B |
| BMP2K | CDADC1 | CASZ1 | CCDC9 |
| BPTF | CDK5R1 | CCDC102A | CCR6 |
| C1orf95 | CDYL | CCDC38 | CD55 |
| CACNA1H | CELF2 | CCDC39 | CDC42BPB |
| CACNG6 | CEP164 | CCDC88B | CDH24 |
| CCDC38 | CHD9 | CCDC9 | CEP68 |
| CCDC6 | CHST12 | CCR6 | CHRM4 |
| CCDC88B | CIITA | CDC42BPB | CHST7 |
| CD69 | CLIP2 | CELSR1 | CKAP4 |
| CECR2 | CMTM7 | CENPF | CLIP2 |
| CELF2 | CPM | CEP68 | COL1A1 |
| CELSR2 | CRB2 | CLIP2 | CRAMP1L |
| CEP164 | CSRNP1 | CNST | CRAT |
| CHD9 | CXCR4 | CPD | CSRNP2 |
| CHST11 | CXCR5 | CRAMP1L | CXCR3 |
| CIITA | DFNB31 | DBNDD1 | CXorf40A |
| CLASP1 | DIP2B | DCBLD2 | DBNDD1 |
| CLIP2 | DLL1 | DENND3 | DCBLD2 |
| CORO2A | DNASE1 | DNAI2 | DHRS13 |
| CRB2 | DOCK11 | DNMBP | EHHADH |
| CREB1 | DOCK9 | EEPD1 | ELF4 |
| CRYBG3 | DTNB | EHHADH | ENTPD7 |
| DCHS1 | DYRK1A | ELF4 | EOMES |
| DDX6 | EGR2 | EPHB2 | ERBB2IP |
| DENND1B | ELF4 | ERBB2IP | ESAM |
| DFNB31 | ELK3 | ESAM | FAAH |
| DGKH | EMILIN2 | FAM105A | FABP5 |
| DIP2B | ENPP1 | FAM179A | FADS2 |
| DLL1 | ETV6 | FAM89A | FAM105A |
| DNAI2 | EXT1 | FARP2 | FAM110A |
| DNMT3A | FAM117A | FBXL2 | FAM132A |
| DOCK11 | FAM134B | FGR | FAM83H |
| DOCK9 | FAM46C | FSCN1 | FARP2 |
| DUSP16 | FAM49B | FUT1 | FBXL2 |
| DYRK1A | FAM91A1 | GBP6 | FGF9 |
| EDARADD | FMNL3 | GFOD1 | FGFRL1 |
| EEPD1 | FOSB | GPR157 | FGR |
| EGR2 | FSCN1 | GYLTL1B | FRAT2 |
| EIF2AK3 | FUT8 | HCN3 | FSCN1 |
| EIF4A2 | GADD45B | HEMGN | GALNT12 |
| EIF4G3 | GDF11 | HIC2 | GNGT2 |
| ELF4 | GHRL | HIF3A | GPR135 |
| EML4 | GLTSCR1 | HINFP | GPR157 |
| EPHB2 | GNA15 | HIP1R | GTPBP1 |
| ETV6 | GNG7 | HOXB6 | GTPBP4 |
| EXOC1 | GPM6B | HTATSF1 | GYLTL1B |
| FAM129B | GPR157 | HTT | HIC1 |
| FAM91A1 | GPR18 | IGSF3 | HIC2 |
| FBXO10 | GYPC | IL9R | HIP1R |
| FCHSD2 | HAAO | IMPACT | HSD17614 |
| FGD6 | HDAC4 | INF2 | HSPB1 |
| FKBP15 | HDAC7 | INTS2 | HTT |
| FMNL3 | HEG1 | IQCE | IGF1R |
| FNBP1 | HIPK2 | IQSEC2 | IGF2BP3 |
| FOSB | HIVEP1 | ITGB3 | IGSF3 |
| FOXK1 | HMBOX1 | ITGB8 | IL12RB1 |
| FRY | IDH2 | KCP | ING1 |
| GDA | IDO1 | KDM3B | INPP5A |
| GHRL | INPP5A | KDM5B | INPP5D |
| GLTSCR1 | INPP5D | KIAA0922 | KAZALD1 |
| GNG7 | IQSEC1 | KIAA1522 | KCNJ1 |
| GPR157 | IRAK2 | KIAA2018 | KDM3B |
| GRAMD1B | ITGB2 | KIF19 | KIAA2018 |
| GXYLT1 | ITPR2 | KLF11 | KLF11 |
| HDAC4 | JMJD1C | KLF13 | KLF13 |
| HEG1 | KDM2B | KLF2 | KLF2 |
| HIPK2 | KIF13B | LATS2 | LCP2 |
| HIVEP1 | KLHL3 | LCP2 | LEPRE1 |
| HK3 | KSR1 | LIME1 | LGALS1 |
| HMBOX1 | LASP1 | MAFB | LGALS3BP |
| IL6R | LRRFIP1 | MAFK | MAFK |
| IL9R | LY6E | MAPKBP1 | MAPK8IP1 |
| IQSEC1 | MANBA | MAST1 | MAPKBP1 |
| ITGAL | MAP3K5 | MCTP2 | MCOLN1 |
| ITGB3 | MBP | MED13L | MED13L |
| ITPR2 | MED13L | MGAT4A | MEPCE |
| JMJD1C | MGAT1 | MGAT5 | MICALL1 |
| KATNAL1 | MICAL3 | MICALL1 | MMP17 |
| KIF13B | MTMR12 | MYCBP2 | MOCS3 |
| KIF20B | MXD1 | MYO5B | MOV10 |
| KSR1 | MYO3B | NAA40 | MSH5 |
| LNPEP | NCEH1 | NAV2 | MTL5 |
| LPGAT1 | NCOA2 | NBEAL2 | MYCBP2 |
| LRIG2 | NCOR2 | NFATC2 | MYO5B |
| LRRFIP1 | NDUFA13 | NFE2L1 | NAV2 |
| LYST | NFATC1 | NFE2L3 | NBEAL2 |
| MAP3K5 | NFKBIA | NFIC | NDRG1 |
| MAP3K8 | NLRC5 | NFIX | NDRG4 |
| MCTP2 | NLRP2 | NFXL1 | NFATC2 |
| MED13L | NPRL3 | NOSTRIN | NINJ1 |
| MEF2A | NRARP | NQO1 | NR3C2 |
| MGAT5 | OGFRL1 | NRP2 | NXPH4 |
| MICAL3 | OSBPL3 | OSBPL6 | PAOX |
| MINK1 | OSBPL8 | PAK6 | PCK2 |
| MXD1 | PCNX | PAPLN | PDE4C |
| MYCBP2 | PFKFB3 | PARM1 | PHRF1 |
| NCOA2 | PHF2 | PBX1 | PIK3CD |
| NCOR2 | PIK3AP1 | PCF11 | PIM3 |
| NLRC5 | PIK3C2B | PCGF3 | PLEKHG2 |
| NOD2 | PIK3CD | PCMTD1 | PLXNB2 |
| NOSTRIN | PIP5K1C | PDE4C | PLXNC1 |
| NRIP1 | PLEKHO2 | PEAR1 | POLD1 |
| NRP2 | POPDC2 | PFKFB4 | PPP1R3E |
| OSBPL8 | PPP1R13B | PHRF1 | PPTC7 |
| PAG1 | PREX1 | PLCG1 | PRR5L |
| PAN3 | PRICKLE1 | PLEKHG2 | PTPDC1 |
| PCMTD1 | PRR5 | PLXNB2 | RAB35 |
| PCNX | PSAP | PPARGC1B | RAB36 |
| PCYT1A | PSTPIP1 | PPL | RAB6B |
| PCYT1B | PTP4A3 | PPM1H | RAI1 |
| PECAM1 | PTPN1 | PRRC2B | RALGDS |
| PIK3C2B | RAB6B | PTPDC1 | RASL11B |
| PIK3R1 | RAB8B | PTPN3 | RASSF2 |
| POFUT1 | RAD54B | PVRL1 | RBMX2 |
| POU2F2 | RALGDS | RAB6B | RDH10 |
| PREX1 | RAP1GAP2 | RAI1 | REPIN1 |
| PRRC2B | RASA3 | RASGRF1 | RERE |
| PVRL1 | RERE | RASSF2 | RGS12 |
| QPRT | RIMKLA | RCBTB1 | RGS14 |
| RAB6B | RNF130 | RERE | RIMKLA |
| RAD54B | RNF19B | RGS3 | RMND5A |
| RAP1GAP2 | RUNX1 | RIMKLA | RND1 |
| RASA2 | RUNX3 | SALL2 | RPS6KA1 |
| RASGRP3 | SECISBP2L | SCN11A | RRAGD |
| RERE | SEMA4B | SEC14L2 | RUNX3 |
| REV1 | SEMA7A | SEC31B | SALL2 |
| RIMKLA | SERTAD1 | SEMA4B | SCN11A |
| RUNX1 | SGK1 | SGK223 | SEC31B |
| SCN8A | SH3BP5 | SH3BP4 | SEMA4B |
| SECISBP2L | SH3PXD2A | SHANK1 | SEMA4D |
| SEMA4B | SIPA1L1 | SHB | SERHL2 |
| SERTAD2 | SLAMF1 | SIN3A | SESN2 |
| SGK1 | SLC4A8 | SIX1 | SETD4 |
| SH3PXD2A | SLC9A3R1 | SLC11A1 | SGTB |
| SHB | SNX18 | SLC12A9 | SIDT2 |
| SIPA1L1 | SNX9 | SLC22A15 | SIN3A |
| SIPA1L2 | SOCS3 | SLC26A8 | SLBP |
| SLC29A3 | ST3GAL1 | SLC30A1 | SLC12A4 |
| SLC4A8 | ST6GALNAC6 | SLC43A2 | SLC12A9 |
| SMAD3 | STAT5B | SLC45A4 | SLC25A34 |
| SMAD7 | TAF3 | SLC4A3 | SLC25A43 |
| SMARCA2 | TBC1D14 | SOAT2 | SLC39A14 |
| SOCS3 | TBC1D9 | SPATA1 | SLC4A2 |
| SPATA13 | TBKBP1 | SRMS | SLC4A3 |
| SPRED2 | TCP11L2 | STARD9 | SP110 |
| ST3GAL1 | TEC | SYNPO | ST6GALNAC6 |
| TAF3 | TMEM173 | TBC1D2 | STARD9 |
| TARSL2 | TMEM176B | TEAD2 | SYNGR3 |
| TBC1D14 | TMEM189 | TFCP2L1 | SYNPO |
| TBC1D9 | TMEM2 | THSD1 | TCF3 |
| TBKBP1 | TMEM201 | TOX | TCTN2 |
| TCF4 | TNFAIP3 | TRPM2 | TEX9 |
| TEF | TNFAIP8 | TRRAP | TMEM108 |
| TMEM131 | TNFRSF14 | TSC1 | TNFRSF12A |
| TNFAIP3 | TNIP1 | TSPAN33 | TRAF4 |
| TNFAIP8 | TNRC18 | TTC28 | TRPM2 |
| TNFRSF14 | TOX2 | TTC39B | TRRAP |
| TNRC18 | TRAF2 | TUFT1 | TSC1 |
| TRAK1 | TRIM2 | USP2 | TSPAN18 |
| TRERF1 | TRIM8 | USP51 | TTC39B |
| TRIM2 | TSNAXIP1 | WDFY1 | UBE2D1 |
| TSNAXIP1 | TSPAN14 | VVHSC1L1 | ULK1 |
| TTC39B | TTC39B | ZBTB38 | UNC119 |
| USP6NL | TUBA1B | ZBTB4 | USP2 |
| USP7 | USP15 | ZC3H12A | WDFY1 |
| VAMP1 | VAMP1 | ZDHHC23 | VVDR6 |
| VAV2 | WDFY1 | ZMIZ1 | WSB2 |
| WDFY1 | WDFY4 | ZNF275 | ZBTB38 |
| WDFY4 | WVVP2 | ZNF280B | ZC3H12A |
| ZBTB38 | ZBTB38 | ZNF546 | ZFP82 |
| ZFYVE26 | ZMIZ1 | ZNF629 | ZMIZ1 |
| ZMIZ1 | ZNF469 | ZNFX1 | ZNF629 |
| ZNF217 | ZNRF1 | ZNRF3 | ZNFX1 |

Genes associated with significantly enriched pathways in FIG. 4e enhancers

| Lymphochip database | | | | | | |
|---|---|---|---|---|---|---|
| pathways (http:// | | | | | | |
| lymphochip.nih.gov/ | | | | | | |
| signaturedb/index.html) | | | | | | |
| NFkB targets = NFkB | GADD45B | NFKBIA | TNFAIP3 | SMARCA2 | | |
| bothOCILy3andLY10 | | | | | | |
| IL0 induced genes = | EGR2 | CXCR5 | PTPN1 | SGK1 | SOCS3 | |
| 1L6_Ly10_Up_all | | | | | | |
| IL10 induced genes = | CXCR5 | NLRC5 | FUT8 | PTPN1 | PFKFB3 | TNFAIP3 |
| 1L10_OCILy3_Up | SGK1 | CIITA | ADRBK1 | SNX9 | BCAR3 | BCL9L |
| | SOCS3 | POU2F2 | BATF3 | | | |
| TGFB induced genes = | GADD45B | SMAD7 | EPHB2 | SGK1 | ST3GAL1 | |
| TGFbeta up epithelial | | | | | | |
| large | | | | | | |
| KRAS induced genes = | INPP4B | GADD45B | MAP3K8 | EPHB2 | SGK1 | ACVR1B |
| KRAS_Up | | | | | | |
| PRDM1 repressed genes = | CXCR5 | GPR18 | INPP5D | CIITA | PAG1 | POU2F2 |
| Blimp_Bcell_repressed | | | | | | |
| Broad institute Molecular | | | | | | |
| signatures Database | | | | | | |
| (http://www. | | | | | | |
| broadinstitute.org/ | | | | | | |
| gsea/msigdb/index.jsp) | ID | | | | | |
| CD40 induced genes = | msig_1313 | GADD45B | CXCR5 | MAP3K8 | NFKBIA | TNFAIP8 |
| BASSO_CD4O_ | | TNFAIP3 | PIK3CD | PTP4A3 | SLAMF1 | TNFAIP3 |
| SIGNALING_UP | | | | | | |
| LPS (TLR4) induced | msig_1707 | BTG1 | GADD45B | EGR2 | NFKBIA | TNFAIP3 |
| genes = SEKI_INFLAM- | | TMEM2 | | | | |
| MATORY_ | | | | | | |
| RESPONSE_LPS_UP | | | | | | |
| TNF induced genes = | msig_994 | BTG1 | ABTB2 | SMAD3 | RNF19B | B4GALT5 |
| ZHANG_RESPONSE_ | | SEMA7A | MXD1 | NFKBIA | TNFAIP8 | TNFAIP3 |
| TO_IKK_INHIBITOR_ | | SGK1 | DUSP16 | IRAK2 | SNX9 | PAG1 |
| AND_TNF_UP | | SOCS3 | HIVEP1 | | | |
| EGF signaling target | msig_968 | TSPAN14 | EXT1 | CDYL | MBP | AMIGO2 |
| genes = AMIT_EGF_ | | CHST11 | TRIO | BCAR3 | LY6E | GRAMD1B |
| RESPONSE_ | | | | | | |
| 480_HELA | | | | | | |
| p53 and p63 target genes = | msig_556 | PPP1R13B | SIPA1L2 | EGR2 | NRARP | SMAD7 |
| PEREZ_TP53_AND_ | | TAF3 | TRIM8 | KSR1 | FAM105A | DFNB31 |
| TP63_TARGETS | | FAM46C | TOX | FRY | | |

Genes associated with significantly enriched pathways in FIG. 4g promoters

| Lymphochip database | | | | | | |
|---|---|---|---|---|---|---|
| pathways (http:// | | | | | | |
| lymphochip.nih.gov/ | | | | | | |
| signaturedb/index.html) | | | | | | |
| KRAS target genes = | COL1A1 | ULK1 | HSPB1 | EPHB2 | BMP1 | TUFT1 |
| KRAS_Up | | | | | | |
| Broad institute Molecular | | | | | | |
| signatures Database | | | | | | |
| (http://www.broadinstitute. | | | | | | |
| org/gsea/msigdb/index.jsp) | ID | | | | | |
| KRAS regulated genes in | msig_398 | SLC4A3 | COL1A1 | KAZALD1 | KLF2 | APP |
| neoplastic transformation = | | INPP5A | TCF3 | NDRG4 | | |
| CHIARADONNA_ | | | | | | |
| NEOPLASTIC_TRAN | | | | | | |
| PEREZ_TP53_AND_ | msig_556 | ULK1 | MAFB | HIC2 | TUFT1 | FAM105A |
| TP63_TARGETS | | TOX | IGF1R | PAK6 | CASZ1 | SEMA4D |
| | | CRAMP1L | | | | |

Genes associated with significantly enriched pathways in FIG. 5e

| Lymphochip database | | | | | | | |
|---|---|---|---|---|---|---|---|
| pathways (http:// | | | | | | | |
| lymphochip.nih.gov/ | | | | | | | |
| signaturedb/index.html) | | | | | | | |
| PRDM1 targets = | FCER1G | FCRLA | MS4A1 | ST6GAL1 | CXCR5 | VPREB3 | CD22 |
| Blimp_Bcell_repressed | NR1H2 | ZFP36L1 | CIITA | BTK | CD19 | PLEK | PAG1 |
| | FCER2 | POU2F2 | | | | | |
| IL10 induced genes = | ST6GAL1 | IL21R | CXCR5 | RB1 | DMD | HMOX1 | ZFP36 |
| IL10_OCILy3_Up | RAD51 | MEF2D | CIITA | BCAR3 | IFITM1 | BCL9L | POU2F2 |
| | PRDM1 | MYB | CCND3 | | | | |
| KRAS target genes | PGLS | PDXK | GADD45B | JAK1 | HSPB1 | PMEPA1 | SNAI1 |
| KRAS_Up | NPTX1 | EVL | NCF2 | SOX4 | ATP2B4 | CADM1 | |
| NFkB_bothOCILy3 | GADD45B | RELB | BCL2L1 | TRAF1 | NFKBIA | NCF2 | IRF4 |
| andLy10 | | | | | | | |
| Broad institute Molecular | | | | | | | |
| signatures Database | | | | | | | |
| (http://www. | | | | | | | |
| broadinstitute.org/ | | | | | | | |
| gsea/msigdb/index.jsp) | ID | | | | | | |
| IL6 induced genes = | msig_1155 | EPB41L2 | GADD45B | MAPKAPK2 | RB1 | DAPK1 | GNA13 |
| BROCKE_APOPTOSIS_ | | SF1 | ZFP36 | TMEM184B | HBEGF | MX1 | SOX2 |
| REVERSED_BY_IL6 | | CADPS | IRF1 | IRF4 | ATP2B4 | ID3 | POU2F2 |
| | | PRDM1 | SLC2A3 | | | | |
| IRF4 induced genes in | msig_1812 | WHSC1 | SSR1 | ST6GAL1 | GNG7 | CD38 | PPP1R2 |
| plasma cells = | | TXNDC5 | IRF4 | TNFRSFI7 | SUB1 | MNAT1 | PRDM1 |
| SHAFFER_IRF4_ | | MYB | | | | | |
| TARGETS_IN_ | | | | | | | |
| ACTIVATED_ | | | | | | | |
| DENDRITIC_CEL | | | | | | | |
| CD40 induced genes | msig_28 | CHMP7 | FOXN3 | EPB41L2 | TUBA1C | FANCA | ITGB1 |
| in GCB-DLBCL = | | PLEKHF2 | CKAP4 | CUX1 | ALDH2 | NRGN | CD38 |
| HOLLMAN_ | | CD22 | SGCB | CAB39L | KDM5D | MAP4K1 | BLK |
| APOPTOSIS_VIA_ | | BTK | ALDH4A1 | NR3C1 | TNFRSFI7 | MRPL34 | PPP2CB |
| CD40_UP | | INT59 | ENDOD1 | NFIB | | | |
| NFkB target genes | msig_587 | GADD45B | TNFRSF21 | BCL2L1 | TNFRSF10B | TRAF1 | MDM2 |
| downregulated after | | TERT | | | | | |
| IKKB inhibition = | | | | | | | |
| DUTTA_APOPTOSIS_ | | | | | | | |
| VIA_NFKB | | | | | | | |

1. A method for diagnosing a follicular lymphoma or a diffuse large B cell lymphoma, or the responsiveness or contraindication to therapy thereto in a subject, the method comprising the steps of: obtaining a biological sample from said subject; and testing said biological sample to detect the presence or absence of a lysine (K)-specific methyltransferase 2D (KMT2D) alteration in said biological sample, wherein the presence of said KMT2D alteration indicates a diagnosis of said follicular lymphoma or diffuse large B cell lymphoma, or poor responsiveness or contraindication to therapy thereto in said subject.

2. (canceled)

3. The method of claim 1, wherein said KMT2D alteration is a mutation in said KMT2D.

4. The method of claim 3, wherein said mutation is a non-sense mutation, missense mutation, or a combination thereof.

5. The method of claim 1, wherein said KMT2D alteration is a change in the level of KMT2D protein or mRNA, relative to a predetermined level.

6.-8. (canceled)

9. The method of claim 1, wherein said therapy is an immunotherapy, a chemotherapy, a radiation therapy, or a combination thereof.

10. The method of claim 1 wherein the therapy is a B cell therapy, anti-CD40 antibody immunotherapy, anti-CD20 antibody immunotherapy or anti-IgM antibody immunotherapy.

11. (canceled)

12. The method of claim 1, wherein said follicular lymphoma is a Grade 1, 2, or 3 follicular lymphoma.

13. The method of claim 1, wherein a tumor associated with said follicular lymphoma is at Stage 1, 2, 3, or 4.

14.-25. (canceled)

26. A method for treating a follicular lymphoma or diffuse large B cell lymphoma in a subject, the method comprising: (a) obtaining a biological sample from said subject; and testing said biological sample to detect the presence or absence of a lysine (K)-specific methyltransferase 2D (KMT2D) alteration in said biological sample, wherein the presence of said KMT2D alteration indicates a response or lack thereof to a therapy; (b) based on the determination of said response or lack thereof to said therapy, administering an effective amount of a therapeutic agent to treat said follicular lymphoma, thereby treating said follicular lymphoma or diffuse large B cell lymphoma in said subject.

27. The method of claim 26 wherein said therapeutic agent is for immunotherapy, chemotherapy, radiation therapy, or any combination thereof.

28. The method of claim 26, wherein said therapeutic agent is a B cell therapy, anti-CD40 antibody therapy, anti-CD20 antibody therapy or anti-IgM antibody therapy.

29. The method of claim 26, wherein said KMT2D alteration is a mutation in said KMT2D.

30. The method of claim 29, wherein said mutation is a non-sense mutation, a missense mutation, or a combination thereof.

31. The method of claim 26, wherein said KMT2D alteration is a change in the level of KMT2D protein or mRNA, relative to a predetermined level.

32.-35. (canceled)

36. The method of claim 26, wherein said follicular lymphoma is a Grade 1, 2, or 3 follicular lymphoma.

37. The method of claim 26, wherein a tumor associated with said follicular lymphoma is at Stage 1, 2, 3, or 4.

38. (canceled)

39. A method for treating a follicular lymphoma or diffuse large B cell lymphoma in a subject, the method comprising: administering to said subject a combination of a molecule that effectively enhances the level of a lysine (K)-specific methyltransferase 2D (KMT2D) or effectively decreases or inhibits the level or activity of a demethylase in said subject, and an anti-CD40 antibody, anti-CD20 antibody, an anti-IgM antibody, or combination thereof, thereby treating said follicular lymphoma or diffuse large B cell lymphoma in said subject.

40. The method of claim 26 wherein the patient with a KMT2D alteration may not be effectively treated with an anti-CD40 antibody, an anti-CD20 antibody, anti-IgM antibody, and any combination thereof.

41. The method of claim 26 wherein anti-CD40 therapy, anti-CD20 antibody therapy, or anti-IgM antibody therapy, or any combination thereof, is contraindicated in a patient found to have a KMT2D alteration.

42.-43. (canceled)

44. The method of claim 39 wherein the demethylase is a H3K4 demethylase.

45. The method of claim 39 wherein the molecule is an inhibitor of JARID1 or LSD1.
